(19) United States
(12) Patent Application Publication
(10) Pub. No.: US 2024/0000941 A1
(43) Pub. Date: Jan. 4, 2024

(54) PROTEIN DERIVATIVES CONTAINING UNNATURAL AMINO ACIDS AND BRANCHED LINKERS

(71) Applicant: BrickBio, Inc., Waltham, MA (US)

(72) Inventor: James Sebastian Italia, Charlestown, MA (US)

(21) Appl. No.: 18/254,308

(22) PCT Filed: Nov. 24, 2021
(86) PCT No.: PCT/US2021/060853

§ 371 Date: May 24, 2023

Related U.S. Application Data
(60) Provisional application No. 63/117,755, filed on Nov. 24, 2020.

(51) Int. Cl. A61K 47/18; A61K 47/54; A61K 47/68

(52) U.S. Cl. CPC A61K 47/183; A61K 47/545; A61K 47/6849

(57) ABSTRACT

The invention relates generally to protein derivatives containing molecules (e.g., payloads) conjugated to unnatural amino acids (UAAs) by branched linkers, and methods of making and using such protein derivatives.

### CROSS-REFERENCE TO RELATED APPLICATIONS

This application claims the benefit of and priority to U.S. Provisional Patent Application No. 63/117,755, filed Nov. 24, 2020, which is incorporated herein by reference in its entirety for all purposes.

### FIELD OF THE INVENTION

The present disclosure relates, in general, to the field of protein derivatives where branched linkers are used to conjugate molecules to unnatural amino acids (UAAs) in a protein of interest.

### BACKGROUND

In nature, proteins are produced in cells via processes known as transcription and translation. During transcription, a gene comprising a series of codons that collectively encode a protein of interest is transcribed into messenger RNA (mRNA). During translation, a ribosome attaches to and moves along the mRNA and incorporates specific amino acids into a polypeptide chain being synthesized (translated) from the mRNA at positions corresponding to the codons to produce the protein. During translation, naturally occurring amino acids coupled to transfer RNAs (tRNAs) enter the ribosome. The tRNAs, which contain an anti-codon sequence, hybridize to their respective codon sequences in mRNA and transfer the amino acid they are carrying into the nascent protein chain at the appropriate position as the protein is synthesized.

Over the last few decades, significant efforts have been made to produce homogenous preparations of site-specifically modified proteins, e.g., mammalian proteins, on commercial scale quantities for use in a variety of applications, including, for example, therapeutics and diagnostics. Furthermore, efforts have been made to produce these modified mammalian proteins in eukaryotic cells (e.g., mammalian cells) because the proteins may be more readily produced in a properly folded and fully active form and/or post-translationally modified in a manner similar to the native protein naturally produced in a mammalian cell.

One approach for producing proteins that contain site-specific modifications involves the site-specific incorporation of one or more unnatural amino acids (UAAs) into a protein of interest. The ability to site-specifically incorporate UAAs into proteins in vivo has become a powerful tool to augment protein function or introduce new chemical functionalities not found in nature. The core elements required for this technology include: an engineered tRNA, an engineered aminoacyl-tRNA synthetase (aaRS) that charges the tRNA with a UAA, and a unique codon, e.g., a stop codon, directing the incorporation of the UAA into the protein as it is being synthesized.

Central to this approach is the use of an engineered tRNA/aaRS pair in which the aaRS charges the tRNA with the UAA of interest without cross-reacting with the tRNAs and amino acids normally present in the expression host cell. This has been accomplished by using an engineered tRNA/aaRS pair derived from an organism in different domain of life as the expression host cell so as to maximize the orthogonality between the engineered tRNA/aaRS pair (e.g., an engineered bacterial tRNA/aaRS pair) and the tRNA/aaRS pairs naturally found in the expression host cell (e.g., mammalian cell). The engineered tRNA, which is charged with the UAA via the aaRS, binds or hybridizes to the unique codon, such as a premature stop codon (UAG, UGA, UAA) present in the mRNA encoding the protein to be expressed. See, for example, FIG. 1, which shows the synthesis of a protein using an endogenous tRNA and an endogenous aaRS from the expression host cell and an engineered orthogonal tRNA and an orthogonal aaRS introduced into the host cell so as to facilitate the incorporation of a UAA into the protein as it is synthesized via the ribosome. To date, a variety of orthogonal tRNA/aaRS pairs have been produced for certain of the naturally occurring amino acids (see, e.g., U.S. Patent Publication US2017/0349891, and Zheng et al. (2018) BIOCHEM. 57:441-445). The approach facilitates the expression of proteins containing site specific modifications such as bioconjugation handles and photoactivatable crosslinkers, which can be used as therapeutics (e.g., antibody drug conjugates (ADCs), bi-specific antibodies (e.g., bispecific monoclonal antibodies), nanobodies, chemokines, vaccines, coagulation factors, hormones, and enzymes).

Conjugation of drugs, payloads, oligonucleotides, half-life extenders, and other molecules to proteins (e.g., antibodies) has been explored as a method to improve therapeutic activity. However, there are limitations to the conjugation methods currently available, for example, with regard to site-selection, specificity, chemical compatibility with a protein and/or payload of interest, and the ability to conjugate multiple payloads of interest. Accordingly, despite the efforts made to date, there remains a need for the identification of suitable conjugation methods for UAA-containing proteins.

### SUMMARY OF THE INVENTION

The invention is based, in part, on the discovery of branched linkers that allow for efficient conjugation of molecules to unnatural amino acids (UAAs) in proteins (e.g., antibodies). The invention is further based, in part, on the discovery of combinations of UAAs, branched linkers for conjugation to those UAAs, and molecules for conjugation to those branched linkers. Among other things, the combinations of UAAs, branched linkers, and molecules allow for the efficient generation of protein conjugates with desirable properties, including, for example, expression yield, drug to antibody ratio (DAR), lack of aggregation, stability, and activity.

Accordingly, in one aspect, the invention provides a derivatized protein comprising: (a) an unnatural amino acid; (b) a parent linker having a first terminus and a second terminus, wherein the first terminus of the parent linker is covalently conjugated to the unnatural amino acid; (c) a branching group, wherein the branching group is covalently conjugated to the second terminus of the parent linker; (d) a plurality of branching linkers, each branching linker comprising a branching unit and a conjugating moiety, wherein each branching linker is covalently conjugated to the branching group via the branching unit present in the branching linker; and (e) a plurality of molecules, wherein each molecule is covalently conjugated to one of the plurality of branching linkers via the conjugating moiety present in the branching linker. In certain embodiments, the protein comprises at least two, at least three, or at least four branching linkers.

In another aspect, the invention provides a derivatized protein comprising: (a) an unnatural amino acid; (b) a parent linker having a first terminus and a second terminus, wherein the first terminus of the parent linker is covalently conjugated to the unnatural amino acid; (c) a branching group, wherein the branching group is covalently conjugated to the second terminus of the parent linker; (d) a first branching linker comprising a first branching unit and a first conjugating moiety, wherein the first branching unit is covalently conjugated to the branching group; (e) a first molecule, wherein the first molecule is covalently conjugated to the first branching linker via the first conjugating moiety; (f) a second branching linker comprising a second branching unit and a second conjugating moiety, wherein the second branching unit is covalently conjugated to the branching group; and (g) a second molecule, wherein the second molecule is covalently conjugated to the second branching linker via the second conjugating moiety. In certain embodiments, the protein may comprise a third or a fourth branching linker.

In another aspect, the invention provides a derivatized protein comprising: (a) an unnatural amino acid; (b) a parent linker having a first terminus and a second terminus, wherein the first terminus of the parent linker is covalently conjugated to the unnatural amino acid; (c) a branching group, wherein the branching group is covalently conjugated to the second terminus of the parent linker; and (d) a plurality of branching linkers, each branching linker comprising a branching unit and a conjugating moiety, wherein each branching linker is covalently conjugated to the branching group via the branching unit present in the branching linker. In certain embodiments, the protein comprises at least two, at least three, or at least four branching linkers.

In another aspect, the invention provides a derivatized protein comprising: (a) an unnatural amino acid; (b) a parent linker having a first terminus and a second terminus, wherein the first terminus of the parent linker is covalently conjugated to the unnatural amino acid; (c) a branching group, wherein the branching group is covalently conjugated to the second terminus of the parent linker; (d) a first branching linker comprising a first branching unit and a first conjugating moiety, wherein the first branching unit is covalently conjugated to the branching group; and (e) a second branching linker comprising a second branching unit and a second conjugating moiety, wherein the second branching unit is covalently conjugated to the branching group. In certain embodiments, the protein may comprise a third or a fourth branching linker.

In another aspect, the invention provides a derivatized protein of Formula I:

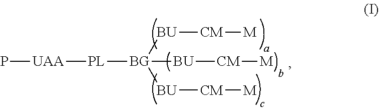

wherein P is a protein;
UAA is an unnatural amino acid disposed within the protein;
PL is a parent linker represented by

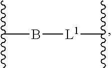

wherein B is a binding unit and L¹ is a chain selected from the group consisting of C₁₋₂₀ alkyl and C₁₋₂₀ heteroalkyl, wherein the chain is optionally substituted by one, two, three or four oxo; BG (branching group) is a polyvalent atom;
each BU (branching unit) independently is selected from the group consisting of C₁₋₂₀ alkyl and C₁₋₂₀ heteroalkyl, wherein each BU is optionally substituted by one, two or three oxo; each CM (conjugating moiety) independently is selected from the group consisting of a bond, NH, S, OR¹ and R¹;
R¹ is selected from the group consisting of phenyl, 5-10 membered cycloalkyl, 5-10 membered cycloalkenyl, 5-10 membered cycloalkynyl, 5-6 membered heterocycloalkyl, and 5-6 membered heteroaryl, wherein R¹ is optionally substituted by an oxo;
each M independently is a molecule; and
each of a, b and c independently is selected from the group consisting of 0, 1, 2 and 3,
wherein the sum of a, b and c is equal to or less than 3.

In another aspect, the invention provides a derivatized protein of Formula I:

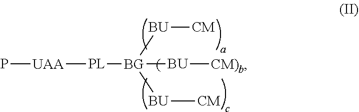

wherein P is a protein;
UAA is an unnatural amino acid disposed within the protein;
PL is a parent linker represented by

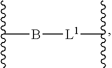

wherein B is a binding unit and L¹ is a chain selected from the group consisting of C₁₋₂₀ alkyl and C₁₋₂₀ heteroalkyl, wherein the chain is optionally substituted by one, two, three or four oxo;
BG (branching group) is a polyvalent atom;
each BU (branching unit) independently is selected from the group consisting of C₁₋₂₀ alkyl
and C₁₋₂₀ heteroalkyl, wherein each BU is optionally substituted by one, two or three oxo;
each CM (conjugating moiety) independently is selected from the group consisting of NH₂, SH, OH, O—(C₁₋₃alkyl), OR¹ and R¹;
R¹ is selected from the group consisting of phenyl, 5-10 membered cycloalkyl, 5-10 membered cycloalkenyl, 5-10 membered cycloalkynyl, 5-6 membered heterocycloalkyl, and 5-6 membered heteroaryl, wherein R¹ is optionally substituted by an oxo;
and each of a, b and c independently is selected from the group consisting of 0, 1, 2 and 3, wherein the sum of a, b and c is equal to or less than 3. In certain embodiments, the CM (conjugating moiety) is selected from the group consisting of thiol, maleimide, tetrazine, sulfohydryl/maleimide reactive group, N-hydroxysuccinimide (NHS), and NHS-ester.

In certain embodiments, the binding unit (B) independently is, or produced by a reaction with, a reactive group selected from the group consisting of dibenzylcyclooctyne (DBCO), (1R,8S,9s)-bicyclo[6.1.0]non-4-yn-9-ylmethanol (BCN), trans-cyclooctene (TCO), azido (N3), alkyne, tetrazine methylcyclopropene, norbornene, hydrazide/hydrazine, and aldehyde. In certain embodiments, the binding unit (B) independently is formed by a 1,3-dipolar cycloaddition reaction, hetero-Diels-Alder reaction, nucleophilic substitution reaction, non-aldol type carbonyl reaction, addition to carbon-carbon multiple bond, oxidation reaction, or click reaction. In certain embodiments, the binding unit (B) independently is formed by a reaction between acetylene and azide, or a reaction between an aldehyde or ketone group and a hydrazine or alkoxyamine.

In certain embodiments, each L¹ independently is selected from the group consisting of C(O)—(CH₂)₂—C(O), and C(O)—(CH₂)₂—C(O)—NH—(CH₂)₂—(O—(CH₂)₂)₃.

In certain embodiments, the polyvalent atom is N or C.

In certain embodiments, the protein comprises one, two, three, four, or more than four unnatural amino acids (UAAs), each of which may by the same or different, and each of which may optionally be covalently conjugated to a corresponding parent linker.

In certain embodiments, the UAA is: (i) a tryptophan analog (e.g., 5-HTP or 5-AzW); (ii) a leucine analog (e.g., LCA or Cys-5-N3); (iii) a tyrosine analog (e.g., OmeY, AzF, or OpropY); or (iv) a pyrrolysine analog (e.g., BocK, CpK, or AzK).

In certain embodiments, the protein comprises one, two, three, four, five, six, seven, eight, nine, ten, eleven, twelve, or more than twelve molecules, each of which may by the same or different.

In certain embodiments, the molecule is a therapeutic agent (e.g., a small molecule or biomolecule, e.g., an antibody or antigen binding fragment thereof). In certain embodiments, the molecule is a radionuclide (e.g., astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium⁸⁸, ⁷⁵selenium, ³⁵sulphur, technicium^{99m} and/or yttrium⁹⁰). In certain embodiments, the molecule is a reporter group (e.g., a detectable label such as a fluorescent label or an optical label, or an enzyme that can convert a substrate into a detectable group). In certain embodiments, the molecule is: AEB, AEVB, AFP, an amatoxin, an auristatin (e.g., auristatin E), a calicheamicin, CC-1065 or a CC-1065 analog, chalicheamicin, combretastatin, DM1, DM4, docetaxel, dolastatin-10, DUBA, a duocarmycin, echinomycin, FAM, maytansine, a maytansinoid, MMAD, MMAE, MMAF, a morpholino-doxorubicin (e.g., cyanomorpholino-doxorubicin), netropsin, an oligonucleotide (e.g., a DNA, RNA, or LNA oligonucleotide), paclitaxel, PBD, a peptide (e.g., a therapeutic peptide), rhizoxin, a small molecule (e.g., a therapeutic small molecule) SN-38, topotecan, a topoisomerase inhibitor, or a toxoid.

In certain embodiments, the protein is selected from the group consisting of:

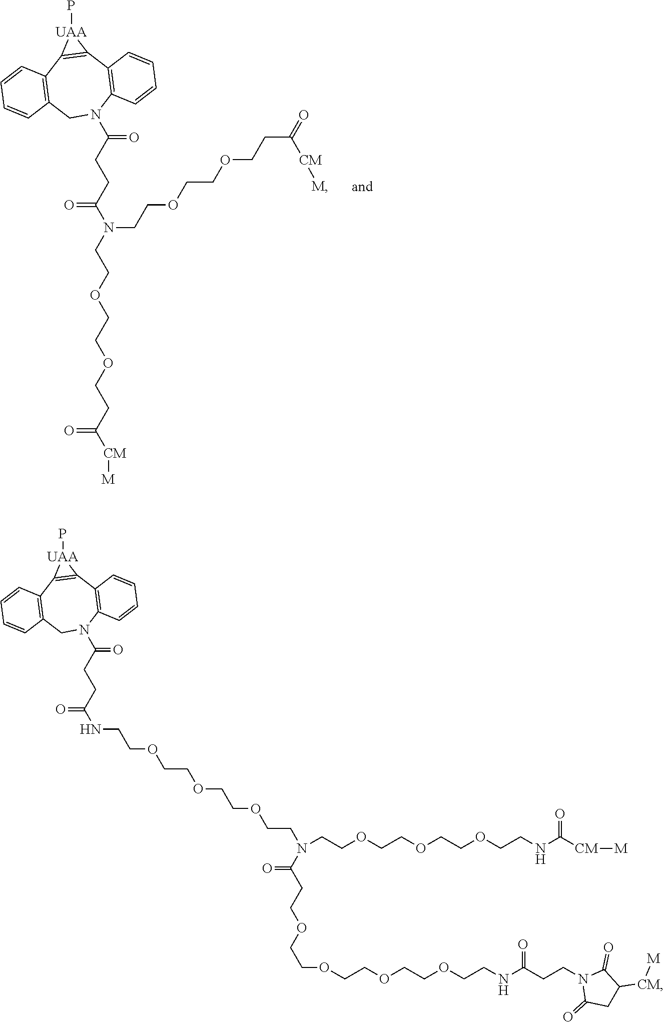

where P is a protein, UAA is an unnatural amino acid disposed within the protein, CM is a conjugating moiety, and M is a molecule (e.g., a therapeutic agent, radionuclide, or reporter group).

In certain embodiments, the protein is selected from the group consisting of:

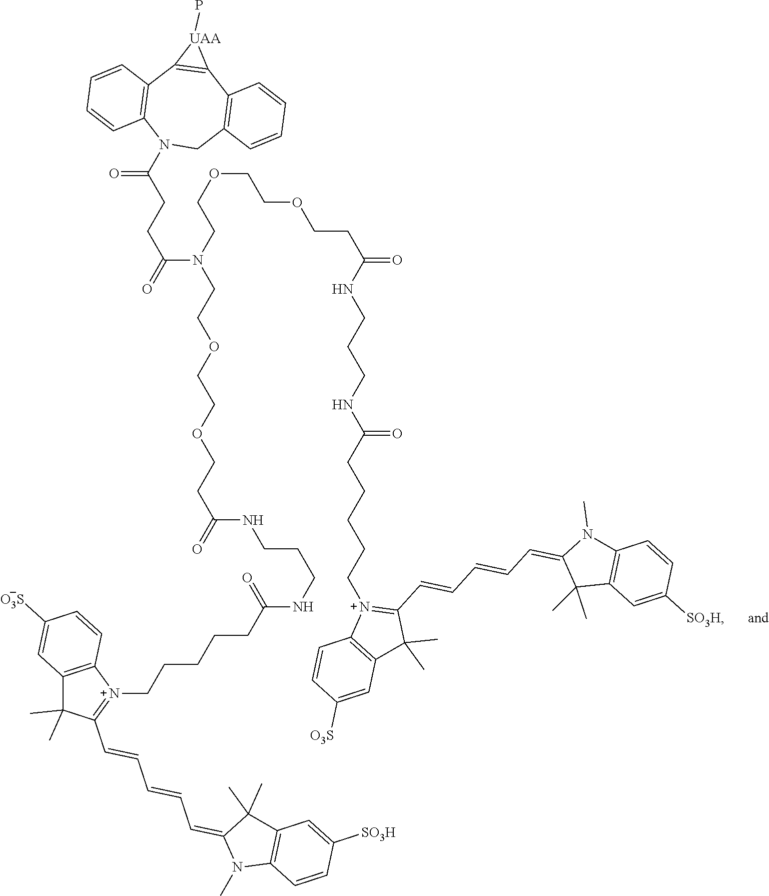

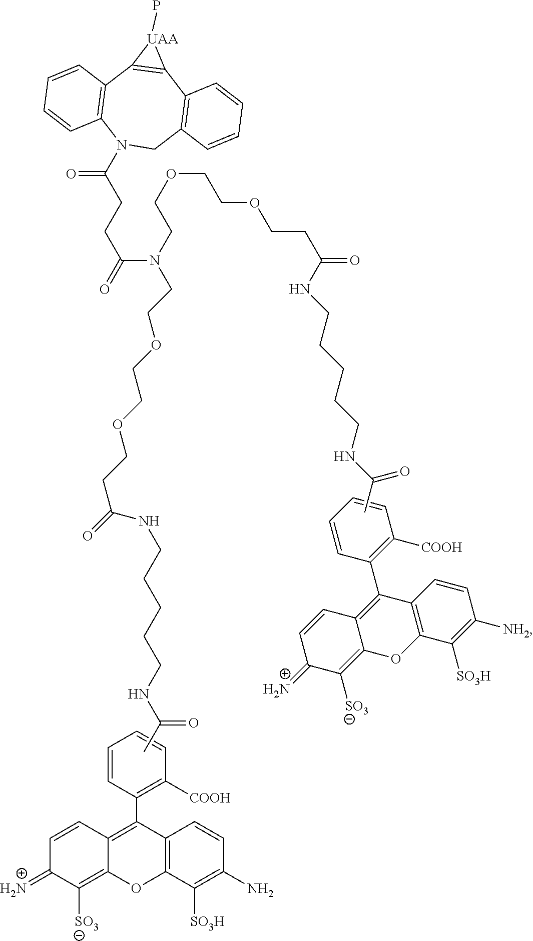

where P is a protein and UAA is an unnatural amino acid disposed within the protein.

In certain embodiments, the protein is selected from the group consisting of:

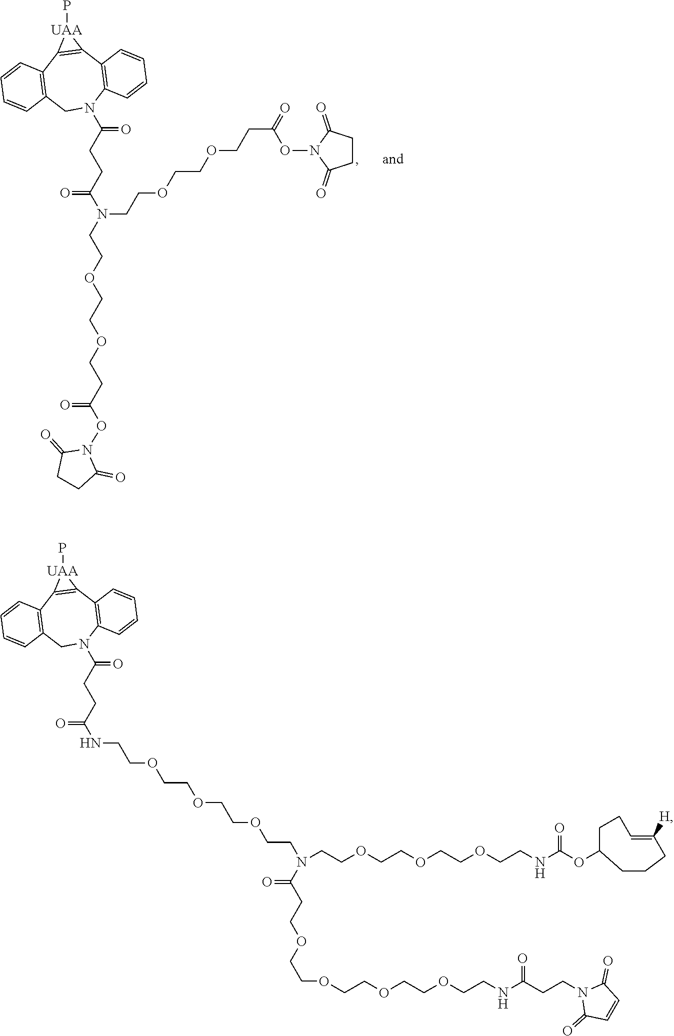

where P is a protein and UAA is an unnatural amino acid disposed within the protein.

In certain embodiments, the protein comprises trastuzumab, or a variant thereof. For example, the protein may comprise trastuzumab or a variant thereof comprising LCA at a position corresponding to T198 of the heavy chain of trastuzumab (e.g., at a position corresponding to T198 in SEQ ID NO: 114).

In another aspect, the invention provides a composition comprising any of the foregoing protein derivatives. In another aspect, the invention provides a pharmaceutical composition comprising any of the foregoing protein derivatives and a pharmaceutically acceptable carrier and/or excipient.

In another aspect, the invention provides a method of producing any of the foregoing protein derivatives. The method comprises culturing a cell with: (i) a nucleic acid comprising a nucleotide sequence encoding a tRNA comprising an anticodon that hybridizes to a codon selected from UAG, UGA, and UAA, and is capable of being charged with the unnatural amino acid (UAA); (ii) a nucleic acid comprising a nucleotide sequence encoding an aminoacyl-tRNA synthetase capable of charging the tRNA with the unnatural amino acid (UAA); and (iii) a nucleic acid comprising a nucleotide sequence encoding a protein (e.g., encoding a heavy chain, a light chain, or a combination of a heavy chain and light chain of the antibody) and comprising the codon selected from UAG, UGA, and UAA; under conditions that permit the tRNA, when expressed in the cell and charged with the unnatural amino acid (UAA), to hybridize to the codon and direct the incorporation of the unnatural amino acid (UAA) into the antibody. The method also comprises conjugating the parental linker to the UAA and/or conjugating the molecule to the conjugating moiety. The method also optionally comprises purifying the protein before and/or after any expression and/or conjugation step.

In certain embodiments, the tRNA is an analog or derivative of a prokaryotic tryptophanyl-tRNA, e.g., an E. coli tryptophanyl-tRNA. For example, the tRNA may comprise a nucleotide sequence selected from any one of SEQ ID NOs: 49-54 or 108-113. In certain embodiments, the aminoacyl-tRNA synthetase is an analog or derivative of a prokaryotic tryptophanyl-tRNA synthetase, e.g., an E. coli tryptophanyl-tRNA synthetase. For example, the aminoacyl-tRNA synthetase may comprise an amino acid sequence selected from any one of SEQ ID NOs: 44-48. In certain embodiments, the codon is UGA. In certain embodiments, the UAA is a tryptophan analog, e.g., a non-naturally occurring tryptophan analog. In certain embodiments, the UAA is 5-HTP or 5-AzW.

In certain embodiments, the tRNA is an analog or derivative of a prokaryotic leucyl-tRNA, e.g., an E. coli leucyl-tRNA. For example, the tRNA may comprise a nucleotide sequence selected from any one of SEQ ID NOs: 16-43. In certain embodiments, the aminoacyl-tRNA synthetase is an analog or derivative of a prokaryotic leucyl-tRNA synthetase, e.g., an E. coli leucyl-tRNA synthetase. For example, the aminoacyl-tRNA synthetase may comprise an amino acid sequence selected from any one of SEQ ID NOs: 1-15. In certain embodiments, the codon is UAG. In certain embodiments, the UAA is a leucine analog, e.g., a non-naturally occurring leucine analog. In certain embodiments, the UAA is LCA or Cys-5-N3.

In certain embodiments, the tRNA is an analog or derivative of a prokaryotic tyrosyl-tRNA, e.g., an E. coli tyrosyl-tRNA. For example, the tRNA may comprise a nucleotide sequence selected from any one of SEQ ID NOs: 68-69 or 104-105. In certain embodiments, the aminoacyl-tRNA synthetase is an analog or derivative of a prokaryotic tyrosyl-tRNA synthetase, e.g., an E. coli tyrosyl-tRNA synthetase. For example, the aminoacyl-tRNA synthetase may comprise the amino acid sequence of SEQ ID NO: 70. In certain embodiments, the codon is UAG. In certain embodiments, the UAA is a tyrosine analog, e.g., a non-naturally occurring tyrosine analog. In certain embodiments, the UAA is OmeY, AzF, or OpropY.

In certain embodiments, the tRNA is an analog or derivative of an archael pyrrolysyl-tRNA, e.g., an M. barkeri pyrrolysyl-tRNA. For example, the tRNA may comprise a nucleotide sequence selected from any one of SEQ ID NOs: 72-100 or 106-107. In certain embodiments, the aminoacyl-tRNA synthetase is an analog or derivative of an archael pyrrolysyl-tRNA synthetase, e.g., an M. barkeri pyrrolysyl-tRNA synthetase. For example, the aminoacyl-tRNA synthetase may comprise the amino acid sequence of SEQ ID NO: 101. In certain embodiments, the codon is UAG. In certain embodiments, the UAA is a pyrrolysine analog, e.g., a non-naturally occurring pyrrolysine analog. In certain embodiments, the UAA is BocK, CpK, or AzK.

In certain embodiments, the cell is a human cell, e.g., a human embryonic kidney (HEK) or a Chinese hamster ovary (CHO) cell.

These and other aspects and features of the invention are described in the following detailed description and claims.

### DESCRIPTION OF THE DRAWINGS

The invention can be more completely understood with reference to the following drawings.

FIG. 1 depicts a schematic overview of genetic code expansion using unnatural amino acids (UAAs).

FIG. 2A depicts a subset of UAAs that are exemplary substrates for a leucyl tRNA-synthetase. FIG. 2B depicts a subset of UAAs that are exemplary substrates for a tryptophanyl tRNA-synthetase.

FIG. 3A and FIG. 3B depict a subset of UAAs that are exemplary substrates for a leucyl tRNA-synthetase.

FIG. 4A depicts UAAs C5Az, LCA, and AzW; FIG. 4B depicts a subset of UAAs that are exemplary substrates for a tyrosyl tRNA-synthetase; and FIG. 4C depicts a subset of UAAs that are exemplary substrates for a pyrrolysyl tRNA-synthetase.

FIG. 5 depicts a preparation of DBCO-2xCy5 Ligand (Compound 209). FIG. 5A depicts DBCO-BIS-NHS (left) and an HIC analysis of DBCO-BIS-NHS (right). The arrow depicts the DBCO-BIS-NHS peak. FIG. 5B depicts Cy5 amine (left) and an HIC analysis of Cy5 amine (right). The arrow depicts the Cy5 amine peak. FIG. 5C depicts DBCO-2xCy5 (left) and an HIC analysis following incubation of DBCO-BIS-NHS with Cy5 amine (right). The left arrow depicts the excess Cy5 amine peak, the middle arrow depicts the unlabeled DBCO-BIS-NHS peak (˜10%) and the right arrow depicts the conjugated DBCO-2xCy5 peak (˜90%).

FIG. 6 depicts confirm of completion of the preparation of DBCO-2xCy5 Ligand (Compound 209). FIG. 6A depicts an HIC analysis of 488-Cadaverine only. The arrow depicts the 488-Cadaverine peak. FIG. 6B depicts an HIC analysis of DBCO-BIS-NHS only (as shown in FIG. 5A, and repeated here for reference). The arrow depicts the DBCO-BIS-NHS peak. FIG. 6C depicts an HIC analysis following incubation of 488-Cadaverine and DBCO-BIS-NHS. The left arrow depicts the excess Cy5 amine peak, the middle arrow depicts the conjugated DBCO-2×488-Cadaverine peak, and the right arrow depicts the unlabeled DBCO-BIS-NHS peak. FIG. 6D depicts 488-Cadaverine. FIG. 6E depicts an HIC analysis of DBCO-2xCy5 compound (as shown in FIG. 5C, and repeated here for reference). The arrows, from left to right, depict: (i) the excess Cy5 amine peak, (ii) the unlabeled DBCO-BIS-NHS peak (˜10%), and (iii) the conjugated DBCO-2xCy5 peak. FIG. 6F depicts HIC analysis after incubation of 488-Cadaverine and DBCO-2xCy5 Ligand. The arrows, from left to right, depict: (i) the excess 488-Cadaverine peak (ii) the excess Cy5 amine peak, (iii) the inactive DBCO-BIS-NHS peak, and (iv) the conjugated DBCO-2xCy5 peak.

FIG. 7 depicts conjugation of trastuzumab with DBCO-2xCy5 Ligand. FIG. 7A depicts HIC analysis of unmodified trastuzumab containing a T198LCA mutation in the heavy chain (TzmAb-T198LCA). FIG. 7B depicts HIC analysis following incubation of TzmAb-T198LCA with DBCO-2xCy5. FIG. 7C depicts mass spectrometry analysis of the heavy chain of TzmAb-T198LCA modified with DBCO-2xCy5. FIG. 7D depicts mass spectrometry analysis of the light chain of TzmAb-T198LCA modified with DBCO-2xCy5 conjugate.

FIG. 8 depicts exemplary conjugation methods. FIG. 8A shows an exemplary reaction between a tryptophan analog unnatural amino acid A-1 and a diazonium linker B-1 to produce a conjugate C-1. FIG. 8B shows an exemplary electron demand Diels-Alder (IEDDA) reaction between a leucine analog unnatural amino acid A-2 or A-3 and tetrazine linker B-2 to produce a conjugate C-2. FIG. 8C shows an exemplary click chemistry reaction between a leucine analog unnatural amino acid A-3 and DBCO linker B-3 to produce a conjugate C-3. FIG. 8D shows an exemplary click chemistry reaction between a tryptophan analog unnatural amino acid A-4 and DBCO linker B-4 to produce a conjugate C-4.

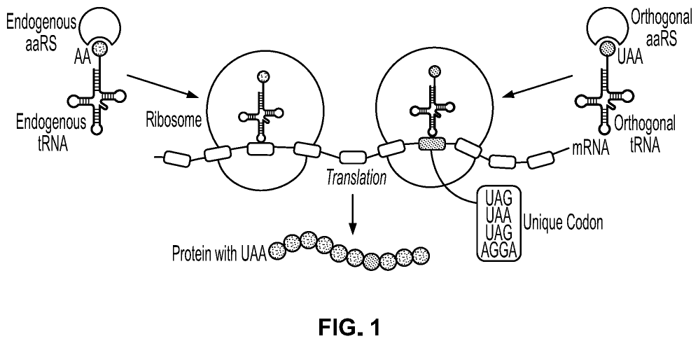

### DETAILED DESCRIPTION

The invention is based, in part, on the discovery of branched linkers that allow for efficient conjugation of molecules to unnatural amino acids (UAAs) in proteins (e.g., antibodies). The invention is further based, in part, on the discovery of combinations of UAAs, branched linkers for conjugation to those UAAs, and molecules for conjugation to those branched linkers. Among other things, the combinations of UAAs, branched linkers, and molecules allow for the efficient generation of protein conjugates with desirable properties, including, for example, expression yield, drug to antibody ratio (DAR), lack of aggregation, stability, and activity.

Accordingly, in one aspect, the invention provides a derivatized protein comprising: (a) an unnatural amino acid; (b) a parent linker having a first terminus and a second terminus, wherein the first terminus of the parent linker is covalently conjugated to the unnatural amino acid; (c) a branching group, wherein the branching group is covalently conjugated to the second terminus of the parent linker; (d) a plurality of branching linkers, each branching linker comprising a branching unit and a conjugating moiety, wherein each branching linker is covalently conjugated to the branching group via the branching unit present in the branching linker; and (e) a plurality of molecules, wherein each molecule is covalently conjugated to one of the plurality of branching linkers via the conjugating moiety present in the branching linker. In certain embodiments, the protein comprises at least two, at least three, or at least four branching linkers.

In another aspect, the invention provides a derivatized protein comprising: (a) an unnatural amino acid; (b) a parent linker having a first terminus and a second terminus, wherein the first terminus of the parent linker is covalently conjugated to the unnatural amino acid; (c) a branching group, wherein the branching group is covalently conjugated to the second terminus of the parent linker; (d) a first branching linker comprising a first branching unit and a first conjugating moiety, wherein the first branching unit is covalently conjugated to the branching group; (e) a first molecule, wherein the first molecule is covalently conjugated to the first branching linker via the first conjugating moiety; (f) a second branching linker comprising a second branching unit and a second conjugating moiety, wherein the second branching unit is covalently conjugated to the branching group; and (g) a second molecule, wherein the second molecule is covalently conjugated to the second branching linker via the second conjugating moiety. In certain embodiments, the protein may comprise a third or a fourth branching linker.

In another aspect, the invention provides a derivatized protein comprising: (a) an unnatural amino acid; (b) a parent linker having a first terminus and a second terminus, wherein the first terminus of the parent linker is covalently conjugated to the unnatural amino acid; (c) a branching group, wherein the branching group is covalently conjugated to the second terminus of the parent linker; and (d) a plurality of branching linkers, each branching linker comprising a branching unit and a conjugating moiety, wherein each branching linker is covalently conjugated to the branching group via the branching unit present in the branching linker. In certain embodiments, the protein comprises at least two, at least three, or at least four branching linkers.

In another aspect, the invention provides a derivatized protein comprising: (a) an unnatural amino acid; (b) a parent linker having a first terminus and a second terminus, wherein the first terminus of the parent linker is covalently conjugated to the unnatural amino acid; (c) a branching group, wherein the branching group is covalently conjugated to the second terminus of the parent linker; (d) a first branching linker comprising a first branching unit and a first conjugating moiety, wherein the first branching unit is covalently conjugated to the branching group; and (e) a second branching linker comprising a second branching unit and a second conjugating moiety, wherein the second branching unit is covalently conjugated to the branching group. In certain embodiments, the protein may comprise a third or a fourth branching linker.

In another aspect, the invention provides a derivatized protein of Formula I:

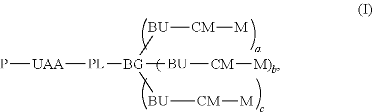

wherein P is a protein;
UAA is an unnatural amino acid;
PL is a parent linker represented by

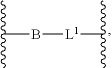

wherein B is a binding unit and L¹ is a chain selected from the group consisting of C₁₋₂₀ alkyl and C₁₋₂₀ heteroalkyl, wherein the chain is optionally substituted by one, two, three or four oxo; BG (branching group) is a polyvalent atom;
each BU (branching unit) independently is selected from the group consisting of C₁₋₂₀ alkyl and C₁₋₂₀ heteroalkyl, wherein each BU is optionally substituted by one, two or three oxo;
each CM (conjugating moiety) independently is selected from the group consisting of a bond, NH, S, OR¹ and R¹;
R¹ is selected from the group consisting of phenyl, 5-10 membered cycloalkyl, 5-10 membered cycloalkenyl, 5-10 membered cycloalkynyl, 5-6 membered heterocycloalkyl, and 5-6 membered heteroaryl, wherein R¹ is optionally substituted by an oxo;
each M independently is a molecule; and
each of a, b and c independently is selected from the group consisting of 0, 1, 2 and 3, wherein the sum of a, b and c is equal to or less than 3.

In another aspect, the invention provides a derivatized protein of Formula I.

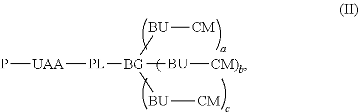

wherein P is a protein;
UAA is an unnatural amino acid;
PL is a parent linker represented by

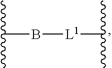

wherein B is a binding unit and L¹ is a chain selected from the group consisting of C₁₋₂₀ alkyl and C₁₋₂₀ heteroalkyl, wherein the chain is optionally substituted by one, two, three or four oxo;
BG (branching group) is a polyvalent atom;
each BU (branching unit) independently is selected from the group consisting of C₁₋₂₀ alkyl and C₁₋₂₀ heteroalkyl, wherein each BU is optionally substituted by one, two or three oxo;
each CM (conjugating moiety) independently is selected from the group consisting of NH₂, SH, OH, O—(C₁₋₃alkyl), OR¹ and R¹;
R¹ is selected from the group consisting of phenyl, 5-10 membered cycloalkyl, 5-10 membered cycloalkenyl, 5-10 membered cycloalkynyl, 5-6 membered heterocycloalkyl, and 5-6 membered heteroaryl, wherein R¹ is optionally substituted by an oxo;
and each of a, b and c independently is selected from the group consisting of 0, 1, 2 and 3, wherein the sum of a, b and c is equal to or less than 3.

Various features and aspects of the invention are discussed in more detail below.

### I. Proteins

Encompassed by the invention are proteins including unnatural amino acids (UAAs) and branched linkers, and methods of making the same.

The incorporation of an unnatural amino acid and/or branched linker can be done for a variety of purposes, including tailoring changes in protein structure and/or function, changing size, acidity, nucleophilicity, hydrogen bonding, hydrophobicity, accessibility of protease target sites, targeting to a moiety (e.g., for a protein array), adding a biologically active molecule, attaching a polymer, attaching a radionuclide, modulating serum half-life, modulating tissue penetration (e.g. tumors), modulating active transport, modulating tissue, cell or organ specificity or distribution, modulating immunogenicity, modulating protease resistance, etc. Proteins that include an unnatural amino acid can have enhanced or even entirely new catalytic or biophysical properties. For example, the following properties are optionally modified by inclusion of an unnatural amino acid and/or branched linker into a protein: toxicity, biodistribution, structural properties, spectroscopic properties, chemical and/or photochemical properties, catalytic ability, half-life (including but not limited to, serum half-life), ability to react with other molecules, including but not limited to, covalently or noncovalently, and the like. The compositions including proteins that include at least one unnatural amino acid and/or branched linker are useful for, including but not limited to, novel therapeutics, diagnostics, enzymes, and binding proteins (e.g., therapeutic antibodies).

A protein may have at least one, for example, at least two, at least three, at least four, at least five, at least six, at least seven, at least eight, at least nine, or at least ten or more UAAs. The UAAs can be the same or different. For example, there can be 1, 2, 3, 4, 5, 6, 7, 8, 9, or 10 or more different sites in the protein that comprise 1, 2, 3, 4, 5, 6, 7, 8, 9, or 10 or more different UAAs. A protein may have at least one, but fewer than all, of a particular amino acid present in the protein substituted with the UAA. For a given protein with more than one UAA, the UAA can be identical or different (for example, the protein can include two or more different types of UAAs, or can include two of the same UAA). For a given protein with more than two UAAs, the UAAs can be the same, different or a combination of a multiple unnatural amino acid of the same kind with at least one different UAA.

In certain embodiments, the protein is an antibody (or a fragment thereof), bispecific antibody, nanobody, affibody, viral protein, chemokine, antigen, blood coagulation factor, hormone, growth factor, enzyme, or any other polypeptide or protein.

Additional examples of therapeutic, diagnostic, and other proteins that can be modified to comprise one or more unnatural amino acids and/or branched linkers are described in U.S. Patent Application Publication Nos. 2003/0082575 and 2005/0009049.

The term protein includes variants having one or more mutations (e.g., amino acid substitutions, deletions, or insertions) relative to a wild-type protein sequence or a protein sequence disclosed herein. In certain embodiments, a protein variant may comprise, consist, or consist essentially of, a single mutation, or a combination of 2, 3, 4, 5, 6, 7, 8, 9, 10, 11, 12, 13, 14, 15 or more than 15 mutations relative to a wild-type protein sequence or a protein sequence disclosed herein. It is contemplated that a protein variant may comprise, consist, or consist essentially 1-15, 1-10, 1-7, 1-6, 1-5, 1-4, 1-3, 1-2, 2-15, 2-10, 2-7, 2-6, 2-5, 2-4, 2-3, 3-15, 3-10, 3-7, 3-6, 3-5, or 4-10, 4-7, 4-6, 4-5, 5-10, 5-7, 5-6, 6-10, 6-7, 7-10, 7-8, or 8-10 mutations relative to a wild-type protein sequence or a protein sequence disclosed herein. A protein variant may comprise a conservative substitution relative to a wild-type sequence or a sequence disclosed herein. As used herein, the term “conservative substitution” refers to a substitution with a structurally similar amino acid. For example, conservative substitutions may include those within the following groups: Ser and Cys; Leu, Ile, and Val; Glu and Asp; Lys and Arg; Phe, Tyr, and Trp; and Gln, Asn, Glu, Asp, and His. Conservative substitutions may also be defined by the BLAST (Basic Local Alignment Search Tool) algorithm, the BLOSUM substitution matrix (e.g., BLOSUM 62 matrix), or the PAM substitution: p matrix (e.g., the PAM 250 matrix).

Throughout the specification, a first position in a first protein, protein fragment, or amino acid sequence is considered to “correspond” with a second position in a second, different protein, protein fragment, or amino acid sequence, if a person of skill in the art would understand the first and second positions to correspond to the same position in the primary, secondary, or tertiary structure of their respective protein, protein fragment, or amino acid sequence. It is understood that the first and second positions may correspond to each other even if they have a different numbered position relative to the N-terminus of their respective protein, protein fragment, or amino acid sequence, or if a different amino acid is present at the first and second positions. Primary, secondary, or tertiary structure analysis of proteins, protein fragments, or amino acid sequences may be performed using any method known in the art, including, for example, sequence analysis software such as BLAST.

Sequence identity may be determined in various ways that are within the skill of a person skilled in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36:290-300; Altschul et al., (1997) NUCLEIC ACIDS RES. 25:3389-3402, incorporated by reference herein) are tailored for sequence similarity searching. For a discussion of basic issues in searching sequence databases see Altschul et al., (1994) NATURE GENETICS 6:119-129, which is fully incorporated by reference herein. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference herein). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent blastp parameter settings may be Q=9; R=2; wink=1; and gapw=32. Searches may also be conducted using the NCBI (National Center for Biotechnology Information) BLAST Advanced Option parameter (e.g.: -G, Cost to open gap [Integer]: default=5 for nucleotides/11 for proteins; −E, Cost to extend gap [Integer]: default=2 for nucleotides/1 for proteins; −q, Penalty for nucleotide mismatch [Integer]: default=−3; −r, reward for nucleotide match [Integer]: default=1; −e, expect value [Real]: default=10; —W, wordsize [Integer]: default=11 for nucleotides/28 for megablast/3 for proteins; −y, Dropoff (X) for blast extensions in bits: default=20 for blastn/7 for others; —X, X dropoff value for gapped alignment (in bits): default=15 for all programs, not applicable to blastn; and —Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty). The equivalent settings in Bestfit protein comparisons are GAP=8 and LEN=2.

In certain embodiments, the protein is an antibody. As used herein, unless otherwise indicated, the term “antibody” is understood to mean an intact antibody (e.g., an intact monoclonal antibody), or a fragment thereof, such as a Fc fragment of an antibody (e.g., an Fc fragment of a monoclonal antibody), or an antigen-binding fragment of an antibody (e.g., an antigen-binding fragment of a monoclonal antibody), including an intact antibody, antigen-binding fragment, or Fc fragment that has been modified, engineered, or chemically conjugated. Examples of antigen-binding fragments include Fab, Fab′, (Fab′)₂, Fv, single chain antibodies (e.g., scFv), minibodies, and diabodies. Examples of antibodies that have been modified or engineered include chimeric antibodies, humanized antibodies, and multispecific antibodies (e.g., bispecific antibodies). An example of a chemically conjugated antibody is an antibody conjugated to a toxin moiety.

Typically, antibodies are multimeric proteins that contain four polypeptide chains. Two of the polypeptide chains are called immunoglobulin heavy chains (H chains), and two of the polypeptide chains are called immunoglobulin light chains (L chains). The immunoglobulin heavy and light chains are connected by an interchain disulfide bond. The immunoglobulin heavy chains are connected by interchain disulfide bonds. A light chain consists of one variable region (V_{L}) and one constant region (C_{L}). The heavy chain consists of one variable region (V_{H}) and at least three constant regions (CH₁, CH₂ and CH₃). The variable regions determine the binding specificity of the antibody.

The variable heavy (VH) and variable light (VL) regions can be further subdivided into regions of hypervariability, termed “complementarity determining regions” (“CDR”), interspersed with regions that are more conserved, termed “framework regions” (FR). Human antibodies have three VH CDRs and three VL CDRs, separated by framework regions FR1-FR4. The extent of the FRs and CDRs has been defined (Kabat, E. A., et al. (1991) SEQUENCES OF PROTEINS OF IMMUNOLOGICAL INTEREST, FIFTH EDITION, U.S. Department of Health and Human Services, NIH Publication No. 91-3242; and Chothia, C. et al. (1987) J. MOL. BIOL. 196:901-917). Each VH and VL is typically composed of three CDRs and four FRs, arranged from amino-terminus to carboxyl-terminus in the following order: FR1, CDR1, FR2, CDR2, FR3, CDR3, and FR4.

An antibody may have (i) a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4, and/or (ii) a light chain constant region chosen from, e.g., the (e.g., human) light chain constant regions of kappa or lambda.

Antibodies contemplated herein may comprise a UAA in a heavy chain or a fragment thereof, for example, in one or more of a heavy chain FR1, CDR1, FR2, CDR2, FR3, CDR3, FR4, or constant region (e.g., an IgG1 constant region). Alternatively or in addition, antibodies contemplated herein may comprise a UAA in a light chain or a fragment thereof, for example, in a light chain FR1, CDR1, FR2, CDR2, FR3, CDR3, FR4, or constant region (e.g., a kappa constant region).

The antibody may be selected from, or may be derived from an antibody selected from, adecatumumab, ascrinvacumab, cixutumumab, conatumumab, daratumumab, drozitumab, duligotumab, durvalumab, dusigitumab, enfortumab, enoticumab, epratuxumab, figitumumab, ganitumab, glembatumumab, intetumumab, ipilimumab, iratumumab, icrucumab, lexatumumab, lucatumumab, mapatumumab, narnatumab, necitumumab, nesvacumab, ofatumumab, olaratumab, panitumumab, patritumab, pritumumab, radretumab, ramucirumab, rilotumumab, robatumumab, seribantumab, tarextumab, teprotumumab, tovetumab, vantictumab, vesencumab, votumumab, zalutumumab, flanvotumab, altumomab, anatumomab, arcitumomab, bectumomab, blinatumomab, detumomab, ibritumomab, minretumomab, mitumomab, moxetumomab, naptumomab, nofetumomab, pemtumomab, pintumomab, racotumomab, satumomab, solitomab, taplitumomab, tenatumomab, tositumomab, tremelimumab, abagovomab, atezolizumab, durvalumab, avelumab, igovomab, oregovomab, capromab, edrecolomab, nacolomab, amatuximab, bavituximab, brentuximab, cetuximab, derlotuximab, dinutuximab, ensituximab, futuximab, girentuximab, indatuximab, isatuximab, margetuximab, rituximab, siltuximab, ublituximab, ecromeximab, abituzumab, alemtuzumab, bevacizumab, bivatuzumab, brontictuzumab, cantuzumab, cantuzumab, citatuzumab, clivatuzumab, dacetuzumab, demcizumab, dalotuzumab, denintuzumab, elotuzumab, emactuzumab, emibetuzumab, enoblituzumab, etaracizumab, farletuzumab, ficlatuzumab, gemtuzumab, imgatuzumab, inotuzumab, labetuzumab, lifastuzumab, lintuzumab, lirilumab, lorvotuzumab, lumretuzumab, matuzumab, milatuzumab, moxetumomab, nimotuzumab, obinutuzumab, ocaratuzumab, otlertuzumab, onartuzumab, oportuzumab, parsatuzumab, pertuzumab, pidilizumab, pinatuzumab, polatuzumab, sibrotuzumab, simtuzumab, tacatuzumab, tigatuzumab, trastuzumab, tucotuzumab, urelumab, vandortuzumab, vanucizumab, veltuzumab, vorsetuzumab, sofituzumab, catumaxomab, ertumaxomab, depatuxizumab, ontuxizumab, blontuvetmab, tamtuvetmab, nivolumab, pembrolizumab, epratuzumab, MEDI9447, urelumab, utomilumab, hu3F8, hu14.18-IL-2, 3F8/OKT3BsAb, lirilumab, BMS-986016 pidilizumab, AMP-224, AMP-514, BMS-936559, atezolizumab, and avelumab.

The antibody may bind an antigen selected from, for example, adenosine A2a receptor (A2aR), A kinase anchor protein 4 (AKAP4), B melanoma antigen (BAGE), brother of the regulator of imprinted sites (BORIS), breakpoint cluster region Abelson tyrosine kinase (BCR/ABL), CA125, CAIX, CD19, CD20, CD22, CD30, CD33, CD52, CD73, CD137, carcinoembryonic antigen (CEA), a claudin (e.g. a claudin 18, e.g., claudin 18.2), CS1, cytotoxic T-lymphocyte-associated antigen 4 (CTLA-4), estrogen receptor binding site associated antigen 9 (EBAG9), epidermal growth factor (EGF), epidermal growth factor receptor (EGFR), EGF-like module receptor 2 (EMR2), epithelial cell adhesion molecule (EpCAM) (17-1A), FR-alpha, G antigen (GAGE), disialoganglioside GD2 (GD2), glycoprotein 100 (gp100), human epidermal growth factor receptor 2 (HER2), hepatocyte growth factor (HGF), human papillomavirus 16 (HPV-16), heat-shock protein 105 (HSP105), isocitrate dehydrogenase type 1 (IDH1), idiotype (NeuGcGM3), indoleamine-2,3-dioxygenase 1 (IDO1), IGF-1, IGF1R, IGG1K, killer cell immunoglobulin-like receptor (KIR), lymphocyte activation gene 3 (LAG-3), lymphocyte antigen 6 complex K (LY6K), Matrix-metalloproteinase-16 (MMP16), melanotransferrin (MFI2), melanoma antigen 3 (MAGE-A3), melanoma antigen C2 (MAGE-C2), melanoma antigen D4 (MAGE-D4), melanoma antigen recognized by T-cells 1 (Melan-A/MART-1), N-methyl-N′-nitroso-guanidine human osteosarcoma transforming gene (MET), mucin 1 (MUC1), mucin 4 (MUC4), mucin 16 (MUC16), New York esophageal squamous cell carcinoma 1 (NY-ESO-1), prostatic acid phosphatase (PAP), programmed cell death receptor 1 (PD-1), programmed cell death receptor ligand 1 (PD-L1), phosphatidylserine, preferentially expressed antigen of melanoma (PRAME), prostate specific antigen (PSA), protein tyrosine kinase 7 (PTK7, also known as colon carcinoma kinase 4 (CCK4)), receptor tyrosine kinase orphan receptor 1 (ROR1), scatter factor receptor kinase, sialyl-Tn, sperm-associated antigen 9 (SPAG-9), synovial sarcoma X-chromosome breakpoint 1 (SSX1), survivin, telomerase, T-cell immunoglobulin domain and mucin domain-3 (TIM-3), vascular endothelial growth factor (VEGF) (e.g., VEGF-A), vascular endothelial growth factor Receptor 2 (VEGFR2), V-domain immunoglobulin-containing suppressor of T-cell activation (VISTA), Wilms' Tumor-1 (WT1), X chromosome antigen 1b (XAGE-1b), 5T4, Mesothelin, Glypican 3 (GPC3), Folate Receptor α (FRα), Prostate Specific Membrane Antigen (PSMA), cMET, CD38, B Cell Maturation Antigen (BCMA), CD123, CLDN6, CLDN9, LRRC15, PRLR (Prolactin Receptor), RING finger protein 43 (RNF43), Uroplakin-1 B (UPK1 B), tumor necrosis factor superfamily member 9 (TNFSF9), tumor necrosis factor receptor superfamily member 21 (TNFSRF21), bone morphogenetic protein receptor type-1B (BMPR1B), Kringle domain-containing transmembrane protein 2 (KREMEN2), Delta-like protein 3 (DLL3), Siglec7 and Siglec9. Additional exemplary cancer antigens include those found on cancer stem cells, e.g., SSEA3, SSEA4, TRA-1-60, TRA-1-81, SSEA1, CD133 (AC133), CD90 (Thy-1), CD326 (EpCAM), Cripto-1 (TDGF1), PODXL-1 (Podocalyxin-like protein 1), ABCG2, CD24, CD49f (Integrin a6), Notch2, CD146 (MCAM), CD10 (Neprilysin), CD117 (c-KIT), CD26 (DPP-4), CXCR4, CD34, CD271, CD13 (Alanine aminopeptidase), CD56 (NCAM), CD105 (Endoglin), LGR5, CD114 (CSF3R), CD54 (ICAM-1), CXCR1, 2, TIM-3 (HAVCR2), CD55 (DAF), DLL4 (Delta-like ligand 4), CD20 (MS4A1), and CD96.

Table 1 shows antibodies and antibody-drug conjugates suitable for use in accordance with the present invention, the antigen bound by the antibody or antibody-drug conjugate, and for certain antibodies, the type of cancer targeted by the antibody or antibody-drug conjugate.

TABLE 1

| Antibody or antibody- | | |
|---|---|---|
| drug conjugate | Cancer Antigen | Cancer Type |
| oregovomab | CA125 | |
| girentuximab | CAIX | |
| obinutuzumab | CD20 | |
| ofatumumab | CD20 | |
| rituximab | CD20 | |
| alemtuzumab | CD52 | |
| Ipilimumab | cytotoxic T-lymphocyte-associated | |
| | antigen 4 (CTLA-4) | |
| tremelimumab | CTLA-4 | |
| Cetuximab | epidermal growth factor receptor | |
| | (EGFR) | |
| necitumumab | EGFR | |
| panitumumab | EGFR | |
| zalutumumab | EGFR | |
| edrecolomab | epithelial cell adhesion molecule | |
| | (EpCAM) (17-1A) | |
| farletuzumab | FR-alpha | |
| Pertuzumab | human epidermal growth factor | |
| | receptor 2 (HER2) | |
| trastuzumab | HER2 | |
| rilotumumab | HGF | |
| figitumumab | IGF-1 | |
| Ganitumab | IGF1R | |
| durvalumab | IGG1K | |
| bavituximab | Phosphatidylserine | |
| onartuzumab | scatter factor receptor kinase | |
| bevacizumab | vascular endothelial growth factor-A | |
| | (VEGF-A) | |
| ramucirumab | vascular endothelial growth factor | |
| | Receptor 2 (VEGFR2) | |
| blinatumomab | CD19 | acute lymphoblastic leukemia |
| | | (ALL) |
| Rituximab; | CD20 | non-Hodgkin's lymphoma |
| ofatumumab; | | (NHL), chronic lymphocytic |
| ibritumomab (e.g., ⁹⁰Y- | | leukemia (CLL) |
| ibritumomab; | | B-cell NHL |
| tositumomab (e.g., ¹³¹I- | | pre-B ALL |
| tositumomab | | |
| brentuximab (e.g., | CD30 | Hodgkin's lymphoma |
| brentuximab vedotin | | |
| gemtuzumab (e.g., | CD33 | acute myelogenous leukemia |
| gemtuzumab ozogamicin | | (AML) |
| Alemtuzumab | CD52 | CLL |
| Ipilimumab | cytotoxic T-lymphocyte-associated | Unresectable or metastatic |
| | antigen 4 (CTLA-4) | melanoma |
| cetuximab; | epidermal growth factor receptor | colorectal cancer (CRC) |
| panitumumab | (EGFR) | Head and Neck |
| Catumaxomab | epithelial cell adhesion molecule | Malignant ascites |
| | (EpCAM) | |
| trastuzumab; | human epidermal growth factor | Breast |
| pertuzumab | receptor 2 (HER2) | |
| nivolumab; | programmed cell death receptor 1 | Metastatic melanoma, non- |
| pembrolizumab | (PD-1) | small cell lung cancer |
| | | (NSCLC) |
| Bevacizumab | vascular endothelial growth factor | Breast, Cervical |
| | (VEGF) | CRC, NSCLC |
| | | renal cell carcinoma (RCC), |
| | | Ovarian |
| | | Glioblastoma |
| Ramucirumab | vascular endothelial growth factor | Gastric |
| | receptor 2 (VEGF-R2) | NSCLC |
| Epratuzumab; | CD22 | acute lymphoblastic leukemia |
| moxetumomab; | | (ALL) |
| inotuzumab (e.g., | | |
| inotuzumab ozogamicin) | | |
| MEDI9447 | CD73 | Advanced solid tumors |
| Urelumab; | CD137 | Advanced solid tumors |
| utomilumab (PF- | | |
| 05082566) | | |
| Elotuzumab | CD2 subset 1 (CS1) | Multiple myeloma |
| Tremelimumab | cytotoxic T-lymphocyte-associated | Malignant mesothelioma |
| | antigen 4 (CTLA-4) | |
| Necitumumab | epidermal growth factor receptor | non-small cell lung cancer |
| | (EGFR) | (NSCLC) |
| dinutuximab; hu3F8; | disialoganglioside GD2 (GD2) | Neuroblastoma |
| hu14.18-IL-2; | | Retinoblastoma |
| 3F8/OKT3BsAb | | Melanoma other solid tumors |
| Racotumomab | Idiotype (NeuGcGM3) | NSCLC, Breast |
| | | Melanoma |
| Lirilumab | killer cell immunoglobulin-like | Lymphoma |
| | receptor (KIR) | |
| BMS-986016 | lymphocyte activation gene 3 (LAG- | Breast, Hematological, |
| | 3) | Advanced solid tumors |
| Onartuzumab | N-methyl-N′-nitroso-guanidine | NSCLC |
| | human osteosarcoma transforming | |
| | gene (MET) | |
| abagovomab; | mucin 16 (MUC16) | Ovarian |
| oregovomab | | |
| pidilizumab; | programmed cell death receptor 1 | B-cell lymphoma |
| AMP-224; AMP-514 | (PD-1) | Melanoma, CRC |
| BMS-936559; | programmed cell death receptor | NSCLC, renal cell carcinoma |
| atezolizumab; | ligand 1 (PD-L1) | (RCC) |
| durvalumab; avelumab | | Bladder, Breast |
| | | Melanoma, squamous cell |
| | | carcinoma of the head and |
| | | neck (SCCHN) |
| naptumomab (e.g., | 5T4 | RCC, CRC |
| naptumomab estafenatox) | | Prostate |

In certain embodiments, the antibody is, or is derived from, trastuzumab (e.g., comprising a heavy chain amino acid sequence of SEQ TD NO: 114 and a light chain amino acid sequence of SEQ TD NO: 115). In certain embodiments, the antibody includes a UAA at one or more positions corresponding to P14, G66, D73, L155, A121, K124, T138, A143, V157, T158, S160, T167, T198, N204, V205, N206, K213, D215, 1256, K277, Y281, K291, K293, N300, or F407 of an antibody heavy chain or heavy chain fragment (e.g., at the corresponding positions in SEQ TD NO: 114). In certain embodiments, the antibody includes a UAA at one or more positions corresponding to V15, T20, R24, S60, S66, K107, T109, V10, A1111, Q147, L154, G157, K169 A193, V205, T206, or 5S208 of an antibody light chain or light chain fragment (e.g., at the corresponding positions in SEQ ID NO: 115). Additional sites for UAA incorporation are described in International (PCT) Application No. PCT/US2021/049953, which is incorporated by reference herein.

In certain embodiments, the antibody has a binding affinity (K_{D}) for a target antigen of at least 20 nM, 15 nM, 10 nM, 9 nM, 8 nM, 7 nM, 6 nM, 5 nM, 4 nM, 3 nM, 2 nM, 1 nM, 0.75 nM, 0.5 nM, 0.1 nM, 0.075 nM, or 0.05 nM or lower, as measured using standard binding assays, for example, ELISA, surface plasmon resonance or bio-layer interferometry. In certain embodiments, the antibody binds a target antigen with a K_{D} of from about 20 nM to about 0.05 nM, from about 20 nM to about 0.075 nM, from about 20 nM to about 0.1 nM, from about 20 nM to about 0.5 nM, from about 20 nM to about 1 nM, from about 10 nM to about 0.05 nM, from about 10 nM to about 0.075 nM, from about 10 nM to about 0.1 nM, from about 10 nM to about 0.5 nM, from about 10 nM to about 1 nM, from about 5 nM to about 0.05 nM, from about 5 nM to about 0.075 nM, from about 5 nM to about 0.1 nM, from about 5 nM to about 0.5 nM, from about 5 nM to about 1 nM, from about 3 nM to about 0.05 nM, from about 3 nM to about 0.075 nM, from about 3 nM to about 0.1 nM, from about 3 nM to about 0.5 nM, from about 3 nM to about 1 nM, from about 3 nM to about 2 nM, from about 2 nM to about 0.05 nM, from about 2 nM to about 0.075 nM, from about 2 nM to about 0.1 nM, from about 2 nM to about 0.5 nM, from about 2 nM to about 1 nM, from about 1 nM to about 0.05 nM, from about 1 nM to about 0.075 nM, from about 1 nM to about 0.1 nM, from about 1 nM to about 0.5 nM, from about 0.5 nM to about 0.05 nM, from about 0.5 nM to about 0.075 nM, from about 0.5 nM to about 0.1 nM, from about 0.1 nM to about 0.05 nM, from about 0.1 nM to about 0.075 nM, or from about 0.075 nM to about 0.05 nM, or from about 0.05 nM to about 0.035 nM, as measured using standard binding assays, for example, ELISA, surface plasmon resonance or bio-layer interferometry.

In certain embodiments, the antibody has a binding affinity (K_{D}) for a target antigen that is within 0.1 fold, 0.2 fold, 0.3 fold, 0.4 fold, 0.5 fold, 1.0 fold, 1.5 fold, 2.0 fold, 3.0 fold, 4.0 fold, 5.0 fold, 6.0 fold, 8.0 fold, or 10.0 fold of the binding affinity for the target antigen of a reference antibody, wherein the reference antibody is an otherwise identical antibody that does not comprise the UAA and/or branched linker, as measured using standard binding assays, for example, ELISA, surface plasmon resonance or bio-layer interferometry.

It is contemplated that a protein (e.g., an antibody) may have comparable or even improved stability relative a reference protein, wherein the reference protein is an otherwise identical protein that does not comprise the UAA, branched linker, and/or molecule conjugated to the UAA.

In certain embodiments, the protein (e.g., antibody) has off-target binding or activity that is within 0.1 fold, 0.2 fold, 0.3 fold, 0.4 fold, 0.5 fold, 1.0 fold, 1.5 fold, 2.0 fold, 3.0 fold, 4.0 fold, 5.0 fold, 6.0 fold, 8.0 fold, or 10.0 fold of the off-target binding or activity of a reference protein, wherein the reference protein is an otherwise identical protein that does not comprise any UAA and/or branched linker, does not comprise the same UAA and/or branched linker, does not comprise the same UAA and/or branched linker at the same position, and/or does not comprise the same molecule conjugated to the UAA and/or branched linker. In certain embodiments, the protein (e.g. antibody) has off-target binding or activity that is 0.1 fold, 0.2 fold, 0.3 fold, 0.4 fold, 0.5 fold, 1.0 fold, 1.5 fold, 2.0 fold, 3.0 fold, 4.0 fold, 5.0 fold, 6.0 fold, 8.0 fold, or 10.0 fold less than the off-target binding or activity of a reference protein, wherein the reference protein is an otherwise identical protein that does not comprise any UAA and/or branched linker, does not comprise the same UAA and/or branched linker, does not comprise the same UAA and/or branched linker at the same position, and/or does not comprise the same molecule conjugated to the UAA and/or branched linker. Off-target binding or activity may be measured by any assays known in the art

In certain embodiments, the protein (e.g., antibody) has an efficacy or therapeutic activity (e.g., IC50) that is within 0.1 fold, 0.2 fold, 0.3 fold, 0.4 fold, 0.5 fold, 1.0 fold, 1.5 fold, 2.0 fold, 3.0 fold, 4.0 fold, 5.0 fold, 6.0 fold, 8.0 fold, or 10.0 fold of the efficacy or therapeutic activity of a reference protein, wherein the reference protein is an otherwise identical protein that does not comprise any UAA and/or branched linker, does not comprise the same UAA and/or branched linker, does not comprise the same UAA and/or branched linker at the same position, and/or does not comprise the same molecule conjugated to the UAA and/or branched linker. In certain embodiments, the protein (e.g., antibody) has an efficacy or therapeutic activity (e.g., IC50) that is 0.1 fold, 0.2 fold, 0.3 fold, 0.4 fold, 0.5 fold, 1.0 fold, 1.5 fold, 2.0 fold, 3.0 fold, 4.0 fold, 5.0 fold, 6.0 fold, 8.0 fold, or 10.0 fold greater than the efficacy or therapeutic activity of a reference protein, wherein the reference protein is an otherwise identical protein that does not comprise any UAA and/or branched linker, does not comprise the same UAA and/or branched linker, does not comprise the same UAA and/or branched linker at the same position, and/or does not comprise the same molecule conjugated to the UAA and/or branched linker. Efficacy or therapeutic activity may be measured by any assays known in the art.

### II. Unnatural Amino Acids (UAAs)

The invention relates to unnatural amino acids (UAAs) and their incorporation into proteins (e.g. antibodies).

As used herein, an unnatural amino acid refers to any amino acid, modified amino acid, or amino acid analogue other than the following twenty genetically encoded alpha-amino acids: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine. See, e.g., Biochemistry by L. Stryer, 3rd ed. 1988, Freeman and Company, New York, for structures of the twenty natural amino acids. The term unnatural amino acid also includes amino acids that occur by modification (e.g. post-translational modifications) of a natural amino acid but are not themselves naturally incorporated into a growing polypeptide chain by the translation complex.

Because unnatural amino acids typically differ from natural amino acids only in the structure of the side chain, unnatural amino acids may, for example, form amide bonds with other amino acids in the same manner in which they are formed in naturally occurring proteins. However, the unnatural amino acids have side chain groups that distinguish them from the natural amino acids. For example, the side chain may comprise an alkyl-, aryl-, acyl-, keto-, azido-, hydroxyl-, hydrazine, cyano-, halo-, hydrazide, alkenyl, alkyl, ether, thiol, seleno-, sulfonyl-, borate, boronate, phospho, phosphono, phosphine, heterocyclic, enone, imine, aldehyde, ester, thioacid, hydroxylamine, amine, and the like, or any combination thereof. Other non-naturally occurring amino acids include, but are not limited to, amino acids comprising a photoactivatable cross-linker, spin-labeled amino acids, fluorescent amino acids, metal binding amino acids, metal-containing amino acids, radioactive amino acids, amino acids with novel functional groups, amino acids that covalently or noncovalently interact with other molecules, photocaged and/or photoisomerizable amino acids, amino acids comprising biotin or a biotin analogue, glycosylated amino acids such as a sugar substituted serine, other carbohydrate modified amino acids, keto-containing amino acids, amino acids comprising polyethylene glycol or polyether, heavy atom substituted amino acids, chemically cleavable and/or photocleavable amino acids, amino acids with an elongated side chains as compared to natural amino acids, including but not limited to, polyethers or long chain hydrocarbons, including but not limited to, greater than about 5 or greater than about 10 carbons, carbon-linked sugar-containing amino acids, redox-active amino acids, amino thioacid containing amino acids, and amino acids comprising one or more toxic moiety.

In addition to unnatural amino acids that contain novel side chains, unnatural amino acids also optionally comprise modified backbone structures.

Many unnatural amino acids are based on natural amino acids, such as tyrosine, glutamine, phenylalanine, and the like. Tyrosine analogs include para-substituted tyrosines, ortho-substituted tyrosines, and meta substituted tyrosines, wherein the substituted tyrosine comprises a keto group (including but not limited to, an acetyl group), a benzoyl group, an amino group, a hydrazine, an hydroxyamine, a thiol group, a carboxy group, an isopropyl group, a methyl group, a C₆-C₂₀ straight chain or branched hydrocarbon, a saturated or unsaturated hydrocarbon, an O-methyl group, a polyether group, a nitro group, or the like. In addition, multiply substituted aryl rings are also contemplated. Glutamine analogs include, but are not limited to, α-hydroxy derivatives, γ-substituted derivatives, cyclic derivatives, and amide substituted glutamine derivatives. Exemplary phenylalanine analogs include, but are not limited to, para-substituted phenylalanines, ortho-substituted phenylalanines, and meta-substituted phenylalanines, wherein the substituent comprises a hydroxy group, a methoxy group, a methyl group, an allyl group, an aldehyde, an azido, an iodo, a bromo, a keto group (including but not limited to, an acetyl group), or the like. Specific examples of unnatural amino acids include, but are not limited to, a p-acetyl-L-phenylalanine, a p-propargyl-phenylalanine, O-methyl-L-tyrosine, an L-3-(2-naphthyl)alanine, a 3-methyl-phenylalanine, an O-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, an isopropyl-L-phenylalanine, and a p-propargyloxy-phenylalanine, and the like.

Examples of structures of a variety of unnatural amino acids are provided in U.S. Patent Application Publication Nos. 2003/0082575 and 2003/0108885, PCT Publication No. WO 2002/085923, and Kiick et al. (2002) PROC. NATL. ACAD. SCI. USA 99:19-24.

Any suitable unnatural amino acid can be used with the methods described herein for incorporation into a protein (e.g., an antibody) of interest.

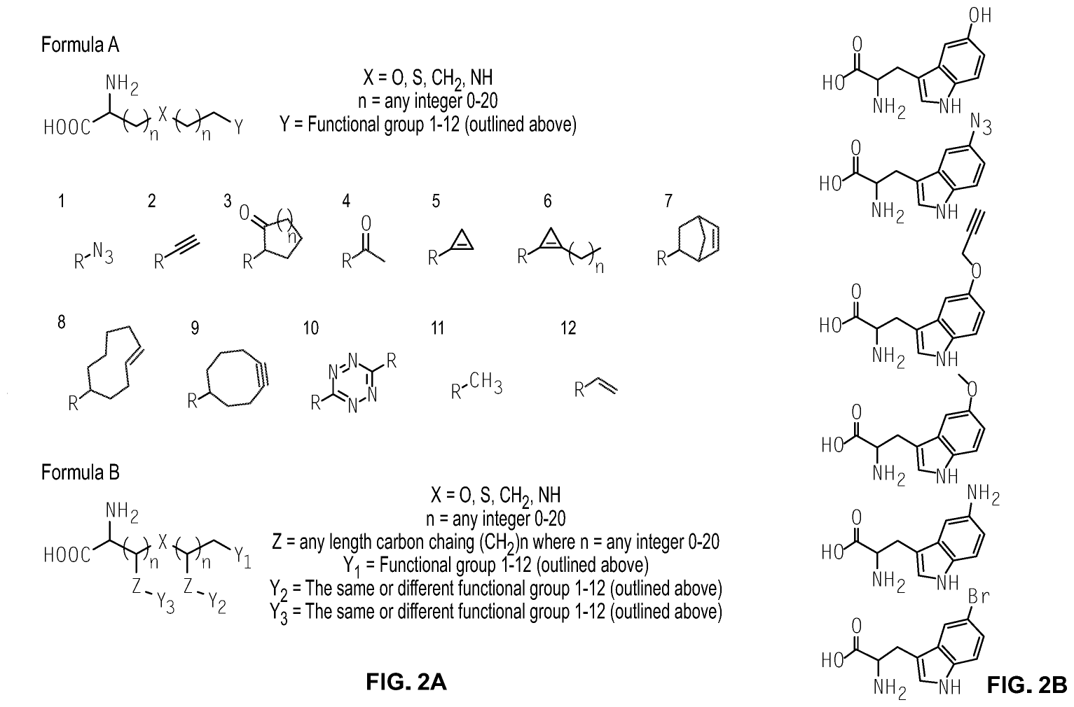

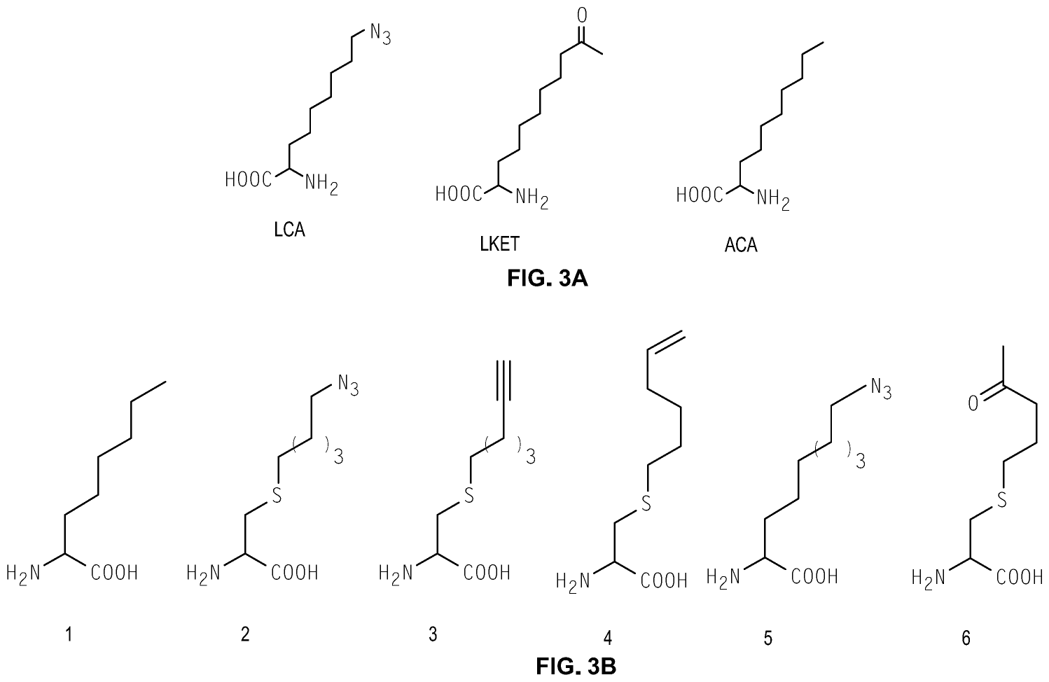

The unnatural amino acid may be a leucine analog (also referred to herein as a derivative). In certain embodiments, the leucine analog is a non-naturally occurring leucine analog. In certain embodiments, the inventions described herein may utilize a leucine analog depicted in FIG. 2A, or a composition comprising the leucine analog. For example, Formula A in FIG. 2A depicts an amino acid analog containing a side chain including a carbon containing chain n units (0-20 units) long. An O, S, CH₂, or NH is present in at position X, and another carbon containing chain of n units (0-20 units) long can follow. A functional group Y is attached to the terminal carbon of second carbon containing chain (for example, functional groups 1-12 as depicted in FIG. 2A, where R represents a linkage to the terminal carbon atom the second carbon containing side chain). In one example, these functional groups can be used for bioconjugation of any amenable ligand to any protein of interest that is amenable to site-specific UAA incorporation. Formula B in FIG. 2A depicts a similar amino acid analog containing an side chains denoted as either Z—Y₂ or Z—Y₃ attached to the second carbon containing chain or the first carbon containing chain, respectively. Z represents a carbon chain comprising (CH₂)n units, where n is any integer from 0-20. Y₂ or Y₃, independently, can be the same or different groups as those of Yi. Exemplary UAAs are included in FIG. 2B. Similarly, inventions described herein may utilize a leucine analog depicted in FIG. 3A (LCA, LKET, or ACA), or a composition comprising the leucine analog depicted in FIG. 3A. Additional exemplary leucine analogs include those selected from linear alkyl halides and linear aliphatic chains comprising a functional group, for example, an alkyne, azide, cyclopropene, methylcyclopropene, alkene, ketone, aldehyde, diazirine, or tetrazine functional group, as well as structures 1-6 shown in FIG. 3B. However, it is contemplated that the amino and carboxylate groups both attached to the first carbon of any amino acid shown in FIG. 2A, 3A, or 3B would constitute portions of peptide bonds when the leucine analog is incorporated into a protein or polypeptide chain.

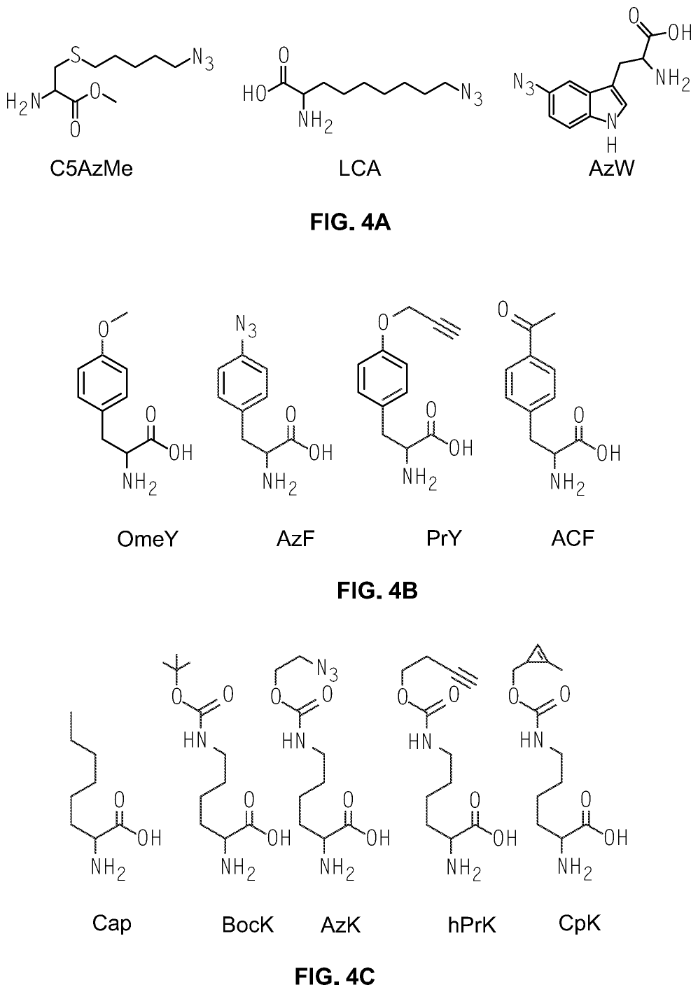

In addition, the leucine analogs set forth in FIG. 4A, referred to as C5AzMe and LCA can be used in the practice of the invention. Methods for preparing leucine analogs, e.g., C5AzMe or LCA, are described in International (PCT) Publication No. WO2021026506.

In certain embodiments, the unnatural amino acid is a tryptophan analog (also referred to herein as a derivative). In certain embodiments, the tryptophan analog is a non-naturally occurring tryptophan analog. Exemplary tryptophan analogs include 5-azidotryptophan, 5-propargyloxytryptophan, 5-aminotryptophan, 5-methoxytryptophan, 5-O-allyltryptophan or 5-bromotryptophan. Additional exemplary tryptophan analogs are depicted in FIG. 2B. However, it is contemplated that the amino and carboxylate groups both attached to the first carbon of the tryptophan analogs in FIG. 2B would constitute portions of peptide bonds when the tryptophan analog is incorporated into a protein or polypeptide chain.

In addition, the tryptophan analog set forth in FIG. 4A, referred to as AzW, can be used in the practice of the invention. Methods for preparing tryptophan analogs, e.g., AzW, are described in International (PCT) Publication No. WO2021026506.

In certain embodiments, the unnatural amino acid is a tyrosine analog (also referred to herein as a derivative). In certain embodiments, the tyrosine analog is a non-naturally occurring tyrosine analog. Exemplary tyrosine analogs include o-methyltyrosine (OmeY), p-azidophenylalanine (AzF), o-propargyltyrosine (OpropY or PrY), and p-acetylphenylalanine (AcF). Exemplary tryptophan analogs are depicted in FIG. 4B.

In certain embodiments, the unnatural amino acid is a pyrrolysine analog (also referred to herein as a derivative). In certain embodiments, the pyrrolysine analog is a non-naturally occurring pyrrolysine analog. Exemplary pyrrolysine analogs include aminocaprylic acid (Cap), H-Lys(Boc)-OH (Boc-Lysine, BocK), azidolysine (AzK), H-propargyl-lysine (hPrK), and cyclopropenelysine (CpK). Exemplary pyrrolysine analogs are depicted in FIG. 4C.

Many unnatural amino acids are commercially available, e.g., from Sigma-Aldrich (St. Louis, Mo., USA), Novabiochem (Darmstadt, Germany), or Peptech (Burlington, Mass., USA). Those that are not commercially available can be synthesized using standard methods known to those of ordinary skill in the art. For organic synthesis techniques, see, e.g., Organic Chemistry by Fessendon and Fessendon, (1982, Second Edition, Willard Grant Press, Boston Mass.); Advanced Organic Chemistry by March (Third Edition, 1985, Wiley and Sons, New York); and Advanced Organic Chemistry by Carey and Sundberg (Third Edition, Parts A and B, 1990, Plenum Press, New York). Additional exemplary publications describing the synthesis of unnatural amino acids appear in PCT Publication No. WO2002/085923, U.S. Patent Application Publication No. 2004/0198637, Matsoukas et al. (1995) J. MED. CHEM. 38:4660-4669, King et al. (1949) J. CHEM. SOC. 3315-3319, Friedman et al. (1959) J. AM. CHEM. SOC. 81:3750-3752, Craig et al. (1988) J. ORG. CHEM. 53:1167-1170, Azoulay et al. (1991) EUR. J. MED. CHEM. 26:201-5, Koskinen et al. (1989) J. ORG. CHEM. 54:1859-1866, Christie et al. (1985) J. ORG. CHEM. 50:1239-1246, Barton et al. (1987) TETRAHEDRON 43:4297-4308, and Subasinghe et al. (1992) J. MED. CHEM. 35:4602-7.

In certain embodiments, where the protein comprises two or more than two UAAs, the protein comprises a first unnatural amino acid (UAA) that is a tryptophan analog (e.g., a non-naturally occurring tryptophan analog) and a second UAA that is a leucine analog (e.g., a non-naturally occurring leucine analog). In certain embodiments, the tryptophan analog is selected from 5-HTP and 5-AzW and/or the leucine analog is selected from LCA and Cys-5-N3.

In certain embodiments, where the protein comprises two or more than two UAAs, the protein comprises a first unnatural amino acid (UAA) that is a tryptophan analog (e.g., a non-naturally occurring tryptophan analog) and a second UAA that is a tyrosine analog (e.g., a non-naturally occurring tyrosine analog). In certain embodiments, the tryptophan analog is selected from 5-HTP and 5-AzW and/or the tyrosine analog is selected from OmeY, AzF, and OpropY UAA.

In certain embodiments, where the protein comprises two or more than two UAAs, the protein comprises a first unnatural amino acid (UAA) that is a tryptophan analog (e.g., a non-naturally occurring tryptophan analog) and a second UAA that is a pyrrolysine analog (e.g., a non-naturally occurring pyrrolysine analog). In certain embodiments, the tryptophan analog is selected from 5-HTP and 5-AzW and/or the pyrrolysine analog is selected from BocK, CpK, AzK, and CpK.

In certain embodiments, the UAA comprises a non-natural aromatic chemical moiety (e.g., a hydroxyl-indole group; an amino-indole group; an aminophenol group; or a hydroxyl-phenol group, e.g., the UAA is 5-hydroxytryptophan (5-HTP), or an analog thereof), and/or the linker comprises a diazonium group (e.g., the linker comprises 4-nitorbenzenediazonium (4NDz); 4-carboxybenzenediazonium (4NeDz) or 4-methoxybenzenediazonium (4MCDz). The UAA and linker may react under conditions suitable to form an azo-linkage via an azo-coupling reaction between the aromatic chemical moiety and the diazonium group. Further methods for conjugation of molecules to UAAs are described, for example, in U.S. Patent Application Publication No. 2018/0360984.

### III. Linkers

The invention relates to linkers, e.g., branched linkers, that enable conjugation of molecules to unnatural amino acids (UAAs) in proteins (e.g. antibodies).

### A. Parent Linker (“PL”)

In certain embodiments, a linker, e.g., a branched linker, contemplated herein includes a parent linker. The parent linker is a chemical moiety with two termini, a first terminus and a second terminus, that is capable of covalently linking together two chemical moieties. Specifically, the parent linker “PL” is capable of, for example, covalently linking an unnatural amino acid and a branching group. An exemplary parent linker has the formula:

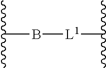

wherein
B is a binding unit; and
L¹ is a chain selected from the group consisting of C₁₋₂₀ alkyl and C₁₋₂₀ heteroalkyl, wherein the chain is optionally substituted by one, two, three or four oxo.

The binding unit (B), as described herein, is capable of conjugating with a reactive group in an unnatural amino acid, for example, via a reaction such as click chemistry. The reactive group in the unnatural amino acid may be, for example, a halogen (e.g., —Cl, —Br, —F, —I), —NH₂, —N₃, —CH₃, C₁₋₆alkyl, C₂₋₆alkenyl, C₂-C₆alkynyl, —OH, —O—(C₁₋₆alkyl)—O—(C₂-C₆alkynyl), a cyclopropene, a methylcyclopropene,

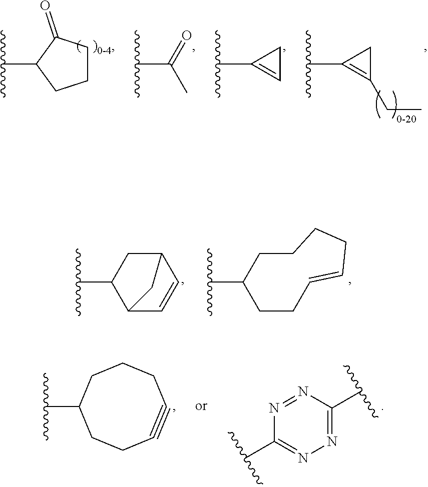

In certain embodiments, the binding unit (B) independently is, or produced by a reaction with, a reactive group selected from the group consisting of dibenzylcyclooctyne (DBCO), (1R,8S,9s)-bicyclo[6.1.0]non-4-yn-9-ylmethanol (BCN), trans-cyclooctene (TCO), azido (N3), alkyne, tetrazine methylcyclopropene, norbornene, hydrazide/hydrazine, and aldehyde. In certain embodiments, the binding unit (B) independently is formed by a 1,3-dipolar cycloaddition reaction, hetero-Diels-Alder reaction, nucleophilic substitution reaction, non-aldol type carbonyl reaction, addition to carbon-carbon multiple bond, oxidation reaction, or click reaction. In certain embodiments, the binding unit (B) independently is formed by a reaction between acetylene and azide, or a reaction between an aldehyde or ketone group and a hydrazine or alkoxyamine. In certain embodiments, the binding unit (B) is a divalent or multivalent linker, known to those of skill in the art. Useful divalent linkers include, but not limited to, alkylene, substituted alkylene, heteroalkylene, substituted heteroalkylene, arylene, substituted arylene, heteroarlyene and substituted heteroarylene linkers.

In certain embodiments, the binding unit (B) may be selected to modulate the release of a UAA or a UAA incorporated in a protein under desired conditions.

In certain embodiments, each L¹ independently is selected from the group consisting of C(O)—(CH₂)₂—C(O), and C(O)—(CH₂)₂—C(O)—NH—(CH₂)₂—(O—(CH₂)₂)₃. In certain embodiments, L¹ is a poly(ethylene glycol) (PEG).

In certain embodiments, the parent linker is, comprises, or is produced from, a peptidyl linker.

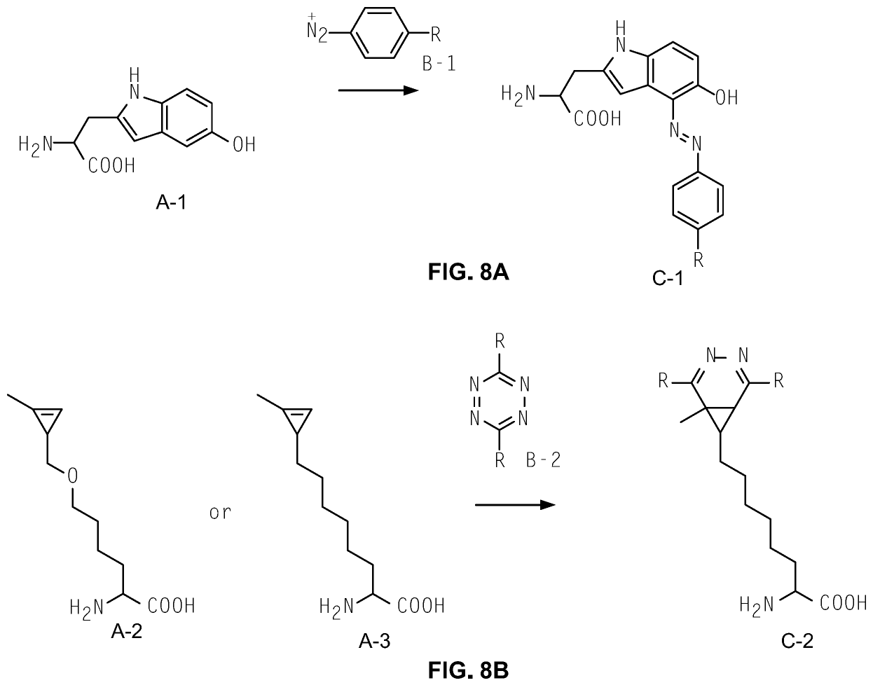

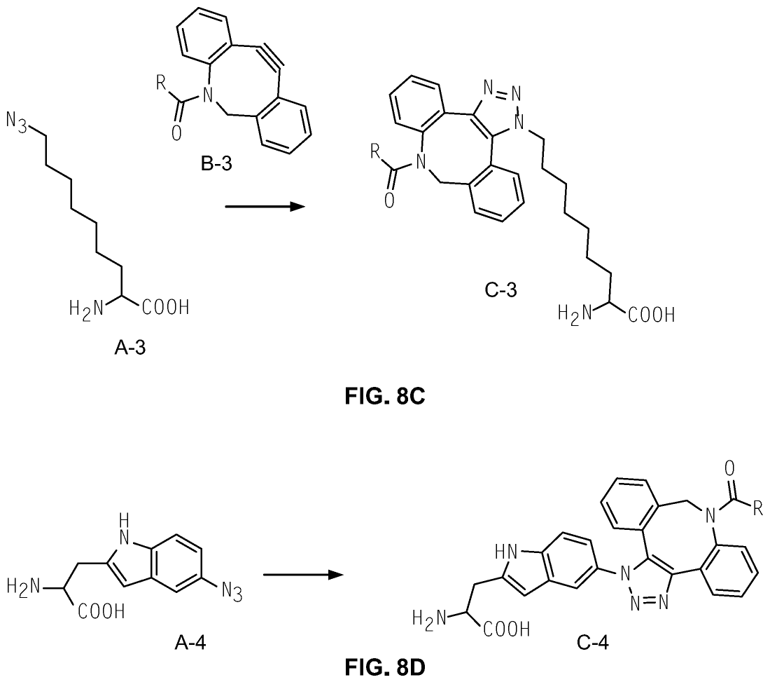

FIG. 8A shows an exemplary reaction between a tryptophan analog unnatural amino acid A-1 and a diazonium linker B-1 to produce a conjugate C-1. FIG. 8B shows an exemplary electron demand Diels-Alder (IEDDA) reaction between a leucine analog unnatural amino acid A-2 or A-3 and tetrazine linker B-2 to produce a conjugate C-2. FIG. 8C shows an exemplary click chemistry reaction between a leucine analog unnatural amino acid A-3 and DBCO linker B-3 to produce a conjugate C-3. FIG. 8D shows an exemplary click chemistry reaction between a tryptophan analog unnatural amino acid A-4 and DBCO linker B-4 to produce a conjugate C-4.

### B. Branching Group (“BG”)

In certain embodiments, a linker, e.g., a branched linker, contemplated herein includes a branching group. The branching group may, for example, be a polyvalent atom. As described herein, the branching group may, for example, be covalently conjugated to multiple chemical moieties, specifically the second terminus of the parent linker and/or the branching unit of the branching linker. In certain embodiments, the polyvalent atom is N or C. In certain embodiments, the polyvalent atom is N. In other certain embodiments, the polyvalent atom is C.

### C. Branching Linker (“BL”)

In certain embodiments, a linker, e.g., a branched linker, contemplated herein includes a branching linker (“BL”). The branching linker is a chemical moiety capable of covalently conjugating with two chemical moieties, for example, a branching group and a molecule, such as a dye, a therapeutic agent, a radionuclide, or a reporter group. The proteins contemplated herein include a plurality of branching linkers. In certain embodiments, each branching linker comprises a branching unit and a conjugating moiety. An example of such a branching linker has the formula:

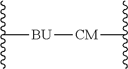

wherein
BU independently is selected from the group consisting of C₁₋₂₀ alkyl and C₁₋₂₀ heteroalkyl, wherein each BU is optionally substituted by one, two or three oxo;
CM independently is selected from the group consisting of a bond, NH, S, OR¹ and R¹; and
R¹ is selected from the group consisting of phenyl, 5-10 membered cycloalkyl, 5-10 membered cycloalkenyl, 5-10 membered cycloalkynyl, 5-6 membered heterocycloalkyl, and 5-6 membered heteroaryl, wherein R¹ is optionally substituted by an oxo.

In certain embodiments, the BU is selected from the group consisting of (CH₂)₂—O—(CH₂)₂—O—(CH₂)₂—O—(CH₂)₂—C(O), (CH₂)₂—O—(CH₂)₂—O—(CH₂)₂—C(O), (CH₂)₂—O—(CH₂)₂—O—(CH₂)₂—O—(CH₂)₂—NH—C(O), and (CH₂)₂—O—(CH₂)₂—O—(CH₂)₂—O—(CH₂)₂—O—(CH₂)₂—NH—C(O). In certain embodiments, the BU is a poly(ethylene glycol) (PEG), optionally substituted by one, two, or three oxo.

In certain embodiments, the CM is selected from the group consisting of thiol, maleimide, tetrazine, sulfohydryl/maleimide reactive group, N-hydroxysuccinimide (NHS), and NHS-ester. In certain embodiments, the CM is a bond.

In certain embodiments, the CM is selected based on orthogonal conjuation with a molecule (M).

Another example of such a branching linker has the formula:

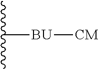

wherein
BU independently is selected from the group consisting of C₁₋₂₀ alkyl and C₁₋₂₀ heteroalkyl, wherein each BU is optionally substituted by one, two or three oxo;
CM independently is selected from the group consisting of a NH₂, SH, OH, O—(C₁₋₃ alkyl), OR¹ and R¹; and
R¹ is selected from the group consisting of phenyl, 5-10 membered cycloalkyl, 5-10 membered cycloalkenyl, 5-10 membered cycloalkynyl, 5-6 membered heterocycloalkyl, and 5-6 membered heteroaryl, wherein R¹ is optionally substituted by an oxo.

In certain embodiments, the BU is selected from the group consisting of (CH₂)₂—O—(CH₂)₂—O—(CH₂)₂—O—(CH₂)₂—C(O), (CH₂)₂—O—(CH₂)₂—O—(CH₂)₂—C(O), (CH₂)₂—O—(CH₂)₂—O—(CH₂)₂—O—(CH₂)₂—NH—C(O), and (CH₂)₂—O—(CH₂)₂—O—(CH₂)₂—O—(CH₂)₂—O—(CH₂)₂—NH—C(O). In certain embodiments, the BU is a poly(ethylene glycol) (PEG), optionally substituted by one, two, or three oxo.

In certain embodiments, the CM is selected from the group consisting of NH₂, SH, OH, and O—CH₃. In certain embodiments, the CM is selected from the group consisting of

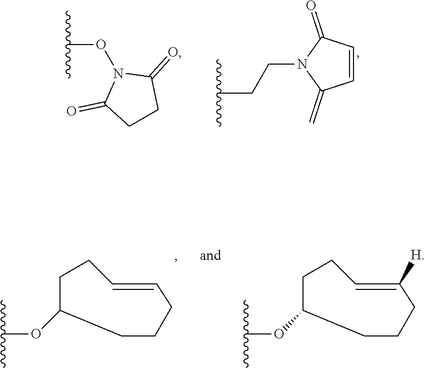

In certain embodiments, CM is, includes, or is produced by a reaction with, a reactive group selected from the group consisting of dibenzylcyclooctyne (DBCO), (1R,8S,9s)-bicyclo[6.1.0]non-4-yn-9-ylmethanol (BCN), trans-cyclooctene (TCO), azido (N3), alkyne, tetrazine methylcyclopropene, norbornene, hydrazide/hydrazine, and aldehyde.

In certain embodiments, the CM may be selected to modulate the release of a molecule (e.g., a payload) under desired conditions.

### D. Exemplary Linkers

The linkers described herein (e.g., a parent linker, a branching linker, or a combination thereof) may be a cleavable linker or a non-cleavable linker. Optionally or in addition, the linker may be a flexible linker or an inflexible linker. The linker may be a length sufficiently long to allow the molecule and the protein to be linked without steric hindrance from one another and sufficiently short to retain the intended activity of the protein. The linker may be sufficiently hydrophilic to avoid or minimize instability or insolubility of the protein. The linker may be sufficiently stable in vivo (e.g., it is not cleaved by serum, enzymes, etc.) to permit the protein to be operative (e.g., selectively operative) in vivo.

The linkers described herein (e.g., parent linker, branching linker) may be from about 1 angstroms (Å) to about 150 Å in length, or from about 1 Å to about 120 Å in length, or from about 5 Å to about 110 Å in length, or from about 10 Å to about 100 Å in length. The linker may be greater than about 2, 3, 4, 5, 6, 7, 8, 9, 10, 11, 12, 13, 14, 15, 16, 17, 18, 19, 20, 25, 27, 30 or greater angstroms in length and/or less than about 110, 100, 90, 85, 80, 75, 70, 65, 60, 55, 50, 45, 43, 42, 41, 40, 39, 38, 37, 36, 35, 34, 33, 32, 31, or fewer Å in length. Furthermore, the linker may be about 5, 10, 15, 20, 25, 30, 35, 40, 45, 50, 55, 60, 65, 70, 75, 80, 85, 90, 95, 100, 110, and 120 Ain length.

The linkers described herein (e.g., a parent linker, a branching linker, or a combination thereof) may include a water soluble polymer. The water soluble polymer may be any structural form including but not limited to linear, forked or branched. Typically, the water soluble polymer is a poly(alkylene glycol), such as poly(ethylene glycol) (PEG), but other water soluble polymers can also be employed. The term “PEG” is used broadly to encompass any polyethylene glycol molecule, without regard to size or to modification at an end of the PEG.

Any molecular mass for a PEG can be used as practically desired, including but not limited to, from about 50 Daltons (Da) to 100,000 Da or more as desired (including but not limited to, sometimes 100 Da to 100,000 Da, 0.1-50 kDa, or 10-40 kDa). Branched chain PEGs, including but not limited to, PEG molecules with each chain having a MW ranging from 1-100 kDa (including but not limited to, 1-50 kDa or 5-20 kDa) can also be used. A contemplated linker may include any appropriate number of PEG units, e.g., from 2 to 24 PEG units, e.g., PEG2, PEG4, PEG6, PEG8, PEG10, PEG12, or PEG24. A wide range of PEG molecules are described in, including but not limited to, the Shearwater Polymers, Inc. catalog, Nektar Therapeutics catalog.

Generally, at least one terminus of the PEG molecule is available for reaction with the UAA. For example, PEG derivatives bearing alkyne and azide moieties for reaction with amino acid side chains can be used to attach PEG to UAAs as described herein. If the UAA comprises an azide, then the PEG will typically contain either an alkyne moiety to effect formation of the [3+2] cycloaddition product or an activated PEG species (i.e., ester, carbonate) containing a phosphine group to effect formation of the amide linkage. Alternatively, if the UAA comprises an alkyne, then the PEG will typically contain an azide moiety to effect formation of the [3+2] Huisgen cycloaddition product. Similarly, if the UAA comprises a tetrazine, then the PEG will typically contain a strained alkene. Alternatively, if the UAA comprises a strained alkene, then the PEG will typically contain a tetrazine. If the UAA comprises a carbonyl group, the PEG will typically comprise a potent nucleophile (including but not limited to, a hydrazide, hydrazine, hydroxylamine, or semicarbazide functionality) in order to effect formation of corresponding hydrazone, oxime, and semicarbazone linkages, respectively. In other alternatives, a reverse of the orientation of the reactive groups described above can be used, i.e., an azide moiety in the UAA can be reacted with a PEG derivative containing an alkyne.

Many other polymers are also suitable for use in the present disclosure. In some examples, polymer backbones that are water-soluble, with from 2 to about 300 termini, are particularly useful. Examples of suitable polymers include, but are not limited to, other poly(alkylene glycols), such as poly(propylene glycol) (“PPG”), copolymers thereof (including but not limited to copolymers of ethylene glycol and propylene glycol), terpolymers thereof, mixtures thereof, and the like. Although the molecular weight of each chain of the polymer backbone can vary, it is typically in the range of from about 800 Da to about 100,000 Da, often from about 6,000 Da to about 80,000 Da.

As understood in the art, PEG and related polymers may include degradable linkages in the polymer backbone or in the linker group between the polymer backbone and one or more of the terminal functional groups of the polymer molecule. For example, ester linkages formed by the reaction of PEG carboxylic acids or activated PEG carboxylic acids with alcohol groups on a biologically active agent generally hydrolyze under physiological conditions to release the agent. Other hydrolytically degradable linkages include, but are not limited to, carbonate linkages; imine linkages resulted from reaction of an amine and an aldehyde; phosphate ester linkages formed by reacting an alcohol with a phosphate group; hydrazone linkages which are reaction product of a hydrazide and an aldehyde; acetal linkages that are the reaction product of an aldehyde and an alcohol; orthoester linkages that are the reaction product of a formate and an alcohol; peptide linkages formed by an amine group, including but not limited to, at an end of a polymer such as PEG, and a carboxyl group of a peptide; and oligonucleotide linkages formed by a phosphoramidite group, including but not limited to, at the end of a polymer, and a 5′ hydroxyl group of an oligonucleotide. Branched linkers may be used in proteins of the disclosure. A number of different cleavable linkers are known to those of skill in the art. The mechanisms for release of an agent from these linker groups include, for example, irradiation of a photolabile bond and acid-catalyzed hydrolysis. The length of the linker may be predetermined or selected depending upon a desired spatial relationship between the protein and the molecule linked to it.

The linkers described herein (e.g., a parent linker, a branching linker, or a combination thereof) may have a wide range of molecular weight or molecular length. Larger or smaller molecular weight linkers may be used to provide a desired spatial relationship or conformation between the protein and the linked entity. Linkers having longer or shorter molecular length may also be used to provide a desired space or flexibility between the protein and the linked entity. Similarly, a linker having a particular shape or conformation may be utilized to impart a particular shape or conformation to the protein or the linked entity, either before or after the protein reaches its target.

Some examples of water-soluble bifunctional linkers have a dumbbell structure that includes: a) an azide, an alkyne, a hydrazine, a hydrazide, a hydroxylamine, a carbonyl, a tetrazine, or a strained alkene-containing moiety on at least a first end of a polymer backbone; and b) at least a second functional group on a second end of the polymer backbone. The second functional group can be the same or different as the first functional group. The second functional group, in some examples, is not reactive with the first functional group. Provided, in some examples, are water-soluble compounds that comprise at least one arm of a branched molecular structure. For example, the branched molecular structure can be dendritic.

Further illustrative linkers include, for example, malC, thioether, AcBut, valine-citrulline peptide, malC-valine-citrulline peptide, hydrazone, and disulfide.

In certain embodiments, coupling of protein and molecule can be accomplished via a crosslinking agent. There are several intermolecular crosslinking agents which can be utilized, see for example, Means and Feeney, CHEMICAL MODIFICATION OF PROTEINS, Holden-Day, 1974, pp. 39-43. Among these reagents are, for example, N-succinimidyl3-(2-pyridyldithio) propionate (SPDP) or N, N′-(1,3-phenylene)bismaleimide (both of which are highly specific for sulfhydryl groups and form irreversible linkages); N, N′-ethylene-bis-(iodoacetamide) or other such reagent having 6 to 11 carbon methylene bridges (which are relatively specific for sulfhydryl groups); and 1, 5-difluoro-2,4-dinitrobenzene (which forms irreversible linkages with amino and tyrosine groups). Other crosslinking agents useful for this purpose include: p,p′-difluoro-N,N′-dinitrodiphenylsulfone (which forms irreversible crosslinkages with amino and phenolic groups); dimethyl adipimidate (which is specific for amino groups); phenol-1,4-disulfonylchloride (which reacts principally with amino groups); hexamethylenediisocyanate or diisothiocyanate, or azophenyl-p-diisocyanate (which reacts principally with amino groups); glutaraldehyde (which reacts with several different side chains) and disdiazobenzidine (which reacts primarily with tyrosine and histidine); N-3-Maleimidopropanoic acid; N-6-Maleimidocaproic acid; N-11-Maleimidoundecanoic acid, 4-(N-maleimidomethyl)cyclohexane-1-carboxy-6-amidocaproic acid; 4-[(Nmaleimidoethyl)carboxamidoethyl(Peg)4 carboxamidomethyl]cyclohexanecarboxylic acid.

The crosslinking agent may be homobifunctional, i.e., having two functional groups that undergo the same reaction. An example of a homobifunctional crosslinking agent is bismaleimidohexane (“BMH”). BMH contains two maleimide functional groups, which react specifically with sulfhydryl-containing compounds under mild conditions (pH 6.5-7.7). The two maleimide groups are connected by a hydrocarbon chain. Therefore, BMH is useful for irreversible crosslinking of polypeptides that contain cysteine residues. Additional commercially available homobifunctional crosslinking agents include: BSOCOES (Bis(2 [Succinimidooxycarbonyloxy]ethyl) sulfone; DPDPB (1,4-Di-(3′-[2pyridyldithio]-propionamido) butane; DSS (disuccinimidyl suberate); DST (disuccinimidyl tartrate); Sulfo DST (sulfodisuccinimidyl tartrate); DSP (dithiobis(succinimidyl propionate); DTSSP (3,3′-Dithiobis(sulfosuccinimidyl propionate); EGS (ethylene glycol bis(succinimidyl succinate)); BASED (Bis(β-[4-azidosalicylamido]-ethyl)disulfide iodinatable); homobifunctional NHS crosslinking reagents (e.g., Bis(NHS)PEO-5 (bis N-succinimidyl-[pentaethylene glycol]ester); and homobifuctional isothiocyanate derivatives of PEG or dextran polymers.

Heterobifunctional crosslinking agents have two different functional groups, for example an amine-reactive group and a thiol-reactive group, that will crosslink two moieties having free amines and thiols, respectively. The most common commercially available heterobifunctional crosslinking agents have an amine reactive N-hydroxysuccinimide ester as one functional group, and a sulfhydryl reactive group as the second functional group. The most common sulfhydryl reactive groups are maleimides, pyridyl disulfides and active halogens. One of the functional groups can be a photoactive aryl nitrene, which upon irradiation reacts with a variety of groups. Exemplary heterobifunctional crosslinking agents include succinimidyl 4-(N maleimidomethyl)cyclohexane-1-carboxylate (“SMCC”), Succinimidyl-4-(N maleimidomethyl)-cyclohexane-1-carboxy(6-amidocaproate) (“LC-SMCC”), N maleimidobenzoyl-N-hydroxysuccinimide ester (“MBS”), and succinimide 4-(p-maleimidophenyl) butyrate (“SMPB”), an extended chain analog of MBS. The succinimidyl group of these crosslinking agents reacts with a primary amine forming an amide bond, and the thiol-reactive maleimide forms a covalent thioether bond with the thiol group (e.g., of a cysteine).

Additional exemplary crosslinking agents include: BS3 ([Bis(sulfosuccinimidyl)suberate], which is a homobifunctional N-hydroxysuccinimide ester that targets accessible primary amines; NHS/EDC (N-hydroxy-succinimide and N-ethyl-′(dimethylaminopropyl)carbodimide, which allows for the conjugation of primary amine groups with carboxyl groups); sulfoEMCS ([N-e-Maleimido-caproic acid]hydrazide, which includes heterobifunctional reactive groups (a maleimide and an NHS-ester) that are reactive toward sulfhydryl and amino groups; hydrazide, which is useful for useful for linking carboxyl groups on exposed carbohydrates to primary amines; SATA (N-succinimidyl-S-acetylthioacetate), which is reactive towards amines and adds protected sulfhydryl groups; monofluoro cyclooctyne (MFCO); bicyclo[6.1.0]nonyne (BCN); N succinimidyl-S-acetylthiopropionate (SATP); maleimido and dibenzocyclooctyne ester (a DBCO ester); and EDC (1-Ethyl-3-[3-dimethylaminopropyl]carbodiimide hydrochloride).

The length of these crosslinking agents can be varied by the use of polymeric regions between the two reactive groups, which typically take the form of chemical linkers such as polymeric ethylene glycol or simple carbon chains, but can also include sugars, amino acids or peptides, or oligonucleotides. Polymer chain lengths of from 5 to 50 nm are typical, but can be shorter or longer as needed. For example, the crosslinking agent may comprise a<2 carbon chain arm, a 2-5 carbon chain arm, or a 3-6 carbon chain arm.

Crosslinking agents often have low solubility in water. A hydrophilic moiety, such as a sulfonate group, may be added to the crosslinking agent to improve its water solubility. Sulfo-MBS and sulfo-SMCC are examples of crosslinking agents modified for water solubility.

Many crosslinking agents yield a conjugate that is essentially non-cleavable under cellular conditions. However, some crosslinking agents contain a covalent bond, such as a disulfide, that is cleavable under cellular conditions. For example, Traut's reagent, dithiobis(succinimidylpropionate) (“DSP”), and N-succinimidyl 3-(2-pyridyldithio) propionate (“SPDP”) are well-known cleavable crosslinking agents. Direct disulfide linkage may also be useful.

Numerous crosslinking agents, including the ones discussed above, are commercially available. Detailed instructions for their use are readily available from the commercial suppliers. A general reference on protein cross-linking and conjugate preparation is: Wong, CHEMISTRY OF PROTEIN CONJUGATION AND CROSS-LINKING, CRC Press (1991).

In certain embodiments, the linker comprises a polypeptide linker that connects or fuses the molecule to the protein. When a polypeptide linker is employed, the linker may comprise hydrophilic amino acid residues, such as Gln, Ser, Gly, Glu, Pro, His and Arg. In certain embodiments, the linker is a peptide containing 1-25 amino acid residues, 1-20 amino acid residues, 2-15 amino acid residues, 3-10 amino acid residues, 3-7 amino acid residues, 4-amino acid residues, 4-20 amino acid residues, 4-15 amino acid residues, 4-10 amino acid residues, 5-25 amino acid residues, 5-20 amino acid residues, 5-15 amino acid residues, or 5-amino acid residues. Exemplary linkers include glycine and serine-rich linkers, e.g., (GlyGlyPro)ₙ, or (GlyGlyGlyGlySer)ₙ, where n is 1-5. In certain embodiments, the linker comprises, consists, or consists essentially of GGGGS (SEQ ID NO: 116). In certain embodiments, the linker comprises, consists, or consists essentially of GGGGSGGGGS (SEQ ID NO: 117). Additional exemplary linker sequences are disclosed, e.g., in George et al. (2003) PROTEIN ENGINEERING 15:871-879, and U.S. Pat. Nos. 5,482,858 and 5,525,491.

In certain embodiments, the protein derivative provided herein comprises a linker compound, or can be creating using one or more of the linker compounds, identified in Table 2.

TABLE 2
Exemplary linker compounds

| Compound No. | Structure |
|---|---|
| 101 | 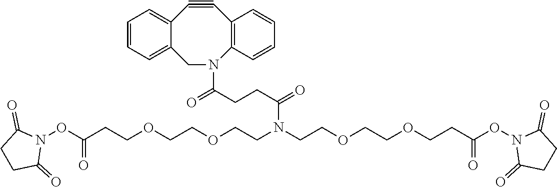 [SMILES: O=C(CCOCCOCCN(CCOCCOCCC(=O)ON1C(=O)CCC1=O)C(=O)CCC(=O)N1Cc2ccccc2C#Cc2ccccc21)ON1C(=O)CCC1=O] |
| 102 | 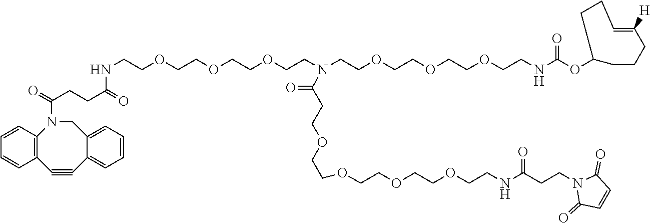 |

### E. Chemical Definitions

The term “oxo” as used herein refers to the radical ═O.

The term “alkyl” as used herein refers to a saturated straight or branched hydrocarbon, such as a straight or branched group of 1-6, 1-4, or 1-3 carbon atoms, referred to herein as C₁₋₆alkyl, C₁₋₄alkyl, and C₁₋₃alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 3-methyl-2-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, etc.

The term “alkenyl” as used herein refers to an unsaturated straight or branched hydrocarbon having at least one carbon-carbon double bond, such as a straight or branched group of 2-6 or 3-4 carbon atoms, referred to herein for example as C₂₋₆alkenyl, and C₃₋₄ alkenyl, respectively. Exemplary alkenyl groups include, but are not limited to, vinyl, allyl, butenyl, pentenyl, etc.

The term “alkynyl” as used herein refers to an unsaturated straight or branched hydrocarbon having at least one carbon-carbon triple bond, such as a straight or branched group of 2-6, or 3-6 carbon atoms, referred to herein as C₂₋₆alkynyl, and C₃₋₆alkynyl, respectively. Exemplary alkynyl groups include, but are not limited to, ethynyl, propynyl, butynyl, pentynyl, hexynyl, methylpropynyl, etc.

The term “alkoxy” as used herein refers to a straight or branched alkyl group attached to an oxygen (alkyl-O—). Exemplary alkoxy groups include, but are not limited to, groups with an alkyl group of 1-6 or 2-6 carbon atoms, referred to herein as C₁₋₆ alkoxy, and C₂-C₆alkoxy, respectively. Exemplary alkoxy groups include, but are not limited to methoxy, ethoxy, isopropoxy, etc.

The term “carbonyl” as used herein refers to the radical —C(O)—.

The term “cycloalkyl” as used herein refers to a monocyclic saturated or partially unsaturated hydrocarbon group of for example 3-6, or 4-6 carbons, referred to herein, e.g., as C₃₋₆cycloalkyl or C₄₋₆cycloalkyl and derived from a cycloalkane. Exemplary cycloalkyl groups include, but are not limited to, cyclohexyl, cyclohexenyl, cyclopentyl, cyclobutyl or, cyclopropyl.

The term “cycloalkyl or cycloalkenyl” as used herein refers to a monocyclic or fused or bridged bicyclic carbocyclic ring system that is not aromatic. Cycloalkenyl rings have one or more units of unsaturation. Exemplary cycloalkyl or cycloalkenyl groups include cyclopropyl, cyclobutyl, cyclopentyl, cyclohexyl, cyclohexenyl, cycloheptyl, cycloheptenyl, norbornyl, adamantyl and decalinyl.

The term “cycloalkynyl” as used herein refers to monovalent, monodentate, non-aromatic hydrocarbon moieties having at least one carbon-atom ring (preferably having from 3 to 7 ring carbon atoms) and at least one carbon-carbon triple bond.

The terms “halo” or “halogen” as used herein refer to F, Cl, Br, or I.

The term “hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl, cycloalkyl, e.g., heterocyclyl, aryl, e.g., heteroaryl, cycloalkenyl, e.g., cycloheteroalkenyl, and the like having from 1 to 5, and particularly from 1 to 3 heteroatoms.

The terms “heteroaryl” or “heteroaromatic group” as used herein refers to a monocyclic aromatic 4-6 membered ring system containing one or more heteroatoms, for example one to three heteroatoms, such as nitrogen, oxygen, and sulfur. Where possible, said heteroaryl ring may be linked to the adjacent radical though carbon or nitrogen. Examples of heteroaryl rings include but are not limited to furan, thiophene, pyrrole, thiazole, oxazole, isothiazole, isoxazole, imidazole, pyrazole, triazole, pyridyl, and pyrimidinyl.

The term “heterocyclyl” or “heterocyclic group” as used herein is art-recognized and refers to saturated or partially unsaturated 4-7 membered ring structures, whose ring structures include one to three heteroatoms, such as nitrogen, oxygen, and sulfur. A heterocycle may be fused to one or more phenyl, partially unsaturated, or saturated rings. Examples of heterocyclyl groups include but are not limited to pyrrolidine, piperidine, morpholine, thiomorpholine, and piperazine.

### IV. Molecules for Conjugation

An unnatural amino acid in a protein (e.g., an antibody) may be used to attach another molecule to the protein. For example, in certain embodiments, a disclosed protein comprises a chemical modification of an unnatural amino acid (UAA), e.g., a conjugation to a molecule. It is contemplated that a protein may comprise one or more UAAs (e.g., one, two, three, four, five, six, seven, eight, nine, ten, or more than ten UAAs, each of which may be the same or different), and similarly, may be conjugated to one or more molecules (e.g., one, two, three, four, five, six, seven, eight, nine, ten, or more than ten molecules, each of which may be the same or different).

Exemplary molecules for conjugation include a label, a dye, a polymer, a water-soluble polymer, a stabilizing agent (e.g., a derivative of polyethylene glycol), a photoactivatable crosslinker, a radionuclide, a cytotoxic compound, a drug, an affinity label, a photoaffinity label, a reactive compound, a resin, a second protein or polypeptide or polypeptide analog (e.g., a therapeutic peptide or polypeptide), an antibody or antibody fragment (e.g., an anti-CD3 antibody or antibody fragment), a metal chelator, a cofactor, a fatty acid, a carbohydrate, a polynucleotide, a DNA (e.g., a DNA oligonucleotide), a RNA (e.g., a DNA oligonucleotide), a LNA (e.g., a LNA oligonucleotide), an antisense polynucleotide, a saccharide, a water-soluble dendrimer, a cyclodextrin, an inhibitory ribonucleic acid (e.g., a small interfering RNA (siRNA), a small nuclear RNA (snRNA), or a non-coding RNA), a biomaterial, a nanoparticle, a spin label, a fluorophore, a metal-containing moiety, a radioactive moiety, a novel functional group, a group that covalently or noncovalently interacts with other molecules, a photocaged moiety, an actinic radiation excitable moiety, a photoisomerizable moiety, biotin, a derivative of biotin, a biotin analogue, a moiety incorporating a heavy atom, a chemically cleavable group, a photocleavable group, an elongated side chain, a carbon-linked sugar, a redox-active agent, an amino thioacid, a toxic moiety, an isotopically labeled moiety, a biophysical probe or biochemical probe (e.g., a PET probe, a fluorescent probe or an EPR probe), a phosphorescent group, a chemiluminescent group, an electron dense group, a magnetic group, an intercalating group, a chromophore, an energy transfer agent, a biologically active agent, a detectable label (e.g., for analysis of uptake in viable cells versus non-viable cells), a small molecule (e.g. a therapeutic small molecule), a quantum dot, a nanotransmitter, an immunomodulatory molecule, a targeting agent, a lipid based structure (e.g., a lipid-based nanoparticle), a microsphere, or any combination of the above.

Additional exemplary molecules for conjugation include any cytotoxic, cytostatic or immunomodulatory drug. Useful classes of cytotoxic or immunomodulatory agents include, for example, antitubulin agents, auristatins, DNA minor groove binders, DNA replication inhibitors, alkylating agents (e.g., platinum complexes such as cis-platin, mono(platinum), bis(platinum) and tri-nuclear platinum complexes and carboplatin), anthracyclines, antibiotics, antifolates, antimetabolites, calmodulin inhibitors, chemotherapy sensitizers, duocarmycins, etoposides, fluorinated pyrimidines, ionophores, lexitropsins, maytansinoids, nitrosoureas, platinols, pore-forming compounds, purine antimetabolites, puromycins, radiation sensitizers, rapamycins, steroids, taxanes, topoisomerase inhibitors, vinca alkaloids, or the like.

Individual cytotoxic or immunomodulatory agents include, for example, an androgen, anthramycin (AMC), asparaginase, 5-azacytidine, azathioprine, bleomycin, busulfan, buthionine sulfoximine, calicheamicin, calicheamicin derivatives, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, DM1, DM4, docetaxel, doxorubicin, etoposide, an estrogen, 5-fluordeoxyuridine, 5-fluorouracil, gemcitabine, gramicidin D, hydroxyurea, idarubicin, ifosfamide, irinotecan, lomustine (CCNU), maytansine, mechlorethamine, melphalan, 6-mercaptopurine, methotrexate, mithramycin, mitomycin C, mitoxantrone, nitroimidazole, paclitaxel, palytoxin, plicamycin, procarbizine, a pyrrolobenzodiazepine, rhizoxin, streptozotocin, tenoposide, 6-thioguanine, thioTEPA, topotecan, vinblastine, vincristine, vinorelbine, VP-16 and VM-26.

In certain embodiments, suitable cytotoxic agents include, for example, DNA minor groove binders (e.g., enediynes and lexitropsins, a CBI compound), duocarmycins, taxanes (e.g., paclitaxel and docetaxel), puromycins, vinca alkaloids, CC-1065, SN-38, topotecan, morpholino-doxorubicin, rhizoxin, cyanomorpholino-doxorubicin, echinomycin, combretastatin, netropsin, epothilone A and B, estramustine, cryptophycins, cemadotin, maytansinoids, discodermolide, eleutherobin, and mitoxantrone.

In certain embodiments, the molecule is an anti-tubulin agent. Examples of anti-tubulin agents include taxanes (e.g., Taxol® (paclitaxel), Taxotere® (docetaxel)), T67 (Tularik) and vinca alkyloids (e.g., vincristine, vinblastine, vindesine, and vinorelbine). Other antitubulin agents include, for example, baccatin derivatives, taxane analogs, epothilones (e.g., epothilone A and B), nocodazole, colchicine and colcimid, estramustine, cryptophycins, cemadotin, maytansinoids, combretastatins, discodermolide, and eleutherobin.

In certain embodiments, the cytotoxic agent is a maytansinoid, another group of anti-tubulin agents. For example, in specific examples, the maytansinoid can be maytansine or DM1.

In certain embodiments, the molecule is an auristatin, such as auristatin E or a derivative thereof. For example, the auristatin E derivative can be an ester formed between auristatin E and a keto acid. For example, auristatin E can be reacted with paraacetyl benzoic acid or benzoylvaleric acid to produce AEB and AEVB, respectively. Other typical auristatin derivatives include AFP, MMAF, and MMAE.

In certain embodiments, the molecule is an antimetabolite. The antimetabolite can be, for example, a purine antagonist (e.g., azothioprine or mycophenolate mofetil), a dihydrofolate reductase inhibitor (e.g., methotrexate), acyclovir, ganciclovir, zidovudine, vidarabine, ribavarin, azidothymidine, cytidine arabinoside, amantadine, dideoxyuridine, iododeoxyuridine, poscarnet, or trifluridine.

In certain embodiments, the payload is tacrolimus, cyclosporine, FU506 or rapamycin. In further examples, the molecule is aldesleukin, alemtuzumab, alitretinoin, allopurinol, altretamine, amifostine, anastrozole, arsenic trioxide, bexarotene, bexarotene, calusterone, capecitabine, celecoxib, cladribine, Darbepoetin alfa, Denileukin diftitox, dexrazoxane, dromostanolone propionate, epirubicin, Epoetin alfa, estramustine, exemestane, Filgrastim, floxuridine, fludarabine, fulvestrant, gemcitabine, gemtuzumab ozogamicin (MYLOTARG), goserelin, idarubicin, ifosfamide, imatinib mesylate, Interferon alfa-2a, irinotecan, letrozole, leucovorin, levamisole, meclorethamine or nitrogen mustard, megestrol, mesna, methotrexate, methoxsalen, mitomycin C, mitotane, nandrolone phenpropionate, oprelvekin, oxaliplatin, pamidronate, pegademase, pegaspargase, pegfilgrastim, pentostatin, pipobroman, plicamycin, porfimer sodium, procarbazine, quinacrine, rasburicase, Rituximab, Sargramostim, streptozocin, tamoxifen, temozolomide, teniposide, testolactone, thioguanine, toremifene, tositumomab, trastuzumab, tretinoin, uracil mustard, valrubicin, vinblastine, vincristine, vinorelbine, or zoledronate.

In certain embodiments, the molecule is an immunomodulatory agent. The immunomodulatory agent can be, for example, ganciclovir, etanercept, tacrolimus, cyclosporine, rapamycin, cyclophosphamide, azathioprine, mycophenolate mofetil or methotrexate. Alternatively, the immunomodulatory agent can be, for example, a glucocorticoid (e.g., cortisol or aldosterone) or a glucocorticoid analogue (e.g., prednisone or dexamethasone). Alternatively, the immunomodulatory agent can be, for example, a Toll-like receptor (TLR) agonist, e.g., a TLR7 or TLR8 agonist, e.g., imiquimod, 852A, hiltonol, resiquimod, 3M-052, CpG oligodeoxynucleotides (CpG ODN), 1V270, or SD-101.

In certain embodiments, the immunomodulatory agent is an anti-inflammatory agent, such as arylcarboxylic derivatives, pyrazole-containing derivatives, oxicam derivatives and nicotinic acid derivatives. Classes of anti-inflammatory agents include, for example, cyclooxygenase inhibitors, 5-lipoxygenase inhibitors, and leukotriene receptor antagonists.

Suitable cyclooxygenase inhibitors include meclofenamic acid, mefenamic acid, carprofen, diclofenac, diflunisal, fenbufen, fenoprofen, indomethacin, ketoprofen, nabumetone, sulindac, tenoxicam and tolmetin. Leukotriene receptor antagonists include calcitriol, and ontazolast.

Suitable lipoxygenase inhibitors include redox inhibitors (e.g., catechol butane derivatives, nordihydroguaiaretic acid (NDGA), masoprocol, phenidone, Ianopalen, indazolinones, naphazatrom, benzofuranol, alkylhydroxylamine), and non-redox inhibitors (e.g., hydroxythiazoles, methoxyalkylthiazoles, benzopyrans and derivatives thereof, methoxytetrahydropyran, boswellic acids and acetylated derivatives of boswellic acids, and quinolinemethoxyphenylacetic acids substituted with cycloalkyl radicals), and precursors of redox inhibitors. Other suitable lipoxygenase inhibitors include antioxidants (e.g., phenols, propyl gallate, flavonoids and/or naturally occurring substrates containing flavonoids, hydroxylated derivatives of the flavones, flavonol, dihydroquercetin, luteolin, galangin, orobol, derivatives of chalcone, 4,2′,4′-trihydroxychalcone, ortho-aminophenols, N-hydroxyureas, benzofuranols, ebselen and species that increase the activity of the reducing selenoenzymes), iron chelating agents (e.g., hydroxamic acids and derivatives thereof, N-hydroxyureas, 2-benzyl-1-naphthol, catechols, hydroxylamines, carnosol trolox C, catechol, naphthol, sulfasalazine, zyleuton, 5-hydroxyanthranilic acid and 4-(omega-arylalkyl)phenylalkanoic acids), imidazole-containing compounds (e.g., ketoconazole and itraconazole), phenothiazines, and benzopyran derivatives. Yet other suitable lipoxygenase inhibitors include inhibitors of eicosanoids (e.g., octadecatetraenoic, eicosatetraenoic, docosapentaenoic, eicosahexaenoic and docosahexaenoic acids and esters thereof, PGE1 (prostaglandin E1), PGA2 (prostaglandin A2), viprostol, 15-monohydroxyeicosatetraenoic, 15-monohydroxy-eicosatrienoic and 15-monohydroxyeicosapentaenoic acids, and leukotrienes B5, C5 and D5), compounds interfering with calcium flows, phenothiazines, diphenylbutylamines, verapamil, fuscoside, curcumin, chlorogenic acid, caffeic acid, 5,8,11,14-eicosatetrayenoic acid (ETYA), hydroxyphenylretinamide, Ionapalen, esculin, diethylcarbamazine, phenantroline, baicalein, proxicromil, thioethers, diallyl sulfide and di-(1-propenyl) sulfide.

Other useful molecules include chemical compounds used in the treatment of cancer. Examples of chemotherapeutic agents include Erlotinib (TARCEVA®, Genentech/OSI Pharm.), Bortezomib (VELCADE®, Millennium Pharm.), Fulvestrant (FASLODEX®, AstraZeneca), Sutent (SU11248, Pfizer), Letrozole (FEMARA®, Novartis), Imatinib mesylate (GLEEVEC®, Novartis), PTK787/ZK 222584 (Novartis), Oxaliplatin (Eloxatin*, Sanofi), 5-FU (5-fluorouracil), Leucovorin, Rapamycin (Sirolimus, RAPAMUNE®, Wyeth), Lapatinib (TYKERB®, GSK572016, Glaxo Smith Kline), Lonafarnib (SCH 66336), Sorafenib (BAY43-9006, Bayer Labs), and Gefitinib (IRESSA®, AstraZeneca), AG1478, AG1571 (SU 5271; Sugen). Further examples of chemotherapeutic agents include alkylating agents such as thiotepa and CYTOXAN® (cyclosphosphamide); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (e.g., bullatacin and bullatacinone); a camptothecin (including the synthetic analog topotecan); bryostatin; callystatin; CC-1065 (including its adozelesin, carzelesin and bizelesin synthetic analogs); cryptophycins (e.g., cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ11 and calicheamicin omegall); dynemicin, including dynemicin A; bisphosphonates, such as clodronate; an esperamicin; as well as neocarzinostatin chromophore and related chromoprotein enediyne antibiotic chromophores). Further anti-cancer agents include aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin, deoxydoxorubicin, epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, and zorubicin. Further anti-cancer agents include anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamniprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (doxetaxel; Rhone-Poulenc Rorer, Antony, France); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Other useful molecules include: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) antiandrogens such as flutamide, nilutamide, bicalutamide, leuprolide, and goserelin; as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-α, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; a topoisomerase 1 inhibitor such as LURTOTECAN®; ABARELIX® rmRH; (ix) anti-angiogenic agents such as bevacizumab (AVASTIN®, Genentech); and (x) pharmaceutically acceptable salts, acids and derivatives of any of the above. Other anti-angiogenic agents include MMP-2 (matrix-metalloproteinase 2) inhibitors, MMP-9 (matrix-metalloproteinase 9) inhibitors, COX-II (cyclooxygenase II) inhibitors, and VEGF receptor tyrosine kinase inhibitors. Examples of VEGF receptor tyrosine kinase inhibitors include 4-(4-bromo-2-fluoroanilino)-6-methoxy-7-(1-methylpiperidin-4-ylmethoxy)quinazoline (ZD6474), 4-(4-fluoro-2-methylindol-5-yloxy)-6-methoxy-7-(3-pyrrolidin-1-ylpropoxy)-quinazoline (AZD2171), vatalanib (PTK787;) and SU11248 (sunitinib).

Additional exemplary molecules for conjugation include an amatoxin, chalicheamicin, DUBA, FAM, MMAD, PBD, and a toxoid.

It is further contemplated that the molecule may itself include additional linkers, e.g., linkers contemplated herein, e.g., a cleavable linker or non-cleavable linker.

In certain embodiments, an unnatural amino acid (UAA) comprises a bioconjugation handle to facilitate conjugation to another molecule. In certain embodiments, a method disclosed herein can be used to site-specifically incorporate two different UAAs, each with a different bioconjugation handle, into a single protein (e.g., a single antibody). In certain embodiments, the two bioconjugation handles can be chosen such that they each can be chemoselectively conjugated to two different labels using mutually orthogonal conjugation chemistries. Such pairs of bioconjugation handles include, for example: azide and alkyne, azide and ketone/aldehyde, azide and cyclopropene, ketone/aldehyde and cyclopropene, 5-hydroxyindole and azide, 5-hydroxyindole and cyclopropene, and 5-hydroxyindole and ketone/aldehyde.

In certain embodiments, when a molecule is conjugated to an antibody, the antibody has an average drug antibody ratio (DAR) of at least 0.5, 0.6, 0.7, 0.8, 0.9, 1.0, 1.5, 1.6, 1.7, 1.8, 1.9, 2.0, 3.5, 3.6, 3.7, 3.8, 3.9, 4.0, 4.5, 5.0, 5.5, 6.0, 6.5, 7.0, 7.5, 8.0, 8.5, 9.0, 9.5, 10.0, 10.5, 11.0, 11.5, 12.0, or greater than 12.0, as measured by hydrophobic interaction chromatography (HIC). In this context, it is understood that drug antibody ratio (DAR) may refer to the ratio of any conjugated molecule to antibody (e.g., a detectable label as well as a drug). In certain embodiments, the antibody has an average drug antibody ratio (DAR) that is within 5%, 10%, 15%, 20%, 25%, 30%, 35%, or 40% of the number of UAAs in the antibody. Similarly, in certain embodiments, when a molecule is conjugated to a protein other than an antibody, the ratio of molecule to protein is at least 0.5, 0.6, 0.7, 0.8, 0.9, 1.0, 1.5, 1.6, 1.7, 1.8, 1.9, 2.0, 3.5, 3.6, 3.7, 3.8, 3.9, 4.0, 4.5, 5.0, 5.5, 6.0, 6.5, 7.0, 7.5, 8.0, 8.5, 9.0, 9.5, 10.0, 10.5, 11.0, 11.5, 12.0, or greater than 12.0, as measured by hydrophobic interaction chromatography (HIC).

In certain embodiments, the protein derivative comprises a compound, or can be creating using one or more of the compounds, identified in Table 3.

TABLE 3
Exemplary compounds

| Compound No. | Structure |
|---|---|
| 201 | 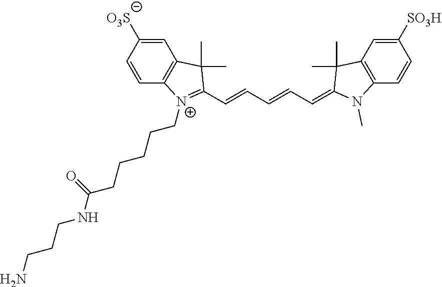 |
| 202 | 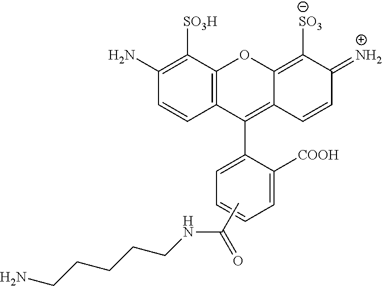 |
| 203 | 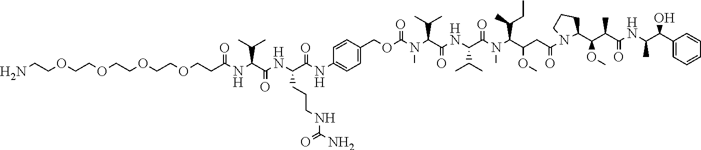 [SMILES: CC[C@H](C)[C@@H](C(CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCOCCOCCOCCN)C(C)C)cc1)C(C)C] |
| 204 | 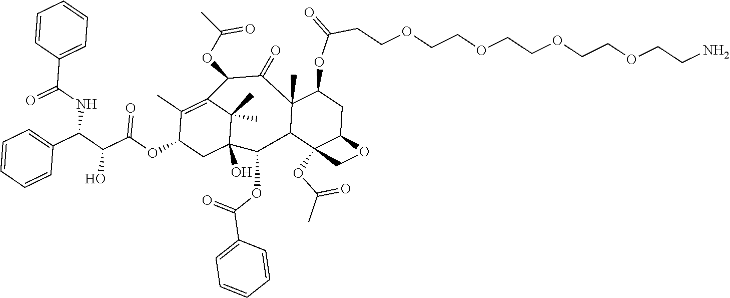 |
| 205 | 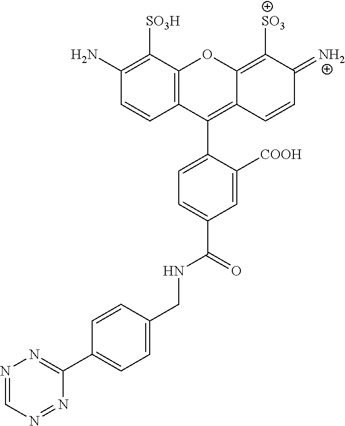 |
| 206 | 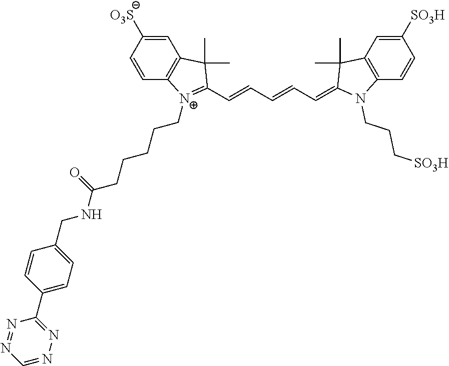 |
| 207 | 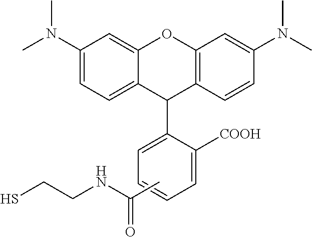 |
| 208 | 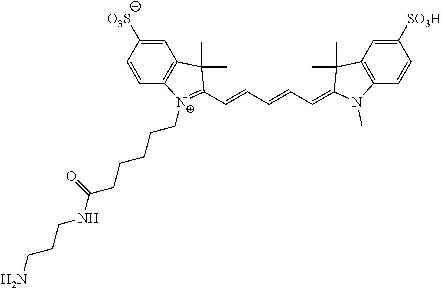 |
| 209 | 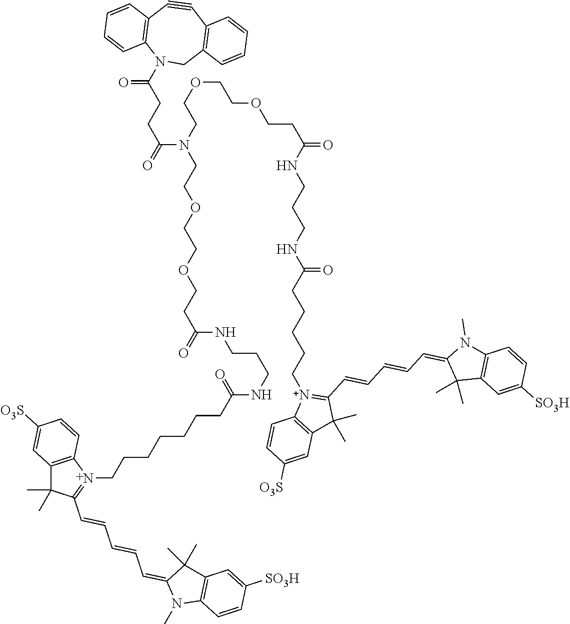 |

Compounds 205 and 206 depict tetrazine fluorophore payloads, while Compound 207 depicts a sulfohydryl-fluorophore payload.

### V. Protein Derivatives

The invention relates to protein derivatives, e.g., proteins modified to include UAAs, linkers, e.g., branched linkers, and/or molecules. Unless indicated otherwise, the terms “protein derivatives” and “derivatized proteins” are used interchangeably herein. In certain embodiments, the protein derivatives are proteins that are expressed or modified to include site-specific homogenous incorporation of one or more unnatural amino acids (UAAs; also referred to as non-natural amino acids, non-canonical amino acids, or nonstandard amino acids).

In certain embodiments, the derivatized protein comprises: (a) an unnatural amino acid; (b) a parent linker having a first terminus and a second terminus, wherein the first terminus of the parent linker is covalently conjugated to the unnatural amino acid; (c) a branching group, wherein the branching group is covalently conjugated to the second terminus of the parent linker; (d) a plurality of branching linkers, each branching linker comprising a branching unit and a conjugating moiety, wherein each branching linker is covalently conjugated to the branching group via the branching unit present in the branching linker; and (e) a plurality of molecules, wherein each molecule is covalently conjugated to one of the plurality of branching linkers via the conjugating moiety present in the branching linker. In certain embodiments, the protein comprises at least two branching linkers. In certain embodiments, the protein comprises at least three branching linkers. In certain embodiments, the protein comprises at least four branching linkers.

In certain embodiments, the derivatized protein comprises: (a) an unnatural amino acid; (b) a parent linker having a first terminus and second terminus, wherein the first terminus of the parent linker is covalently conjugated to the unnatural amino acid; (c) a branching group, wherein the branching group is covalently conjugated to the second terminus of the parent linker; and (d) a plurality of branching linkers, each branching linker comprising a branching unit and a conjugating moiety, wherein each branching linker is covalently conjugated to the branching group via the branching unit present in the branching linker. In certain embodiments, the protein comprises at least two branching linkers. In certain embodiments, the protein comprises at least three branching linkers. In certain embodiments, the protein comprises at least four branching linkers.

An exemplary derivatized protein has the formula:

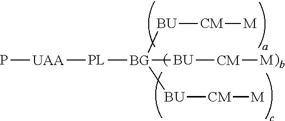

wherein P is a protein; UAA is an unnatural amino acid; PL is a parent linker represented by

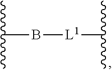

wherein B is a binding unit and L¹ is a chain selected from the group consisting of C₁₋₂₀ alkyl and C₁₋₂₀ heteroalkyl, wherein the chain is optionally substituted by one, two, three or four oxo; BG (branching group) is a polyvalent atom; each BU (branching unit) independently is selected from the group consisting of C₁₋₂₀ alkyl and C₁₋₂₀ heteroalkyl, wherein each BU is optionally substituted by one, two or three oxo; each CM (conjugating moiety) independently is selected from the group consisting of a bond, NH, S, OR¹ and R¹; R¹ is selected from the group consisting of phenyl, 5-10 membered cycloalkyl, 5-10 membered cycloalkenyl, 5-10 membered cycloalkynyl, 5-6 membered heterocycloalkyl, and 5-6 membered heteroaryl, wherein R¹ is optionally substituted by an oxo; each M independently is a molecule; and each of a, b and c independently is selected from the group consisting of 0, 1, 2 and 3, wherein the sum of a, b and c is equal to or less than 3.

Another exemplary derivatized protein has the formula:

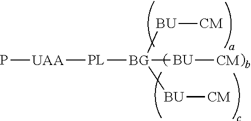

wherein P is a protein; UAA is an unnatural amino acid; PL is a parent linker represented by

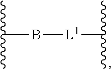

wherein B is a binding unit and L¹ is a chain selected from the group consisting of C₁₋₂₀ alkyl and C₁₋₂₀ heteroalkyl, wherein the chain is optionally substituted by one, two, three or four oxo; BG (branching group) is a polyvalent atom; each BU (branching unit) independently is selected from the group consisting of C₁₋₂₀ alkyl and C₁₋₂₀ heteroalkyl, wherein each BU is optionally substituted by one, two or three oxo; each CM (conjugating moiety) independently is selected from the group consisting of NH₂, SH, OH, O—(C₁₋₃alkyl), OR¹ and R¹; R¹ is selected from the group consisting of phenyl, 5-10 membered cycloalkyl, 5-10 membered cycloalkenyl, 5-10 membered cycloalkynyl, 5-6 membered heterocycloalkyl, and 5-6 membered heteroaryl, wherein R¹ is optionally substituted by an oxo; and each of a, b and c independently is selected from the group consisting of 0, 1, 2 and 3, wherein the sum of a, b and c is equal to or less than 3.

In certain embodiments, the protein is selected from the group consisting of:

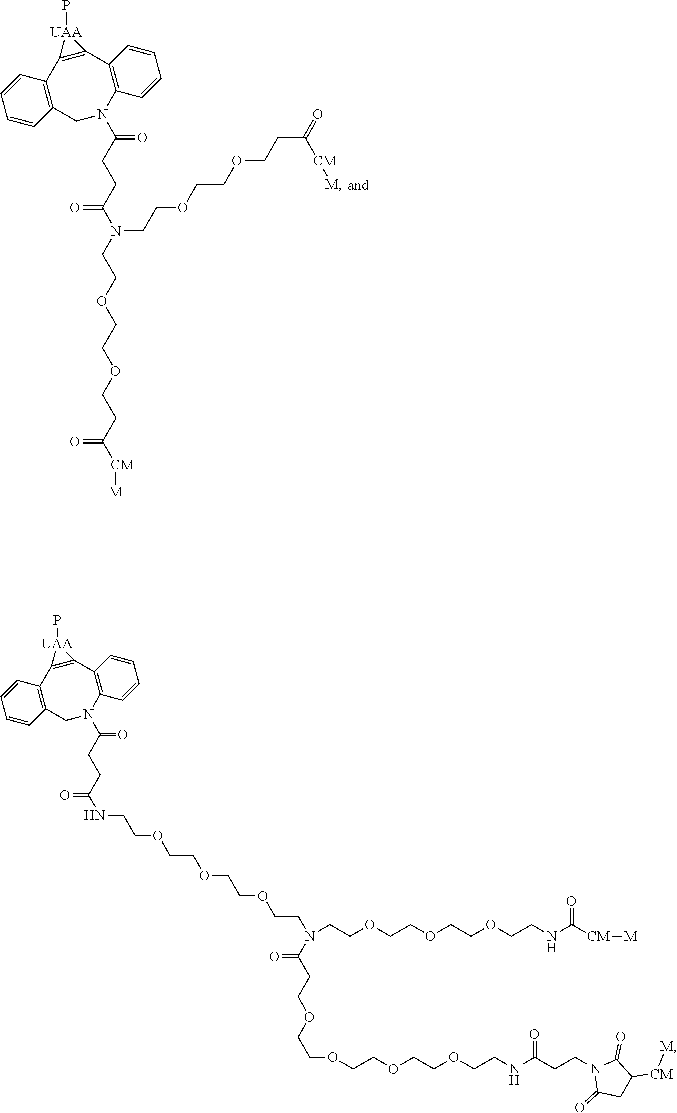

where P represents a protein, UAA represents an unnatural amino acid disposed within the protein, CM represents a conjugating moiety, and M represents a molecule (e.g., a therapeutic agent, radionuclide, or reporter group).

In certain embodiments, the protein is selected from the group consisting of:

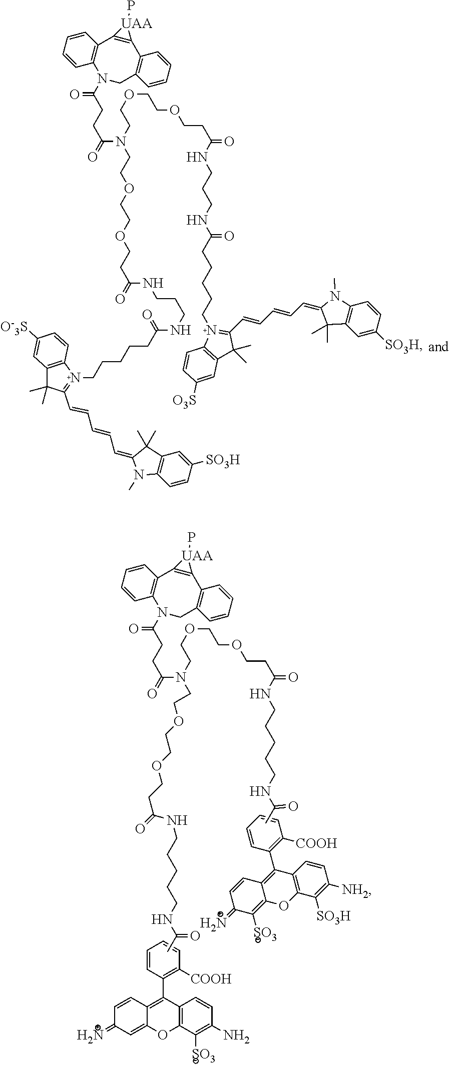

where P represents a protein and UAA represents an unnatural amino acid disposed within the protein.

In certain embodiments, the protein is selected from the group consisting of:

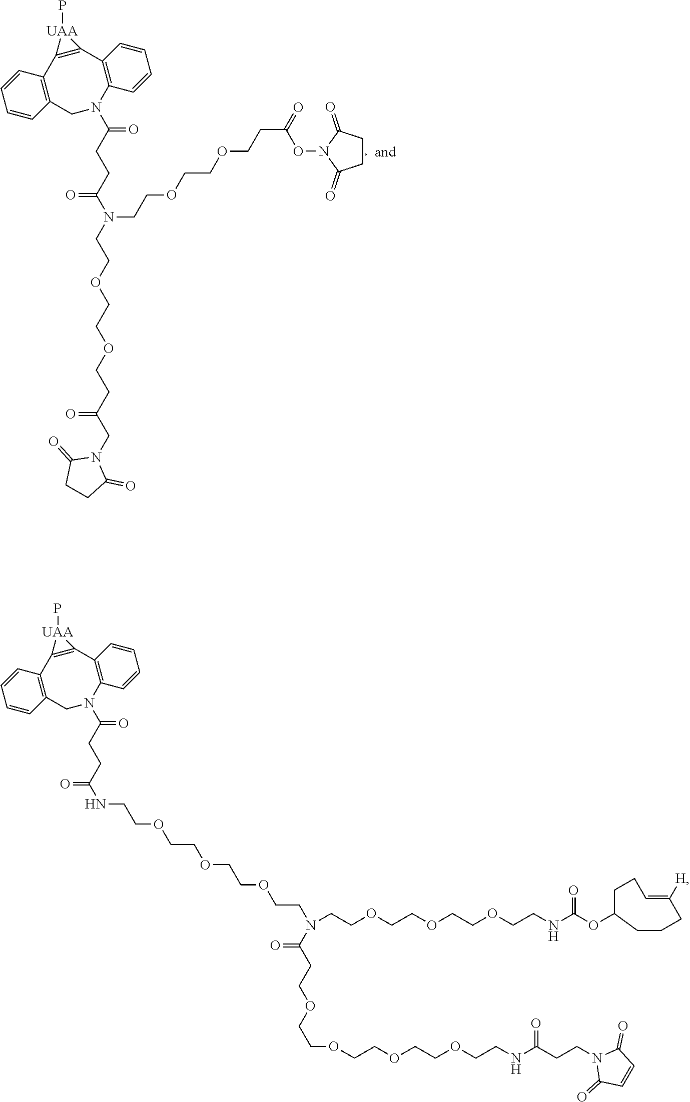

where P represents a protein and UAA represents an unnatural amino acid disposed within the protein.

In certain embodiments, the protein is selected from the group consisting of:

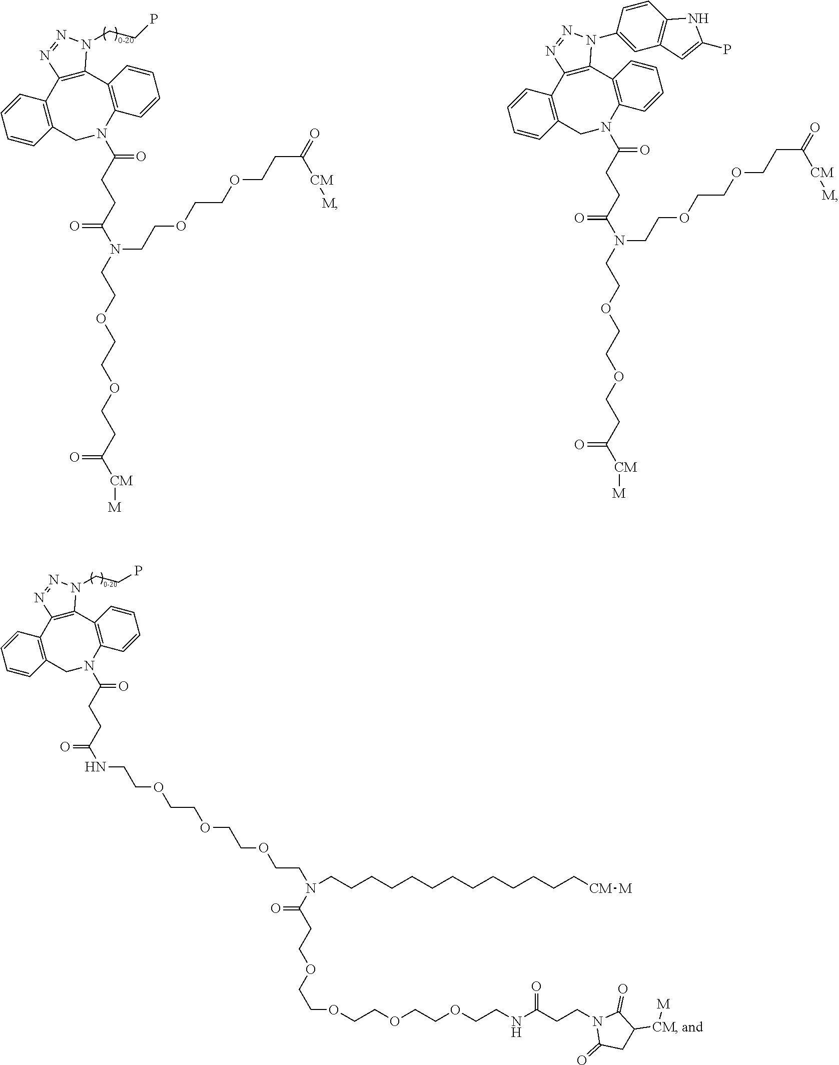

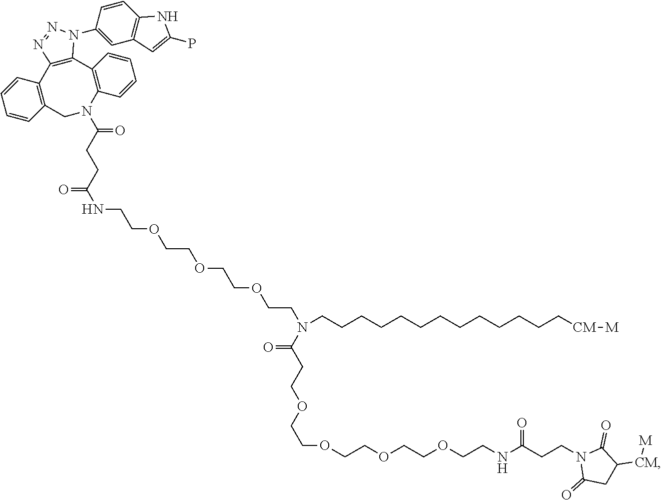

where P represents a protein and a portion of the UAA disposed within the protein is depicted.

In certain embodiments, the protein is selected from the group consisting of:

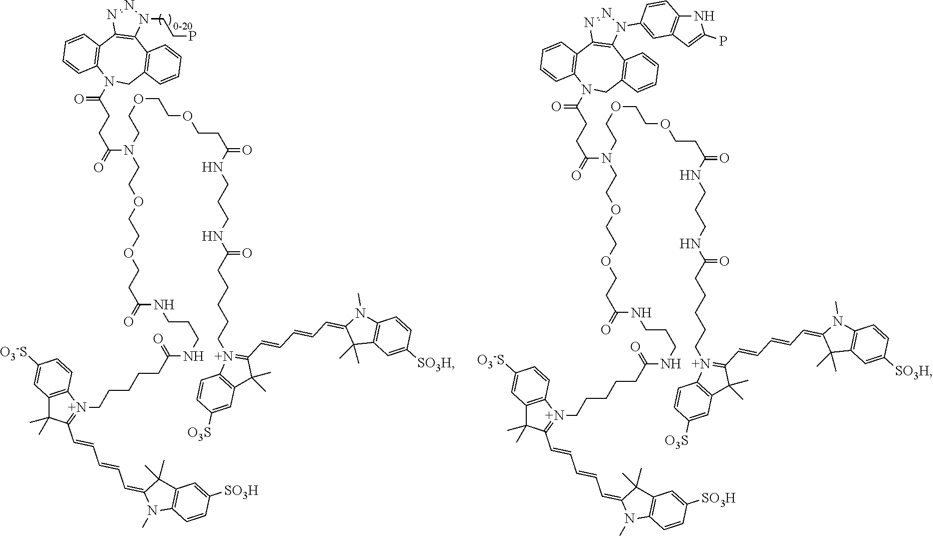

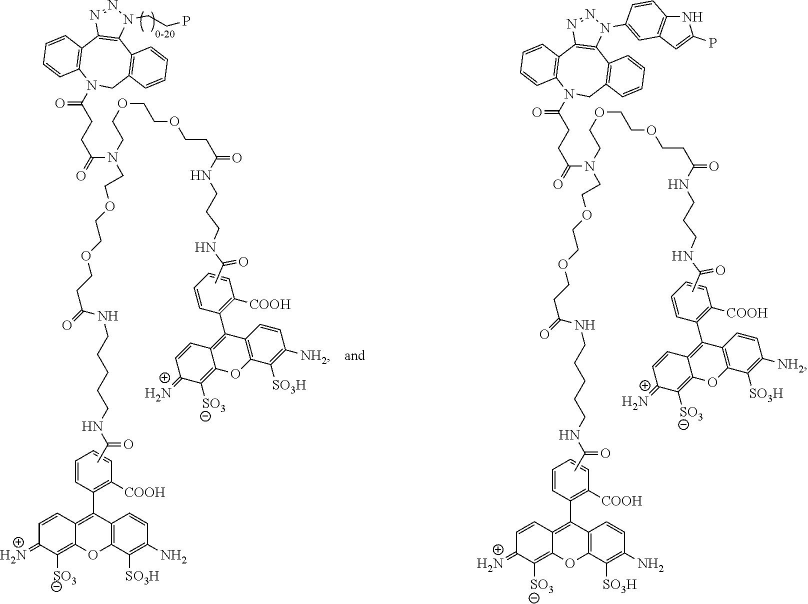

where P represents a protein and a portion of the UAA disposed within the protein is depicted.

In certain embodiments, the protein is selected from the group consisting of:

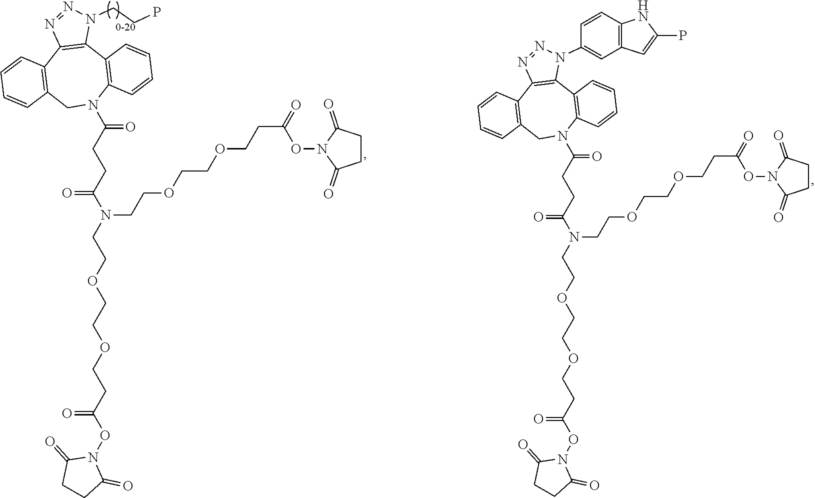

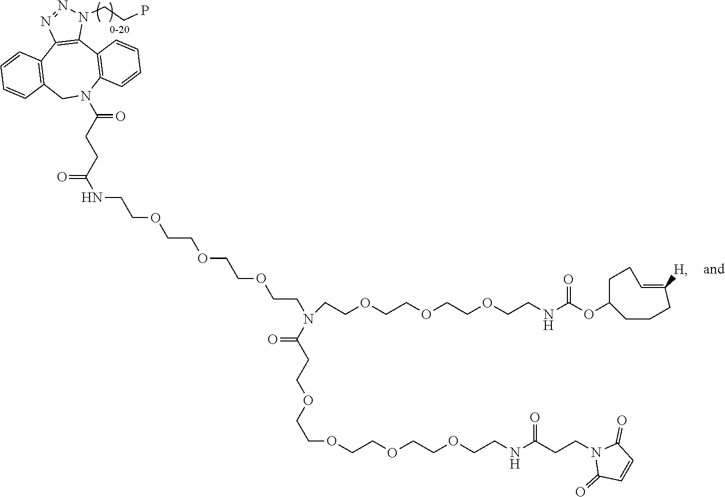

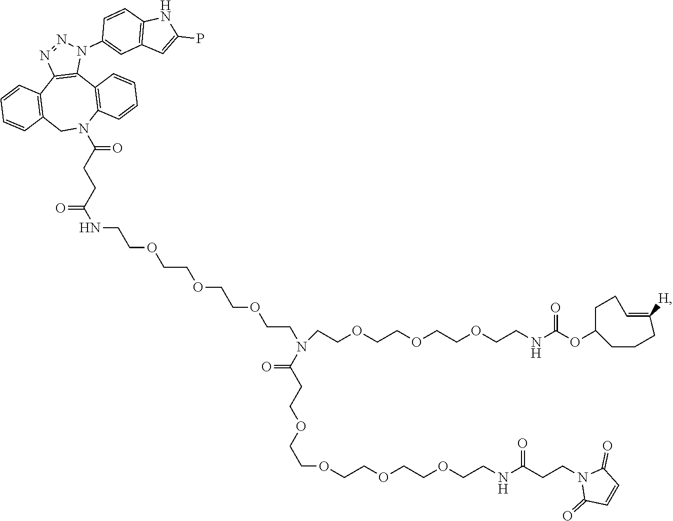

where P represents a protein and a portion of the UAA disposed within the protein is depicted.

In certain embodiments, the protein comprises trastuzumab, or a variant thereof. For example, the protein may comprise trastuzumab or a variant thereof comprising LCA at a position corresponding to T198 of the heavy chain of trastuzumab (e.g., at a position corresponding to T198 in SEQ ID NO: 114), also referred to herein as Tzmab T198.

Derivatized proteins can be prepared in a number of ways based on the teachings contained herein and synthetic procedures known in the art.

In one example, a linker (e.g., a linker identified in Table 1) is first conjugated to an exposed UAA (e.g., LCA) on a protein surface and the linker is then subsequently conjugated to one or more corresponding molecules (“M” such as a therapeutic agent, a radionuclide, and a reporter group). In another example, the linker is first conjugated to one more molecules, and the linker is then subsequently conjugated to the UAA. Different permutations can occur in linkers such as Compound 102, where the TCO may be first conjugated, followed by DBCO conjugation to an azide UAA on the protein, followed by thiol-maleimide conjugation.

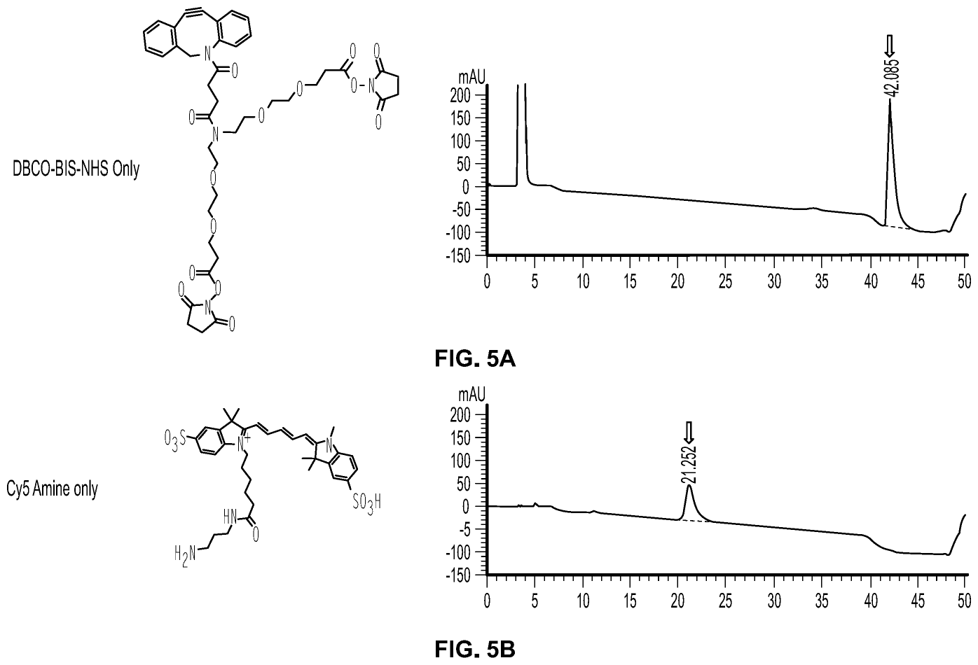

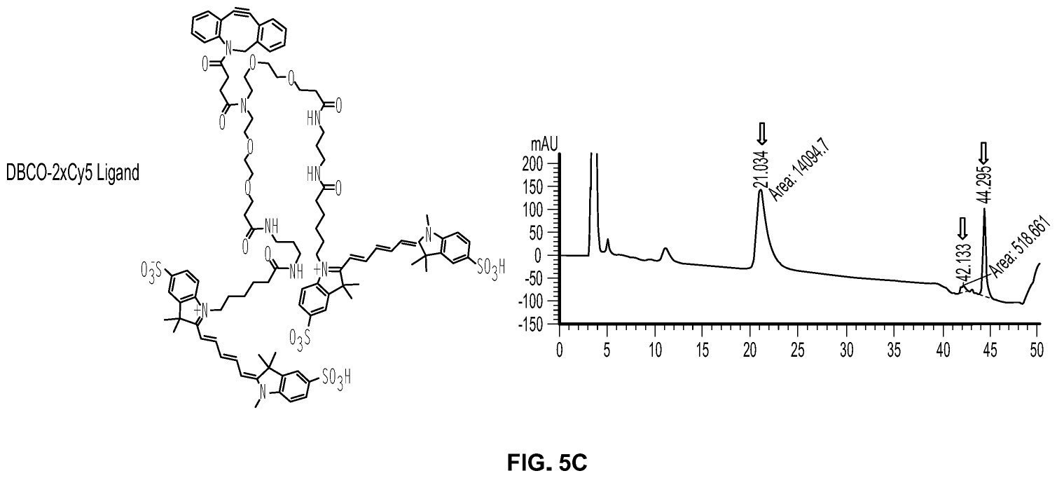

FIG. 5 demonstrates how NHS-Esters facilitate conjugation of molecules of interest (e.g., the fluorescent dye Cy5) to a linker via amine-groups. DBCO can be further used to conjugate the linker to a protein. In one example, a derivatized protein (e.g., Tzmab T198) is labeled with excess of a linker moiety (e.g., a linker comprising DBCO). In certain embodiments, the linker moiety had previously been conjugated to a molecule (e.g., one more Cy5 molecules, e.g., DBCO-2xCy5).

It is understood that the order of labeling may be altered based on the orthogonality of UAAs, linkers, and/or molecules (“M”) and, in certain embodiments, it may be necessary to have intermediate dialysis and/or purification of excess labeling reagents to prevent cross-reactivity.

In certain embodiments, the protein derivative comprises a structure depicted in Table 4.

TABLE 4
Exemplary compounds. Tet—tetrazine; Mal—maleimide;
NHS—N-Hydroxysuccinimide.

| Compound No. | Structure |
|---|---|
| 210 | 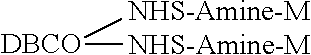 |
| 211 | 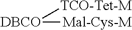 |
| 212 | 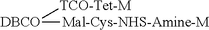 |

Compound 210 schematically depicts a DBCO with two NHS functional groups. These can be conjugated with cognate amine containing functional groups either before, after, or before and after the DBCO is conjugated to a UAA-containing protein (e.g., an antibody).

Compound 211 schematically depicts a DBCO with a TCO functional group and a maleimide functional group. These can be conjugated with cognate tetrazine and sulfohydryl containing functional groups either before, after, or before and after the DBCO is conjugated to a UAA-containing protein (e.g., an antibody). In some cases, a modified strategy can be performed due to the commercial unavailability of free-thiol containing fluorophores (e.g., Compound 207). For example, in Compound 212, the sulfohydryl containing functional group can be further conjugated to NHS and/or amine containing functional groups.

### VI. Methods of Making Proteins

tRNAS, aminoacyl-tRNA synthetases, and/or unnatural amino acids disclosed herein may be used to incorporate an unnatural amino acid into a protein of interest using any appropriate translation system.

The term “translation system” refers to a system including components necessary to incorporate an amino acid into a growing polypeptide chain (protein). Components of a translation system can include, e.g., ribosomes, tRNA's, synthetases, mRNA and the like. Translation systems may be cellular or cell-free, and may be prokaryotic or eukaryotic. For example, translation systems may include, or be derived from, a non-eukaryotic cell, e.g., a bacterium (such as E. coli), a eukaryotic cell, e.g., a yeast cell, a mammalian cell, a plant cell, an algae cell, a fungus cell, or an insect cell.

Translation systems include host cells or cell lines, e.g., host cells or cell lines contemplated herein. To express a polypeptide of interest with an unnatural amino acid in a host cell, one may clone a polynucleotide encoding the polypeptide into an expression vector that contains, for example, a promoter to direct transcription, a transcription/translation terminator, and if for a nucleic acid encoding a protein, a ribosome binding site for translational initiation.

Translation systems also include whole cell preparations such as permeabilized cells or cell cultures wherein a desired nucleic acid sequence can be transcribed to mRNA and the mRNA translated. Cell-free translation systems are commercially available and many different types and systems are well-known. Examples of cell-free systems include, but are not limited to, prokaryotic lysates such as Escherichia coli lysates, and eukaryotic lysates such as wheat germ extracts, insect cell lysates, rabbit reticulocyte lysates, rabbit oocyte lysates and human cell lysates. Reconstituted translation systems may also be used. Reconstituted translation systems may include mixtures of purified translation factors as well as combinations of lysates or lysates supplemented with purified translation factors such as initiation factor-1 (IF-1), IF-2, IF-3 (α or β), elongation factor T (EF-Tu), or termination factors. Cell-free systems may also be coupled transcription/translation systems wherein DNA is introduced to the system, transcribed into mRNA and the mRNA is translated.

The invention provides methods of expressing a protein containing an unnatural amino acid and methods of producing a protein with one, or more, unnatural amino acids at specified positions in the protein. The methods comprise incubating a translation system (e.g., culturing or growing a host cell or cell line, e.g., a host cell or cell line disclosed herein) under conditions that permit incorporation of the unnatural amino acid into the protein being expressed in the cell. The translation system may be contacted with (e.g. the cell culture medium may be contacted with) one, or more, unnatural amino acids (e.g., leucyl or tryptophanyl analogs) under conditions suitable for incorporation of the one, or more, unnatural amino acids into the protein.

In certain embodiments, the protein is expressed from a nucleic acid sequence comprising a premature stop codon. The translation system (e.g., host cell or cell line) may, for example, contain a leucyl-tRNA synthetase mutein (e.g., a leucyl-tRNA synthetase mutein disclosed herein) capable of charging a suppressor leucyl tRNA (e.g., a suppressor leucyl tRNA disclosed herein) with an unnatural amino acid (e.g., a leucyl analog) which is incorporated into the protein at a position corresponding to the premature stop codon. In certain embodiments, the leucyl suppressor tRNA comprises an anticodon sequence that hybridizes to the premature stop codon and permits the unnatural amino to be incorporated into the protein at the position corresponding to the premature stop codon.

In certain embodiments, the protein is expressed from a nucleic acid sequence comprising a premature stop codon. The translation system (e.g., host cell or cell line) may, for example, contain a tryptophanyl-tRNA synthetase mutein (e.g., a tryptophanyl-tRNA synthetase mutein disclosed herein) capable of charging a suppressor tryptophanyl tRNA (e.g., a suppressor tryptophanyl tRNA disclosed herein) with an unnatural amino acid (e.g., a tryptophan analog) which is incorporated into the protein at a position corresponding to the premature stop codon. In certain embodiments, the tryptophanyl suppressor tRNA comprises an anticodon sequence that hybridizes to the premature stop codon and permits the unnatural amino to be incorporated into the protein at the position corresponding to the premature stop codon.

In certain embodiments, a protein (e.g., an antibody containing a UAA) is expressed or produced in a eukaryotic cell (e.g., a mammalian cell). Features may distinguish proteins produced in prokaryotic cells (e.g., bacteria) from those produced in eukaryotic cells (e.g., mammalian cells). For example, proteins produced in mammalian cells may undergo post-translational modifications, e.g., modifications that are dependent upon enzymes located in organelles, e.g., the endoplasmic reticulum or Golgi apparatus. For example, disulfide bond formation in the endoplasmic reticulum may influence protein conformation and/or stabilization. Additional examples of such post-translational modifications include, without limitation, sulfation, amidation, palmitation, and glycosylation (e.g., N-linked glycosylation and O-linked glycosylation). Accordingly, in certain embodiments, a protein (e.g., an antibody containing a UAA) comprises one or more post-translational modifications selected from sulfation, amidation, palmitation, and glycosylation (e.g., N-linked glycosylation and O-linked glycosylation).

In certain embodiments, the expression yield of a protein comprising the UAA, for example, when expressed by a host cell or cell line, is at least 10%, 20%, 30%, 40%, 50%, 60%, 70%, 80%, or 90% of the expression yield of a reference protein. For example, in certain embodiments, the amount of protein comprising the UAA expressed by the host cell or cell line is at least 10%, 20%, 30%, 40%, 50%, 60%, 70%, 80%, or 90% of the amount of a reference protein expressed by the same cell or a similar cell. In certain embodiments, the reference protein is an protein that does not comprise the UAA but is otherwise identical to the protein comprising the UAA. For example, the reference protein may comprise a wild-type amino acid sequence, or comprise a wild-type amino acid residue at the position corresponding to the UAA. Protein expression may be measured by any method known in the art, including for example, Western blot or ELISA. Expression may be measured by measuring protein concentration (e.g., by ultraviolet (UV) absorption at 280 nm or Bradford assay) in a solution of defined volume and purity following purification of the protein.

In certain embodiments, a disclosed method further comprises purifying the protein. Specific expression and purification conditions will vary depending upon the expression system employed. Purification techniques known in the art include, e.g., those employing affinity tags such as glutathione-S-transferase (GST) or histidine tags. In certain embodiments, an antibody may be purified by contacting the antibody with protein A and/or protein G. In certain embodiments, following protein G purification (e.g., following only protein G purification) less than 10%, 9%, 8%, 7%, 6%, 5%, 4%, 3%, 2%, or 1% of the antibody is aggregated, as measured by size exclusion chromatography (SEC).

In certain embodiments, a disclosed method further comprises conjugating a molecule or payload to a UAA in the protein. In certain embodiments, the method comprises conjugating the molecule or payload to the UAA within 5 minutes to 48 hours at room temperature (e.g., for less than 48 hours, less than 36 hours, less than 24 hours, less than 12 hours, less than 6 hours, less than 1 hour, less than 30 minutes, less than 15 minutes, or less than 10 minutes).

### VI. Aminoacyl-tRNA Synthetases

The invention relates to engineered aminoacyl-tRNA synthetases (or aaRSs) capable of charging a tRNA with an unnatural amino acid for incorporation into a protein (e.g., an antibody). As used herein, the term “aminoacyl-tRNA synthetase” refers to any enzyme, or a functional fragment thereof, that charges, or is capable of charging, a tRNA with an amino acid (e.g., an unnatural amino acid) for incorporation into a protein. As used herein, the term “functional fragment” of an aminoacyl-tRNA synthetase refers to fragment of a full-length aminoacyl-tRNA synthetase that retains, for example, at least 10%, at least 20%, at least 30%, at least 40%, at least 50%, at least 60%, at least 70%, at least 80%, at least 90%, at least 95%, or 100% of the enzymatic activity of the corresponding full-length tRNA synthetase (e.g., a naturally occurring tRNA synthetase). Aminoacyl-tRNA synthetase enzymatic activity may be assayed by any method known in the art. For example, in vitro aminoacylation assays are described in Hoben et al. (1985) METHODS ENZYMOL. 113:55-59 and in U.S. Patent Application Publication No. 2003/0228593 and cell-based aminoacylation assays are described in U.S. Patent Application Publication Nos. 2003/0082575 and 2005/0009049. In certain embodiments, the functional fragment comprises at least 100, 150, 200, 250, 300, 350, 400, 450, 500, 550, 600, 650, 700, 750, or 800 consecutive amino acids present in a full-length tRNA synthetase (e.g., a naturally occurring aminoacyl-tRNA synthetase).

The term aminoacyl-tRNA synthetase includes variants (i.e., muteins) having one or more mutations (e.g., amino acid substitutions, deletions, or insertions) relative to a wild-type aminoacyl-tRNA synthetase sequence. In certain embodiments, an aminoacyl-tRNA synthetase mutein may comprise, consist, or consist essentially of, a single mutation (e.g., a mutation contemplated herein), or a combination of 2, 3, 4, 5, 6, 7, 8, 9, 10, 11, 12, 13, 14, 15 or more than 15 mutations (e.g., mutations contemplated herein). It is contemplated that an aminoacyl-tRNA synthetase mutein may comprise, consist, or consist essentially 1-15, 1-10, 1-7, 1-6, 1-5, 1-4, 1-3, 1-2, 2-15, 2-10, 2-7, 2-6, 2-5, 2-4, 2-3, 3-15, 3-10, 3-7, 3-6, 3-5, or 4-10, 4-7, 4-6, 4-5, 5-10, 5-7, 5-6, 6-10, 6-7, 7-10, 7-8, or 8-10 mutations (e.g., mutations contemplated herein). An aminoacyl-tRNA synthetase mutein may comprise a conservative substitution relative to a wild-type sequence or a sequence disclosed herein.

In certain embodiments, the substrate specificity of the aminoacyl-tRNA synthetase mutein is altered relative to a corresponding (or template) wild-type aminoacyl-tRNA synthetase such that only a desired unnatural amino acid, but not any of the common amino acids, is charged to the substrate tRNA.

An aminoacyl-tRNA synthetase may be derived from a bacterial source, e.g., Escherichia coli, Thermus thermophilus, or Bacillus stearothermphilus. An aminoacyl-tRNA synthetase may also be derived from an archaeal source, e.g., from the Methanosarcinacaea or Desulfitobacterium families, any of the M barkeri (Mb), M. alvus (Ma), M mazei (Mm) or D. hafnisense (Dh) families, Methanobacterium thermoautotrophicum, Haloferax volcanii, Halobacterium species NRC-1, or Archaeoglobus fulgidus. In other embodiments, eukaryotic sources can also be used, for example, plants, algae, protists, fungi, yeasts, or animals (e.g., mammals, insects, arthropods, etc.). As used herein, the terms “derivative” or “derived from” refer to a component that is isolated from or made using information from a specified molecule or organism. As used herein, the term “analog” refers to a component (e.g., a tRNA, tRNA synthetase, or unnatural amino acid) that is derived from or analogous with (in terms of structure and/or function) a reference component (e.g., a wild-type tRNA, a wild-type tRNA synthetase, or a natural amino acid). In certain embodiments, derivatives or analogs have at least 40%, 50%, 60%, 70%, 80%, 90%, 100% or more of a given activity as a reference or originator component (e.g., wild type component).

It is contemplated that the aminoacyl-tRNA synthetase may aminoacylate a substrate tRNA in vitro or in vivo, and can be provided to a translation system (e.g., an in vitro translation system or a cell) as a polypeptide or protein, or as a polynucleotide that encodes the aminoacyl-tRNA synthetase.

In certain embodiments, the aminoacyl-tRNA synthetase is derived from an E. coli leucyl-tRNA synthetase and, for example, the aminoacyl-tRNA synthetase preferentially aminoacylates an E. coli leucyl tRNA (or a variant thereof) with a leucine analog over the naturally-occurring leucine amino acid.

For example, the aminoacyl-tRNA synthetase may comprise SEQ ID NO: 1, or an amino acid sequence that has at least 85%, 90%, 95%, 96%, 97%, 98%, or 99% sequence identity to SEQ ID NO: 1. In certain embodiments, the aminoacyl-tRNA synthetase comprises SEQ ID NO: 1, or a functional fragment or variant thereof, and with one, two, three, four, five or more of the following mutations: (i) a substitution of a glutamine residue at a position corresponding to position 2 of SEQ ID NO: 1, e.g., a substitution by glutamic acid (Q2E); (ii) a substitution of a glutamic acid residue at a position corresponding to position 20 of SEQ ID NO: 1, e.g., a substitution by lysine (E20K), methionine (E20M), or valine (E20V); (iii) a substitution of a methionine residue at a position corresponding to position 40 of SEQ ID NO: 1, e.g., a substitution by isoleucine (M40I) or valine (M40V); (iv) a substitution of a leucine residue at a position corresponding to position 41 of SEQ ID NO: 1, e.g., a substitution by serine (L41S), valine (L41V), or alanine (L41A); (v) a substitution of a threonine residue at a position corresponding to position 252 of SEQ ID NO: 1, e.g., a substitution by alanine (T252A) or arginine (T252R); (vi) a substitution of a tyrosine residue at a position corresponding to position 499 of SEQ ID NO: 1, e.g., a substitution by isoleucine (Y499I), serine (Y499S), alanine (Y499A), or histidine (Y499H); (vii) a substitution of a tyrosine residue at a position corresponding to position 527 of SEQ ID NO: 1, e.g., a substitution by alanine (Y527A), leucine (Y527L), isoleucine (Y527I), valine (Y527V), or glycine (Y527G); or (viii) a substitution of a histidine residue at a position corresponding to position 537 of SEQ ID NO: 1, e.g., a substitution by glycine (H537G), or any combination of the foregoing.

In certain embodiments, the aminoacyl-tRNA synthetase comprises (i) at least one substitution (e.g., a substitution with a hydrophobic amino acid) at a position corresponding to His537 of SEQ ID NO: 1, (ii) at least one amino acid substitution selected from E20V, E20M, L41V, L41A, Y499H, Y499A, Y527I, Y527V, Y527G, and any combination thereof, (iii) at least one amino acid substitution selected from E20K and L41S and any combination thereof and at least one amino acid substitution selected from M40I, T252A, Y499I, and Y527A, and any combination thereof, or (iv) a combination of two or more of (i), (ii) and (iii), for example, (i) and (ii), (i) and (iii), (ii) and (iii) and (i), (ii) and (iii).

In certain embodiments, the aminoacyl-tRNA synthetase comprises a substitution of a glutamic acid residue at a position corresponding to position 20 of SEQ ID NO: 1, e.g., a substitution with an amino acid other than a Glu or Lys, e.g., a substitution with a hydrophobic amino acid (e.g., Leu, Val, or Met). In certain embodiments, the aminoacyl-tRNA synthetase comprises a substitution of a leucine residue at a position corresponding to position 41 of SEQ ID NO: 1, e.g., a substitution with an amino acid other than a Leu or Ser, e.g., a substitution with a hydrophobic amino acid other than Leu (e.g., Gly, Ala, Val, or Met). In certain embodiments, the aminoacyl-tRNA synthetase comprises a substitution of a tyrosine residue at a position corresponding to position 499 of SEQ ID NO: 1, e.g., a substitution with a small hydrophobic amino acid (e.g., Gly, Ala, or Val) or a substitution with a positively charged amino acid (e.g., Lys, Arg, or His). In certain embodiments, the aminoacyl-tRNA synthetase comprises a substitution of a tyrosine residue at a position corresponding to position 527 of SEQ ID NO: 1, e.g., a substitution with a hydrophobic amino acid other than Ala or Leu (e.g., Gly, Ile, Met, or Val). In certain embodiments, the tRNA synthetase mutein comprises L41V.

In certain embodiments, the aminoacyl-tRNA synthetase comprises a combination of mutations selected from: (i) Q2E, E20K, M40I, L41S, T252A, Y499I, Y527A, and H537G; (ii) Q2E, E20K, M40V, L41S, T252R, Y499S, Y527L, and H537G; (iii) Q2E, M40I, T252A, Y499I, Y527A, and H537G; (iv) Q2E, E20M, M40I, L41S, T252A, Y499I, Y527A, and H537G; (v) Q2E, E20V, M40I, L41S, T252A, Y4991, Y527A, and H537G; (vi) Q2E, E20K, M40I, L41V, T252A, Y499I, Y527A, and H537G; (vii) Q2E, E20K, M40I, L41A, T252A, Y499I, Y527A, and H537G; (viii) Q2E, E20K, M40I, L41S, T252A, Y499A, Y527A, and H537G; (ix) Q2E, E20K, M40I, L41S, T252A, Y499H, Y527A, and H537G; (x) Q2E, E20K, M40I, L41S, T252A, Y499I, Y527I, and H537G; (xi) Q2E, E20K, M40I, L41S, T252A, Y499I, Y527V, and H537G; (xii) Q2E, E20K, M40I, L41S, T252A, Y499I, Y527G, and H537G; (xiii) E20K, M40I, L41S, T252A, Y499I, Y527A, and H537G; (xiv) E20M, M40I, L41S, T252A, Y499I, Y527A, and H537G; (xv) E20V, M40I, L41S, T252A, Y499I, Y527A, and H537G; (xvi) E20K, M40I, L41V, T252A, Y499I, Y527A, and H537G; (vii) E20K, M40I, L41A, T252A, Y499I, Y527A, and H537G; (xviii) E20K, M40I, L41S, T252A, Y499A, Y527A, and H537G; (xix) E20K, M40I, L41S, T252A, Y499H, Y527A, and H537G; (xx) E20K, M401, L41S, T252A, Y4991, Y5271, and H537G; (xxi) E20K, M40I, L41S, T252A, Y499I, Y527V, and H537G; and (xxii) E20K, M40I, L41S, T252A, Y499I, Y527G, and H537G.

In certain embodiments, the aminoacyl-tRNA synthetase comprises the amino acid sequence of any one of SEQ ID NOs: 2-13, or an amino acid sequence that has at least 85%, 90%, 95%, 96%, 97%, 98%, or 99% sequence identity to any one of SEQ ID NOs: 2-13.

In certain embodiments, the tRNA synthetase mutein comprises the amino acid sequence of SEQ ID NO: 14, wherein X₂ is Q or E, X₂₀ is E, K, V or M, X₄₀ is M, I, or V, X₄₁ is L, S, V, or A, X₂₅₂ is T, A, or R, X₄₉₉ is Y, A, I, H, or S, X₅₂₇ is Y, A, I, L, or V, and X₅₃₇ is H or G, and the tRNA synthetase mutein comprises at least one mutation (for example, 2, 3, 4, 5, 6, 7, 8, 9, or more mutations) relative to SEQ ID NO: 1. In certain embodiments, the tRNA synthetase mutein comprises the amino acid sequence of SEQ ID NO: 15, wherein X₂₀ is K, V or M, X₄₁ is S, V, or A, X₄₉₉ is A, I, or H, and X₅₂₇ is A, I, or V, and the tRNA synthetase mutein comprises at least one mutation relative to SEQ ID NO: 1.

In certain embodiments, the aminoacyl-tRNA synthetase is derived from an E. coli tryptophanyl-tRNA synthetase and, for example, the aminoacyl-tRNA synthetase preferentially aminoacylates an E. coli tryptophanyl tRNA (or a variant thereof) with a tryptophan analog over the naturally-occurring tryptophan amino acid.

For example, the aminoacyl-tRNA synthetase may comprise SEQ ID NO: 44, or an amino acid sequence that has at least 85%, 90%, 95%, 96%, 97%, 98%, or 99% sequence identity to SEQ ID NO: 44. In certain embodiments, the aminoacyl-tRNA synthetase comprises SEQ ID NO: 44, or a functional fragment or variant thereof, but with one or more of the following mutations: (i) a substitution of a serine residue at a position corresponding to position 8 of SEQ ID NO: 44, e.g., a substitution by alanine (S8A); (ii) a substitution of a valine residue at a position corresponding to position 144 of SEQ ID NO: 44, e.g., a substitution by serine (V144S), glycine (V144G) or alanine (V144A); (iii) a substitution of a valine residue at a position corresponding to position 146 of SEQ ID NO: 44, e.g., a substitution by alanine (V146A), isoleucine (V146I), or cysteine (V146C). In certain embodiments, the aminoacyl-tRNA synthetase comprises a combination of mutations selected from: (i) S8A, V144S, and V146A, (ii) S8A, V144G, and V146I, (iii) S8A, V144A, and V146A, and (iv) S8A, V144G, and V146C.

In certain embodiments, the aminoacyl-tRNA synthetase comprises the amino acid sequence of any one of SEQ ID NOs: 45-48, or an amino acid sequence that has at least 85%, 90%, 95%, 96%, 97%, 98%, or 99% sequence identity to any one of SEQ ID NOs: 45-48.

In certain embodiments, the aminoacyl-tRNA synthetase is derived from an E. coli tyrosyl-tRNA synthetase and, for example, the aminoacyl-tRNA synthetase preferentially aminoacylates an E. coli tyrosyl tRNA (or a variant thereof) with a tyrosine analog over the naturally-occurring tryptophan amino acid. For example, the aminoacyl-tRNA synthetase may comprise SEQ ID NO: 70, or an amino acid sequence that has at least 85%, 90%, 95%, 96%, 97%, 98%, or 99% sequence identity to SEQ ID NO: 70, or a functional fragment or variant thereof.

In certain embodiments, the aminoacyl-tRNA synthetase is derived from an M barkeri pyrrolysyl-tRNA synthetase and, for example, the aminoacyl-tRNA synthetase preferentially aminoacylates an M. barkeri pyrrolysyl tRNA (or a variant thereof) with a pyrrolysine analog over the naturally-occurring pyrrolysine amino acid. For example, the aminoacyl-tRNA synthetase may comprise SEQ ID NO: 101, or an amino acid sequence that has at least 85%, 90%, 95%, 96%, 97%, 98%, or 99% sequence identity to SEQ ID NO: 101, or a functional fragment or variant thereof.

Methods for producing proteins, e.g., aminoacyl-tRNA synthetases, are known in the art. For example, DNA molecules encoding a protein of interest can be synthesized chemically or by recombinant DNA methodologies. The resulting DNA molecules encoding the protein interest can be ligated to other appropriate nucleotide sequences, including, for example, expression control sequences, to produce conventional gene expression constructs (i.e., expression vectors) encoding the desired protein. Production of defined gene constructs is within routine skill in the art.

Nucleic acids encoding desired proteins (e.g., aminoacyl-tRNA synthetases) can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells. Transformed host cells can be grown under conditions that permit the host cells to express the desired protein.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed protein may be secreted. The expressed protein may accumulate in refractile or inclusion bodies, which can be harvested after disruption of the cells by French press or sonication. The refractile bodies then are solubilized, and the protein may be refolded and/or cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon. Optionally, the vector or gene construct may contain enhancers and introns. The gene construct can be introduced into eukaryotic host cells using conventional techniques.

A protein of interest (e.g., an aminoacyl-tRNA synthetase) can be produced by growing (culturing) a host cell transfected with an expression vector encoding such a protein under conditions that permit expression of the protein. Following expression, the protein can be harvested and purified or isolated using techniques known in the art, e.g., affinity tags such as glutathione-S-transferase (GST) or histidine tags.

Additional methods for producing aminoacyl-tRNA synthetases, and for altering the substrate specificity of the synthetase can be found in U.S. Patent Application Publication Nos. 2003/0108885 and 2005/0009049, Hamano-Takaku et al. (2000) JOURNAL OF BIOL. CHEM. 275(51):40324-40328, Kiga et al. (2002) PROC. NATL. ACAD. SCI. USA 99(15): 9715-9723, and Francklyn et al. (2002) RNA, 8:1363-1372.

The invention also encompasses nucleic acids encoding aminoacyl-tRNA synthetases disclosed herein. For example, nucleotide sequences encoding leucyl-tRNA synthetase muteins disclosed herein are depicted in SEQ ID NOs: 55-67. Accordingly, the invention provides a nucleic acid comprising the nucleotide sequence of any one of SEQ ID NOs: 55-67, or a nucleotide sequence that has at least 85%, 90%, 95%, 96%, 97%, 98%, or 99% sequence identity to any one of SEQ ID NOs: 55-67. The invention also provides a nucleic acid comprising a nucleotide sequence encoding the amino acid sequence encoded by any one of SEQ ID NOs: 55-67, or a nucleotide sequence that has at least 85%, 90%, 95%, 96%, 97%, 98%, or 99% sequence identity to a nucleotide sequence encoding the amino acid sequence encoded by any one of SEQ ID NOs: 55-67.

A nucleotide sequence encoding a tryptophanyl-tRNA synthetase disclosed herein is depicted in SEQ ID NO: 103. Accordingly, the invention provides a nucleic acid comprising the nucleotide sequence of SEQ ID NO: 103, or a nucleotide sequence that has at least 85%, 90%, 95%, 96%, 97%, 98%, or 99% sequence identity to SEQ ID NO: 103. The invention also provides a nucleic acid comprising a nucleotide sequence encoding the amino acid sequence encoded by SEQ ID NO: 103, or a nucleotide sequence that has at least 85%, 90%, 95%, 96%, 97%, 98%, or 99% sequence identity to a nucleotide sequence encoding the amino acid sequence encoded by SEQ ID NO: 103.

A nucleotide sequence encoding a tyrosyl-tRNA synthetase disclosed herein is depicted in SEQ ID NO: 71. Accordingly, the invention provides a nucleic acid comprising the nucleotide sequence of SEQ ID NO: 71, or a nucleotide sequence that has at least 85%, 90%, 95%, 96%, 97%, 98%, or 99% sequence identity to SEQ ID NO: 71. The invention also provides a nucleic acid comprising a nucleotide sequence encoding the amino acid sequence encoded by SEQ ID NO: 71, or a nucleotide sequence that has at least 85%, 90%, 95%, 96%, 97%, 98%, or 99% sequence identity to a nucleotide sequence encoding the amino acid sequence encoded by SEQ ID NO: 71.

A nucleotide sequence encoding a pyrrolysyl-tRNA synthetase disclosed herein is depicted in SEQ ID NO: 102. Accordingly, the invention provides a nucleic acid comprising the nucleotide sequence of SEQ ID NO: 102, or a nucleotide sequence that has at least 85%, 90%, 95%, 96%, 97%, 98%, or 99% sequence identity to SEQ ID NO: 102. The invention also provides a nucleic acid comprising a nucleotide sequence encoding the amino acid sequence encoded by SEQ ID NO: 102, or a nucleotide sequence that has at least 85%, 90%, 95%, 96%, 97%, 98%, or 99% sequence identity to a nucleotide sequence encoding the amino acid sequence encoded by SEQ ID NO: 102.

VIII. tRNAs

The invention relates to transfer RNAs (tRNAs) that mediate the incorporation of unnatural amino acids into proteins (e.g., antibodies).

During protein synthesis, a tRNA molecule delivers an amino acid to a ribosome for incorporation into a growing protein (polypeptide) chain. tRNAs typically are about 70 to 100 nucleotides in length. Active tRNAs contain a 3′ CCA sequence that may be transcribed into the tRNA during its synthesis or may be added later during post-transcriptional processing. During aminoacylation, the amino acid that is attached to a given tRNA molecule is covalently attached to the 2′ or 3′ hydroxyl group of the 3′-terminal ribose to form an aminoacyl-tRNA (aa-tRNA). It is understood that an amino acid can spontaneously migrate from the 2′-hydroxyl group to the 3′-hydroxyl group and vice versa, but it is incorporated into a growing protein chain at the ribosome from the 3′-OH position. A loop at the other end of the folded aa-tRNA molecule contains a sequence of three bases known as the anticodon. When this anticodon sequence hybridizes or base-pairs with a complementary three-base codon sequence in a ribosome-bound mRNA, the aa-tRNA binds to the ribosome and its amino acid is incorporated into the polypeptide chain being synthesized by the ribosome. Because all tRNAs that base-pair with a specific codon are aminoacylated with a single specific amino acid, the translation of the genetic code is affected by tRNAs. Each of the 61 non-termination codons in an mRNA directs the binding of its cognate aa-tRNA and the addition of a single specific amino acid to the growing polypeptide chain being synthesized by the ribosome. The term “cognate” refers to components that function together, e.g., a tRNA and an aminoacyl-tRNA synthetase.

Suppressor tRNAs are modified tRNAs that alter the reading of a mRNA in a given translation system. For example, a suppressor tRNA may read through a codon such as a stop codon, a four base codon, or a rare codon. The use of the word in suppressor is based on the fact, that under certain circumstance, the modified tRNA “suppresses” the typical phenotypic effect of the codon in the mRNA. Suppressor tRNAs typically contain a mutation (modification) in either the anticodon, changing codon specificity, or at some position that alters the aminoacylation identity of the tRNA. The term “suppression activity” refers to the ability of a tRNA, e.g., a suppressor tRNA, to read through a codon (e.g., a premature stop codon) that would not be read through by the endogenous translation machinery in a system of interest.

In certain embodiments, a tRNA (e.g., a suppressor tRNA) contains a modified anticodon region, such that the modified anticodon hybridizes with a different codon than the corresponding naturally occurring anticodon.

In certain embodiments, a tRNA comprises an anticodon that hybridizes to a codon selected from UAG (i.e., an “amber” termination codon), UGA (i.e., an “opal” termination codon), and UAA (i.e., an “ochre” termination codon).

In certain embodiments, a tRNA comprises an anticodon that hybridizes to a non-standard codon, e.g., a 4- or 5-nucleotide codon. Examples of four base codons include AGGA, CUAG, UAGA, and CCCU. Examples of five base codons include AGGAC, CCCCU, CCCUC, CUAGA, CUACU, and UAGGC. tRNAs comprising an anticodon that hybridizes to a non-standard codon, e.g., a 4- or 5-nucleotide codon, and methods of using such tRNAs to incorporate unnatural amino acids into proteins are described, for example, in Moore et al. (2000) J. MOL. BIOL. 298:195; Hohsaka et al. (1999) J. AM. CHEM. SOC. 121:12194; Anderson et al. (2002) CHEMISTRY AND BIOLOGY 9:237-244; Magliery (2001) J. MOL. BIOL. 307: 755-769; and PCT Publication No. WO2005/007870.

As used herein, the term “tRNA” includes variants having one or more mutations (e.g., nucleotide substitutions, deletions, or insertions) relative to a reference (e.g., a wild-type) tRNA sequence. In certain embodiments, a tRNA may comprise, consist, or consist essentially of, a single mutation (e.g., a mutation contemplated herein), or a combination of 2, 3, 4, 5, 6, 7, 8, 9, 10, 11, 12, 13, 14, 15 or more than 15 mutations (e.g., mutations contemplated herein). It is contemplated that a tRNA may comprise, consist, or consist essentially 1-15, 1-10, 1-7, 1-6, 1-5, 1-4, 1-3, 1-2, 2-15, 2-10, 2-7, 2-6, 2-5, 2-4, 2-3, 3-15, 3-10, 3-7, 3-6, 3-5, or 3-4 mutations (e.g., mutations contemplated herein).

In certain embodiments, a variant suppressor tRNA has increased activity to incorporate an unnatural amino acid (e.g., an unnatural amino acid contemplated herein) into a mammalian protein relative to a counterpart wild-type suppressor tRNA (in this context, a wild-type suppressor tRNA refers to a suppressor tRNA that corresponds to a wild-type tRNA molecule but for any modifications to the anti-codon region to impart suppression activity). The activity of the variant suppressor tRNA may be increased relative to the wild type suppressor tRNA, for example, by about 2.5 to about 200 fold, about 2.5 to about 150 fold, about 2.5 to about 100 fold about 2.5 to about 80 fold, about 2.5 to about 60 fold, about 2.5 to about 40 fold, about 2.5 to about 20 fold, about 2.5 to about 10 fold, about 2.5 to about fold, about 5 to about 200 fold, about 5 to about 150 fold, about 5 to about 100 fold, about to about 80 fold, about 5 to about 60 fold, about 5 to about 40 fold, about 5 to about 20 fold, about 5 to about 10 fold, about 10 to about 200 fold, about 10 to about 150 fold, about to about 100 fold, about 10 to about 80 fold, about 10 to about 60 fold, about 10 to about fold, about 10 to about 20 fold, about 20 to about 200 fold, about 20 to about 150 fold, about 20 to about 100 fold, about 20 to about 80 fold, about 20 to about 60 fold, about 20 to about 40 fold, about 40 to about 200 fold, about 40 to about 150 fold, about 40 to about 100 fold, about 40 to about 80 fold, about 40 to about 60 fold, about 60 to about 200 fold, about 60 to about 150 fold, about 60 to about 100 fold, about 60 to about 80 fold, about 80 to about 200 fold, about 80 to about 150 fold, about 80 to about 100 fold, about 100 to about 200 fold, about 100 to about 150 fold, or about 150 to about 200 fold.

It is contemplated that the tRNA may function in vitro or in vivo and can be provided to a translation system (e.g., an in vitro translation system or a cell) as a mature tRNA (e.g., an aminoacylated tRNA), or as a polynucleotide that encodes the tRNA.

A tRNA may be derived from a bacterial source, e.g., Escherichia coli, Thermus thermophilus, or Bacillus stearothermphilus. A tRNA may also be derived from an archaeal source, e.g., from the Methanosarcinacaea or Desulfitobacterium families, any of the M barkeri (Mb), M alvus (Ma), M. mazei (Mm) or D. hafnisense (Dh) families, Methanobacterium thermoautotrophicum, Haloferax volcanii, Halobacterium species NRC-1, or Archaeoglobus fulgidus. In other embodiments, eukaryotic sources can also be used, for example, plants, algae, protists, fungi, yeasts, or animals (e.g., mammals, insects, arthropods, etc.).

In certain embodiments, the tRNA is derived from an E. coli leucyl tRNA and, for example, is preferentially charged with a leucine analog over the naturally-occurring leucine amino acid by an aminoacyl-tRNA synthetase derived from an E. coli leucyl-tRNA synthetase, e.g., an aminoacyl-tRNA synthetase contemplated herein.

For example, the tRNA may comprise, consist essentially of, or consist of the nucleotide sequence of any one of SEQ ID NOs: 16-43, or a nucleotide sequence that has at least 85%, 90%, 95%, 96%, 97%, 98%, or 99% sequence identity to any one of SEQ ID NOs: 16-43.

In certain embodiments, the tRNA is derived from an E. coli tryptophanyl tRNA and, for example, is preferentially charged with a tryptophan analog over the naturally-occurring tryptophan amino acid by an aminoacyl-tRNA synthetase derived from an E. coli tryptophanyl-tRNA synthetase, e.g., an aminoacyl-tRNA synthetase contemplated herein.

For example, the tRNA may comprise, consist essentially of, or consist of the nucleotide sequence of any one of SEQ ID NOs: 49-54 or 108-113, or a nucleotide sequence that has at least 85%, 90%, 95%, 96%, 97%, 98%, or 99% sequence identity to any one of SEQ ID NOs: 49-54 or 108-113.

In certain embodiments, the tRNA is derived from an E. coli tyrosyl tRNA and, for example, is preferentially charged with a tyrosine analog over the naturally-occurring tyrosine amino acid by an aminoacyl-tRNA synthetase derived from an E. coli tyrosyl-tRNA synthetase, e.g., an aminoacyl-tRNA synthetase contemplated herein.

For example, the tRNA may comprise, consist essentially of, or consist of the nucleotide sequence of any one of SEQ ID NOs: 68-69 or 104-105, or a nucleotide sequence that has at least 85%, 90%, 95%, 96%, 97%, 98%, or 99% sequence identity to any one of SEQ ID NOs: 68-69 or 104-105.

In certain embodiments, the tRNA is derived from aM barkeri pyrrolysyl tRNA and, for example, is preferentially charged with a pyrrolysine analog over the naturally-occurring pyrrolysine amino acid by an aminoacyl-tRNA synthetase derived from a M barkeri pyrrolysyl-tRNA synthetase, e.g., an aminoacyl-tRNA synthetase contemplated herein.

For example, the tRNA may comprise, consist essentially of, or consist of the nucleotide sequence of any one of SEQ ID NOs: 72-100 or 106-107, or a nucleotide sequence that has at least 85%, 90%, 95%, 96%, 97%, 98%, or 99% sequence identity to any one of SEQ ID NOs: 72-100 or 106-107.

It is understood that, throughout the description, in each instance where a tRNA comprises, consists essentially of, or consists of a nucleotide sequence including one or more thymines (T), a tRNA is also contemplated that comprises, consists essentially of, or consists of the same nucleotide sequence including a uracil (U) in place of one or more of the thymines (T), or a uracil (U) in place of all the thymines (T). Similarly, in each instance where a tRNA comprises, consists essentially of, or consists of a nucleotide sequence including one or more uracils (U), a tRNA is also contemplated that comprises, consists essentially of, or consists of a nucleotide sequence including a thymine (T) in place of the one or more of the uracils (U), or a thymine (T) in place of all the uracils (U). In addition, additional modifications to the bases can be present.

Methods for producing recombinant tRNA are described in U.S. Patent Application Publication Nos. 2003/0108885 and 2005/0009049, Forster et al. (2003) PROC. NATL. ACAD. SCI. USA 100(11):6353-6357, and Feng et al. (2003), PROC. NATL. ACAD. SCI. USA 100(10): 5676-5681.

A tRNA may be aminoacylated (i.e., charged) with a desired unnatural amino acid (UAA) by any method, including enzymatic or chemical methods.

Enzymatic molecules capable of charging a tRNA include aminoacyl-tRNA synthetases, e.g., aminoacyl-tRNA synthetases disclosed herein. Additional enzymatic molecules capable of charging tRNA include ribozymes, for example, as described in Illangakekare et al. (1995) SCIENCE 267:643-647, Lohse et al. (1996) NATURE 381:442-444, Murakami et al. (2003) CHEMISTRY AND BIOLOGY 10:1077-1084, U.S. Patent Application Publication No. 2003/0228593.

Chemical aminoacylation methods include those described in Hecht (1992) ACC. CHEM. RES. 25:545, Heckler et al. (1988) BIOCHEM. 1988, 27:7254, Hecht et al. (1978) J. BIOL. CHEM. 253:4517, Cornish et al. (1995) ANGEW. CHEM. INT. ED. ENGL. 34:621, Robertson et al. (1991) J. AM. CHEM. SOC. 113:2722, Noren et al. (1989) SCIENCE 244:182, Bain et al. (1989) J. AM. CHEM. SOC. 111:8013, Bain et al. (1992) NATURE 356:537, Gallivan et al. (1997) CHEM. BIOL. 4:740, Turcatti et al. (1996) J. BIOL. CHEM. 271:19991, Nowak et al. (1995) SCIENCE 268:439, Saks et al. (1996) J. BIOL. CHEM. 271:23169, and Hohsaka et al. (1999) J. AM. CHEM. SOC. 121:34.

### IX. Vectors

Proteins, tRNAs, aminoacyl-tRNA synthetases, or any other molecules of interest may be expressed in a cell of interest by incorporating a gene encoding the molecule into an appropriate expression vector. As used herein, “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system.

Proteins, tRNAs, aminoacyl-tRNA synthetases, or any other molecules of interest may be introduced to a cell of interest by incorporating a gene encoding the molecule into an appropriate transfer vector. The term “transfer vector” refers to a vector comprising a recombinant polynucleotide which can be used to deliver the polynucleotide to the interior of a cell. It is understood that a vector may be both an expression vector and a transfer vector.

Vectors (e.g., expression vectors or transfer vectors) include all those known in the art, such as cosmids, plasmids (e.g., naked or contained in liposomes), retrotransposons (e.g. piggyback, sleeping beauty), and viruses (e.g., lentiviruses, retroviruses, adenoviruses, and adeno-associated viruses) that incorporate the recombinant polynucleotide of interest.

Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both (including but not limited to, shuttle vectors) and selection markers for both prokaryotic and eukaryotic systems.

In certain embodiments, the vector comprises a regulatory sequence or promoter operably linked to the nucleotide sequence encoding the protein, the suppressor tRNA and/or the tRNA synthetase. The term “operably linked” refers to a linkage of polynucleotide elements in a functional relationship. A nucleic acid sequence is “operably linked” when it is placed into a functional relationship with another nucleic acid sequence. For instance, a promoter or enhancer is operably linked to a gene if it affects the transcription of the gene. Operably linked nucleotide sequences are typically contiguous. However, as enhancers generally function when separated from the promoter by several kilobases and intronic sequences may be of variable lengths, some polynucleotide elements may be operably linked but not directly flanked and may even function in trans from a different allele or chromosome.

Exemplary promoters which may be employed include, but are not limited to, the retroviral LTR, the SV40 promoter, the human cytomegalovirus (CMV) promoter, the U6 promoter, the EF1α promoter, the CAG promoter, the H1 promoter, the UbiC promoter, the PGK promoter, the 7SK promoter, a pol II promoter, a pol III promoter, or any other promoter (e.g., cellular promoters such as eukaryotic cellular promoters including, but not limited to, the histone, pol III, and β-actin promoters). Other viral promoters which may be employed include, but are not limited to, adenovirus promoters, TK promoters, and B19 parvovirus promoters. The selection of a suitable promoter will be apparent to those skilled in the art from the teachings contained herein. In certain embodiments, a vector comprises a nucleotide sequence encoding an aminoacyl-tRNA synthetase operably linked to a CMV or an EF1α promoter and/or a nucleotide sequence encoding a suppressor tRNA operably linked to a U6 or an H1 promoter.

In certain embodiments, the vector is a viral vector. The term “virus” is used herein to refer to an obligate intracellular parasite having no protein-synthesizing or energy-generating mechanism. Exemplary viral vectors include retroviral vectors (e.g., lentiviral vectors), adenoviral vectors, adeno-associated viral vectors, herpesviruses vectors, epstein-barr virus (EBV) vectors, polyormavirus vectors (e.g., simian vacuolating virus 40 (SV40) vectors), poxvirus vectors, and pseudotype virus vectors.

The virus may be a RNA virus (having a genome that is composed of RNA) or a DNA virus (having a genome composed of DNA). In certain embodiments, the viral vector is a DNA virus vector. Exemplary DNA viruses include parvoviruses (e.g., adeno-associated viruses), adenoviruses, asfarviruses, herpesviruses (e.g., herpes simplex virus 1 and 2 (HSV-1 and HSV-2), epstein-barr virus (EBV), cytomegalovirus (CMV)), papillonoviruses (e.g., HPV), polyomaviruses (e.g., simian vacuolating virus 40 (SV40)), and poxviruses (e.g., vaccinia virus, cowpox virus, smallpox virus, fowl pox virus, sheeppox virus, myxoma virus). In certain embodiments, the viral vector is a RNA virus vector. Exemplary RNA viruses include bunyaviruses (e.g., hantavirus), coronaviruses, flaviviruses (e.g., yellow fever virus, west nile virus, dengue virus), hepatitis viruses (e.g., hepatitis A virus, hepatitis C virus, hepatitis E virus), influenza viruses (e.g., influenza virus type A, influenza virus type B, influenza virus type C), measles virus, mumps virus, noroviruses (e.g., Norwalk virus), poliovirus, respiratory syncytial virus (RSV), retroviruses (e.g., human immunodeficiency virus-1 (HIV-1)) and toroviruses.

### X. Host Cells and Cell Lines

Also encompassed by the invention are host cells or cell lines (e.g., prokaryotic or eukaryotic host cells or cell lines) that include a tRNA, aminoacyl-tRNA synthetase, unnatural amino acid, nucleic acid, and/or vector disclosed herein. The nucleic acid encoding the engineered tRNA and aminoacyl-tRNA synthetase can be expressed in an expression host cell either as an autonomously replicating vector within the expression host cell (e.g., a plasmid, or viral particle) or via a stable integrated element or series of stable integrated elements in the genome of the expression host cell, e.g., a mammalian host cell.

Host cells are genetically engineered (including but not limited to, transformed, transduced or transfected), for example, using nucleic acids or vectors disclosed herein. For example, in certain embodiments, one or more vectors include coding regions for an orthogonal tRNA, an orthogonal aminoacyl-tRNA synthetase, and, optionally, a protein (e.g., an antibody) to be modified by the inclusion of one or more UAAs, which are operably linked to gene expression control elements that are functional in the desired host cell or cell line. For example, the genes encoding tRNA synthetase and tRNA and an optional selectable marker (e.g., an antibiotic resistance gene, e.g., a puromycin resistance cassette) can be integrated in a transfer vector (e.g., a plasmid, which can be linearized prior to transfection), where for example, the genes encoding the tRNA synthetase can be under the control of a polymerase II promoter (e.g., CMV, EF1α, UbiC, or PGK, e.g., CMV or EF1α) and the genes encoding the tRNA can be under the control of a polymerase III promoter (e.g., U6, 7SK, or H1, e.g., U6). The vectors are transfected into cells and/or microorganisms by standard methods including electroporation or infection by viral vectors, and clones can be selected via expression of the selectable marker (for example, by antibiotic resistance).

As used herein, the term “orthogonal” refers to a molecule (e.g., an orthogonal tRNA or an orthogonal aminoacyl-tRNA synthetase) that is used with reduced efficiency by an expression system of interest (e.g., an endogenous cellular translation system). For example, an orthogonal tRNA in a translation system of interest is aminoacylated by any endogenous aminoacyl-tRNA synthetase of the translation system of interest with reduced or even zero efficiency, when compared to aminoacylation of an endogenous tRNA by an endogenous aminoacyl-tRNA synthetase. In another example, an orthogonal aminoacyl-tRNA synthetase aminoacylates any endogenous tRNA in the translation system of interest with reduced or even zero efficiency, as compared to aminoacylation of an endogenous tRNA by an endogenous aminoacyl-tRNA synthetase.

Exemplary prokaryotic host cells or cell lines include cells derived from a bacteria, e.g., Escherichia coli, Thermus thermophilus, Bacillus stearothermophilus, Pseudomonas fluorescens, Pseudomonas aeruginosa, and Pseudomonas putida. Exemplary eukaryotic host cells or cell lines include cells derived from a plant (e.g., a complex plant such as a monocot or dicot), an algae, a protist, a fungus, a yeast (including Saccharomyces cerevisiae), or an animal (including a mammal, an insect, an arthropod, etc.). Additional exemplary host cells or cell lines include HEK293, HEK293T, Expi293, CHO, CHOK1, Sf9, Sf21, HeLa, U20S, A549, HT1080, CAD, P19, NIH 3T3, L929, N2a, MCF-7, Y79, SO-RB50, HepG2, DUKX-X11, J558L, BHK, COS, Vero, NSO, or ESCs. It is understood that a host cell or cell line can include individual colonies, isolated populations (monoclonal), or a heterogeneous mixture of cells.

A contemplated cell or cell line includes, for example, one or multiple copies of an orthogonal tRNA/aminoacyl-tRNA synthetase pair, optionally stably maintained in the cell's genome or another piece of DNA maintained by the cell. For example, the cell or cell line may contain one or more copies of (i) a tryptophanyl tRNA/aminoacyl-tRNA synthetase pair (wild type or engineered) stably maintained by the cell, and/or (ii) a leucyl tRNA/aminoacyl-tRNA synthetase pair (wild-type or engineered) stably maintained by the cell.

For example, in certain embodiments, the cell line is a stable cell line and the cell line comprises a genome having stably integrated therein (i) a nucleic acid sequence encoding an aminoacyl-tRNA synthetase (e.g., a prokaryotic tryptophanyl-tRNA synthetase mutein capable of charging a tRNA with an unnatural amino acid or a prokaryotic leucyl-tRNA synthetase mutein capable of charging a tRNA with an unnatural amino acid, e.g., a tRNA synthetase mutein disclosed herein); and/or (ii) a nucleic acid sequence encoding a suppressor tRNA (e.g., prokaryotic suppressor tryptophanyl-tRNA capable of being charged with an unnatural amino acid or prokaryotic suppressor leucyl-tRNA capable of being charged with an unnatural amino acid, e.g., a suppressor tRNA disclosed herein).

Methods to introduce a nucleic acid encoding a tRNA and/or an aminoacyl-tRNA synthetase into the genome of a cell of interest, or to stably maintain the nucleic acid in DNA replicated by the cell that is outside of the genome, are well known in the art.

The nucleic acid encoding the tRNA and/or an aminoacyl-tRNA synthetase can be provided to the cell in an expression vector, transfer vector, or DNA cassette, e.g., an expression vector, transfer vector, or DNA cassette disclosed herein. The expression vector transfer vector, or DNA cassette encoding the tRNA and/or aminoacyl-tRNA synthetase can contain one or more copies of the tRNA and/or aminoacyl-tRNA synthetase optionally under the control of an inducible or constitutively active promoter. The expression vector, transfer vector, or DNA cassette may, for example, contain other standard components (enhancers, terminators, etc.). It is contemplated that the nucleic acid encoding the tRNA and the nucleic acid encoding the aminoacyl-tRNA synthetase may be on the same or different vector, may be present in the same or different ratios, and may be introduced into the cell, or stably integrated in the cellular genome, at the same time or sequentially.

One or multiple copies of a DNA cassette encoding the tRNA and/or aminoacyl-tRNA synthetase can be integrated into a host cell genome or stably maintained in the cell using a transposon system (e.g., PiggyBac), a viral vector (e.g., a lentiviral vector or other retroviral vector), CRISPR/Cas9 based recombination, electroporation and natural recombination, a BxB1 recombinase system, or using a replicating/maintained piece of DNA (such as one derived from Epstein-Barr virus).

In order to select for cell lines which stably maintain the nucleic acid encoding the tRNA and/or aminoacyl-tRNA synthetase and/or are efficient at incorporating UAAs into a protein (e.g., an antibody) of interest, a selectable marker can be used. Exemplary selectable markers include zeocin, puromycin, neomycin, dihydrofolate reductase (DHFR), glutamine synthetase (GS), mCherry-EGFP fusion, or other fluorescent proteins. In certain embodiments, a gene encoding a selectable marker protein (or a gene encoding a protein required for a detectable function, e.g., viability, in the presence of the selectable marker) may include a premature stop codon, such that the protein will only be expressed if the cell line is capable of incorporating a UAA at the site of the premature stop codon.

In certain embodiments, to develop a host cell or cell line including two or more tRNA/aminoacyl-tRNA synthetase pairs, one can use multiple identical or distinct UAA directing codons in order to identify host cells or cell lines which have incorporated multiple copies of the two or more tRNA/aminoacyl-tRNA synthetase pairs through iterative rounds of genomic integration and selection. Host cells or cell lines which contain enhanced UAA incorporation efficiency, low background, and decreased toxicity can first be isolated via a selectable marker containing one or more stop codons. Subsequently, the host cells or cell lines can be subjected to a selection scheme to identify host cells or cell lines which contain the desired copies of tRNA/aminoacyl-tRNA synthetase pairs and express a gene of interest (either genomically integrated or not) containing one or more stop codons. Protein expression may be assayed using any method known in the art, including for example, Western blot using an antibody that binds the protein of interest or a C-terminal tag.

The host cells or cell lines be cultured in conventional nutrient media modified as appropriate for such activities as, for example, screening steps, activating promoters or selecting transformants. These cells can optionally be cultured into transgenic organisms. Other useful references, e.g. for cell isolation and culture (e.g., for subsequent nucleic acid isolation) include Freshney (1994) Culture of Animal Cells, a Manual of Basic Technique, third edition, Wiley-Liss, New York; Payne et al. (1992) Plant Cell and Tissue Culture in Liquid Systems John Wiley & Sons, Inc. New York, N.Y.; Gamborg and Phillips (eds.) (1995) Plant Cell, Tissue and Organ Culture; Fundamental Methods Springer Lab Manual, Springer-Verlag (Berlin Heidelberg N.Y.) and Atlas and Parks (eds.) The Handbook of Microbiological Media (1993) CRC Press, Boca Raton, Fla.

The production of an exemplary cell line capable of producing proteins (e.g., antibodies) incorporating a UAA is described in Roy et al. (2020) MABS 12(1), e1684749).

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

SEQUENCE LISTING

| SEQ | | |
|---|---|---|
| ID | | |
| NO | Description | Sequence |
| 1 | E. coli | MQEQYRPEEIESKVQLHWDEKRTFEVTEDESKEKYYCLSMLPYPSGR |
| | LeuRS | LHMGHVRNYTIGDVIARYQRMLGKNVLQPIGWDAFGLPAEGAAVKNN |
| | | TAPAPWTYDNIAYMKNQLKMLGFGYDWSRELATCTPEYYRWEQKFFT |
| | | ELYKKGLVYKKTSAVNWCPNDQTVLANEQVIDGCCWRCDTKVERKEI |
| | | PQWFIKITAYADELLNDLDKLDHWPDTVKTMQRNWIGRSEGVEITEN |
| | | VNDYDNTLTVYTTRPDTFMGCTYLAVAAGHPLAQKAAENNPELAAFI |
| | | DECRNTKVAEAEMATMEKKGVDTGFKAVHPLTGEEIPVWAANFVLME |
| | | YGTGAVMAVPGHDQRDYEFASKYGLNIKPVILAADGSEPDLSQQALT |
| | | EKGVLFNSGEFNGLDHEAAFNAIADKLTAMGVGERKVNYRLRDWGVS |
| | | RQRYWGAPIPMVTLEDGTVMPTPDDQLPVILPEDVVMDGITSPIKAD |
| | | PEWAKTTVNGMPALRETDTFDTFMESSWYYARYTCPQYKEGMLDSEA |
| | | ANYWLPVDIYIGGIEHAIMHLLYFRFFHKLMRDAGMVNSDEPAKQLL |
| | | CQGMVLADAFYYVGENGERNWVSPVDAIVERDEKGRIVKAKDAAGHE |
| | | LVYTGMSKMSKSKNNGIDPQVMVERYGADTVRLFMMFASPADMTLEW |
| | | QESGVEGANRFLKRVWKLVYEHTAKGDVAALNVDALTENQKALRRDV |
| | | HKTIAKVTDDIGRRQTENTAIAAIMELMNKLAKAPTDGEQDRALMQE |
| | | ALLAVVRMLNPFTPHICFTLWQELKGEGDIDNAPWPVADEKAMVEDS |
| | | TLVVVQVNGKVRAKITVPVDATEEQVRERAGQEHLVAKYLDGVTVRK |
| | | VIYVPGKLLNLVVGGPV |
| 2 | LeuRS.v1 | MEEQYRPEEIESKVQLHWDKKRTFEVTEDESKEKYYCLSISPYPSGR |
| | | LHMGHVRNYTIGDVIARYQRMLGKNVLQPIGWDAFGLPAEGAAVKNN |
| | | TAPAPWTYDNIAYMKNQLKMLGFGYDWSRELATCTPEYYRWEQKFFT |
| | | ELYKKGLVYKKTSAVNWCPNDQTVLANEQVIDGCCWRCDTKVERKEI |
| | | PQWFIKITAYADELLNDLDKLDHWPDTVKTMQRNWIGRSEGVEITEN |
| | | VNDYDNTLTVYTTRPDAFMGCTYLAVAAGHPLAQKAAENNPELAAFI |
| | | DECRNTKVAEAEMATMEKKGVDTGFKAVHPLTGEEIPVWAANFVLME |
| | | YGTGAVMAVPGHDQRDYEFASKYGLNIKPVILAADGSEPDLSQQALT |
| | | EKGVLFNSGEFNGLDHEAAFNAIADKLTAMGVGERKVNYRLRDWGVS |
| | | RQRYWGAPIPMVTLEDGTVMPTPDDQLPVILPEDVVMDGITSPIKAD |
| | | PEWAKTTVNGMPALRETDTFDTFMESSWIYARYTCPQYKEGMLDSEA |
| | | ANYWLPVDIAIGGIEHAIMGLLYFRFFHKLMRDAGMVNSDEPAKQLL |
| | | CQGMVLADAFYYVGENGERNWVSPVDAIVERDEKGRIVKAKDAAGHE |
| | | LVYTGMSKMSKSKNNGIDPQVMVERYGADTVRLFMMFASPADMTLEW |
| | | QESGVEGANRFLKRVWKLVYEHTAKGDVAALNVDALTENQKALRRDV |
| | | HKTIAKVTDDIGRRQTFNTAIAAIMELMNKLAKAPTDGEQDRALMQE |
| | | ALLAVVRMLNPFTPHICFTLWQELKGEGDIDNAPWPVADEKAMVEDS |
| | | TLVVVQVNGKVRAKITVPVDATEEQVRERAGQEHLVAKYLDGVTVRK |
| | | VIYVPGKLLNLVVGGPV |
| 3 | LeuRS.v2 | MEEQYRPEEIESKVQLHWDKKRTFEVTEDESKEKYYCLSVSPYPSGR |
| | | LHMGHVRNYTIGDVIARYQRMLGKNVLQPIGWDAFGLPAEGAAVKNN |
| | | TAPAPWTYDNIAYMKNQLKMLGFGYDWSRELATCTPEYYRWEQKFFT |
| | | ELYKKGLVYKKTSAVNWCPNDQTVLANEQVIDGCCWRCDTKVERKEI |
| | | PQWFIKITAYADELLNDLDKLDHWPDTVKTMQRNWIGRSEGVEITEN |
| | | VNDYDNTLTVYTTRPDRFMGCTYLAVAAGHPLAQKAAENNPELAAFI |
| | | DECRNTKVAEAEMATMEKKGVDTGFKAVHPLTGEEIPVWAANFVLME |
| | | YGTGAVMAVPGHDQRDYEFASKYGLNIKPVILAADGSEPDLSQQALT |
| | | EKGVLFNSGEFNGLDHEAAFNAIADKLTAMGVGERKVNYRLRDWGVS |
| | | RQRYWGAPIPMVTLEDGTVMPTPDDQLPVILPEDVVMDGITSPIKAD |
| | | PEWAKTTVNGMPALRETDTFDTFMESSWSYARYTCPQYKEGMLDSEA |
| | | ANYWLPVDILIGGIEHAIMGLLYFRFFHKLMRDAGMVNSDEPAKQLL |
| | | CQGMVLADAFYYVGENGERNWVSPVDAIVERDEKGRIVKAKDAAGHE |
| | | LVYTGMSKMSKSKNNGIDPQVMVERYGADTVRLEMMFASPADMTLEW |
| | | QESGVEGANRFLKRVWKLVYEHTAKGDVAALNVDALTENQKALRRDV |
| | | HKTIAKVTDDIGRRQTENTAIAAIMELMNKLAKAPTDGEQDRALMQE |
| | | ALLAVVRMLNPFTPHICFTLWQELKGEGDIDNAPWPVADEKAMVEDS |
| | | TLVVVQVNGKVRAKITVPVDATEEQVRERAGQEHLVAKYLDGVTVRK |
| | | VIYVPGKLLNLVVGGPV |
| 4 | LeuRS.v3 | MEEQYRPEEIESKVQLHWDEKRTFEVTEDESKEKYYCLSILPYPSGR |
| | | LHMGHVRNYTIGDVIARYQRMLGKNVLQPIGWDAFGLPAEGAAVKNN |
| | | TAPAPWTYDNIAYMKNQLKMLGFGYDWSRELATCTPEYYRWEQKFFT |
| | | ELYKKGLVYKKTSAVNWCPNDQTVLANEQVIDGCCWRCDTKVERKEI |
| | | PQWFIKITAYADELLNDLDKLDHWPDTVKTMQRNWIGRSEGVEITEN |
| | | VNDYDNTLTVYTTRPDAFMGCTYLAVAAGHPLAQKAAENNPELAAFI |
| | | DECRNTKVAEAEMATMEKKGVDTGFKAVHPLTGEEIPVWAANFVLME |
| | | YGTGAVMAVPGHDQRDYEFASKYGLNIKPVILAADGSEPDLSQQALT |
| | | EKGVLFNSGEFNGLDHEAAFNAIADKLTAMGVGERKVNYRLRDWGVS |
| | | RQRYWGAPIPMVTLEDGTVMPTPDDQLPVILPEDVVMDGITSPIKAD |
| | | PEWAKTTVNGMPALRETDTFDTFMESSWIYARYTCPQYKEGMLDSEA |
| | | ANYWLPVDIAIGGIEHAIMGLLYFRFFHKLMRDAGMVNSDEPAKQLL |
| | | CQGMVLADAFYYVGENGERNWVSPVDAIVERDEKGRIVKAKDAAGHE |
| | | LVYTGMSKMSKSKNNGIDPQVMVERYGADTVRLFMMFASPADMTLEW |
| | | QESGVEGANRFLKRVWKLVYEHTAKGDVAALNVDALTENQKALRRDV |
| | | HKTIAKVTDDIGRRQTFNTAIAAIMELMNKLAKAPTDGEQDRALMQE |
| | | ALLAVVRMLNPFTPHICFTLWQELKGEGDIDNAPWPVADEKAMVEDS |
| | | TLVVVQVNGKVRAKITVPVDATEEQVRERAGQEHLVAKYLDGVTVRK |
| | | VIYVPGKLLNLVVGGPV |
| 5 | V9 | MEEQYRPEEIESKVQLHWDMKRTFEVTEDESKEKYYCLSISPYPSGR |
| | | LHMGHVRNYTIGDVIARYQRMLGKNVLQPIGWDAFGLPAEGAAVKNN |
| | | TAPAPWTYDNIAYMKNQLKMLGFGYDWSRELATCTPEYYRWEQKFFT |
| | | ELYKKGLVYKKTSAVNWCPNDQTVLANEQVIDGCCWRCDTKVERKEI |
| | | PQWFIKITAYADELLNDLDKLDHWPDTVKTMQRNWIGRSEGVEITEN |
| | | VNDYDNTLTVYTTRPDAFMGCTYLAVAAGHPLAQKAAENNPELAAFI |
| | | DECRNTKVAEAEMATMEKKGVDTGFKAVHPLTGEEIPVWAANFVLME |
| | | YGTGAVMAVPGHDQRDYEFASKYGLNIKPVILAADGSEPDLSQQALT |
| | | EKGVLFNSGEFNGLDHEAAFNAIADKLTAMGVGERKVNYRLRDWGVS |
| | | RQRYWGAPIPMVTLEDGTVMPTPDDQLPVILPEDVVMDGITSPIKAD |
| | | PEWAKTTVNGMPALRETDTFDTFMESSWIYARYTCPQYKEGMLDSEA |
| | | ANYWLPVDIAIGGIEHAIMGLLYFRFFHKLMRDAGMVNSDEPAKQLL |
| | | CQGMVLADAFYYVGENGERNWVSPVDAIVERDEKGRIVKAKDAAGHE |
| | | LVYTGMSKMSKSKNNGIDPQVMVERYGADTVRLFMMFASPADMTLEW |
| | | QESGVEGANRFLKRVWKLVYEHTAKGDVAALNVDALTENQKALRRDV |
| | | HKTIAKVTDDIGRRQTENTAIAAIMELMNKLAKAPTDGEQDRALMQE |
| | | ALLAVVRMLNPFTPHICFTLWQELKGEGDIDNAPWPVADEKAMVEDS |
| | | TLVVVQVNGKVRAKITVPVDATEEQVRERAGQEHLVAKYLDGVTVRK |
| | | VIYVPGKLLNLVVGGPV |
| 6 | V10 | MEEQYRPEEIESKVQLHWDVKRTFEVTEDESKEKYYCLSISPYPSGR |
| | | LHMGHVRNYTIGDVIARYQRMLGKNVLQPIGWDAFGLPAEGAAVKNN |
| | | TAPAPWTYDNIAYMKNQLKMLGFGYDWSRELATCTPEYYRWEQKFFT |
| | | ELYKKGLVYKKTSAVNWCPNDQTVLANEQVIDGCCWRCDTKVERKEI |
| | | PQWFIKITAYADELLNDLDKLDHWPDTVKTMQRNWIGRSEGVEITEN |
| | | VNDYDNTLTVYTTRPDAFMGCTYLAVAAGHPLAQKAAENNPELAAFI |
| | | DECRNTKVAEAEMATMEKKGVDTGFKAVHPLTGEEIPVWAANFVLME |
| | | YGTGAVMAVPGHDQRDYEFASKYGLNIKPVILAADGSEPDLSQQALT |
| | | EKGVLFNSGEFNGLDHEAAFNAIADKLTAMGVGERKVNYRLRDWGVS |
| | | RQRYWGAPIPMVTLEDGTVMPTPDDQLPVILPEDVVMDGITSPIKAD |
| | | PEWAKTTVNGMPALRETDTFDTFMESSWIYARYTCPQYKEGMLDSEA |
| | | ANYWLPVDIAIGGIEHAIMGLLYFRFFHKLMRDAGMVNSDEPAKQLL |
| | | CQGMVLADAFYYVGENGERNWVSPVDAIVERDEKGRIVKAKDAAGHE |
| | | LVYTGMSKMSKSKNNGIDPQVMVERYGADTVRLFMMFASPADMTLEW |
| | | QESGVEGANRFLKRVWKLVYEHTAKGDVAALNVDALTENQKALRRDV |
| | | HKTIAKVTDDIGRRQTFNTAIAAIMELMNKLAKAPTDGEQDRALMQE |
| | | ALLAVVRMLNPFTPHICFTLWQELKGEGDIDNAPWPVADEKAMVEDS |
| | | TLVVVQVNGKVRAKITVPVDATEEQVRERAGQEHLVAKYLDGVTVRK |
| | | VIYVPGKLLNLVVGGPV |
| 7 | V26 | MEEQYRPEEIESKVQLHWDKKRTFEVTEDESKEKYYCLSIVPYPSGR |
| | | LHMGHVRNYTIGDVIARYQRMLGKNVLQPIGWDAFGLPAEGAAVKNN |
| | | TAPAPWTYDNIAYMKNQLKMLGFGYDWSRELATCTPEYYRWEQKFFT |
| | | ELYKKGLVYKKTSAVNWCPNDQTVLANEQVIDGCCWRCDTKVERKEI |
| | | PQWFIKITAYADELLNDLDKLDHWPDTVKTMQRNWIGRSEGVEITEN |
| | | VNDYDNTLTVYTTRPDAFMGCTYLAVAAGHPLAQKAAENNPELAAFI |
| | | DECRNTKVAEAEMATMEKKGVDTGFKAVHPLTGEEIPVWAANFVLME |
| | | YGTGAVMAVPGHDQRDYEFASKYGLNIKPVILAADGSEPDLSQQALT |
| | | EKGVLFNSGEFNGLDHEAAFNAIADKLTAMGVGERKVNYRLRDWGVS |
| | | RQRYWGAPIPMVTLEDGTVMPTPDDQLPVILPEDVVMDGITSPIKAD |
| | | PEWAKTTVNGMPALRETDTFDTFMESSWIYARYTCPQYKEGMLDSEA |
| | | ANYWLPVDIAIGGIEHAIMGLLYFRFFHKLMRDAGMVNSDEPAKQLL |
| | | CQGMVLADAFYYVGENGERNWVSPVDAIVERDEKGRIVKAKDAAGHE |
| | | LVYTGMSKMSKSKNNGIDPQVMVERYGADTVRLFMMFASPADMTLEW |
| | | QESGVEGANRFLKRVWKLVYEHTAKGDVAALNVDALTENQKALRRDV |
| | | HKTIAKVTDDIGRRQTFNTAIAAIMELMNKLAKAPTDGEQDRALMQE |
| | | ALLAVVRMLNPFTPHICFTLWQELKGEGDIDNAPWPVADEKAMVEDS |
| | | TLVVVQVNGKVRAKITVPVDATEEQVRERAGQEHLVAKYLDGVTVRK |
| | | VIYVPGKLLNLVVGGPV |
| 8 | V29 | MEEQYRPEEIESKVQLHWDKKRTFEVTEDESKEKYYCLSIAPYPSGR |
| | | LHMGHVRNYTIGDVIARYQRMLGKNVLQPIGWDAFGLPAEGAAVKNN |
| | | TAPAPWTYDNIAYMKNQLKMLGFGYDWSRELATCTPEYYRWEQKFFT |
| | | ELYKKGLVYKKTSAVNWCPNDQTVLANEQVIDGCCWRCDTKVERKEI |
| | | PQWFIKITAYADELLNDLDKLDHWPDTVKTMQRNWIGRSEGVEITEN |
| | | VNDYDNTLTVYTTRPDAFMGCTYLAVAAGHPLAQKAAENNPELAAFI |
| | | DECRNTKVAEAEMATMEKKGVDTGFKAVHPLTGEEIPVWAANFVLME |
| | | YGTGAVMAVPGHDQRDYEFASKYGLNIKPVILAADGSEPDLSQQALT |
| | | EKGVLFNSGEFNGLDHEAAFNAIADKLTAMGVGERKVNYRLRDWGVS |
| | | RQRYWGAPIPMVTLEDGTVMPTPDDQLPVILPEDVVMDGITSPIKAD |
| | | PEWAKTTVNGMPALRETDTFDTFMESSWIYARYTCPQYKEGMLDSEA |
| | | ANYWLPVDIAIGGIEHAIMGLLYFRFFHKLMRDAGMVNSDEPAKQLL |
| | | CQGMVLADAFYYVGENGERNWVSPVDAIVERDEKGRIVKAKDAAGHE |
| | | LVYTGMSKMSKSKNNGIDPQVMVERYGADTVRLFMMFASPADMTLEW |
| | | QESGVEGANRFLKRVWKLVYEHTAKGDVAALNVDALTENQKALRRDV |
| | | HKTIAKVTDDIGRRQTFNTAIAAIMELMNKLAKAPTDGEQDRALMQE |
| | | ALLAVVRMLNPFTPHICFTLWQELKGEGDIDNAPWPVADEKAMVEDS |
| | | TLVVVQVNGKVRAKITVPVDATEEQVRERAGQEHLVAKYLDGVTVRK |
| | | VIYVPGKLLNLVVGGPV |
| 9 | V48 | MEEQYRPEEIESKVQLHWDKKRTFEVTEDESKEKYYCLSISPYPSGR |
| | | LHMGHVRNYTIGDVIARYQRMLGKNVLQPIGWDAFGLPAEGAAVKNN |
| | | TAPAPWTYDNIAYMKNQLKMLGFGYDWSRELATCTPEYYRWEQKFFT |
| | | ELYKKGLVYKKTSAVNWCPNDQTVLANEQVIDGCCWRCDTKVERKEI |
| | | PQWFIKITAYADELLNDLDKLDHWPDTVKTMQRNWIGRSEGVEITEN |
| | | VNDYDNTLTVYTTRPDAFMGCTYLAVAAGHPLAQKAAENNPELAAFI |
| | | DECRNTKVAEAEMATMEKKGVDTGFKAVHPLTGEEIPVWAANFVLME |
| | | YGTGAVMAVPGHDQRDYEFASKYGLNIKPVILAADGSEPDLSQQALT |
| | | EKGVLFNSGEFNGLDHEAAFNAIADKLTAMGVGERKVNYRLRDWGVS |
| | | RQRYWGAPIPMVTLEDGTVMPTPDDQLPVILPEDVVMDGITSPIKAD |
| | | PEWAKTTVNGMPALRETDTFDTFMESSWAYARYTCPQYKEGMLDSEA |
| | | ANYWLPVDIAIGGIEHAIMGLLYFRFFHKLMRDAGMVNSDEPAKQLL |
| | | CQGMVLADAFYYVGENGERNWVSPVDAIVERDEKGRIVKAKDAAGHE |
| | | LVYTGMSKMSKSKNNGIDPQVMVERYGADTVRLFMMFASPADMTLEW |
| | | QESGVEGANRFKRVWKLVYEHTAKGDVAALNVDALTENQKALRRDVH |
| | | KTIAKVTDDIGRRQTENTAIAAIMELMNKLAKAPTDGEQDRALMQEA |
| | | LLAVVRMLNPFTPHICFTLWQELKGEGDIDNAPWPVADEKAMVEDST |
| | | LVVVQVNGKVRAKITVPVDATEEQVRERAGQEHLVAKYLDGVTVRKV |
| | | IYVPGKLLNLVVGGPV |
| 10 | V50 | MEEQYRPEEIESKVQLHWDKKRTFEVTEDESKEKYYCLSISPYPSGR |
| | | LHMGHVRNYTIGDVIARYQRMLGKNVLQPIGWDAFGLPAEGAAVKNN |
| | | TAPAPWTYDNIAYMKNQLKMLGFGYDWSRELATCTPEYYRWEQKFFT |
| | | ELYKKGLVYKKTSAVNWCPNDQTVLANEQVIDGCCWRCDTKVERKEI |
| | | PQWFIKITAYADELLNDLDKLDHWPDTVKTMQRNWIGRSEGVEITEN |
| | | VNDYDNTLTVYTTRPDAFMGCTYLAVAAGHPLAQKAAENNPELAAFI |
| | | DECRNTKVAEAEMATMEKKGVDTGFKAVHPLTGEEIPVWAANFVLME |
| | | YGTGAVMAVPGHDQRDYEFASKYGLNIKPVILAADGSEPDLSQQALT |
| | | EKGVLENSGEFNGLDHEAAFNAIADKLTAMGVGERKVNYRLRDWGVS |
| | | RQRYWGAPIPMVTLEDGTVMPTPDDQLPVILPEDVVMDGITSPIKAD |
| | | PEWAKTTVNGMPALRETDTFDTFMESSWHYARYTCPQYKEGMLDSEA |
| | | ANYWLPVDIAIGGIEHAIMGLLYFRFFHKLMRDAGMVNSDEPAKQLL |
| | | CQGMVLADAFYYVGENGERNWVSPVDAIVERDEKGRIVKAKDAAGHE |
| | | LVYTGMSKMSKSKNNGIDPQVMVERYGADTVRLFMMFASPADMTLEW |
| | | QESGVEGANRFLKRVWKLVYEHTAKGDVAALNVDALTENQKALRRDV |
| | | HKTIAKVTDDIGRRQTENTAIAAIMELMNKLAKAPTDGEQDRALMQE |
| | | ALLAVVRMLNPFTPHICFTLWQELKGEGDIDNAPWPVADEKAMVEDS |
| | | TLVVVQVNGKVRAKITVPVDATEEQVRERAGQEHLVAKYLDGVTVRK |
| | | VIYVPGKLLNLVVGGPV |
| 11 | V54 | MEEQYRPEEIESKVQLHWDKKRTFEVTEDESKEKYYCLSISPYPSGR |
| | | LHMGHVRNYTIGDVIARYQRMLGKNVLQPIGWDAFGLPAEGAAVKNN |
| | | TAPAPWTYDNIAYMKNQLKMLGFGYDWSRELATCTPEYYRWEQKFFT |
| | | ELYKKGLVYKKTSAVNWCPNDQTVLANEQVIDGCCWRCDTKVERKEI |
| | | PQWFIKITAYADELLNDLDKLDHWPDTVKTMQRNWIGRSEGVEITEN |
| | | VNDYDNTLTVYTTRPDAFMGCTYLAVAAGHPLAQKAAENNPELAAFI |
| | | DECRNTKVAEAEMATMEKKGVDTGFKAVHPLTGEEIPVWAANFVLME |
| | | YGTGAVMAVPGHDQRDYEFASKYGLNIKPVILAADGSEPDLSQQALT |
| | | EKGVLENSGEFNGLDHEAAFNAIADKLTAMGVGERKVNYRLRDWGVS |
| | | RQRYWGAPIPMVTLEDGTVMPTPDDQLPVILPEDVVMDGITSPIKAD |
| | | PEWAKTTVNGMPALRETDTFDTFMESSWIYARYTCPQYKEGMLDSEA |
| | | ANYWLPVDIIIGGIEHAIMGLLYFRFFHKLMRDAGMVNSDEPAKQLL |
| | | CQGMVLADAFYYVGENGERNWVSPVDAIVERDEKGRIVKAKDAAGHE |
| | | LVYTGMSKMSKSKNNGIDPQVMVERYGADTVRLFMMFASPADMTLEW |
| | | QESGVEGANRFLKRVWKLVYEHTAKGDVAALNVDALTENQKALRRDV |
| | | HKTIAKVTDDIGRRQTFNTAIAAIMELMNKLAKAPTDGEQDRALMQE |
| | | ALLAVVRMLNPFTPHICFTLWQELKGEGDIDNAPWPVADEKAMVEDS |
| | | TLVVVQVNGKVRAKITVPVDATEEQVRERAGQEHLVAKYLDGVTVRK |
| | | VIYVPGKLLNLVVGGPV |
| 12 | V55 | MEEQYRPEEIESKVQLHWDKKRTFEVTEDESKEKYYCLSISPYPSGR |
| | | LHMGHVRNYTIGDVIARYQRMLGKNVLQPIGWDAFGLPAEGAAVKNN |
| | | TAPAPWTYDNIAYMKNQLKMLGFGYDWSRELATCTPEYYRWEQKFFT |
| | | ELYKKGLVYKKTSAVNWCPNDQTVLANEQVIDGCCWRCDTKVERKEI |
| | | PQWFIKITAYADELLNDLDKLDHWPDTVKTMQRNWIGRSEGVEITEN |
| | | VNDYDNTLTVYTTRPDAFMGCTYLAVAAGHPLAQKAAENNPELAAFI |
| | | DECRNTKVAEAEMATMEKKGVDTGFKAVHPLTGEEIPVWAANFVLME |
| | | YGTGAVMAVPGHDQRDYEFASKYGLNIKPVILAADGSEPDLSQQALT |
| | | EKGVLFNSGEFNGLDHEAAFNAIADKLTAMGVGERKVNYRLRDWGVS |
| | | RQRYWGAPIPMVTLEDGTVMPTPDDQLPVILPEDVVMDGITSPIKAD |
| | | PEWAKTTVNGMPALRETDTFDTFMESSWIYARYTCPQYKEGMLDSEA |
| | | ANYWLPVDIVIGGIEHAIMGLLYFRFFHKLMRDAGMVNSDEPAKQLL |
| | | CQGMVLADAFYYVGENGERNWVSPVDAIVERDEKGRIVKAKDAAGHE |
| | | LVYTGMSKMSKSKNNGIDPQVMVERYGADTVRLEMMFASPADMTLEW |
| | | QESGVEGANRFLKRVWKLVYEHTAKGDVAALNVDALTENQKALRRDV |
| | | HKTIAKVTDDIGRRQTFNTAIAAIMELMNKLAKAPTDGEQDRALMQE |
| | | ALLAVVRMLNPFTPHICFTLWQELKGEGDIDNAPWPVADEKAMVEDS |
| | | TLVVVQVNGKVRAKITVPVDATEEQVRERAGQEHLVAKYLDGVTVRK |
| | | VIYVPGKLLNLVVGGPV |
| 13 | V56 | MEEQYRPEEIESKVQLHWDKKRTFEVTEDESKEKYYCLSISPYPSGR |
| | | LHMGHVRNYTIGDVIARYQRMLGKNVLQPIGWDAFGLPAEGAAVKNN |
| | | TAPAPWTYDNIAYMKNQLKMLGFGYDWSRELATCTPEYYRWEQKFFT |
| | | ELYKKGLVYKKTSAVNWCPNDQTVLANEQVIDGCCWRCDTKVERKEI |
| | | PQWFIKITAYADELLNDLDKLDHWPDTVKTMQRNWIGRSEGVEITEN |
| | | VNDYDNTLTVYTTRPDAFMGCTYLAVAAGHPLAQKAAENNPELAAFI |
| | | DECRNTKVAEAEMATMEKKGVDTGFKAVHPLTGEEIPVWAANFVLME |
| | | YGTGAVMAVPGHDQRDYEFASKYGLNIKPVILAADGSEPDLSQQALT |
| | | EKGVLFNSGEFNGLDHEAAFNAIADKLTAMGVGERKVNYRLRDWGVS |
| | | RQRYWGAPIPMVTLEDGTVMPTPDDQLPVILPEDVVMDGITSPIKAD |
| | | PEWAKTTVNGMPALRETDTFDTEMESSWIYARYTCPQYKEGMLDSEA |
| | | ANYWLPVDIGIGGIEHAIMGLLYFRFFHKLMRDAGMVNSDEPAKQLL |
| | | CQGMVLADAFYYVGENGERNWVSPVDAIVERDEKGRIVKAKDAAGHE |
| | | LVYTGMSKMSKSKNNGIDPQVMVERYGADTVRLEMMFASPADMTLEW |
| | | QESGVEGANRFLKRVWKLVYEHTAKGDVAALNVDALTENQKALRRDV |
| | | HKTIAKVTDDIGRRQTFNTAIAAIMELMNKLAKAPTDGEQDRALMQE |
| | | ALLAVVRMLNPFTPHICFTLWQELKGEGDIDNAPWPVADEKAMVEDS |
| | | TLVVVQVNGKVRAKITVPVDATEEQVRERAGQEHLVAKYLDGVTVRK |
| | | VIYVPGKLLNLVVGGPV |
| 14 | | MX₂EQYRPEEIESKVQLHWDX₂₀KRTFEVTEDESKEKYYCLSX₄₀X₄₁P |
| | | YPSGRLHMGHVRNYTIGDVIARYQRMLGKNVLQPIGWDAFGLPAEGA |
| | | AVKNNTAPAPWTYDNIAYMKNQLKMLGFGYDWSRELATCTPEYYRWE |
| | | QKFFTELYKKGLVYKKTSAVNWCPNDQTVLANEQVIDGCCWRCDTKV |
| | | ERKEIPQWFIKITAYADELLNDLDKLDHWPDTVKTMQRNWIGRSEGV |
| | | EITFNVNDYDNTLTVYTTRPDX₂₅₂FMGCTYLAVAAGHPLAQKAAENN |
| | | PELAAFIDECRNTKVAEAEMATMEKKGVDTGFKAVHPLTGEEIPVWA |
| | | ANFVLMEYGTGAVMAVPGHDQRDYEFASKYGLNIKPVILAADGSEPD |
| | | LSQQALTEKGVLFNSGEFNGLDHEAAFNAIADKLTAMGVGERKVNYR |
| | | LRDWGVSRQRYWGAPIPMVTLEDGTVMPTPDDQLPVILPEDVVMDGI |
| | | TSPIKADPEWAKTTVNGMPALRETDTFDTFMESSWX₄₉₉YARYTCPQY |
| | | KEGMLDSEAANYWLPVDIX₅₂₇IGGIEHAIMX₅₃₇LLYFRFFHKLMRDA |
| | | GMVNSDEPAKQLLCQGMVLADAFYYVGENGERNWVSPVDAIVERDEK |
| | | GRIVKAKDAAGHELVYTGMSKMSKSKNNGIDPQVMVERYGADTVRLF |
| | | MMFASPADMTLEWQESGVEGANRFLKRVWKLVYEHTAKGDVAALNVD |
| | | ALTENQKALRRDVHKTIAKVTDDIGRRQTENTAIAAIMELMNKLAKA |
| | | PTDGEQDRALMQEALLAVVRMLNPFTPHICFTLWQELKGEGDIDNAP |
| | | WPVADEKAMVEDSTLVVVQVNGKVRAKITVPVDATEEQVRERAGQEH |
| | | LVAKYLDGVTVRKVIYVPGKLLNLVVGGPV |
| | | wherein X₂ is Q or E, X₂₀ is E, K, V or M, X₄₀ is |
| | | M, I, or V, X₄₁ is L, S, V, or A, X₂₅₂ is T, A, |
| | | or R, X₄₉₉ is Y, A, I, H, or S, X₅₂₇ is Y, A, I, |
| | | L, or V, and X₅₃₇ is H or G |
| 15 | | MEEQYRPEEIESKVQLHWDX₂₀KRTFEVTEDESKEKYYCLSIX₄₁PYP |
| | | SGRLHMGHVRNYTIGDVIARYQRMLGKNVLQPIGWDAFGLPAEGAAV |
| | | KNNTAPAPWTYDNIAYMKNQLKMLGFGYDWSRELATCTPEYYRWEQK |
| | | FFTELYKKGLVYKKTSAVNWCPNDQTVLANEQVIDGCCWRCDTKVER |
| | | KEIPQWFIKITAYADELLNDLDKLDHWPDTVKTMQRNWIGRSEGVEI |
| | | TFNVNDYDNTLTVYTTRPDAFMGCTYLAVAAGHPLAQKAAENNPELA |
| | | AFIDECRNTKVAEAEMATMEKKGVDTGFKAVHPLTGEEIPVWAANFV |
| | | LMEYGTGAVMAVPGHDQRDYEFASKYGLNIKPVILAADGSEPDLSQQ |
| | | ALTEKGVLFNSGEFNGLDHEAAFNAIADKLTAMGVGERKVNYRLRDW |
| | | GVSRQRYWGAPIPMVTLEDGTVMPTPDDQLPVILPEDVVMDGITSPI |
| | | KADPEWAKTTVNGMPALRETDTFDTFMESSWX₄₉₉YARYTCPQYKEGM |
| | | LDSEAANYWLPVDIX₅₂₇IGGIEHAIMGLLYFRFFHKLMRDAGMVNSD |
| | | EPAKQLLCQGMVLADAFYYVGENGERNWVSPVDAIVERDEKGRIVKA |
| | | KDAAGHELVYTGMSKMSKSKNNGIDPQVMVERYGADTVRLFMMFASP |
| | | ADMTLEWQESGVEGANRFLKRVWKLVYEHTAKGDVAALNVDALTENQ |
| | | KALRRDVHKTIAKVTDDIGRRQTFNTAIAAIMELMNKLAKAPTDGEQ |
| | | DRALMQEALLAVVRMLNPFTPHICFTLWQELKGEGDIDNAPWPVADE |
| | | KAMVEDSTLVVVQVNGKVRAKITVPVDATEEQVRERAGQEHLVAKYL |
| | | DGVTVRKVIYVPGKLLNLVVGGPV |
| | | wherein X₂₀ is K, V or M, X₄₁ is S, V, or A, X₄₉₉ |
| | | is A, I, or H, and X₅₂₇ is A, I, or V |
| 16 | Leu- | GCCCGGATGGTGGAATCGGTAGACACAAGGGATTctaAATCCCTCGG |
| | tRNA.wtCUA | CGTTCGCGCTGTGCGGGTTCAAGTCCCGCTCCGGGtA |
| 17 | Leu- | GCCCGGATGGTGGAATCGGTAGACACAAGGGATTtcaAATCCCTCGG |
| | tRNA.wtUCA | CGTTCGCGCTGTGCGGGTTCAAGTCCCGCTCCGGGtA |
| 18 | Leu- | GCCCGGATGGTGGAATCGGTAGACACAAGGGATTttaAATCCCTCGG |
| | tRNA.wtUUA | CGTTCGCGCTGTGCGGGTTCAAGTCCCGCTCCGGGtA |
| 19 | Leu- | GCCCGGATGGTGGAATCGGTAGACACAAGGGACTctaAATCCCTCGG |
| | tRNA.h1CUA | CGTTCGCGCTGTGCGGGTTCAAGTCCCGCTCCGGGCA |
| 20 | Leu- | GCCCGGATGGTGGAATCGGTAGACACAAGGGACTtcaAATCCCTCGG |
| | tRNA.h1UCA | CGTTCGCGCTGTGCGGGTTCAAGTCCCGCTCCGGGCA |
| 21 | Leu- | GCCCGGATGGTGGAATCGGTAGACACAAGGGACTttaAATCCCTCGG |
| | tRNA.h1UUA | CGTTCGCGCTGTGCGGGTTCAAGTCCCGCTCCGGGCA |
| 22 | Leu- | GCCCGGATGGTGGAATCGGTAGACACAAGGGATTctaAATCCCTCGG |
| | tRNA.wtCUA_ | CGTTCGCGCTGTGCGGGTTCAAGTCCCGCTCCGGGtACCA |
| | cca | |
| 23 | Leu- | GCCCGGATGGTGGAATCGGTAGACACAAGGGATTtcaAATCCCTCGG |
| | tRNA.wtUCA_ | CGTTCGCGCTGTGCGGGTTCAAGTCCCGCTCCGGGLACCA |
| | cca | |
| 24 | Leu- | GCCCGGATGGTGGAATCGGTAGACACAAGGGATTttaAATCCCTCGG |
| | tRNA.wtUUA_ | CGTTCGCGCTGTGCGGGTTCAAGTCCCGCTCCGGGtACCA |
| | cca | |
| 25 | Leu- | GCCCGGATGGTGGAATCGGTAGACACAAGGGACTctaAATCCCTCGG |
| | tRNA.h1CUA_ | CGTTCGCGCTGTGCGGGTTCAAGTCCCGCTCCGGGCACCA |
| | cca | |
| 26 | Leu- | GCCCGGATGGTGGAATCGGTAGACACAAGGGACTtcaAATCCCTCGG |
| | tRNA.h1UCA_ | CGTTCGCGCTGTGCGGGTTCAAGTCCCGCTCCGGGCACCA |
| | cca | |
| 27 | Leu- | GCCCGGATGGTGGAATCGGTAGACACAAGGGACTttaAATCCCTCGG |
| | tRNA.h1UUA_ | CGTTCGCGCTGTGCGGGTTCAAGTCCCGCTCCGGGCACCA |
| | cca | |
| 28 | Leu- | GCCCGGATGGTGGAATCGGTAGACACAAGGGACTnnnAATCCCTCGG |
| | tRNA.003. | CGTTCGCGCTGTGCGGGTTCAAGTCCCGCTCCGGGCACCA |
| | 30 | wherein nnn is CTA, TCA, or TTA |
| 29 | Leu- | GGGCGTGTGGTGGAATCGGTAGACACAAGGGATTnnnAATCCCTCGG |
| | tRNA.003. | CGTTCGCGCTGTGCGGGTTCAAGTCCCGCCGCGCCCACCA |
| | 31 | wherein nnn is CTA, TCA, or TTA |
| 30 | Leu- | GGGCGCGTGGTGGAATCGGTAGACACAAGGGATTnnnAATCCCTCGG |
| | tRNA.003. | CGTTCGCGCTGTGCGGGTTCAAGTCCCGCCGCGCCCACCA |
| | 32 | wherein nnn is CTA, TCA, or TTA |
| 31 | Leu- | GGGCATGTGGTGGAATCGGTAGACACAAGGGATTnnnAATCCCTCGG |
| | tRNA.003. | CGTTCGCGCTGTGCGGGTTCAAGTCCCGCCATGCCCACCA |
| | 33 | wherein nnn is CTA, TCA, or TTA |
| 32 | Leu- | GGGCACGTGGTGGAATCGGTAGACACAAGGGATTnnnAATCCCTCGG |
| | tRNA.003. | CGTTCGCGCTGTGCGGGTTCAAGTCCCGCCGTGCCCACCA |
| | 34 | wherein nnn is CTA, TCA, or TTA |
| 33 | Leu- | GGGGGTGTGGTGGAATCGGTAGACACAAGGGATTnnnAATCCCTCGG |
| | RNA.003. | CGTTCGCGCTGTGCGGGTTCAAGTCCCGCCGCCCCCACCA |
| | 35 | wherein nnn is CTA, TCA, or TTA |
| 34 | Leu- | GGGGGCGTGGTGGAATCGGTAGACACAAGGGATTnnnAATCCCTCGG |
| | tRNA.003. | CGTTCGCGCTGTGCGGGTTCAAGTCCCGCCGTCCCCACCA |
| | 36 | wherein nnn is CTA, TCA, or TTA |
| 35 | Leu- | GGGGATGTGGTGGAATCGGTAGACACAAGGGATTnnnAATCCCTCGG |
| | RNA.003. | CGTTCGCGCTGTGCGGGTTCAAGTCCCGCCGTCCCCACCA |
| | 37 | wherein nnn is CTA, TCA, or TTA |
| 36 | Leu- | GGGGACGTGGTGGAATCGGTAGACACAAGGGATTnnnAATCCCTCGG |
| | tRNA.003. | CGTTCGCGCTGTGCGGGTTCAAGTCCCGCCGTCCCCACCA |
| | 38 | wherein nnn is CTA, TCA, or TTA |
| 37 | Leu- | GCCCGTATGGTGGAATCGGTAGACACAAGGGATTnnnAATCCCTCGG |
| | tRNA.003. | CGTTCGCGCTGTGCGGGTTCAAGTCCCGCTGCGGGCACCA |
| | 39 | wherein nnn is CTA, TCA, or TTA |
| 38 | Leu- | GGGATAGTGGTGGAATCGGTAGACACAAGGGATTnnnAATCCCTCGG |
| | RNA.003. | CGTTCGCGCTGTGCGGGTTCAAGTCCCGCCTATCCCACCA |
| | 40 | wherein nnn is CTA, TCA, or TTA |
| 39 | Leu- | GGGCATGTGGTGGAATCGGTAGACACAAGGGATTnnnAATCCCTCGG |
| | tRNA.003. | CGTTCGCGCTGTGCGGGTTCAAGTCCCGCCGTGCCCACCA |
| | 41 | wherein nnn is CTA, TCA, or TTA |
| 40 | Leu- | GGGCAGATGGTGGAATCGGTAGACACAAGGGATTnnnAATCCCTCGG |
| | tRNA.003. | CGTTCGCGCTGTGCGGGTTCAAGTCCCGCTCTGCCCACCA |
| | 42 | wherein nnn is CTA, TCA, or TTA |
| 41 | Leu- | GGGCGTATGGTGGAATCGGTAGACACAAGGGATTnnnAATCCCTCGG |
| | tRNA.003. | CGTTCGCGCTGTGCGGGTTCAAGTCCCGCTGCGCCCACCA |
| | 43 | wherein nnn is CTA, TCA, or TTA |
| 42 | Leu- | GGGCAAGTGGTGGAATCGGTAGACACAAGGGATTnnnAATCCCTCGG |
| | tRNA.003. | CGTTCGCGCTGTGCGGGTTCAAGTCCCGCCTTGCCCACCA |
| | 44 | wherein nnn is CTA, TCA, or TTA |
| 43 | Leu- | GCACACATGGTGGAATCGGTAGACACAAGGGATTnnnAATCCCTCGG |
| | tRNA.003. | CGTTCGCGCTGTGCGGGTTCAAGTCCCGCTGTGTGCACCA |
| | 45 | wherein nnn is CTA, TCA, or TTA |
| 44 | E. coli | MTKPIVFSGAQPSGELTIGNYMGALRQWVNMQDDYHCIYCIVDQHAI |
| | TrpRS.wt | TVRQDAQKLRKATLDTLALYLACGIDPEKSTIFVQSHVPEHAQLGWA |
| | | LNCYTYFGELSRMTQFKDKSARYAENINAGLEDYPVLMAADILLYQT |
| | | NLVPVGEDQKQHLELSRDIAQRFNALYGEIFKVPEPFIPKSGARVMS |
| | | LLEPTKKMSKSDDNRNNVIGLLEDPKSVVKKIKRAVTDSDEPPVVRY |
| | | DVQNKAGVSNLLDILSAVTGQSIPELEKQFEGKMYGHLKGEVADAVS |
| | | GMLTELQERYHRFRNDEAFLQQVMKDGAEKASAHASRTLKAVYEAIG |
| | | FVAKP |
| 45 | TrpRS.h14 | MTKPIVFAGAQPSGELTIGNYMGALRQWVNMQDDYHCIYCIVDQHAI |
| | | TVRQDAQKLRKATLDTLALYLACGIDPEKSTIFVQSHVPEHAQLGWA |
| | | LNCYTYFGELSRMTQFKDKSARYAENINAGLEDYPVLMAADILLYQT |
| | | NLGPCGEDQKQHLELSRDIAQRFNALYGEIFKVPEPFIPKSGARVMS |
| | | LLEPTKKMSKSDDNRNNVIGLLEDPKSVVKKIKRAVTDSDEPPVVRY |
| | | DVQNKAGVSNLLDILSAVTGQSIPELEKQFEGKMYGHLKGEVADAVS |
| | | GMLTELQERYHRFRNDEAFLQQVMKDGAEKASAHASRTLKAVYEAIG |
| | | FVAKP |
| 46 | TrpRS.h13 | MTKPIVFAGAQPSGELTIGNYMGALRQWVNMQDDYHCIYCIVDQHAI |
| | | TVRQDAQKLRKATLDTLALYLACGIDPEKSTIFVQSHVPEHAQLGWA |
| | | LNCYTYFGELSRMTQFKDKSARYAENINAGLFDYPVLMAADILLYQT |
| | | NLAPAGEDQKQHLELSRDIAQRFNALYGEIFKVPEPFIPKSGARVMS |
| | | LLEPTKKMSKSDDNRNNVIGLLEDPKSVVKKIKRAVTDSDEPPVVRY |
| | | DVQNKAGVSNLLDILSAVTGQSIPELEKQFEGKMYGHLKGEVADAVS |
| | | GMLTELQERYHRFRNDEAFLQQVMKDGAEKASAHASRTLKAVYEAIG |
| | | FVAKP |
| 47 | TrpRS.h9 | MTKPIVFAGAQPSGELTIGNYMGALRQWVNMQDDYHCIYCIVDQHAI |
| | | TVRQDAQKLRKATLDTLALYLACGIDPEKSTIFVQSHVPEHAQLGWA |
| | | LNCYTYFGELSRMTQFKDKSARYAENINAGLEDYPVLMAADILLYQT |
| | | NLSPAGEDQKQHLELSRDIAQRFNALYGEIFKVPEPFIPKSGARVMS |
| | | LLEPTKKMSKSDDNRNNVIGLLEDPKSVVKKIKRAVTDSDEPPVVRY |
| | | DVQNKAGVSNLLDILSAVTGQSIPELEKQFEGKMYGHLKGEVADAVS |
| | | GMLTELQERYHRFRNDEAFLQQVMKDGAEKASAHASRTLKAVYEAIG |
| | | FVAKP |
| 48 | TrpRS.h10 | MTKPIVFAGAQPSGELTIGNYMGALRQWVNMQDDYHCIYCIVDQHAI |
| | | TVRQDAQKLRKATLDTLALYLACGIDPEKSTIFVQSHVPEHAQLGWA |
| | | LNCYTYFGELSRMTQFKDKSARYAENINAGLEDYPVLMAADILLYQT |
| | | NLGPAGEDQKQHLELSRDIAQRFNALYGEIFKVPEPFIPKSGARVMS |
| | | LLEPTKKMSKSDDNRNNVIGLLEDPKSVVKKIKRAVTDSDEPPVVRY |
| | | DVQNKAGVSNLLDILSAVTGQSIPELEKQFEGKMYGHLKGEVADAVS |
| | | GMLTELQERYHRFRNDEAFLQQVMKDGAEKASAHASRTLKAVYEAIG |
| | | FVAKP |
| 49 | E. coli Trp- | AGGGGCGTAGTTCAATTGGTAGAGCACCGGTCTccaAAACCGGGTGT |
| | tRNA | TGGGAGTTCGAGTCTCTCCGCCCCTGCCA |
| 50 | Trp- | AGGGGCGTAGTTCAATTGGTAGAGCACCGGTCTctaAAACCGGGTGT |
| | tRNACUA | TGGGAGTTCGAGTCTCTCCGCCCCTGCCA |
| 51 | Trp- | AGGGGCGTAGTTCAATTGGTAGAGCACCGGTCTtcaAAACCGGGTGT |
| | tRNAUCA | TGGGAGTTCGAGTCTCTCCGCCCCTGCCA |
| 52 | Trp- | AGGGGCGTAGTTCAATTGGTAGAGCACCGGTCTttaAAACCGGGTGT |
| | tRNAUUA | TGGGAGTTCGAGTCTCTCCGCCCCTGCCA |
| 53 | Trp- | TGGGGTATCGCCAAGCGGTAAGGCACCGGATTCnnnTTCCGGCATTC |
| | tRNA.001.4 | CGAGGTTCGAATCCTCGTACCCCAGCCA |
| | | wherein nnn is CTA, TCA, or TTA |
| 54 | Trp- | AGGGGCATAGCTCAAGCGGTAAAGCACCGGACTnnnAAACCGGCAGT |
| | tRNA.001.5 | CCGAAGTTCGAATCCCCCCACCCCAGCCA |
| | | wherein nnn is CTA, TCA, or TTA |
| 55 | E. coli | ATGCAGGAACAGTATCGCCCGGAAGAAATTGAAAGCAAAGTGCAGCT |
| | LeuRS | GCATTGGGATGAAAAACGCACCTTTGAAGTGACCGAAGATGAAAGCA |
| | | AAGAAAAATATTATTGCCTGAGCATGCTGCCGTATCCGAGCGGCCGC |
| | | CTGCATATGGGCCATGTGCGCAACTATACCATTGGCGATGTGATTGC |
| | | GCGCTATCAGCGCATGCTGGGCAAAAACGTGCTGCAGCCGATTGGCT |
| | | GGGATGCGTTTGGCCTGCCGGCGGAAGGCGCGGCGGTGAAAAACAAC |
| | | ACCGCGCCGGCGCCGTGGACCTATGATAACATTGCGTATATGAAAAA |
| | | CCAGCTGAAAATGCTGGGCTTTGGCTATGATTGGAGCCGCGAACTGG |
| | | CGACCTGCACCCCGGAATATTATCGCTGGGAACAGAAATTTTTTACC |
| | | GAACTGTATAAAAAAGGCCTGGTGTATAAAAAAACCAGCGCGGTGAA |
| | | CTGGTGCCCGAACGATCAGACCGTGCTGGCGAACGAACAGGTGATTG |
| | | ATGGCTGCTGCTGGCGCTGCGATACCAAAGTGGAACGCAAAGAAATT |
| | | CCGCAGTGGTTTATTAAAATTACCGCGTATGCGGATGAACTGCTGAA |
| | | CGATCTGGATAAACTGGATCATTGGCCGGATACCGTGAAAACCATGC |
| | | AGCGCAACTGGATTGGCCGCAGCGAAGGCGTGGAAATTACCTTTAAC |
| | | GTGAACGATTATGATAACACCCTGACCGTGTATACCACCCGCCCGGA |
| | | TACCTTTATGGGCTGCACCTATCTGGCGGTGGCGGCGGGCCATCCGC |
| | | TGGCGCAGAAAGCGGCGGAAAACAACCCGGAACTGGCGGCGTTTATT |
| | | GATGAATGCCGCAACACCAAAGTGGCGGAAGCGGAAATGGCGACCAT |
| | | GGAAAAAAAAGGCGTGGATACCGGCTTTAAAGCGGTGCATCCGCTGA |
| | | CCGGCGAAGAAATTCCGGTGTGGGCGGCGAACTTTGTGCTGATGGAA |
| | | TATGGCACCGGCGCGGTGATGGCGGTGCCGGGCCATGATCAGCGCGA |
| | | TTATGAATTTGCGAGCAAATATGGCCTGAACATTAAACCGGTGATTC |
| | | TGGCGGCGGATGGCAGCGAACCGGATCTGAGCCAGCAGGCGCTGACC |
| | | GAAAAAGGCGTGCTGTTTAACAGCGGCGAATTTAACGGCCTGGATCA |
| | | TGAAGCGGCGTTTAACGCGATTGCGGATAAACTGACCGCGATGGGCG |
| | | TGGGCGAACGCAAAGTGAACTATCGCCTGCGCGATTGGGGCGTGAGC |
| | | CGCCAGCGCTATTGGGGCGCGCCGATTCCGATGGTGACCCTGGAAGA |
| | | TGGCACCGTGATGCCGACCCCGGATGATCAGCTGCCGGTGATTCTGC |
| | | CGGAAGATGTGGTGATGGATGGCATTACCAGCCCGATTAAAGCGGAT |
| | | CCGGAATGGGCGAAAACCACCGTGAACGGCATGCCGGCGCTGCGCGA |
| | | AACCGATACCTTTGATACCTTTATGGAAAGCAGCTGGTATTATGCGC |
| | | GCTATACCTGCCCGCAGTATAAAGAAGGCATGCTGGATAGCGAAGCG |
| | | GCGAACTATTGGCTGCCGGTGGATATTTATATTGGCGGCATTGAACA |
| | | TGCGATTATGCATCTGCTGTATTTTCGCTTTTTTCATAAACTGATGC |
| | | GCGATGCGGGCATGGTGAACAGCGATGAACCGGCGAAACAGCTGCTG |
| | | TGCCAGGGCATGGTGCTGGCGGATGCGTTTTATTATGTGGGCGAAAA |
| | | CGGCGAACGCAACTGGGTGAGCCCGGTGGATGCGATTGTGGAACGCG |
| | | ATGAAAAAGGCCGCATTGTGAAAGCGAAAGATGCGGCGGGCCATGAA |
| | | CTGGTGTATACCGGCATGAGCAAAATGAGCAAAAGCAAAAACAACGG |
| | | CATTGATCCGCAGGTGATGGTGGAACGCTATGGCGCGGATACCGTGC |
| | | GCCTGTTTATGATGTTTGCGAGCCCGGCGGATATGACCCTGGAATGG |
| | | CAGGAAAGCGGCGTGGAAGGCGCGAACCGCTTTCTGAAACGCGTGTG |
| | | GAAACTGGTGTATGAACATACCGCGAAAGGCGATGTGGCGGCGCTGA |
| | | ACGTGGATGCGCTGACCGAAAACCAGAAAGCGCTGCGCCGCGATGTG |
| | | CATAAAACCATTGCGAAAGTGACCGATGATATTGGCCGCCGCCAGAC |
| | | CTTTAACACCGCGATTGCGGCGATTATGGAACTGATGAACAAACTGG |
| | | CGAAAGCGCCGACCGATGGCGAACAGGATCGCGCGCTGATGCAGGAA |
| | | GCGCTGCTGGCGGTGGTGCGCATGCTGAACCCGTTTACCCCGCATAT |
| | | TTGCTTTACCCTGTGGCAGGAACTGAAAGGCGAAGGCGATATTGATA |
| | | ACGCGCCGTGGCCGGTGGCGGATGAAAAAGCGATGGTGGAAGATAGC |
| | | ACCCTGGTGGTGGTGCAGGTGAACGGCAAAGTGCGCGCGAAAATTAC |
| | | CGTGCCGGTGGATGCGACCGAAGAACAGGTGCGCGAACGCGCGGGCC |
| | | AGGAACATCTGGTGGCGAAATATCTGGATGGCGTGACCGTGCGCAAA |
| | | GTGATTTATGTGCCGGGCAAACTGCTGAACCTGGTGGTGGGCGGCCC |
| | | GGTG |
| 56 | LeuRS.v1 | ATGGAAGAACAGTATCGCCCGGAAGAAATTGAAAGCAAAGTGCAGCT |
| | | GCATTGGGATAAAAAACGCACCTTTGAAGTGACCGAAGATGAAAGCA |
| | | AAGAAAAATATTATTGCCTGAGCATTAGCCCGTATCCGAGCGGCCGC |
| | | CTGCATATGGGCCATGTGCGCAACTATACCATTGGCGATGTGATTGC |
| | | GCGCTATCAGCGCATGCTGGGCAAAAACGTGCTGCAGCCGATTGGCT |
| | | GGGATGCGTTTGGCCTGCCGGCGGAAGGCGCGGCGGTGAAAAACAAC |
| | | ACCGCGCCGGCGCCGTGGACCTATGATAACATTGCGTATATGAAAAA |
| | | CCAGCTGAAAATGCTGGGCTTTGGCTATGATTGGAGCCGCGAACTGG |
| | | CGACCTGCACCCCGGAATATTATCGCTGGGAACAGAAATTTTTTACC |
| | | GAACTGTATAAAAAAGGCCTGGTGTATAAAAAAACCAGCGCGGTGAA |
| | | CTGGTGCCCGAACGATCAGACCGTGCTGGCGAACGAACAGGTGATTG |
| | | ATGGCTGCTGCTGGCGCTGCGATACCAAAGTGGAACGCAAAGAAATT |
| | | CCGCAGTGGTTTATTAAAATTACCGCGTATGCGGATGAACTGCTGAA |
| | | CGATCTGGATAAACTGGATCATTGGCCGGATACCGTGAAAACCATGC |
| | | AGCGCAACTGGATTGGCCGCAGCGAAGGCGTGGAAATTACCTTTAAC |
| | | GTGAACGATTATGATAACACCCTGACCGTGTATACCACCCGCCCGGA |
| | | TGCGTTTATGGGCTGCACCTATCTGGCGGTGGCGGCGGGCCATCCGC |
| | | TGGCGCAGAAAGCGGCGGAAAACAACCCGGAACTGGCGGCGTTTATT |
| | | GATGAATGCCGCAACACCAAAGTGGCGGAAGCGGAAATGGCGACCAT |
| | | GGAAAAAAAAGGCGTGGATACCGGCTTTAAAGCGGTGCATCCGCTGA |
| | | CCGGCGAAGAAATTCCGGTGTGGGCGGCGAACTTTGTGCTGATGGAA |
| | | TATGGCACCGGCGCGGTGATGGCGGTGCCGGGCCATGATCAGCGCGA |
| | | TTATGAATTTGCGAGCAAATATGGCCTGAACATTAAACCGGTGATTC |
| | | TGGCGGCGGATGGCAGCGAACCGGATCTGAGCCAGCAGGCGCTGACC |
| | | GAAAAAGGCGTGCTGTTTAACAGCGGCGAATTTAACGGCCTGGATCA |
| | | TGAAGCGGCGTTTAACGCGATTGCGGATAAACTGACCGCGATGGGCG |
| | | TGGGCGAACGCAAAGTGAACTATCGCCTGCGCGATTGGGGCGTGAGC |
| | | CGCCAGCGCTATTGGGGCGCGCCGATTCCGATGGTGACCCTGGAAGA |
| | | TGGCACCGTGATGCCGACCCCGGATGATCAGCTGCCGGTGATTCTGC |
| | | CGGAAGATGTGGTGATGGATGGCATTACCAGCCCGATTAAAGCGGAT |
| | | CCGGAATGGGCGAAAACCACCGTGAACGGCATGCCGGCGCTGCGCGA |
| | | AACCGATACCTTTGATACCTTTATGGAAAGCAGCTGGATTTATGCGC |
| | | GCTATACCTGCCCGCAGTATAAAGAAGGCATGCTGGATAGCGAAGCG |
| | | GCGAACTATTGGCTGCCGGTGGATATTGCGATTGGCGGCATTGAACA |
| | | TGCGATTATGGGCCTGCTGTATTTTCGCTTTTTTCATAAACTGATGC |
| | | GCGATGCGGGCATGGTGAACAGCGATGAACCGGCGAAACAGCTGCTG |
| | | TGCCAGGGCATGGTGCTGGCGGATGCGTTTTATTATGTGGGCGAAAA |
| | | CGGCGAACGCAACTGGGTGAGCCCGGTGGATGCGATTGTGGAACGCG |
| | | ATGAAAAAGGCCGCATTGTGAAAGCGAAAGATGCGGCGGGCCATGAA |
| | | CTGGTGTATACCGGCATGAGCAAAATGAGCAAAAGCAAAAACAACGG |
| | | CATTGATCCGCAGGTGATGGTGGAACGCTATGGCGCGGATACCGTGC |
| | | GCCTGTTTATGATGTTTGCGAGCCCGGCGGATATGACCCTGGAATGG |
| | | CAGGAAAGCGGCGTGGAAGGCGCGAACCGCTTTCTGAAACGCGTGTG |
| | | GAAACTGGTGTATGAACATACCGCGAAAGGCGATGTGGCGGCGCTGA |
| | | ACGTGGATGCGCTGACCGAAAACCAGAAAGCGCTGCGCCGCGATGTG |
| | | CATAAAACCATTGCGAAAGTGACCGATGATATTGGCCGCCGCCAGAC |
| | | CTTTAACACCGCGATTGCGGCGATTATGGAACTGATGAACAAACTGG |
| | | CGAAAGCGCCGACCGATGGCGAACAGGATCGCGCGCTGATGCAGGAA |
| | | GCGCTGCTGGCGGTGGTGCGCATGCTGAACCCGTTTACCCCGCATAT |
| | | TTGCTTTACCCTGTGGCAGGAACTGAAAGGCGAAGGCGATATTGATA |
| | | ACGCGCCGTGGCCGGTGGCGGATGAAAAAGCGATGGTGGAAGATAGC |
| | | ACCCTGGTGGTGGTGCAGGTGAACGGCAAAGTGCGCGCGAAAATTAC |
| | | CGTGCCGGTGGATGCGACCGAAGAACAGGTGCGCGAACGCGCGGGCC |
| | | AGGAACATCTGGTGGCGAAATATCTGGATGGCGTGACCGTGCGCAAA |
| | | GTGATTTATGTGCCGGGCAAACTGCTGAACCTGGTGGTGGGCGGCCC |
| | | GGTG |
| 57 | LeuRS.v2 | ATGGAAGAACAGTATCGCCCGGAAGAAATTGAAAGCAAAGTGCAGCT |
| | | GCATTGGGATAAAAAACGCACCTTTGAAGTGACCGAAGATGAAAGCA |
| | | AAGAAAAATATTATTGCCTGAGCGTGAGCCCGTATCCGAGCGGCCGC |
| | | CTGCATATGGGCCATGTGCGCAACTATACCATTGGCGATGTGATTGC |
| | | GCGCTATCAGCGCATGCTGGGCAAAAACGTGCTGCAGCCGATTGGCT |
| | | GGGATGCGTTTGGCCTGCCGGCGGAAGGCGCGGCGGTGAAAAACAAC |
| | | ACCGCGCCGGCGCCGTGGACCTATGATAACATTGCGTATATGAAAAA |
| | | CCAGCTGAAAATGCTGGGCTTTGGCTATGATTGGAGCCGCGAACTGG |
| | | CGACCTGCACCCCGGAATATTATCGCTGGGAACAGAAATTTTTTACC |
| | | GAACTGTATAAAAAAGGCCTGGTGTATAAAAAAACCAGCGCGGTGAA |
| | | CTGGTGCCCGAACGATCAGACCGTGCTGGCGAACGAACAGGTGATTG |
| | | ATGGCTGCTGCTGGCGCTGCGATACCAAAGTGGAACGCAAAGAAATT |
| | | CCGCAGTGGTTTATTAAAATTACCGCGTATGCGGATGAACTGCTGAA |
| | | CGATCTGGATAAACTGGATCATTGGCCGGATACCGTGAAAACCATGC |
| | | AGCGCAACTGGATTGGCCGCAGCGAAGGCGTGGAAATTACCTTTAAC |
| | | GTGAACGATTATGATAACACCCTGACCGTGTATACCACCCGCCCGGA |
| | | TCGCTTTATGGGCTGCACCTATCTGGCGGTGGCGGCGGGCCATCCGC |
| | | TGGCGCAGAAAGCGGCGGAAAACAACCCGGAACTGGCGGCGTTTATT |
| | | GATGAATGCCGCAACACCAAAGTGGCGGAAGCGGAAATGGCGACCAT |
| | | GGAAAAAAAAGGCGTGGATACCGGCTTTAAAGCGGTGCATCCGCTGA |
| | | CCGGCGAAGAAATTCCGGTGTGGGCGGCGAACTTTGTGCTGATGGAA |
| | | TATGGCACCGGCGCGGTGATGGCGGTGCCGGGCCATGATCAGCGCGA |
| | | TTATGAATTTGCGAGCAAATATGGCCTGAACATTAAACCGGTGATTC |
| | | TGGCGGCGGATGGCAGCGAACCGGATCTGAGCCAGCAGGCGCTGACC |
| | | GAAAAAGGCGTGCTGTTTAACAGCGGCGAATTTAACGGCCTGGATCA |
| | | TGAAGCGGCGTTTAACGCGATTGCGGATAAACTGACCGCGATGGGCG |
| | | TGGGCGAACGCAAAGTGAACTATCGCCTGCGCGATTGGGGCGTGAGC |
| | | CGCCAGCGCTATTGGGGCGCGCCGATTCCGATGGTGACCCTGGAAGA |
| | | TGGCACCGTGATGCCGACCCCGGATGATCAGCTGCCGGTGATTCTGC |
| | | CGGAAGATGTGGTGATGGATGGCATTACCAGCCCGATTAAAGCGGAT |
| | | CCGGAATGGGCGAAAACCACCGTGAACGGCATGCCGGCGCTGCGCGA |
| | | AACCGATACCTTTGATACCTTTATGGAAAGCAGCTGGAGCTATGCGC |
| | | GCTATACCTGCCCGCAGTATAAAGAAGGCATGCTGGATAGCGAAGCG |
| | | GCGAACTATTGGCTGCCGGTGGATATTCTGATTGGCGGCATTGAACA |
| | | TGCGATTATGGGCCTGCTGTATTTTCGCTTTTTTCATAAACTGATGC |
| | | GCGATGCGGGCATGGTGAACAGCGATGAACCGGCGAAACAGCTGCTG |
| | | TGCCAGGGCATGGTGCTGGCGGATGCGTTTTATTATGTGGGCGAAAA |
| | | CGGCGAACGCAACTGGGTGAGCCCGGTGGATGCGATTGTGGAACGCG |
| | | ATGAAAAAGGCCGCATTGTGAAAGCGAAAGATGCGGCGGGCCATGAA |
| | | CTGGTGTATACCGGCATGAGCAAAATGAGCAAAAGCAAAAACAACGG |
| | | CATTGATCCGCAGGTGATGGTGGAACGCTATGGCGCGGATACCGTGC |
| | | GCCTGTTTATGATGTTTGCGAGCCCGGCGGATATGACCCTGGAATGG |
| | | CAGGAAAGCGGCGTGGAAGGCGCGAACCGCTTTCTGAAACGCGTGTG |
| | | GAAACTGGTGTATGAACATACCGCGAAAGGCGATGTGGCGGCGCTGA |
| | | ACGTGGATGCGCTGACCGAAAACCAGAAAGCGCTGCGCCGCGATGTG |
| | | CATAAAACCATTGCGAAAGTGACCGATGATATTGGCCGCCGCCAGAC |
| | | CTTTAACACCGCGATTGCGGCGATTATGGAACTGATGAACAAACTGG |
| | | CGAAAGCGCCGACCGATGGCGAACAGGATCGCGCGCTGATGCAGGAA |
| | | GCGCTGCTGGCGGTGGTGCGCATGCTGAACCCGTTTACCCCGCATAT |
| | | TTGCTTTACCCTGTGGCAGGAACTGAAAGGCGAAGGCGATATTGATA |
| | | ACGCGCCGTGGCCGGTGGCGGATGAAAAAGCGATGGTGGAAGATAGC |
| | | ACCCTGGTGGTGGTGCAGGTGAACGGCAAAGTGCGCGCGAAAATTAC |
| | | CGTGCCGGTGGATGCGACCGAAGAACAGGTGCGCGAACGCGCGGGCC |
| | | AGGAACATCTGGTGGCGAAATATCTGGATGGCGTGACCGTGCGCAAA |
| | | GTGATTTATGTGCCGGGCAAACTGCTGAACCTGGTGGTGGGCGGCCC |
| | | GGTG |
| 58 | LeuRS.v3 | ATGGAAGAACAGTATCGCCCGGAAGAAATTGAAAGCAAAGTGCAGCT |
| | | GCATTGGGATGAAAAACGCACCTTTGAAGTGACCGAAGATGAAAGCA |
| | | AAGAAAAATATTATTGCCTGAGCATTCTGCCGTATCCGAGCGGCCGC |
| | | CTGCATATGGGCCATGTGCGCAACTATACCATTGGCGATGTGATTGC |
| | | GCGCTATCAGCGCATGCTGGGCAAAAACGTGCTGCAGCCGATTGGCT |
| | | GGGATGCGTTTGGCCTGCCGGCGGAAGGCGCGGCGGTGAAAAACAAC |
| | | ACCGCGCCGGCGCCGTGGACCTATGATAACATTGCGTATATGAAAAA |
| | | CCAGCTGAAAATGCTGGGCTTTGGCTATGATTGGAGCCGCGAACTGG |
| | | CGACCTGCACCCCGGAATATTATCGCTGGGAACAGAAATTTTTTACC |
| | | GAACTGTATAAAAAAGGCCTGGTGTATAAAAAAACCAGCGCGGTGAA |
| | | CTGGTGCCCGAACGATCAGACCGTGCTGGCGAACGAACAGGTGATTG |
| | | ATGGCTGCTGCTGGCGCTGCGATACCAAAGTGGAACGCAAAGAAATT |
| | | CCGCAGTGGTTTATTAAAATTACCGCGTATGCGGATGAACTGCTGAA |
| | | CGATCTGGATAAACTGGATCATTGGCCGGATACCGTGAAAACCATGC |
| | | AGCGCAACTGGATTGGCCGCAGCGAAGGCGTGGAAATTACCTTTAAC |
| | | GTGAACGATTATGATAACACCCTGACCGTGTATACCACCCGCCCGGA |
| | | TGCGTTTATGGGCTGCACCTATCTGGCGGTGGCGGCGGGCCATCCGC |
| | | TGGCGCAGAAAGCGGCGGAAAACAACCCGGAACTGGCGGCGTTTATT |
| | | GATGAATGCCGCAACACCAAAGTGGCGGAAGCGGAAATGGCGACCAT |
| | | GGAAAAAAAAGGCGTGGATACCGGCTTTAAAGCGGTGCATCCGCTGA |
| | | CCGGCGAAGAAATTCCGGTGTGGGCGGCGAACTTTGTGCTGATGGAA |
| | | TATGGCACCGGCGCGGTGATGGCGGTGCCGGGCCATGATCAGCGCGA |
| | | TTATGAATTTGCGAGCAAATATGGCCTGAACATTAAACCGGTGATTC |
| | | TGGCGGCGGATGGCAGCGAACCGGATCTGAGCCAGCAGGCGCTGACC |
| | | GAAAAAGGCGTGCTGTTTAACAGCGGCGAATTTAACGGCCTGGATCA |
| | | TGAAGCGGCGTTTAACGCGATTGCGGATAAACTGACCGCGATGGGCG |
| | | TGGGCGAACGCAAAGTGAACTATCGCCTGCGCGATTGGGGCGTGAGC |
| | | CGCCAGCGCTATTGGGGCGCGCCGATTCCGATGGTGACCCTGGAAGA |
| | | TGGCACCGTGATGCCGACCCCGGATGATCAGCTGCCGGTGATTCTGC |
| | | CGGAAGATGTGGTGATGGATGGCATTACCAGCCCGATTAAAGCGGAT |
| | | CCGGAATGGGCGAAAACCACCGTGAACGGCATGCCGGCGCTGCGCGA |
| | | AACCGATACCTTTGATACCTTTATGGAAAGCAGCTGGATTTATGCGC |
| | | GCTATACCTGCCCGCAGTATAAAGAAGGCATGCTGGATAGCGAAGCG |
| | | GCGAACTATTGGCTGCCGGTGGATATTGCGATTGGCGGCATTGAACA |
| | | TGCGATTATGGGCCTGCTGTATTTTCGCTTTTTTCATAAACTGATGC |
| | | GCGATGCGGGCATGGTGAACAGCGATGAACCGGCGAAACAGCTGCTG |
| | | TGCCAGGGCATGGTGCTGGCGGATGCGTTTTATTATGTGGGCGAAAA |
| | | CGGCGAACGCAACTGGGTGAGCCCGGTGGATGCGATTGTGGAACGCG |
| | | ATGAAAAAGGCCGCATTGTGAAAGCGAAAGATGCGGCGGGCCATGAA |
| | | CTGGTGTATACCGGCATGAGCAAAATGAGCAAAAGCAAAAACAACGG |
| | | CATTGATCCGCAGGTGATGGTGGAACGCTATGGCGCGGATACCGTGC |
| | | GCCTGTTTATGATGTTTGCGAGCCCGGCGGATATGACCCTGGAATGG |
| | | CAGGAAAGCGGCGTGGAAGGCGCGAACCGCTTTCTGAAACGCGTGTG |
| | | GAAACTGGTGTATGAACATACCGCGAAAGGCGATGTGGCGGCGCTGA |
| | | ACGTGGATGCGCTGACCGAAAACCAGAAAGCGCTGCGCCGCGATGTG |
| | | CATAAAACCATTGCGAAAGTGACCGATGATATTGGCCGCCGCCAGAC |
| | | CTTTAACACCGCGATTGCGGCGATTATGGAACTGATGAACAAACTGG |
| | | CGAAAGCGCCGACCGATGGCGAACAGGATCGCGCGCTGATGCAGGAA |
| | | GCGCTGCTGGCGGTGGTGCGCATGCTGAACCCGTTTACCCCGCATAT |
| | | TTGCTTTACCCTGTGGCAGGAACTGAAAGGCGAAGGCGATATTGATA |
| | | ACGCGCCGTGGCCGGTGGCGGATGAAAAAGCGATGGTGGAAGATAGC |
| | | ACCCTGGTGGTGGTGCAGGTGAACGGCAAAGTGCGCGCGAAAATTAC |
| | | CGTGCCGGTGGATGCGACCGAAGAACAGGTGCGCGAACGCGCGGGCC |
| | | AGGAACATCTGGTGGCGAAATATCTGGATGGCGTGACCGTGCGCAAA |
| | | GTGATTTATGTGCCGGGCAAACTGCTGAACCTGGTGGTGGGCGGCCC |
| | | GGTG |
| 59 | V9 | ATGGAAGAACAGTATCGCCCGGAAGAAATTGAAAGCAAAGTGCAGCT |
| | | GCATTGGGATATGAAACGCACCTTTGAAGTGACCGAAGATGAAAGCA |
| | | AAGAAAAATATTATTGCCTGAGCATTAGCCCGTATCCGAGCGGCCGC |
| | | CTGCATATGGGCCATGTGCGCAACTATACCATTGGCGATGTGATTGC |
| | | GCGCTATCAGCGCATGCTGGGCAAAAACGTGCTGCAGCCGATTGGCT |
| | | GGGATGCGTTTGGCCTGCCGGCGGAAGGCGCGGCGGTGAAAAACAAC |
| | | ACCGCGCCGGCGCCGTGGACCTATGATAACATTGCGTATATGAAAAA |
| | | CCAGCTGAAAATGCTGGGCTTTGGCTATGATTGGAGCCGCGAACTGG |
| | | CGACCTGCACCCCGGAATATTATCGCTGGGAACAGAAATTTTTTACC |
| | | GAACTGTATAAAAAAGGCCTGGTGTATAAAAAAACCAGCGCGGTGAA |
| | | CTGGTGCCCGAACGATCAGACCGTGCTGGCGAACGAACAGGTGATTG |
| | | ATGGCTGCTGCTGGCGCTGCGATACCAAAGTGGAACGCAAAGAAATT |
| | | CCGCAGTGGTTTATTAAAATTACCGCGTATGCGGATGAACTGCTGAA |
| | | CGATCTGGATAAACTGGATCATTGGCCGGATACCGTGAAAACCATGC |
| | | AGCGCAACTGGATTGGCCGCAGCGAAGGCGTGGAAATTACCTTTAAC |
| | | GTGAACGATTATGATAACACCCTGACCGTGTATACCACCCGCCCGGA |
| | | TGCGTTTATGGGCTGCACCTATCTGGCGGTGGCGGCGGGCCATCCGC |
| | | TGGCGCAGAAAGCGGCGGAAAACAACCCGGAACTGGCGGCGTTTATT |
| | | GATGAATGCCGCAACACCAAAGTGGCGGAAGCGGAAATGGCGACCAT |
| | | GGAAAAAAAAGGCGTGGATACCGGCTTTAAAGCGGTGCATCCGCTGA |
| | | CCGGCGAAGAAATTCCGGTGTGGGCGGCGAACTTTGTGCTGATGGAA |
| | | TATGGCACCGGCGCGGTGATGGCGGTGCCGGGCCATGATCAGCGCGA |
| | | TTATGAATTTGCGAGCAAATATGGCCTGAACATTAAACCGGTGATTC |
| | | TGGCGGCGGATGGCAGCGAACCGGATCTGAGCCAGCAGGCGCTGACC |
| | | GAAAAAGGCGTGCTGTTTAACAGCGGCGAATTTAACGGCCTGGATCA |
| | | TGAAGCGGCGTTTAACGCGATTGCGGATAAACTGACCGCGATGGGCG |
| | | TGGGCGAACGCAAAGTGAACTATCGCCTGCGCGATTGGGGCGTGAGC |
| | | CGCCAGCGCTATTGGGGCGCGCCGATTCCGATGGTGACCCTGGAAGA |
| | | TGGCACCGTGATGCCGACCCCGGATGATCAGCTGCCGGTGATTCTGC |
| | | CGGAAGATGTGGTGATGGATGGCATTACCAGCCCGATTAAAGCGGAT |
| | | CCGGAATGGGCGAAAACCACCGTGAACGGCATGCCGGCGCTGCGCGA |
| | | AACCGATACCTTTGATACCTTTATGGAAAGCAGCTGGATTTATGCGC |
| | | GCTATACCTGCCCGCAGTATAAAGAAGGCATGCTGGATAGCGAAGCG |
| | | GCGAACTATTGGCTGCCGGTGGATATTGCGATTGGCGGCATTGAACA |
| | | TGCGATTATGGGCCTGCTGTATTTTCGCTTTTTTCATAAACTGATGC |
| | | GCGATGCGGGCATGGTGAACAGCGATGAACCGGCGAAACAGCTGCTG |
| | | TGCCAGGGCATGGTGCTGGCGGATGCGTTTTATTATGTGGGCGAAAA |
| | | CGGCGAACGCAACTGGGTGAGCCCGGTGGATGCGATTGTGGAACGCG |
| | | ATGAAAAAGGCCGCATTGTGAAAGCGAAAGATGCGGCGGGCCATGAA |
| | | CTGGTGTATACCGGCATGAGCAAAATGAGCAAAAGCAAAAACAACGG |
| | | CATTGATCCGCAGGTGATGGTGGAACGCTATGGCGCGGATACCGTGC |
| | | GCCTGTTTATGATGTTTGCGAGCCCGGCGGATATGACCCTGGAATGG |
| | | CAGGAAAGCGGCGTGGAAGGCGCGAACCGCTTTCTGAAACGCGTGTG |
| | | GAAACTGGTGTATGAACATACCGCGAAAGGCGATGTGGCGGCGCTGA |
| | | ACGTGGATGCGCTGACCGAAAACCAGAAAGCGCTGCGCCGCGATGTG |
| | | CATAAAACCATTGCGAAAGTGACCGATGATATTGGCCGCCGCCAGAC |
| | | CTTTAACACCGCGATTGCGGCGATTATGGAACTGATGAACAAACTGG |
| | | CGAAAGCGCCGACCGATGGCGAACAGGATCGCGCGCTGATGCAGGAA |
| | | GCGCTGCTGGCGGTGGTGCGCATGCTGAACCCGTTTACCCCGCATAT |
| | | TTGCTTTACCCTGTGGCAGGAACTGAAAGGCGAAGGCGATATTGATA |
| | | ACGCGCCGTGGCCGGTGGCGGATGAAAAAGCGATGGTGGAAGATAGC |
| | | ACCCTGGTGGTGGTGCAGGTGAACGGCAAAGTGCGCGCGAAAATTAC |
| | | CGTGCCGGTGGATGCGACCGAAGAACAGGTGCGCGAACGCGCGGGCC |
| | | AGGAACATCTGGTGGCGAAATATCTGGATGGCGTGACCGTGCGCAAA |
| | | GTGATTTATGTGCCGGGCAAACTGCTGAACCTGGTGGTGGGCGGCCC |
| | | GGTG |
| 60 | V10 | ATGGAAGAACAGTATCGCCCGGAAGAAATTGAAAGCAAAGTGCAGCT |
| | | GCATTGGGATGTGAAACGCACCTTTGAAGTGACCGAAGATGAAAGCA |
| | | AAGAAAAATATTATTGCCTGAGCATTAGCCCGTATCCGAGCGGCCGC |
| | | CTGCATATGGGCCATGTGCGCAACTATACCATTGGCGATGTGATTGC |
| | | GCGCTATCAGCGCATGCTGGGCAAAAACGTGCTGCAGCCGATTGGCT |
| | | GGGATGCGTTTGGCCTGCCGGCGGAAGGCGCGGCGGTGAAAAACAAC |
| | | ACCGCGCCGGCGCCGTGGACCTATGATAACATTGCGTATATGAAAAA |
| | | CCAGCTGAAAATGCTGGGCTTTGGCTATGATTGGAGCCGCGAACTGG |
| | | CGACCTGCACCCCGGAATATTATCGCTGGGAACAGAAATTTTTTACC |
| | | GAACTGTATAAAAAAGGCCTGGTGTATAAAAAAACCAGCGCGGTGAA |
| | | CTGGTGCCCGAACGATCAGACCGTGCTGGCGAACGAACAGGTGATTG |
| | | ATGGCTGCTGCTGGCGCTGCGATACCAAAGTGGAACGCAAAGAAATT |
| | | CCGCAGTGGTTTATTAAAATTACCGCGTATGCGGATGAACTGCTGAA |
| | | CGATCTGGATAAACTGGATCATTGGCCGGATACCGTGAAAACCATGC |
| | | AGCGCAACTGGATTGGCCGCAGCGAAGGCGTGGAAATTACCTTTAAC |
| | | GTGAACGATTATGATAACACCCTGACCGTGTATACCACCCGCCCGGA |
| | | TGCGTTTATGGGCTGCACCTATCTGGCGGTGGCGGCGGGCCATCCGC |
| | | TGGCGCAGAAAGCGGCGGAAAACAACCCGGAACTGGCGGCGTTTATT |
| | | GATGAATGCCGCAACACCAAAGTGGCGGAAGCGGAAATGGCGACCAT |
| | | GGAAAAAAAAGGCGTGGATACCGGCTTTAAAGCGGTGCATCCGCTGA |
| | | CCGGCGAAGAAATTCCGGTGTGGGCGGCGAACTTTGTGCTGATGGAA |
| | | TATGGCACCGGCGCGGTGATGGCGGTGCCGGGCCATGATCAGCGCGA |
| | | TTATGAATTTGCGAGCAAATATGGCCTGAACATTAAACCGGTGATTC |
| | | TGGCGGCGGATGGCAGCGAACCGGATCTGAGCCAGCAGGCGCTGACC |
| | | GAAAAAGGCGTGCTGTTTAACAGCGGCGAATTTAACGGCCTGGATCA |
| | | TGAAGCGGCGTTTAACGCGATTGCGGATAAACTGACCGCGATGGGCG |
| | | TGGGCGAACGCAAAGTGAACTATCGCCTGCGCGATTGGGGCGTGAGC |
| | | CGCCAGCGCTATTGGGGCGCGCCGATTCCGATGGTGACCCTGGAAGA |
| | | TGGCACCGTGATGCCGACCCCGGATGATCAGCTGCCGGTGATTCTGC |
| | | CGGAAGATGTGGTGATGGATGGCATTACCAGCCCGATTAAAGCGGAT |
| | | CCGGAATGGGCGAAAACCACCGTGAACGGCATGCCGGCGCTGCGCGA |
| | | AACCGATACCTTTGATACCTTTATGGAAAGCAGCTGGATTTATGCGC |
| | | GCTATACCTGCCCGCAGTATAAAGAAGGCATGCTGGATAGCGAAGCG |
| | | GCGAACTATTGGCTGCCGGTGGATATTGCGATTGGCGGCATTGAACA |
| | | TGCGATTATGGGCCTGCTGTATTTTCGCTTTTTTCATAAACTGATGC |
| | | GCGATGCGGGCATGGTGAACAGCGATGAACCGGCGAAACAGCTGCTG |
| | | TGCCAGGGCATGGTGCTGGCGGATGCGTTTTATTATGTGGGCGAAAA |
| | | CGGCGAACGCAACTGGGTGAGCCCGGTGGATGCGATTGTGGAACGCG |
| | | ATGAAAAAGGCCGCATTGTGAAAGCGAAAGATGCGGCGGGCCATGAA |
| | | CTGGTGTATACCGGCATGAGCAAAATGAGCAAAAGCAAAAACAACGG |
| | | CATTGATCCGCAGGTGATGGTGGAACGCTATGGCGCGGATACCGTGC |
| | | GCCTGTTTATGATGTTTGCGAGCCCGGCGGATATGACCCTGGAATGG |
| | | CAGGAAAGCGGCGTGGAAGGCGCGAACCGCTTTCTGAAACGCGTGTG |
| | | GAAACTGGTGTATGAACATACCGCGAAAGGCGATGTGGCGGCGCTGA |
| | | ACGTGGATGCGCTGACCGAAAACCAGAAAGCGCTGCGCCGCGATGTG |
| | | CATAAAACCATTGCGAAAGTGACCGATGATATTGGCCGCCGCCAGAC |
| | | CTTTAACACCGCGATTGCGGCGATTATGGAACTGATGAACAAACTGG |
| | | CGAAAGCGCCGACCGATGGCGAACAGGATCGCGCGCTGATGCAGGAA |
| | | GCGCTGCTGGCGGTGGTGCGCATGCTGAACCCGTTTACCCCGCATAT |
| | | TTGCTTTACCCTGTGGCAGGAACTGAAAGGCGAAGGCGATATTGATA |
| | | ACGCGCCGTGGCCGGTGGCGGATGAAAAAGCGATGGTGGAAGATAGC |
| | | ACCCTGGTGGTGGTGCAGGTGAACGGCAAAGTGCGCGCGAAAATTAC |
| | | CGTGCCGGTGGATGCGACCGAAGAACAGGTGCGCGAACGCGCGGGCC |
| | | AGGAACATCTGGTGGCGAAATATCTGGATGGCGTGACCGTGCGCAAA |
| | | GTGATTTATGTGCCGGGCAAACTGCTGAACCTGGTGGTGGGCGGCCC |
| | | GGTG |
| 61 | V26 | ATGGAAGAACAGTATCGCCCGGAAGAAATTGAAAGCAAAGTGCAGCT |
| | | GCATTGGGATAAAAAACGCACCTTTGAAGTGACCGAAGATGAAAGCA |
| | | AAGAAAAATATTATTGCCTGAGCATTGTGCCGTATCCGAGCGGCCGC |
| | | CTGCATATGGGCCATGTGCGCAACTATACCATTGGCGATGTGATTGC |
| | | GCGCTATCAGCGCATGCTGGGCAAAAACGTGCTGCAGCCGATTGGCT |
| | | GGGATGCGTTTGGCCTGCCGGCGGAAGGCGCGGCGGTGAAAAACAAC |
| | | ACCGCGCCGGCGCCGTGGACCTATGATAACATTGCGTATATGAAAAA |
| | | CCAGCTGAAAATGCTGGGCTTTGGCTATGATTGGAGCCGCGAACTGG |
| | | CGACCTGCACCCCGGAATATTATCGCTGGGAACAGAAATTTTTTACC |
| | | GAACTGTATAAAAAAGGCCTGGTGTATAAAAAAACCAGCGCGGTGAA |
| | | CTGGTGCCCGAACGATCAGACCGTGCTGGCGAACGAACAGGTGATTG |
| | | ATGGCTGCTGCTGGCGCTGCGATACCAAAGTGGAACGCAAAGAAATT |
| | | CCGCAGTGGTTTATTAAAATTACCGCGTATGCGGATGAACTGCTGAA |
| | | CGATCTGGATAAACTGGATCATTGGCCGGATACCGTGAAAACCATGC |
| | | AGCGCAACTGGATTGGCCGCAGCGAAGGCGTGGAAATTACCTTTAAC |
| | | GTGAACGATTATGATAACACCCTGACCGTGTATACCACCCGCCCGGA |
| | | TGCGTTTATGGGCTGCACCTATCTGGCGGTGGCGGCGGGCCATCCGC |
| | | TGGCGCAGAAAGCGGCGGAAAACAACCCGGAACTGGCGGCGTTTATT |
| | | GATGAATGCCGCAACACCAAAGTGGCGGAAGCGGAAATGGCGACCAT |
| | | GGAAAAAAAAGGCGTGGATACCGGCTTTAAAGCGGTGCATCCGCTGA |
| | | CCGGCGAAGAAATTCCGGTGTGGGCGGCGAACTTTGTGCTGATGGAA |
| | | TATGGCACCGGCGCGGTGATGGCGGTGCCGGGCCATGATCAGCGCGA |
| | | TTATGAATTTGCGAGCAAATATGGCCTGAACATTAAACCGGTGATTC |
| | | TGGCGGCGGATGGCAGCGAACCGGATCTGAGCCAGCAGGCGCTGACC |
| | | GAAAAAGGCGTGCTGTTTAACAGCGGCGAATTTAACGGCCTGGATCA |
| | | TGAAGCGGCGTTTAACGCGATTGCGGATAAACTGACCGCGATGGGCG |
| | | TGGGCGAACGCAAAGTGAACTATCGCCTGCGCGATTGGGGCGTGAGC |
| | | CGCCAGCGCTATTGGGGCGCGCCGATTCCGATGGTGACCCTGGAAGA |
| | | TGGCACCGTGATGCCGACCCCGGATGATCAGCTGCCGGTGATTCTGC |
| | | CGGAAGATGTGGTGATGGATGGCATTACCAGCCCGATTAAAGCGGAT |
| | | CCGGAATGGGCGAAAACCACCGTGAACGGCATGCCGGCGCTGCGCGA |
| | | AACCGATACCTTTGATACCTTTATGGAAAGCAGCTGGATTTATGCGC |
| | | GCTATACCTGCCCGCAGTATAAAGAAGGCATGCTGGATAGCGAAGCG |
| | | GCGAACTATTGGCTGCCGGTGGATATTGCGATTGGCGGCATTGAACA |
| | | TGCGATTATGGGCCTGCTGTATTTTCGCTTTTTTCATAAACTGATGC |
| | | GCGATGCGGGCATGGTGAACAGCGATGAACCGGCGAAACAGCTGCTG |
| | | TGCCAGGGCATGGTGCTGGCGGATGCGTTTTATTATGTGGGCGAAAA |
| | | CGGCGAACGCAACTGGGTGAGCCCGGTGGATGCGATTGTGGAACGCG |
| | | ATGAAAAAGGCCGCATTGTGAAAGCGAAAGATGCGGCGGGCCATGAA |
| | | CTGGTGTATACCGGCATGAGCAAAATGAGCAAAAGCAAAAACAACGG |
| | | CATTGATCCGCAGGTGATGGTGGAACGCTATGGCGCGGATACCGTGC |
| | | GCCTGTTTATGATGTTTGCGAGCCCGGCGGATATGACCCTGGAATGG |
| | | CAGGAAAGCGGCGTGGAAGGCGCGAACCGCTTTCTGAAACGCGTGTG |
| | | GAAACTGGTGTATGAACATACCGCGAAAGGCGATGTGGCGGCGCTGA |
| | | ACGTGGATGCGCTGACCGAAAACCAGAAAGCGCTGCGCCGCGATGTG |
| | | CATAAAACCATTGCGAAAGTGACCGATGATATTGGCCGCCGCCAGAC |
| | | CTTTAACACCGCGATTGCGGCGATTATGGAACTGATGAACAAACTGG |
| | | CGAAAGCGCCGACCGATGGCGAACAGGATCGCGCGCTGATGCAGGAA |
| | | GCGCTGCTGGCGGTGGTGCGCATGCTGAACCCGTTTACCCCGCATAT |
| | | TTGCTTTACCCTGTGGCAGGAACTGAAAGGCGAAGGCGATATTGATA |
| | | ACGCGCCGTGGCCGGTGGCGGATGAAAAAGCGATGGTGGAAGATAGC |
| | | ACCCTGGTGGTGGTGCAGGTGAACGGCAAAGTGCGCGCGAAAATTAC |
| | | CGTGCCGGTGGATGCGACCGAAGAACAGGTGCGCGAACGCGCGGGCC |
| | | AGGAACATCTGGTGGCGAAATATCTGGATGGCGTGACCGTGCGCAAA |
| | | GTGATTTATGTGCCGGGCAAACTGCTGAACCTGGTGGTGGGCGGCCC |
| | | GGTG |
| 62 | V29 | ATGGAAGAACAGTATCGCCCGGAAGAAATTGAAAGCAAAGTGCAGCT |
| | | GCATTGGGATAAAAAACGCACCTTTGAAGTGACCGAAGATGAAAGCA |
| | | AAGAAAAATATTATTGCCTGAGCATTGCGCCGTATCCGAGCGGCCGC |
| | | CTGCATATGGGCCATGTGCGCAACTATACCATTGGCGATGTGATTGC |
| | | GCGCTATCAGCGCATGCTGGGCAAAAACGTGCTGCAGCCGATTGGCT |
| | | GGGATGCGTTTGGCCTGCCGGCGGAAGGCGCGGCGGTGAAAAACAAC |
| | | ACCGCGCCGGCGCCGTGGACCTATGATAACATTGCGTATATGAAAAA |
| | | CCAGCTGAAAATGCTGGGCTTTGGCTATGATTGGAGCCGCGAACTGG |
| | | CGACCTGCACCCCGGAATATTATCGCTGGGAACAGAAATTTTTTACC |
| | | GAACTGTATAAAAAAGGCCTGGTGTATAAAAAAACCAGCGCGGTGAA |
| | | CTGGTGCCCGAACGATCAGACCGTGCTGGCGAACGAACAGGTGATTG |
| | | ATGGCTGCTGCTGGCGCTGCGATACCAAAGTGGAACGCAAAGAAATT |
| | | CCGCAGTGGTTTATTAAAATTACCGCGTATGCGGATGAACTGCTGAA |
| | | CGATCTGGATAAACTGGATCATTGGCCGGATACCGTGAAAACCATGC |
| | | AGCGCAACTGGATTGGCCGCAGCGAAGGCGTGGAAATTACCTTTAAC |
| | | GTGAACGATTATGATAACACCCTGACCGTGTATACCACCCGCCCGGA |
| | | TGCGTTTATGGGCTGCACCTATCTGGCGGTGGCGGCGGGCCATCCGC |
| | | TGGCGCAGAAAGCGGCGGAAAACAACCCGGAACTGGCGGCGTTTATT |
| | | GATGAATGCCGCAACACCAAAGTGGCGGAAGCGGAAATGGCGACCAT |
| | | GGAAAAAAAAGGCGTGGATACCGGCTTTAAAGCGGTGCATCCGCTGA |
| | | CCGGCGAAGAAATTCCGGTGTGGGCGGCGAACTTTGTGCTGATGGAA |
| | | TATGGCACCGGCGCGGTGATGGCGGTGCCGGGCCATGATCAGCGCGA |
| | | TTATGAATTTGCGAGCAAATATGGCCTGAACATTAAACCGGTGATTC |
| | | TGGCGGCGGATGGCAGCGAACCGGATCTGAGCCAGCAGGCGCTGACC |
| | | GAAAAAGGCGTGCTGTTTAACAGCGGCGAATTTAACGGCCTGGATCA |
| | | TGAAGCGGCGTTTAACGCGATTGCGGATAAACTGACCGCGATGGGCG |
| | | TGGGCGAACGCAAAGTGAACTATCGCCTGCGCGATTGGGGCGTGAGC |
| | | CGCCAGCGCTATTGGGGCGCGCCGATTCCGATGGTGACCCTGGAAGA |
| | | TGGCACCGTGATGCCGACCCCGGATGATCAGCTGCCGGTGATTCTGC |
| | | CGGAAGATGTGGTGATGGATGGCATTACCAGCCCGATTAAAGCGGAT |
| | | CCGGAATGGGCGAAAACCACCGTGAACGGCATGCCGGCGCTGCGCGA |
| | | AACCGATACCTTTGATACCTTTATGGAAAGCAGCTGGATTTATGCGC |
| | | GCTATACCTGCCCGCAGTATAAAGAAGGCATGCTGGATAGCGAAGCG |
| | | GCGAACTATTGGCTGCCGGTGGATATTGCGATTGGCGGCATTGAACA |
| | | TGCGATTATGGGCCTGCTGTATTTTCGCTTTTTTCATAAACTGATGC |
| | | GCGATGCGGGCATGGTGAACAGCGATGAACCGGCGAAACAGCTGCTG |
| | | TGCCAGGGCATGGTGCTGGCGGATGCGTTTTATTATGTGGGCGAAAA |
| | | CGGCGAACGCAACTGGGTGAGCCCGGTGGATGCGATTGTGGAACGCG |
| | | ATGAAAAAGGCCGCATTGTGAAAGCGAAAGATGCGGCGGGCCATGAA |
| | | CTGGTGTATACCGGCATGAGCAAAATGAGCAAAAGCAAAAACAACGG |
| | | CATTGATCCGCAGGTGATGGTGGAACGCTATGGCGCGGATACCGTGC |
| | | GCCTGTTTATGATGTTTGCGAGCCCGGCGGATATGACCCTGGAATGG |
| | | CAGGAAAGCGGCGTGGAAGGCGCGAACCGCTTTCTGAAACGCGTGTG |
| | | GAAACTGGTGTATGAACATACCGCGAAAGGCGATGTGGCGGCGCTGA |
| | | ACGTGGATGCGCTGACCGAAAACCAGAAAGCGCTGCGCCGCGATGTG |
| | | CATAAAACCATTGCGAAAGTGACCGATGATATTGGCCGCCGCCAGAC |
| | | CTTTAACACCGCGATTGCGGCGATTATGGAACTGATGAACAAACTGG |
| | | CGAAAGCGCCGACCGATGGCGAACAGGATCGCGCGCTGATGCAGGAA |
| | | GCGCTGCTGGCGGTGGTGCGCATGCTGAACCCGTTTACCCCGCATAT |
| | | TTGCTTTACCCTGTGGCAGGAACTGAAAGGCGAAGGCGATATTGATA |
| | | ACGCGCCGTGGCCGGTGGCGGATGAAAAAGCGATGGTGGAAGATAGC |
| | | ACCCTGGTGGTGGTGCAGGTGAACGGCAAAGTGCGCGCGAAAATTAC |
| | | CGTGCCGGTGGATGCGACCGAAGAACAGGTGCGCGAACGCGCGGGCC |
| | | AGGAACATCTGGTGGCGAAATATCTGGATGGCGTGACCGTGCGCAAA |
| | | GTGATTTATGTGCCGGGCAAACTGCTGAACCTGGTGGTGGGCGGCCC |
| | | GGTG |
| 63 | V48 | ATGGAAGAACAGTATCGCCCGGAAGAAATTGAAAGCAAAGTGCAGCT |
| | | GCATTGGGATAAAAAACGCACCTTTGAAGTGACCGAAGATGAAAGCA |
| | | AAGAAAAATATTATTGCCTGAGCATTAGCCCGTATCCGAGCGGCCGC |
| | | CTGCATATGGGCCATGTGCGCAACTATACCATTGGCGATGTGATTGC |
| | | GCGCTATCAGCGCATGCTGGGCAAAAACGTGCTGCAGCCGATTGGCT |
| | | GGGATGCGTTTGGCCTGCCGGCGGAAGGCGCGGCGGTGAAAAACAAC |
| | | ACCGCGCCGGCGCCGTGGACCTATGATAACATTGCGTATATGAAAAA |
| | | CCAGCTGAAAATGCTGGGCTTTGGCTATGATTGGAGCCGCGAACTGG |
| | | CGACCTGCACCCCGGAATATTATCGCTGGGAACAGAAATTTTTTACC |
| | | GAACTGTATAAAAAAGGCCTGGTGTATAAAAAAACCAGCGCGGTGAA |
| | | CTGGTGCCCGAACGATCAGACCGTGCTGGCGAACGAACAGGTGATTG |
| | | ATGGCTGCTGCTGGCGCTGCGATACCAAAGTGGAACGCAAAGAAATT |
| | | CCGCAGTGGTTTATTAAAATTACCGCGTATGCGGATGAACTGCTGAA |
| | | CGATCTGGATAAACTGGATCATTGGCCGGATACCGTGAAAACCATGC |
| | | AGCGCAACTGGATTGGCCGCAGCGAAGGCGTGGAAATTACCTTTAAC |
| | | GTGAACGATTATGATAACACCCTGACCGTGTATACCACCCGCCCGGA |
| | | TGCGTTTATGGGCTGCACCTATCTGGCGGTGGCGGCGGGCCATCCGC |
| | | TGGCGCAGAAAGCGGCGGAAAACAACCCGGAACTGGCGGCGTTTATT |
| | | GATGAATGCCGCAACACCAAAGTGGCGGAAGCGGAAATGGCGACCAT |
| | | GGAAAAAAAAGGCGTGGATACCGGCTTTAAAGCGGTGCATCCGCTGA |
| | | CCGGCGAAGAAATTCCGGTGTGGGCGGCGAACTTTGTGCTGATGGAA |
| | | TATGGCACCGGCGCGGTGATGGCGGTGCCGGGCCATGATCAGCGCGA |
| | | TTATGAATTTGCGAGCAAATATGGCCTGAACATTAAACCGGTGATTC |
| | | TGGCGGCGGATGGCAGCGAACCGGATCTGAGCCAGCAGGCGCTGACC |
| | | GAAAAAGGCGTGCTGTTTAACAGCGGCGAATTTAACGGCCTGGATCA |
| | | TGAAGCGGCGTTTAACGCGATTGCGGATAAACTGACCGCGATGGGCG |
| | | TGGGCGAACGCAAAGTGAACTATCGCCTGCGCGATTGGGGCGTGAGC |
| | | CGCCAGCGCTATTGGGGCGCGCCGATTCCGATGGTGACCCTGGAAGA |
| | | TGGCACCGTGATGCCGACCCCGGATGATCAGCTGCCGGTGATTCTGC |
| | | CGGAAGATGTGGTGATGGATGGCATTACCAGCCCGATTAAAGCGGAT |
| | | CCGGAATGGGCGAAAACCACCGTGAACGGCATGCCGGCGCTGCGCGA |
| | | AACCGATACCTTTGATACCTTTATGGAAAGCAGCTGGGCGTATGCGC |
| | | GCTATACCTGCCCGCAGTATAAAGAAGGCATGCTGGATAGCGAAGCG |
| | | GCGAACTATTGGCTGCCGGTGGATATTGCGATTGGCGGCATTGAACA |
| | | TGCGATTATGGGCCTGCTGTATTTTCGCTTTTTTCATAAACTGATGC |
| | | GCGATGCGGGCATGGTGAACAGCGATGAACCGGCGAAACAGCTGCTG |
| | | TGCCAGGGCATGGTGCTGGCGGATGCGTTTTATTATGTGGGCGAAAA |
| | | CGGCGAACGCAACTGGGTGAGCCCGGTGGATGCGATTGTGGAACGCG |
| | | ATGAAAAAGGCCGCATTGTGAAAGCGAAAGATGCGGCGGGCCATGAA |
| | | CTGGTGTATACCGGCATGAGCAAAATGAGCAAAAGCAAAAACAACGG |
| | | CATTGATCCGCAGGTGATGGTGGAACGCTATGGCGCGGATACCGTGC |
| | | GCCTGTTTATGATGTTTGCGAGCCCGGCGGATATGACCCTGGAATGG |
| | | CAGGAAAGCGGCGTGGAAGGCGCGAACCGCTTTAAACGCGTGTGGAA |
| | | ACTGGTGTATGAACATACCGCGAAAGGCGATGTGGCGGCGCTGAACG |
| | | TGGATGCGCTGACCGAAAACCAGAAAGCGCTGCGCCGCGATGTGCAT |
| | | AAAACCATTGCGAAAGTGACCGATGATATTGGCCGCCGCCAGACCTT |
| | | TAACACCGCGATTGCGGCGATTATGGAACTGATGAACAAACTGGCGA |
| | | AAGCGCCGACCGATGGCGAACAGGATCGCGCGCTGATGCAGGAAGCG |
| | | CTGCTGGCGGTGGTGCGCATGCTGAACCCGTTTACCCCGCATATTTG |
| | | CTTTACCCTGTGGCAGGAACTGAAAGGCGAAGGCGATATTGATAACG |
| | | CGCCGTGGCCGGTGGCGGATGAAAAAGCGATGGTGGAAGATAGCACC |
| | | CTGGTGGTGGTGCAGGTGAACGGCAAAGTGCGCGCGAAAATTACCGT |
| | | GCCGGTGGATGCGACCGAAGAACAGGTGCGCGAACGCGCGGGCCAGG |
| | | AACATCTGGTGGCGAAATATCTGGATGGCGTGACCGTGCGCAAAGTG |
| | | ATTTATGTGCCGGGCAAACTGCTGAACCTGGTGGTGGGCGGCCCGGT |
| | | G |
| 64 | V50 | ATGGAAGAACAGTATCGCCCGGAAGAAATTGAAAGCAAAGTGCAGCT |
| | | GCATTGGGATAAAAAACGCACCTTTGAAGTGACCGAAGATGAAAGCA |
| | | AAGAAAAATATTATTGCCTGAGCATTAGCCCGTATCCGAGCGGCCGC |
| | | CTGCATATGGGCCATGTGCGCAACTATACCATTGGCGATGTGATTGC |
| | | GCGCTATCAGCGCATGCTGGGCAAAAACGTGCTGCAGCCGATTGGCT |
| | | GGGATGCGTTTGGCCTGCCGGCGGAAGGCGCGGCGGTGAAAAACAAC |
| | | ACCGCGCCGGCGCCGTGGACCTATGATAACATTGCGTATATGAAAAA |
| | | CCAGCTGAAAATGCTGGGCTTTGGCTATGATTGGAGCCGCGAACTGG |
| | | CGACCTGCACCCCGGAATATTATCGCTGGGAACAGAAATTTTTTACC |
| | | GAACTGTATAAAAAAGGCCTGGTGTATAAAAAAACCAGCGCGGTGAA |
| | | CTGGTGCCCGAACGATCAGACCGTGCTGGCGAACGAACAGGTGATTG |
| | | ATGGCTGCTGCTGGCGCTGCGATACCAAAGTGGAACGCAAAGAAATT |
| | | CCGCAGTGGTTTATTAAAATTACCGCGTATGCGGATGAACTGCTGAA |
| | | CGATCTGGATAAACTGGATCATTGGCCGGATACCGTGAAAACCATGC |
| | | AGCGCAACTGGATTGGCCGCAGCGAAGGCGTGGAAATTACCTTTAAC |
| | | GTGAACGATTATGATAACACCCTGACCGTGTATACCACCCGCCCGGA |
| | | TGCGTTTATGGGCTGCACCTATCTGGCGGTGGCGGCGGGCCATCCGC |
| | | TGGCGCAGAAAGCGGCGGAAAACAACCCGGAACTGGCGGCGTTTATT |
| | | GATGAATGCCGCAACACCAAAGTGGCGGAAGCGGAAATGGCGACCAT |
| | | GGAAAAAAAAGGCGTGGATACCGGCTTTAAAGCGGTGCATCCGCTGA |
| | | CCGGCGAAGAAATTCCGGTGTGGGCGGCGAACTTTGTGCTGATGGAA |
| | | TATGGCACCGGCGCGGTGATGGCGGTGCCGGGCCATGATCAGCGCGA |
| | | TTATGAATTTGCGAGCAAATATGGCCTGAACATTAAACCGGTGATTC |
| | | TGGCGGCGGATGGCAGCGAACCGGATCTGAGCCAGCAGGCGCTGACC |
| | | GAAAAAGGCGTGCTGTTTAACAGCGGCGAATTTAACGGCCTGGATCA |
| | | TGAAGCGGCGTTTAACGCGATTGCGGATAAACTGACCGCGATGGGCG |
| | | TGGGCGAACGCAAAGTGAACTATCGCCTGCGCGATTGGGGCGTGAGC |
| | | CGCCAGCGCTATTGGGGCGCGCCGATTCCGATGGTGACCCTGGAAGA |
| | | TGGCACCGTGATGCCGACCCCGGATGATCAGCTGCCGGTGATTCTGC |
| | | CGGAAGATGTGGTGATGGATGGCATTACCAGCCCGATTAAAGCGGAT |
| | | CCGGAATGGGCGAAAACCACCGTGAACGGCATGCCGGCGCTGCGCGA |
| | | AACCGATACCTTTGATACCTTTATGGAAAGCAGCTGGCATTATGCGC |
| | | GCTATACCTGCCCGCAGTATAAAGAAGGCATGCTGGATAGCGAAGCG |
| | | GCGAACTATTGGCTGCCGGTGGATATTGCGATTGGCGGCATTGAACA |
| | | TGCGATTATGGGCCTGCTGTATTTTCGCTTTTTTCATAAACTGATGC |
| | | GCGATGCGGGCATGGTGAACAGCGATGAACCGGCGAAACAGCTGCTG |
| | | TGCCAGGGCATGGTGCTGGCGGATGCGTTTTATTATGTGGGCGAAAA |
| | | CGGCGAACGCAACTGGGTGAGCCCGGTGGATGCGATTGTGGAACGCG |
| | | ATGAAAAAGGCCGCATTGTGAAAGCGAAAGATGCGGCGGGCCATGAA |
| | | CTGGTGTATACCGGCATGAGCAAAATGAGCAAAAGCAAAAACAACGG |
| | | CATTGATCCGCAGGTGATGGTGGAACGCTATGGCGCGGATACCGTGC |
| | | GCCTGTTTATGATGTTTGCGAGCCCGGCGGATATGACCCTGGAATGG |
| | | CAGGAAAGCGGCGTGGAAGGCGCGAACCGCTTTCTGAAACGCGTGTG |
| | | GAAACTGGTGTATGAACATACCGCGAAAGGCGATGTGGCGGCGCTGA |
| | | ACGTGGATGCGCTGACCGAAAACCAGAAAGCGCTGCGCCGCGATGTG |
| | | CATAAAACCATTGCGAAAGTGACCGATGATATTGGCCGCCGCCAGAC |
| | | CTTTAACACCGCGATTGCGGCGATTATGGAACTGATGAACAAACTGG |
| | | CGAAAGCGCCGACCGATGGCGAACAGGATCGCGCGCTGATGCAGGAA |
| | | GCGCTGCTGGCGGTGGTGCGCATGCTGAACCCGTTTACCCCGCATAT |
| | | TTGCTTTACCCTGTGGCAGGAACTGAAAGGCGAAGGCGATATTGATA |
| | | ACGCGCCGTGGCCGGTGGCGGATGAAAAAGCGATGGTGGAAGATAGC |
| | | ACCCTGGTGGTGGTGCAGGTGAACGGCAAAGTGCGCGCGAAAATTAC |
| | | CGTGCCGGTGGATGCGACCGAAGAACAGGTGCGCGAACGCGCGGGCC |
| | | AGGAACATCTGGTGGCGAAATATCTGGATGGCGTGACCGTGCGCAAA |
| | | GTGATTTATGTGCCGGGCAAACTGCTGAACCTGGTGGTGGGCGGCCC |
| | | GGTG |
| 65 | V54 | ATGGAAGAACAGTATCGCCCGGAAGAAATTGAAAGCAAAGTGCAGCT |
| | | GCATTGGGATAAAAAACGCACCTTTGAAGTGACCGAAGATGAAAGCA |
| | | AAGAAAAATATTATTGCCTGAGCATTAGCCCGTATCCGAGCGGCCGC |
| | | CTGCATATGGGCCATGTGCGCAACTATACCATTGGCGATGTGATTGC |
| | | GCGCTATCAGCGCATGCTGGGCAAAAACGTGCTGCAGCCGATTGGCT |
| | | GGGATGCGTTTGGCCTGCCGGCGGAAGGCGCGGCGGTGAAAAACAAC |
| | | ACCGCGCCGGCGCCGTGGACCTATGATAACATTGCGTATATGAAAAA |
| | | CCAGCTGAAAATGCTGGGCTTTGGCTATGATTGGAGCCGCGAACTGG |
| | | CGACCTGCACCCCGGAATATTATCGCTGGGAACAGAAATTTTTTACC |
| | | GAACTGTATAAAAAAGGCCTGGTGTATAAAAAAACCAGCGCGGTGAA |
| | | CTGGTGCCCGAACGATCAGACCGTGCTGGCGAACGAACAGGTGATTG |
| | | ATGGCTGCTGCTGGCGCTGCGATACCAAAGTGGAACGCAAAGAAATT |
| | | CCGCAGTGGTTTATTAAAATTACCGCGTATGCGGATGAACTGCTGAA |
| | | CGATCTGGATAAACTGGATCATTGGCCGGATACCGTGAAAACCATGC |
| | | AGCGCAACTGGATTGGCCGCAGCGAAGGCGTGGAAATTACCTTTAAC |
| | | GTGAACGATTATGATAACACCCTGACCGTGTATACCACCCGCCCGGA |
| | | TGCGTTTATGGGCTGCACCTATCTGGCGGTGGCGGCGGGCCATCCGC |
| | | TGGCGCAGAAAGCGGCGGAAAACAACCCGGAACTGGCGGCGTTTATT |
| | | GATGAATGCCGCAACACCAAAGTGGCGGAAGCGGAAATGGCGACCAT |
| | | GGAAAAAAAAGGCGTGGATACCGGCTTTAAAGCGGTGCATCCGCTGA |
| | | CCGGCGAAGAAATTCCGGTGTGGGCGGCGAACTTTGTGCTGATGGAA |
| | | TATGGCACCGGCGCGGTGATGGCGGTGCCGGGCCATGATCAGCGCGA |
| | | TTATGAATTTGCGAGCAAATATGGCCTGAACATTAAACCGGTGATTC |
| | | TGGCGGCGGATGGCAGCGAACCGGATCTGAGCCAGCAGGCGCTGACC |
| | | GAAAAAGGCGTGCTGTTTAACAGCGGCGAATTTAACGGCCTGGATCA |
| | | TGAAGCGGCGTTTAACGCGATTGCGGATAAACTGACCGCGATGGGCG |
| | | TGGGCGAACGCAAAGTGAACTATCGCCTGCGCGATTGGGGCGTGAGC |
| | | CGCCAGCGCTATTGGGGCGCGCCGATTCCGATGGTGACCCTGGAAGA |
| | | TGGCACCGTGATGCCGACCCCGGATGATCAGCTGCCGGTGATTCTGC |
| | | CGGAAGATGTGGTGATGGATGGCATTACCAGCCCGATTAAAGCGGAT |
| | | CCGGAATGGGCGAAAACCACCGTGAACGGCATGCCGGCGCTGCGCGA |
| | | AACCGATACCTTTGATACCTTTATGGAAAGCAGCTGGATTTATGCGC |
| | | GCTATACCTGCCCGCAGTATAAAGAAGGCATGCTGGATAGCGAAGCG |
| | | GCGAACTATTGGCTGCCGGTGGATATTATTATTGGCGGCATTGAACA |
| | | TGCGATTATGGGCCTGCTGTATTTTCGCTTTTTTCATAAACTGATGC |
| | | GCGATGCGGGCATGGTGAACAGCGATGAACCGGCGAAACAGCTGCTG |
| | | TGCCAGGGCATGGTGCTGGCGGATGCGTTTTATTATGTGGGCGAAAA |
| | | CGGCGAACGCAACTGGGTGAGCCCGGTGGATGCGATTGTGGAACGCG |
| | | ATGAAAAAGGCCGCATTGTGAAAGCGAAAGATGCGGCGGGCCATGAA |
| | | CTGGTGTATACCGGCATGAGCAAAATGAGCAAAAGCAAAAACAACGG |
| | | CATTGATCCGCAGGTGATGGTGGAACGCTATGGCGCGGATACCGTGC |
| | | GCCTGTTTATGATGTTTGCGAGCCCGGCGGATATGACCCTGGAATGG |
| | | CAGGAAAGCGGCGTGGAAGGCGCGAACCGCTTTCTGAAACGCGTGTG |
| | | GAAACTGGTGTATGAACATACCGCGAAAGGCGATGTGGCGGCGCTGA |
| | | ACGTGGATGCGCTGACCGAAAACCAGAAAGCGCTGCGCCGCGATGTG |
| | | CATAAAACCATTGCGAAAGTGACCGATGATATTGGCCGCCGCCAGAC |
| | | CTTTAACACCGCGATTGCGGCGATTATGGAACTGATGAACAAACTGG |
| | | CGAAAGCGCCGACCGATGGCGAACAGGATCGCGCGCTGATGCAGGAA |
| | | GCGCTGCTGGCGGTGGTGCGCATGCTGAACCCGTTTACCCCGCATAT |
| | | TTGCTTTACCCTGTGGCAGGAACTGAAAGGCGAAGGCGATATTGATA |
| | | ACGCGCCGTGGCCGGTGGCGGATGAAAAAGCGATGGTGGAAGATAGC |
| | | ACCCTGGTGGTGGTGCAGGTGAACGGCAAAGTGCGCGCGAAAATTAC |
| | | CGTGCCGGTGGATGCGACCGAAGAACAGGTGCGCGAACGCGCGGGCC |
| | | AGGAACATCTGGTGGCGAAATATCTGGATGGCGTGACCGTGCGCAAA |
| | | GTGATTTATGTGCCGGGCAAACTGCTGAACCTGGTGGTGGGCGGCCC |
| | | GGTG |
| 66 | V55 | ATGGAAGAACAGTATCGCCCGGAAGAAATTGAAAGCAAAGTGCAGCT |
| | | GCATTGGGATAAAAAACGCACCTTTGAAGTGACCGAAGATGAAAGCA |
| | | AAGAAAAATATTATTGCCTGAGCATTAGCCCGTATCCGAGCGGCCGC |
| | | CTGCATATGGGCCATGTGCGCAACTATACCATTGGCGATGTGATTGC |
| | | GCGCTATCAGCGCATGCTGGGCAAAAACGTGCTGCAGCCGATTGGCT |
| | | GGGATGCGTTTGGCCTGCCGGCGGAAGGCGCGGCGGTGAAAAACAAC |
| | | ACCGCGCCGGCGCCGTGGACCTATGATAACATTGCGTATATGAAAAA |
| | | CCAGCTGAAAATGCTGGGCTTTGGCTATGATTGGAGCCGCGAACTGG |
| | | CGACCTGCACCCCGGAATATTATCGCTGGGAACAGAAATTTTTTACC |
| | | GAACTGTATAAAAAAGGCCTGGTGTATAAAAAAACCAGCGCGGTGAA |
| | | CTGGTGCCCGAACGATCAGACCGTGCTGGCGAACGAACAGGTGATTG |
| | | ATGGCTGCTGCTGGCGCTGCGATACCAAAGTGGAACGCAAAGAAATT |
| | | CCGCAGTGGTTTATTAAAATTACCGCGTATGCGGATGAACTGCTGAA |
| | | CGATCTGGATAAACTGGATCATTGGCCGGATACCGTGAAAACCATGC |
| | | AGCGCAACTGGATTGGCCGCAGCGAAGGCGTGGAAATTACCTTTAAC |
| | | GTGAACGATTATGATAACACCCTGACCGTGTATACCACCCGCCCGGA |
| | | TGCGTTTATGGGCTGCACCTATCTGGCGGTGGCGGCGGGCCATCCGC |
| | | TGGCGCAGAAAGCGGCGGAAAACAACCCGGAACTGGCGGCGTTTATT |
| | | GATGAATGCCGCAACACCAAAGTGGCGGAAGCGGAAATGGCGACCAT |
| | | GGAAAAAAAAGGCGTGGATACCGGCTTTAAAGCGGTGCATCCGCTGA |
| | | CCGGCGAAGAAATTCCGGTGTGGGCGGCGAACTTTGTGCTGATGGAA |
| | | TATGGCACCGGCGCGGTGATGGCGGTGCCGGGCCATGATCAGCGCGA |
| | | TTATGAATTTGCGAGCAAATATGGCCTGAACATTAAACCGGTGATTC |
| | | TGGCGGCGGATGGCAGCGAACCGGATCTGAGCCAGCAGGCGCTGACC |
| | | GAAAAAGGCGTGCTGTTTAACAGCGGCGAATTTAACGGCCTGGATCA |
| | | TGAAGCGGCGTTTAACGCGATTGCGGATAAACTGACCGCGATGGGCG |
| | | TGGGCGAACGCAAAGTGAACTATCGCCTGCGCGATTGGGGCGTGAGC |
| | | CGCCAGCGCTATTGGGGCGCGCCGATTCCGATGGTGACCCTGGAAGA |
| | | TGGCACCGTGATGCCGACCCCGGATGATCAGCTGCCGGTGATTCTGC |
| | | CGGAAGATGTGGTGATGGATGGCATTACCAGCCCGATTAAAGCGGAT |
| | | CCGGAATGGGCGAAAACCACCGTGAACGGCATGCCGGCGCTGCGCGA |
| | | AACCGATACCTTTGATACCTTTATGGAAAGCAGCTGGATTTATGCGC |
| | | GCTATACCTGCCCGCAGTATAAAGAAGGCATGCTGGATAGCGAAGCG |
| | | GCGAACTATTGGCTGCCGGTGGATATTGTGATTGGCGGCATTGAACA |
| | | TGCGATTATGGGCCTGCTGTATTTTCGCTTTTTTCATAAACTGATGC |
| | | GCGATGCGGGCATGGTGAACAGCGATGAACCGGCGAAACAGCTGCTG |
| | | TGCCAGGGCATGGTGCTGGCGGATGCGTTTTATTATGTGGGCGAAAA |
| | | CGGCGAACGCAACTGGGTGAGCCCGGTGGATGCGATTGTGGAACGCG |
| | | ATGAAAAAGGCCGCATTGTGAAAGCGAAAGATGCGGCGGGCCATGAA |
| | | CTGGTGTATACCGGCATGAGCAAAATGAGCAAAAGCAAAAACAACGG |
| | | CATTGATCCGCAGGTGATGGTGGAACGCTATGGCGCGGATACCGTGC |
| | | GCCTGTTTATGATGTTTGCGAGCCCGGCGGATATGACCCTGGAATGG |
| | | CAGGAAAGCGGCGTGGAAGGCGCGAACCGCTTTCTGAAACGCGTGTG |
| | | GAAACTGGTGTATGAACATACCGCGAAAGGCGATGTGGCGGCGCTGA |
| | | ACGTGGATGCGCTGACCGAAAACCAGAAAGCGCTGCGCCGCGATGTG |
| | | CATAAAACCATTGCGAAAGTGACCGATGATATTGGCCGCCGCCAGAC |
| | | CTTTAACACCGCGATTGCGGCGATTATGGAACTGATGAACAAACTGG |
| | | CGAAAGCGCCGACCGATGGCGAACAGGATCGCGCGCTGATGCAGGAA |
| | | GCGCTGCTGGCGGTGGTGCGCATGCTGAACCCGTTTACCCCGCATAT |
| | | TTGCTTTACCCTGTGGCAGGAACTGAAAGGCGAAGGCGATATTGATA |
| | | ACGCGCCGTGGCCGGTGGCGGATGAAAAAGCGATGGTGGAAGATAGC |
| | | ACCCTGGTGGTGGTGCAGGTGAACGGCAAAGTGCGCGCGAAAATTAC |
| | | CGTGCCGGTGGATGCGACCGAAGAACAGGTGCGCGAACGCGCGGGCC |
| | | AGGAACATCTGGTGGCGAAATATCTGGATGGCGTGACCGTGCGCAAA |
| | | GTGATTTATGTGCCGGGCAAACTGCTGAACCTGGTGGTGGGCGGCCC |
| | | GGTG |
| 67 | V56 | ATGGAAGAACAGTATCGCCCGGAAGAAATTGAAAGCAAAGTGCAGCT |
| | | GCATTGGGATAAAAAACGCACCTTTGAAGTGACCGAAGATGAAAGCA |
| | | AAGAAAAATATTATTGCCTGAGCATTAGCCCGTATCCGAGCGGCCGC |
| | | CTGCATATGGGCCATGTGCGCAACTATACCATTGGCGATGTGATTGC |
| | | GCGCTATCAGCGCATGCTGGGCAAAAACGTGCTGCAGCCGATTGGCT |
| | | GGGATGCGTTTGGCCTGCCGGCGGAAGGCGCGGCGGTGAAAAACAAC |
| | | ACCGCGCCGGCGCCGTGGACCTATGATAACATTGCGTATATGAAAAA |
| | | CCAGCTGAAAATGCTGGGCTTTGGCTATGATTGGAGCCGCGAACTGG |
| | | CGACCTGCACCCCGGAATATTATCGCTGGGAACAGAAATTTTTTACC |
| | | GAACTGTATAAAAAAGGCCTGGTGTATAAAAAAACCAGCGCGGTGAA |
| | | CTGGTGCCCGAACGATCAGACCGTGCTGGCGAACGAACAGGTGATTG |
| | | ATGGCTGCTGCTGGCGCTGCGATACCAAAGTGGAACGCAAAGAAATT |
| | | CCGCAGTGGTTTATTAAAATTACCGCGTATGCGGATGAACTGCTGAA |
| | | CGATCTGGATAAACTGGATCATTGGCCGGATACCGTGAAAACCATGC |
| | | AGCGCAACTGGATTGGCCGCAGCGAAGGCGTGGAAATTACCTTTAAC |
| | | GTGAACGATTATGATAACACCCTGACCGTGTATACCACCCGCCCGGA |
| | | TGCGTTTATGGGCTGCACCTATCTGGCGGTGGCGGCGGGCCATCCGC |
| | | TGGCGCAGAAAGCGGCGGAAAACAACCCGGAACTGGCGGCGTTTATT |
| | | GATGAATGCCGCAACACCAAAGTGGCGGAAGCGGAAATGGCGACCAT |
| | | GGAAAAAAAAGGCGTGGATACCGGCTTTAAAGCGGTGCATCCGCTGA |
| | | CCGGCGAAGAAATTCCGGTGTGGGCGGCGAACTTTGTGCTGATGGAA |
| | | TATGGCACCGGCGCGGTGATGGCGGTGCCGGGCCATGATCAGCGCGA |
| | | TTATGAATTTGCGAGCAAATATGGCCTGAACATTAAACCGGTGATTC |
| | | TGGCGGCGGATGGCAGCGAACCGGATCTGAGCCAGCAGGCGCTGACC |
| | | GAAAAAGGCGTGCTGTTTAACAGCGGCGAATTTAACGGCCTGGATCA |
| | | TGAAGCGGCGTTTAACGCGATTGCGGATAAACTGACCGCGATGGGCG |
| | | TGGGCGAACGCAAAGTGAACTATCGCCTGCGCGATTGGGGCGTGAGC |
| | | CGCCAGCGCTATTGGGGCGCGCCGATTCCGATGGTGACCCTGGAAGA |
| | | TGGCACCGTGATGCCGACCCCGGATGATCAGCTGCCGGTGATTCTGC |
| | | CGGAAGATGTGGTGATGGATGGCATTACCAGCCCGATTAAAGCGGAT |
| | | CCGGAATGGGCGAAAACCACCGTGAACGGCATGCCGGCGCTGCGCGA |
| | | AACCGATACCTTTGATACCTTTATGGAAAGCAGCTGGATTTATGCGC |
| | | GCTATACCTGCCCGCAGTATAAAGAAGGCATGCTGGATAGCGAAGCG |
| | | GCGAACTATTGGCTGCCGGTGGATATTGGCATTGGCGGCATTGAACA |
| | | TGCGATTATGGGCCTGCTGTATTTTCGCTTTTTTCATAAACTGATGC |
| | | GCGATGCGGGCATGGTGAACAGCGATGAACCGGCGAAACAGCTGCTG |
| | | TGCCAGGGCATGGTGCTGGCGGATGCGTTTTATTATGTGGGCGAAAA |
| | | CGGCGAACGCAACTGGGTGAGCCCGGTGGATGCGATTGTGGAACGCG |
| | | ATGAAAAAGGCCGCATTGTGAAAGCGAAAGATGCGGCGGGCCATGAA |
| | | CTGGTGTATACCGGCATGAGCAAAATGAGCAAAAGCAAAAACAACGG |
| | | CATTGATCCGCAGGTGATGGTGGAACGCTATGGCGCGGATACCGTGC |
| | | GCCTGTTTATGATGTTTGCGAGCCCGGCGGATATGACCCTGGAATGG |
| | | CAGGAAAGCGGCGTGGAAGGCGCGAACCGCTTTCTGAAACGCGTGTG |
| | | GAAACTGGTGTATGAACATACCGCGAAAGGCGATGTGGCGGCGCTGA |
| | | ACGTGGATGCGCTGACCGAAAACCAGAAAGCGCTGCGCCGCGATGTG |
| | | CATAAAACCATTGCGAAAGTGACCGATGATATTGGCCGCCGCCAGAC |
| | | CTTTAACACCGCGATTGCGGCGATTATGGAACTGATGAACAAACTGG |
| | | CGAAAGCGCCGACCGATGGCGAACAGGATCGCGCGCTGATGCAGGAA |
| | | GCGCTGCTGGCGGTGGTGCGCATGCTGAACCCGTTTACCCCGCATAT |
| | | TTGCTTTACCCTGTGGCAGGAACTGAAAGGCGAAGGCGATATTGATA |
| | | ACGCGCCGTGGCCGGTGGCGGATGAAAAAGCGATGGTGGAAGATAGC |
| | | ACCCTGGTGGTGGTGCAGGTGAACGGCAAAGTGCGCGCGAAAATTAC |
| | | CGTGCCGGTGGATGCGACCGAAGAACAGGTGCGCGAACGCGCGGGCC |
| | | AGGAACATCTGGTGGCGAAATATCTGGATGGCGTGACCGTGCGCAAA |
| | | GTGATTTATGTGCCGGGCAAACTGCTGAACCTGGTGGTGGGCGGCCC |
| | | GGTG |
| 68 | | GTGGGGTTCCCGAGCGGCCAAAGGGAGCAGACTtcAAATCTGCCGTC |
| | | ACAGACTTCGAAGGTTCGAATCCTTCCCCCACCA |
| 69 | | GTGGGGTTCCCGAGCGGCCAAAGGGAGCAGACTnnnAATCTGCCGTC |
| | | ACAGACTTCGAAGGTTCGAATCCTTCCCCCACCA |
| | | wherein nnn is CTA, TCA, or TTA |
| 70 | | MASSNLIKQLQERGLVAQVTDEEALAERLAQGPIALVCGFDPTADSL |
| | | HLGHLVPLLCLKRFQQAGHKPVALVGGATGLIGDPSFKAAERKLNTE |
| | | ETVQEWVDKIRKQVAPFLDEDCGENSAIAANNYDWFGNMNVLTFLRD |
| | | IGKHFSVNQMINKEAVKQRLNREDQGISFTEFSYNLLQGYSMACLNK |
| | | QYGVVLQIGGSDQWGNITSGIDLTRRLHQNQVFGLTVPLITKADGTK |
| | | FGKTEGGAVWLDPKKTSPYKFYQFWINTADADVYRFLKFFTEMSIEE |
| | | INALEEEDKNSGKAPRAQYVLAEQVTRLVHGEEGLQAAKRITECLES |
| | | GSLSALSEADFEQLAQDGVPMVEMEKGADLMQALVDSELQPSRGQAR |
| | | KTIASNAITINGEKQSDPEYFFKEEDRLFGRFTLLRRGKKNYCLICW |
| | | KGPV |
| 71 | | atggcaagcagtaacttgattaaacaattgcaagagcgggggctggt |
| | | agcccaggtgacggacgaggaagcgttagcagagcgactggcgcaag |
| | | gcccgatcgcactcGTGtgtggcttcgatcctaccgctgacagcttg |
| | | catttggggcatcttgttccattgttatgcctgaaacgcttccagca |
| | | ggcgggccacaagccggttgcgctggtaggcggcgcgacgggtctga |
| | | ttggcgacccgagcttcaaagctgccgagcgtaagctgaacaccgaa |
| | | gaaactgttcaggagtgggtggacaaaatccgtaagcaggttgcccc |
| | | gttcctcgatttcgactgtggagaaaactctgctatcgcggccaata |
| | | attatgactggttcggcaatatgaatgtgctgaccttcctgcgcgat |
| | | attggcaaacacttctccgttaaccagatgatcaacaaagaagcggt |
| | | taagcagcgtctcaaccgtgaagAtcaggggatttcgttcactgagt |
| | | tttcctacaacctgctGcagggttatAgtatggcctgtTTGaacaaa |
| | | cagtacggtgtggtgctgcaaattggtggttctgaccaatggggtaa |
| | | catcacttctggtatcgacctgacccgtcgtctgcatcagaatcagg |
| | | tgtttggcctgaccgttccgctgatcactaaagcagatggcaccaaa |
| | | tttggtaaaactgaaggcggcgcagtctggttggacccgaagaaaac |
| | | cagcccgtacaaattctaccagttctggatcaacactgcggatgccg |
| | | acgtttaccgcttcctgaagttcttcacctttatgagcattgaagag |
| | | atcaacgccctggaagaagaagataaaaacagcggtaaagcaccgcg |
| | | cgcccagtatgtactggcggagcaggtgactcgtctggttcacggtg |
| | | aagaaggtttacaggcggcaaaacgtattaccgaatgcctgttcagc |
| | | ggttctttgagtgcgctgagtgaagcggacttcgaacagctggcgca |
| | | ggacggcgtaccgatggttgagatggaaaagggcgcagacctgatgc |
| | | aggcactggtcgattctgaactgcaaccttcccgtggtcaggcacgt |
| | | aaaactatcgcctccaatgccatcaccattaacggtgaaaaacagtc |
| | | cgatcctgaatacttctttaaagaagaagatcgtctgtttggtcgtt |
| | | ttaccttactgcgtcgcggtaaaaagaattactgtctgatttgctgg |
| | | aaagggcccgtttaa |
| 72 | | GGAAACCTGATCATGTAGATCGAACGGACTCTAAATCCGTTCAGCCG |
| | | GGTTAGATTCCCGGGGTTTCCG |
| 73 | | GGAAACCTGATCATGTAGATCGAACGGACTnnnAATCCGTTCAGCCG |
| | | GGTTAGATTCCCGGGGTTTCCG |
| 74 | | gGAAACCugaucauguagaucgaacggacucuaaauccguucagccg |
| | | gguuagauucccggGGUUUCcgcca |
| 75 | | gGGCGGCugaucauguagaucgaacggacucuaaauccguucagccg |
| | | gguuagauucccggGCUGCCcgcca |
| 76 | | gGGUGACugaucauguagaucgaacggacucuaaauccguucagccg |
| | | gguuagauucccggGUUGCCcgcca |
| 77 | | gGGGGGCugaucauguagaucgaacggacucuaaauccguucagccg |
| | | gguuagauucccggGCUCCCcgcca |
| 78 | | gGGCGGCugaucauguagaucgaacggacucuaaauccguucagccg |
| | | gguuagauucccggGUUGCCcgcca |
| 79 | | gGGCGCCugaucauguagaucgaacggacucuaaauccguucagccg |
| | | gguuagauucccggGGCGCCcgcca |
| 80 | | gGGGAGGugaucauguagaucgaacggacucuaaauccguucagccg |
| | | gguuagauucccggCCUCCCcgcca |
| 81 | | gGGGACCugaucauguagaucgaacggacucuaaauccguucagccg |
| | | gguuagauucccggGGUCCCcgcca |
| 82 | | gGCCGGGugaucauguagaucgaacggacucuaaauccguucagccg |
| | | gguuagauucccggCCUGGCcgcca |
| 83 | | gGGGACCugaucauguagaucgaacggacucuaaauccguucagccg |
| | | gguuagauucccggGGUCCUcgcca |
| 84 | | gGGGCCCugaucauguagaucgaacggacucuaaauccguucagccg |
| | | gguuagauucccggGGGUCCcgcca |
| 85 | | gGGGGCCugaucauguagaucgaacggacucuaaauccguucagccg |
| | | gguuagauucccggGGCUCCcgcca |
| 86 | | gGGGUCCugaucauguagaucgaacggacucuaaauccguucagccg |
| | | gguuagauucccggGGAUCCcgcca |
| 87 | | gGGGACCugaucauguagaucgaacggacucuaaauccguucagccg |
| | | gguuagauucccggGGUUCCcgcca |
| 88 | | gGGGAGGugaucauguagaucgaacggacucuaaauccguucagccg |
| | | gguuagauucccggUCUUCCcgcca |
| 89 | | gGGGGGGugaucauguagaucgaacggacucuaaauccguucagccg |
| | | gguuagauucccggCCCCCUcgcca |
| 90 | | gGUGGGGugaucauguagaucgaacggacucuaaauccguucagccg |
| | | gguuagauucccggCCCUACcgcca |
| 91 | | gGGGGUCugaucauguagaucgaacggacucuaaauccguucagccg |
| | | gguuagauucccggGACUCCcgcca |
| 92 | | gGGUCCCugaucauguagaucgaacggacucuaaauccguucagccg |
| | | gguuagauucccggGGGGUCcgcca |
| 93 | | gGGGACCugaucauguagaucgaacggacucuaaauccguucagccg |
| | | gguuagauucccggGGUUCUcgcca |
| 94 | | gGGCGGCugaucauguagaucgaacggacucuaaauccguucagccg |
| | | gguuagauucccggGCCGCCcgcca |
| 95 | | gAGCACCugaucauguagaucgaacggacucuaaauccguucagccg |
| | | gguuagauucccggGGUGCUcgcca |
| 96 | | gGGGGGGugaucauguagaucgaacggacucuaaauccguucagccg |
| | | gguuagauucccggCCCCCCcgcca |
| 97 | | gAGGGGGugaucauguagaucgaacggacucuaaauccguucagccg |
| | | gguuagauucccggCCCCCUcgcca |
| 98 | | gGGAGCCugaucauguagaucgaacggacucuaaauccguucagccg |
| | | gguuagauucccggGGUUCCcgcca |
| 99 | | gGGAGCCugaucauguagaucgaacggacucuaaauccguucagccg |
| | | gguuagauucccggGGUUCCcgcca |
| 100 | | gAGGACCugaucauguagaucgaacggacucuaaauccguucagccg |
| | | gguuagauucccggGGUUCUcgcca |
| 101 | | MDKKPLDVLISATGLWMSRTGTLHKIKHHEVSRSKIYIEMACGDHLV |
| | | VNNSRSCRTARAFRHHKYRKTCKRCRVSDEDINNFLTRSTESKNSVK |
| | | VRVVSAPKVKKAMPKSVSRAPKPLENSVSAKASTNTSRSVPSPAKST |
| | | PNSSVPASAPAPSLTRSQLDRVEALLSPEDKISLNMAKPFRELEPEL |
| | | VTRRKNDFQRLYTNDREDYLGKLERDITKFFVDRGFLEIKSPILIPA |
| | | EYVERMGINNDTELSKQIFRVDKNLCLRPMLAPTLYNYLRKLDRILP |
| | | GPIKIFEVGPCYRKESDGKEHLEEFTMVNFCQMGSGCTRENLEALIK |
| | | EFLDYLEIDFEIVGDSCMVFGDTLDIMHGDLELSSAVVGPVSLDREW |
| | | GIDKPWIGAGFGLERLLKVMHGFKNIKRASRSESYYNGISTNL |
| 102 | | ATGGATAAAAAACCATTAGATGTTTTAATATCTGCGACCGGGCTCTG |
| | | GATGTCCAGGACTGGCACGCTCCACAAAATCAAGCACCATGAGGTCT |
| | | CAAGAAGTAAAATATACATTGAAATGGCGTGTGGAGACCATCTTGTT |
| | | GTGAATAATTCCAGGAGTTGTAGAACAGCCAGAGCATTCAGACATCA |
| | | TAAGTACAGAAAAACCTGCAAACGATGTAGGGTTTCGGACGAGGATA |
| | | TCAATAATTTTCTCACAAGATCAACCGAAAGCAAAAACAGTGTGAAA |
| | | GTTAGGGTAGTTTCTGCTCCAAAGGTCAAAAAAGCTATGCCGAAATC |
| | | AGTTTCAAGGGCTCCGAAGCCTCTGGAAAATTCTGTTTCTGCAAAGG |
| | | CATCAACGAACACATCCAGATCTGTACCTTCGCCTGCAAAATCAACT |
| | | CCAAATTCGTCTGTTCCCGCATCGGCTCCTGCTCCTTCACTTACAAG |
| | | AAGCCAGCTTGATAGGGTTGAGGCTCTCTTAAGTCCAGAGGATAAAA |
| | | TTTCTCTAAATATGGCAAAGCCTTTCAGGGAACTTGAGCCTGAACTT |
| | | GTGACAAGAAGAAAAAACGATTTTCAGCGGCTCTATACCAATGATAG |
| | | AGAAGACTACCTCGGTAAACTCGAACGTGATATTACGAAATTTTTCG |
| | | TAGACCGGGGTTTTCTGGAGATAAAGTCTCCTATCCTTATTCCGGCG |
| | | GAATACGTGGAGAGAATGGGTATTAATAATGATACTGAACTTTCAAA |
| | | ACAGATCTTCCGGGTGGATAAAAATCTCTGCTTGAGGCCAATGCTTG |
| | | CCCCGACTCTTTACAACTATCTGCGAAAACTCGATAGGATTTTACCA |
| | | GGCCCAATAAAAATTTTCGAAGTCGGACCTTGTTACCGGAAAGAGTC |
| | | TGACGGCAAAGAGCACCTGGAAGAATTTACTATGGTGAACTTCTGTC |
| | | AGATGGGTTCGGGATGTACTCGGGAAAATCTTGAAGCTCTCATCAAA |
| | | GAGTTTCTGGACTATCTGGAAATCGACTTCGAAATCGTAGGAGATTC |
| | | CTGTATGGTCTTGGGGATACTCTTGATATAATGCACGGGGACCTGG |
| | | AGCTTTCTTCGGCAGTCGTCGGGCCAGTTTCTCTTGATAGAGAATGG |
| | | GGTATTGACAAACCATGGATAGGTGCAGGTTTTGGTCTTGAACGCTT |
| | | GCTCAAGGTTATGCACGGCTTTAAAAACATTAAGAGGGCATCAAGGT |
| | | CCGAATCTTACTATAATGGGATTTCAACCAATCTGTAA |
| 103 | TrpRS.h14 | ATGACTAAGCCCATCGTTTTTgctGGCGCACAGCCCTCAGGTGAATT |
| | | GACCATTGGTAACTACATGGGTGCGCTGCGTCAGTGGGTAAACATGC |
| | | AGGATGACTACCATTGCATTTACTGTATCGTTGACCAACACGCGATC |
| | | ACCGTGCGCCAGGATGCACAGAAGCTGCGTAAAGCGACGCTGGATAC |
| | | GCTGGCCTTGTATCTGGCTTGTGGTATCGATCCTGAGAAAAGCACCA |
| | | TTTTTGTTCAGTCCCACGTGCCGGAACATGCACAGTTAGGCTGGGCA |
| | | CTGAACTGCTATACCTACTTCGGCGAACTGAGTCGCATGACGCAGTT |
| | | TAAAGATAAATCTGCGCGTTATGCCGAGAACATCAACGCTGGTCTGT |
| | | TTGACTATCCGGTGCTGATGGCAGCGGACATCCTGCTGTATCAAACT |
| | | AATCTGggtccttgtGGTGAAGACCAGAAACAGCACCTCGAACTGAG |
| | | CCGCGATATTGCCCAGCGTTTCAACGCGCTGTATGGCGAGATCTTTA |
| | | AGGTGCCGGAGCCGTTTATTCCGAAATCTGGCGCGCGCGTAATGTCG |
| | | CTGCTGGAGCCGACCAAGAAGATGTCCAAGTCTGACGATAATCGCAA |
| | | TAACGTTATCGGCCTGCTGGAAGATCCGAAATCGGTAGTGAAGAAAA |
| | | TCAAACGTGCGGTCACTGACTCCGACGAGCCGCCGGTAGTTCGCTAC |
| | | GATGTGCAGAACAAAGCGGGCGTTTCCAACCTGTTGGATATCCTTTC |
| | | AGCGGTAACGGGCCAGAGCATCCCAGAACTGGAAAAACAGTTCGAAG |
| | | GCAAGATGTATGGTCATCTGAAAGGTGAAGTGGCTGATGCCGTTTCC |
| | | GGTATGCTGACTGAATTGCAGGAACGCTATCACCGTTTCCGCAACGA |
| | | TGAAGCCTTCCTGCAACAGGTGATGAAAGATGGCGCGGAAAAAGCCA |
| | | GCGCGCACGCTTCCCGTACGCTAAAAGCGGTGTACGAAGCGATTGGT |
| | | TTTGTGGCGAAGCCGTAA |
| 104 | | GTGGGGTTCCCGAGCGGCCAAAGGGAGCAGACTtcAAATCTGCCGTC |
| | | ACAGACTTCGAAGGTTCGAATCCTTCCCCCACCACCA |
| 105 | | GTGGGGTTCCCGAGCGGCCAAAGGGAGCAGACTnnnAATCTGCCGTC |
| | | ACAGACTTCGAAGGTTCGAATCCTTCCCCCACCACCA |
| | | wherein nnn is CTA, TCA, or TTA |
| 106 | | GGAAACCTGATCATGTAGATCGAACGGACTCTAAATCCGTTCAGCCG |
| | | GGTTAGATTCCCGGGGTTTCCGCCA |
| 107 | | GGAAACCTGATCATGTAGATCGAACGGACTnnnAATCCGTTCAGCCG |
| | | GGTTAGATTCCCGGGGTTTCCGCCA |
| 108 | | AGGGGCGTAGTTCAATTGGTAGAGCACCGGTCTccaAAACCGGGTGT |
| | | TGGGAGTTCGAGTCTCTCCGCCCCTG |
| 109 | | AGGGGCGTAGTTCAATTGGTAGAGCACCGGTCTctaAAACCGGGTGT |
| | | TGGGAGTTCGAGTCTCTCCGCCCCTG |
| 110 | | AGGGGCGTAGTTCAATTGGTAGAGCACCGGTCTtcaAAACCGGGTGT |
| | | TGGGAGTTCGAGTCTCTCCGCCCCTG |
| 111 | | AGGGGCGTAGTTCAATTGGTAGAGCACCGGTCTttaAAACCGGGTGT |
| | | TGGGAGTTCGAGTCTCTCCGCCCCTG |
| 112 | | TGGGGTATCGCCAAGCGGTAAGGCACCGGATTCnnnTTCCGGCATTC |
| | | CGAGGTTCGAATCCTCGTACCCCAG |
| | | wherein nnn is CTA, TCA, or TTA |
| 113 | | AGGGGCATAGCTCAAGCGGTAAAGCACCGGACTnnnAAACCGGCAGT |
| | | CCGAAGTTCGAATCCCCCCACCCCAG |
| | | wherein nnn is CTA, TCA, or TTA |
| 114 | | EVQLVESGGGLVQPGGSLRLSCAASGFNIKDTYIHWVRQAPGKGLEW |
| | | VARIYPTNGYTRYADSVKGRFTISADTSKNTAYLQMNSLRAEDTAVY |
| | | YCSRWGGDGFYAMDYWGQGTLVTVSSASTKGPSVFPLAPSSKSTSGG |
| | | TAALGCLVKDYFPEPVTVSWNSGALTSGVHTFPAVLQSSGLYSLSSV |
| | | VTVPSSSLGTQTYICNVNHKPSNTKVDKKVEPKSCDKTHTCPPCPAP |
| | | ELLGGPSVFLFPPKPKDTLMISRTPEVTCVVVDVSHEDPEVKENWYV |
| | | DGVEVHNAKTKPREEQYNSTYRVVSVLTVLHQDWLNGKEYKCKVSNK |
| | | ALPAPIEKTISKAKGQPREPQVYTLPPSREEMTKNQVSLTCLVKGFY |
| | | PSDIAVEWESNGQPENNYKTTPPVLDSDGSFFLYSKLTVDKSRWQQG |
| | | NVFSCSVMHEALHNHYTQKSLSLSPGK |
| 115 | | DIQMTQSPSSLSASVGDRVTITCRASQDVNTAVAWYQQKPGKAPKLL |
| | | IYSASFLYSGVPSRFSGSRSGTDFTLTISSLQPEDFATYYCQQHYTT |
| | | PPTFGQGTKVEIKRTVAAPSVFIFPPSDEQLKSGTASVVCLLNNFYP |
| | | REAKVQWKVDNALQSGNSQESVTEQDSKDSTYSLSSTLTLSKADYEK |
| | | HKVYACEVTHQGLSSPVTKSENRGEC |

### EXAMPLES

The following Examples are merely illustrative and are not intended to limit the scope or content of the invention in any way.

### Example 1—Generation of UAA Containing Antibodies

Antibody expression was performed using the Expi293 Expression System according to the manufacturer's instructions. Briefly, before transfection, cells were split to a density of 2.7×10⁶ to 3.0×10⁶ cells/ml. A total of 1 mg of plasmid mix (equal parts suppressor plasmid, heavy chain plasmid, and light chain plasmid) was used for transfection into 1 L cell culture. Suppressor plasmid contained anywhere from 1 to 20 copies of leucyl tRNA (Leu-tRNA.h1; SEQ ID NO: 19) or leucyl synthetase (LeuRS.v1; SEQ ID NO: 2). Heavy chain plasmids contained a HC-T198-TAG mutation which facilitated the incorporation of LCA. Plasmids were diluted in 50 ml Opti-MEM medium. A PEI stock solution was incubated at room temperature for 3 minutes, subsequently diluted to achieve a 6:1 PEI:DNA final ratio, and incubated for 15 minutes at room temperature. The Plasmid:PEI complex was then added dropwise to the culture. At the time of transfection, 0.25 to 1 mM LCA (an unnatural amino acid/leucine analog) was added to the cells. Cells were incubated on an orbital shaking platform at 37° C. with 8% CO2 at a speed of 80 to 125 rpm for 5 to 8 days. Protein was purified using a PrismA column (Cytiva: 17549801). It is expected that similar protocols can be used with other compatible UAA/suppressor plasmid/codon mutant combinations (e.g., to incorporate the tryptophan analog HTP).

For hydrophobic interaction chromatography (HIC) analysis, samples were analyzed with a MabPac Butyl column. Mobile phase A was 1.5 M ammonium sulfate, 25 mM sodium phosphate dibasic, pH 7 and mobile phase B was 25 mM sodium phosphate, pH 7. The samples were eluted with a flow rate of 0.8 mL/min and at a column temperature of 25° C. The average drug antibody ratio (DAR) and distribution profiles were determined by peak area percentage of each species.

### Example 2—Preparation of DBCO-2xCy5 Ligand (Compound 209)

A stock solution of 140 mM Cy5 amine was prepared in pH 8.5 sodium bicarbonate and a stock solution of 10 mM DBCO-BIS—NHS linker was prepared in 75% DMSO. DBCO-BIS-NHS only (FIG. 5A) and Cy5 amine only (FIG. 5B) were subjected to HIC analysis to determine baseline peaks prior to co-incubation. Cy5 amine stock solution was added in 14× molar excess for a final concentration of 5 mM DBCO and 70 mM Cy5 amine and conjugated for 18 hours. After incubation, the DBCO-2xCy5 ligand was analyzed by HIC analysis. Results are shown in FIG. 5C, which depicts the new target peak, excess Cy5 amine (as expected), and a minor peak remaining at the DBCO-Bis-NHS retention time.

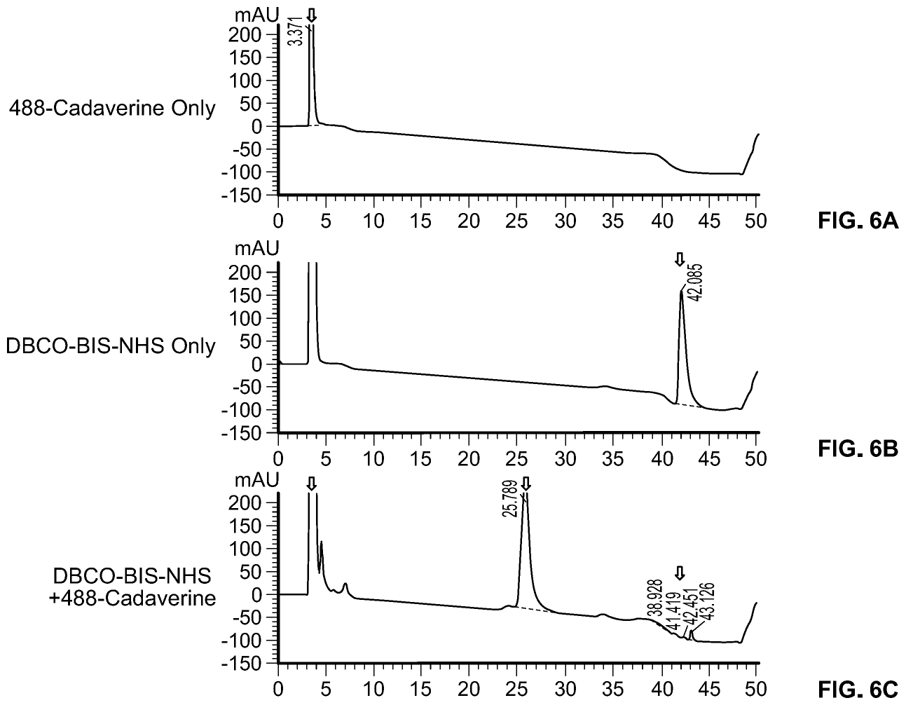

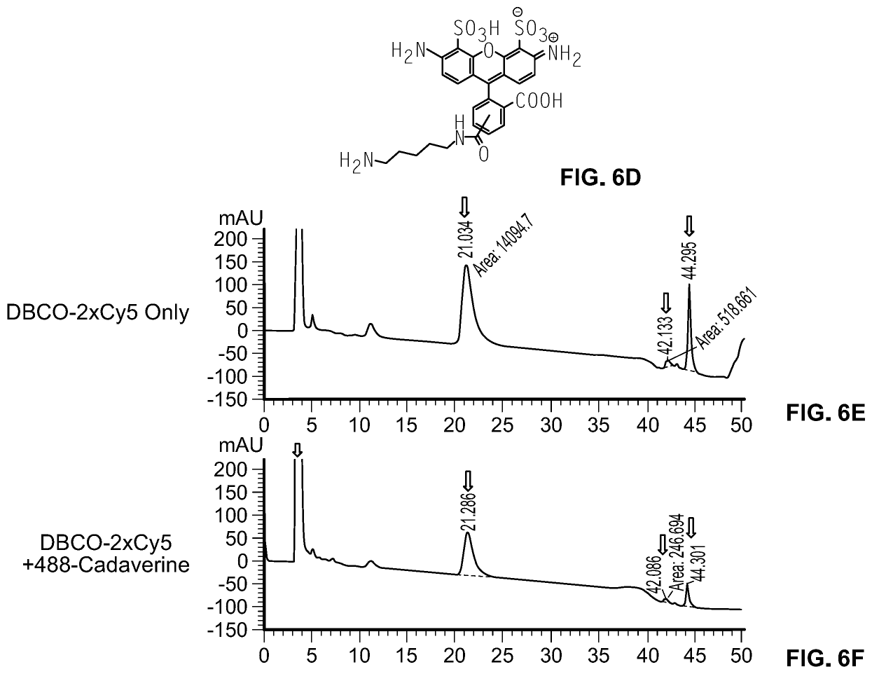

The DBCO-2xCy5 ligand was subjected to a second round of NHS conjugation in order to clarify whether the reaction went to completion and whether the residual peak is an inactive species. A second fluor-amine, 488-Cadaverine, with different hydrophobic and fluorescent properties, was used. 488-Cadaverine was either added to unreacted DBCO-Bis-NHS or DBCO-2xCy5 Ligand at the same ratios as used in FIGS. 5A-5C. In particular, 488-Cadaverine at a final concentration of 50 mM and unreacted DBCO-Bis-NHS at a final concentration of 1.25 mM were incubation for 6 hours, and 488-Cadaverine at a final concentration of 50 mM and DBCO-2xCy5 Ligand at a final concentration of 1.25 mM were incubation for 6 hours. FIG. 6D depicts the 488-Cadaverine structure. FIG. 6A depicts HIC analysis of 488-Cadaverine stock solution only. FIG. 6B depicts HIC analysis of DBCO-BIS-NHS only (as seen in FIG. 5A, and repeated here for reference). FIG. 6C depicts HIC analysis after incubation of 488-Cadaverine and DBCO-BIS-NHS under the same conditions as used in FIGS. 5A-5C. The middle peak (˜25 minutes retention time (RT), green arrow) is predicted to be the DBCO-2xCadaverine final product, demonstrating that DBCO-Bis-NHS is reactive with this amine-fluor as well. FIG. 6E shows the DBCO-2xCy5 compound (previously seen in FIG. 5C, and repeated here for reference). FIG. 6F depicts HIC analysis after incubation of 488-Cadaverine and DBCO-2xCy5 Ligand. No significant change in the distribution of peaks was see in FIG. 6F relative to FIG. 6E, suggesting that the DBCO-2xCy5 preparation went to completion.

### Example 3—Conjugation of Trastuzumab T198-LCA with DBCO-2xCy5 Ligand

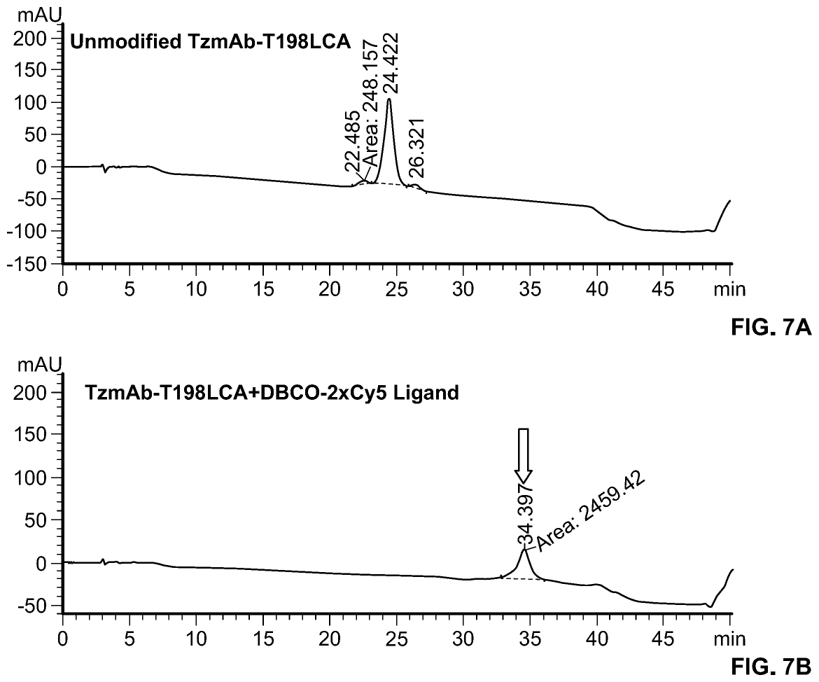

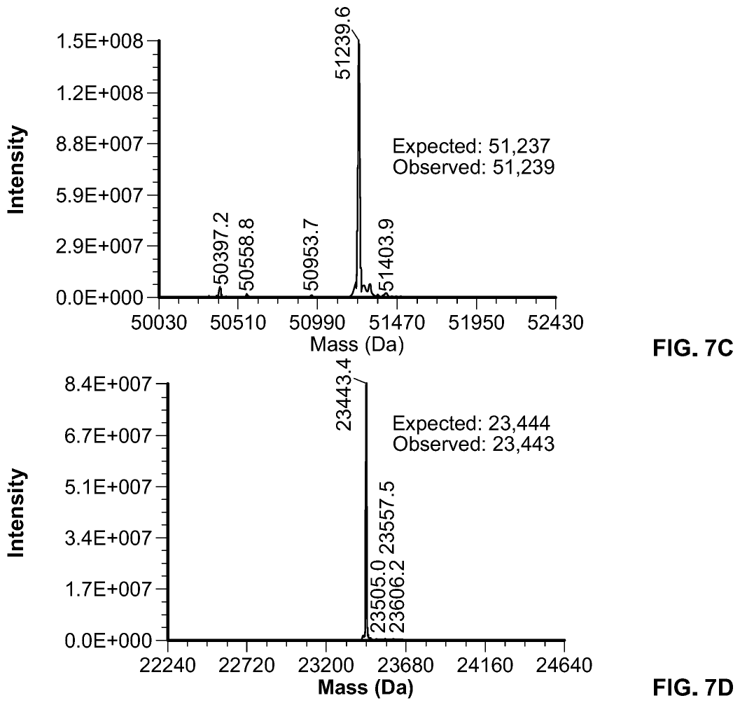

6 μM of trastuzumab (TzmAb) containing a T198LCA mutation in the heavy chain was labeled with 10× molar excess of DBCO-2xCy5 for 3 hours. FIG. 7A depicts HIC analysis of unmodified TzmAb-T198LCA. FIG. 7B depicts HIC analysis following modification. The expected shift retention time shift was observed in FIG. 7B, indicating that DBCO-2xCy5 was conjugated to the antibody. Additionally, mass spectrometry was performed to confirm that the trastuzumab heavy chain was selectively modified with DBCO-2xCy5. For the mass spectrometry analysis, proteins were denatured, reduced, and PNGase treated prior to analysis via electrospray ionization mass spectrometry (ESI-MS). Results are shown in FIG. 7C for the heavy chain and FIG. 7D for the light chain). No significant off-target peaks were observed.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A derivatized protein comprising:
(a) an unnatural amino acid;
(b) a parent linker having a first terminus and a second terminus, wherein the first terminus of the parent linker is covalently conjugated to the unnatural amino acid;
(c) a branching group, wherein the branching group is covalently conjugated to the second terminus of the parent linker;
(d) a plurality of branching linkers, each branching linker comprising a branching unit and a conjugating moiety, wherein each branching linker is covalently conjugated to the branching group via the branching unit present in the branching linker; and
(e) a plurality of molecules, wherein each molecule is covalently conjugated to one of the plurality of branching linkers via the conjugating moiety present in the branching linker.

2. A derivatized protein comprising:
(a) an unnatural amino acid;
(b) a parent linker having a first terminus and second terminus, wherein the first terminus of the parent linker is covalently conjugated to the unnatural amino acid;
(c) a branching group, wherein the branching group is covalently conjugated to the second terminus of the parent linker; and
(d) a plurality of branching linkers, each branching linker comprising a branching unit and a conjugating moiety, wherein each branching linker is covalently conjugated to the branching group via the branching unit present in the branching linker.

3. The protein of claim 1 or 2, wherein the protein comprises at least two branching linkers.

4. The protein of claim 1 or 2, wherein the protein comprises at least three branching linkers.

5. A derivatized protein of Formula I: 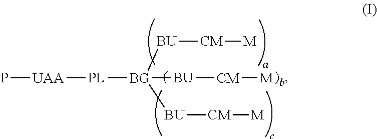
wherein
P is a protein;
UAA is an unnatural amino acid;
PL is a parent linker represented by 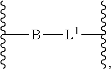
wherein B is a binding unit and L¹ is a chain selected from the group consisting of C₁₋₂₀ alkyl and C₁₋₂₀ heteroalkyl, wherein the chain is optionally substituted by one, two, three or four oxo;
BG (branching group) is a polyvalent atom;
each BU (branching unit) independently is selected from the group consisting of C₁₋₂₀ alkyl and C₁₋₂₀ heteroalkyl, wherein each BU is optionally substituted by one, two or three oxo;
each CM (conjugating moiety) independently is selected from the group consisting of a bond, NH, S, OR¹ and R¹;
R¹ is selected from the group consisting of phenyl, 5-10 membered cycloalkyl, 5-10 membered cycloalkenyl, 5-10 membered cycloalkynyl, 5-6 membered heterocycloalkyl, and 5-6 membered heteroaryl, wherein R¹ is optionally substituted by an oxo;
each M independently is a molecule; and
each of a, b and c independently is selected from the group consisting of 0, 1, 2 and 3,
wherein the sum of a, b and c is equal to or less than 3.

6. A derivatized protein of Formula II: 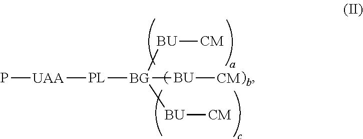
wherein
P is a protein;
UAA is an unnatural amino acid;
PL is a parent linker represented by 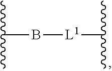
wherein B is a binding unit and L¹ is a chain selected from the group consisting of C₁₋₂₀ alkyl and C₁₋₂₀ heteroalkyl, wherein the chain is optionally substituted by one, two, three or four oxo;
BG (branching group) is a polyvalent atom;
each BU (branching unit) independently is selected from the group consisting of C₁₋₂₀ alkyl and C₁₋₂₀ heteroalkyl, wherein each BU is optionally substituted by one, two or three oxo;
each CM (conjugating moiety) independently is selected from the group consisting of NH₂, SH, OH, O—(C₁₋₃alkyl), OR¹ and R¹;
R¹ is selected from the group consisting of phenyl, 5-10 membered cycloalkyl, 5-10 membered cycloalkenyl, 5-10 membered cycloalkynyl, 5-6 membered heterocycloalkyl, and 5-6 membered heteroaryl, wherein R¹ is optionally substituted by an oxo; and
each of a, b and c independently is selected from the group consisting of 0, 1, 2 and 3, wherein the sum of a, b and c is equal to or less than 3.

7. The protein of claim 6, wherein the CM is selected from the group consisting of thiol, maleimide, tetrazine, sulfohydryl/maleimide reactive group, N-hydroxysuccinimide (NHS), and NHS-ester.

8. The protein of any one of claims 5-7, wherein the binding unit (B) independently is produced by reaction with a reactive group selected from the group consisting of dibenzylcyclooctyne (DBCO), (1R,8S,9s)-bicyclo[6.1.0]non-4-yn-9-ylmethanol (BCN), trans-cyclooctene (TCO), azido (N3), alkyne, tetrazine methylcyclopropene, norbornene, hydrazide/hydrazine, and aldehyde.

9. The protein of any one of claims 5-7, wherein the binding unit (B) independently is formed by a 1,3-dipolar cycloaddition reaction, hetero-Diels-Alder reaction, nucleophilic substitution reaction, non-aldol type carbonyl reaction, addition to carbon-carbon multiple bond, oxidation reaction, or click reaction.

10. The protein of any one of claims 5-7, wherein the binding unit (B) independently is formed by a reaction between acetylene and azide, or a reaction between an aldehyde or ketone group and a hydrazine or alkoxyamine.

11. The protein of any one of claims 5-10, wherein each L¹ independently is selected from the group consisting of C(O)—(CH₂)₂—C(O), and C(O)—(CH₂)₂—C(O)—NH—(CH₂)₂—(O—(CH₂)₂)₃.

12. The protein of any one of claims 5-11, wherein the polyvalent atom is N or C.

13. The protein of any one of claims 5-12, wherein each M independently is selected from the group consisting of a therapeutic agent, a radionuclide, or a reporter group.

14. The protein of claim 13, wherein the therapeutic agent is a small molecule or biomolecule.

15. The protein of claim 14, wherein the biomolecule is an antibody or antigen binding fragment thereof.

16. The protein of claim 13, wherein the radionuclide is radioisotope selected from the group consisting of astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰.

17. The protein of claim 13, wherein the reporter group is a detectable label (e.g., a fluorescent label or an optically detectable label).

18. The protein of claim 13, wherein the reporter group is an enzyme that can convert a substrate into a detectable group.

19. The protein of any one of claims 1-18, wherein the unnatural amino acid (UAA) is a tryptophan analog.

20. The protein of claim 19, wherein the tryptophan analog is selected from 5-HTP and 5-AzW.

21. The protein of any one of claims 1-18, wherein the unnatural amino acid (UAA) is a leucine analog.

22. The protein of claim 21 wherein the leucine analog is selected from LCA and Cys-5-N3.

23. The protein of any one of claims 1-18, wherein the unnatural amino acid (UAA) is a tyrosine analog.

24. The protein of claim 23, wherein the tyrosine analog is selected from OmeY, AzF, and OpropY.

25. The protein of any one of claims 1-18, wherein the unnatural amino acid (UAA) is a pyrrolysine analog.

26. The protein of claim 25, wherein the pyrrolysine analog is selected from BocK, CpK, and AzK.

27. The protein of any one of claims 1, 3-25, wherein the protein is selected from the group consisting of: 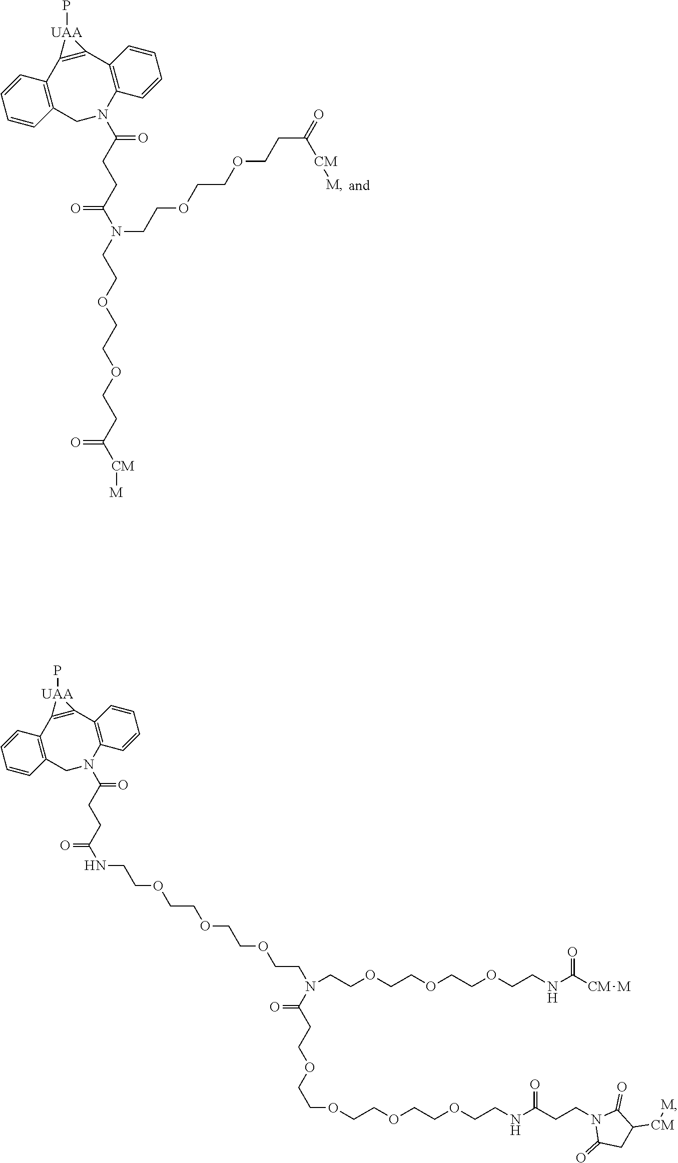
where P is a protein, UAA is an unnatural amino acid disposed within the protein, CM is a conjugating moiety, and M is a molecule (e.g., a therapeutic agent, radionuclide, or reporter group).

28. The protein of claim 27, wherein the protein is selected from the group consisting of:

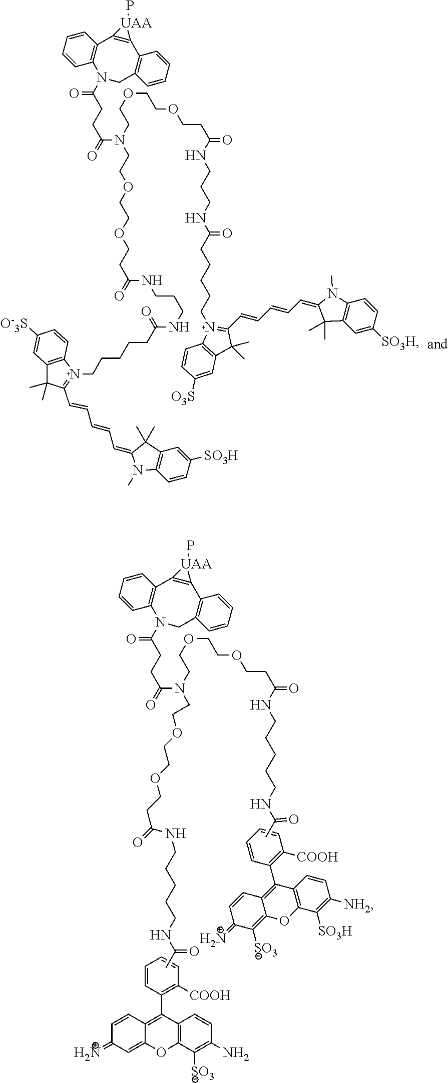

29. The protein of any one of claims 2-25, wherein the protein is selected from the group consisting of: 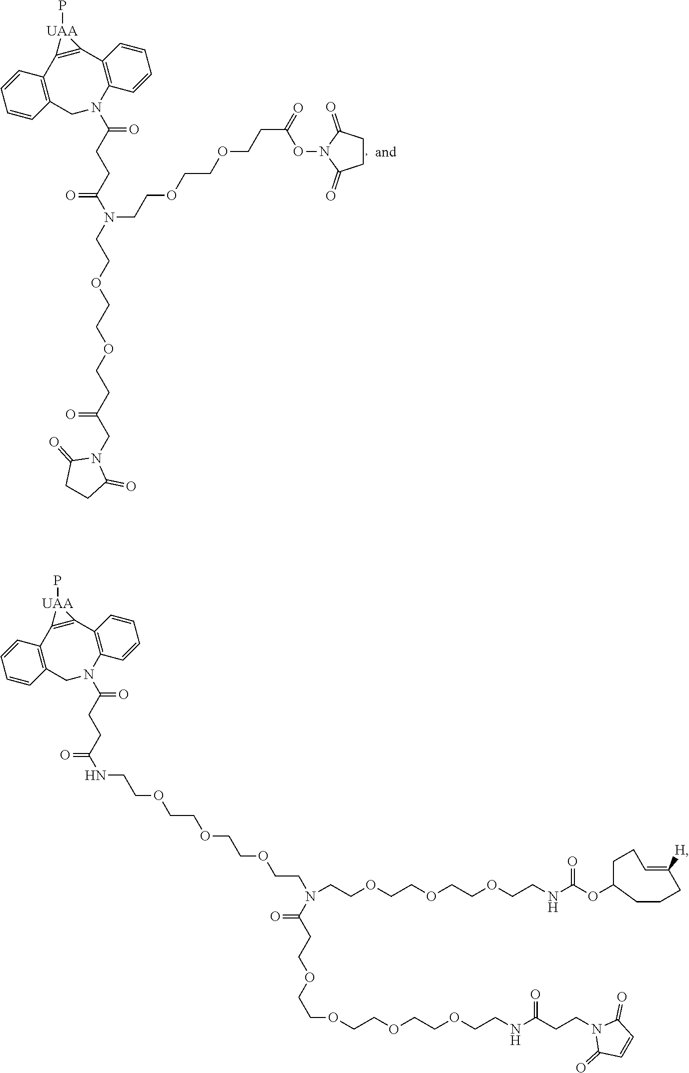
where P is a protein and UAA is an unnatural amino acid disposed within the protein).

30. The protein of any one of claims 1-29, wherein the protein comprises trastuzumab, or a variant thereof.

31. The protein of claim 30, wherein the protein comprises trastuzumab or a variant thereof, the UAA is LCA, and the LCA is present a position corresponding to T198 of the heavy chain of trastuzumab (e.g., at a position corresponding to T198 in SEQ ID NO: 114).

31. A composition comprising the protein of any one of claims 1-31.

32. A pharmaceutical composition comprising the protein of any one of claims 1-32.


SEQUENCE LISTING (119 sequences)

SEQ ID NO: 1 - protein, 863 aa, Escherichia coli
MQEQYRPEEIESKVQLHWDEKRTFEVTEDESKEKYYCLSMLPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDTFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWYYARYTCPQYKEGMLDSEAANYWLPVDIYIGGIEHAIMHLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 2 - protein, 863 aa, Artificial Sequence
  Description of Artificial Sequence Synthetic polypeptide
MEEQYRPEEIESKVQLHWDKKRTFEVTEDESKEKYYCLSISPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIAIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 3 - protein, 863 aa, Artificial Sequence
  Description of Artificial Sequence Synthetic polypeptide
MEEQYRPEEIESKVQLHWDKKRTFEVTEDESKEKYYCLSVSPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDRFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWSYARYTCPQYKEGMLDSEAANYWLPVDILIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 4 - protein, 863 aa, Artificial Sequence
  Description of Artificial Sequence Synthetic polypeptide
MEEQYRPEEIESKVQLHWDEKRTFEVTEDESKEKYYCLSILPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIAIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 5 - protein, 863 aa, Artificial Sequence
  Description of Artificial Sequence Synthetic polypeptide
MEEQYRPEEIESKVQLHWDMKRTFEVTEDESKEKYYCLSISPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIAIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 6 - protein, 863 aa, Artificial Sequence
  Description of Artificial Sequence Synthetic polypeptide
MEEQYRPEEIESKVQLHWDVKRTFEVTEDESKEKYYCLSISPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIAIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 7 - protein, 863 aa, Artificial Sequence
  Description of Artificial Sequence Synthetic polypeptide
MEEQYRPEEIESKVQLHWDKKRTFEVTEDESKEKYYCLSIVPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIAIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 8 - protein, 863 aa, Artificial Sequence
  Description of Artificial Sequence Synthetic polypeptide
MEEQYRPEEIESKVQLHWDKKRTFEVTEDESKEKYYCLSIAPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIAIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 9 - protein, 862 aa, Artificial Sequence
  Description of Artificial Sequence Synthetic polypeptide
MEEQYRPEEIESKVQLHWDKKRTFEVTEDESKEKYYCLSISPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWAYARYTCPQYKEGMLDSEAANYWLPVDIAIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 10 - protein, 863 aa, Artificial Sequence
  Description of Artificial Sequence Synthetic polypeptide
MEEQYRPEEIESKVQLHWDKKRTFEVTEDESKEKYYCLSISPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWHYARYTCPQYKEGMLDSEAANYWLPVDIAIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 11 - protein, 863 aa, Artificial Sequence
  Description of Artificial Sequence Synthetic polypeptide
MEEQYRPEEIESKVQLHWDKKRTFEVTEDESKEKYYCLSISPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIIIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 12 - protein, 863 aa, Artificial Sequence
  Description of Artificial Sequence Synthetic polypeptide
MEEQYRPEEIESKVQLHWDKKRTFEVTEDESKEKYYCLSISPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIVIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 13 - protein, 863 aa, Artificial Sequence
  Description of Artificial Sequence Synthetic polypeptide
MEEQYRPEEIESKVQLHWDKKRTFEVTEDESKEKYYCLSISPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIGIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 14 - protein, 863 aa, Artificial Sequence
  Description of Artificial Sequence Synthetic polypeptide
  MOD_RES (2)..(2): Q or E
  MOD_RES (20)..(20): E, K, V or M
  MOD_RES (40)..(40): M, I or V
  MOD_RES (41)..(41): L, S, V or A
  MOD_RES (252)..(252): T, A or R
  MOD_RES (499)..(499): Y, A, I, H or S
  MOD_RES (527)..(527): Y, A, I, L or V
  MOD_RES (537)..(537): H or G
MXEQYRPEEIESKVQLHWDXKRTFEVTEDESKEKYYCLSXXPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDXFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWXYARYTCPQYKEGMLDSEAANYWLPVDIXIGGIEHAIMXLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 15 - protein, 863 aa, Artificial Sequence
  Description of Artificial Sequence Synthetic polypeptide
  MOD_RES (20)..(20): K, V or M
  MOD_RES (41)..(41): S, V or A
  MOD_RES (499)..(499): A, I or H
  MOD_RES (527)..(527): A, I or V
MEEQYRPEEIESKVQLHWDXKRTFEVTEDESKEKYYCLSIXPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWXYARYTCPQYKEGMLDSEAANYWLPVDIXIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 16 - DNA, 84 bp, Artificial Sequence
  Description of Artificial Sequence Synthetic oligonucleotide
gcccggatggtggaatcggtagacacaagggattctaaatccctcggcgttcgcgctgtgcgggttcaagtcccgctccgggta

SEQ ID NO: 17 - DNA, 84 bp, Artificial Sequence
  Description of Artificial Sequence Synthetic oligonucleotide
gcccggatggtggaatcggtagacacaagggatttcaaatccctcggcgttcgcgctgtgcgggttcaagtcccgctccgggta

SEQ ID NO: 18 - DNA, 84 bp, Artificial Sequence
  Description of Artificial Sequence Synthetic oligonucleotide
gcccggatggtggaatcggtagacacaagggattttaaatccctcggcgttcgcgctgtgcgggttcaagtcccgctccgggta

SEQ ID NO: 19 - DNA, 84 bp, Artificial Sequence
  Description of Artificial Sequence Synthetic oligonucleotide
gcccggatggtggaatcggtagacacaagggactctaaatccctcggcgttcgcgctgtgcgggttcaagtcccgctccgggca

SEQ ID NO: 20 - DNA, 84 bp, Artificial Sequence
  Description of Artificial Sequence Synthetic oligonucleotide
gcccggatggtggaatcggtagacacaagggacttcaaatccctcggcgttcgcgctgtgcgggttcaagtcccgctccgggca

SEQ ID NO: 21 - DNA, 84 bp, Artificial Sequence
  Description of Artificial Sequence Synthetic oligonucleotide
gcccggatggtggaatcggtagacacaagggactttaaatccctcggcgttcgcgctgtgcgggttcaagtcccgctccgggca

SEQ ID NO: 22 - DNA, 87 bp, Artificial Sequence
  Description of Artificial Sequence Synthetic oligonucleotide
gcccggatggtggaatcggtagacacaagggattctaaatccctcggcgttcgcgctgtgcgggttcaagtcccgctccgggtacca

SEQ ID NO: 23 - DNA, 87 bp, Artificial Sequence
  Description of Artificial Sequence Synthetic oligonucleotide
gcccggatggtggaatcggtagacacaagggatttcaaatccctcggcgttcgcgctgtgcgggttcaagtcccgctccgggtacca

SEQ ID NO: 24 - DNA, 87 bp, Artificial Sequence
  Description of Artificial Sequence Synthetic oligonucleotide
gcccggatggtggaatcggtagacacaagggattttaaatccctcggcgttcgcgctgtgcgggttcaagtcccgctccgggtacca

SEQ ID NO: 25 - DNA, 87 bp, Artificial Sequence
  Description of Artificial Sequence Synthetic oligonucleotide
gcccggatggtggaatcggtagacacaagggactctaaatccctcggcgttcgcgctgtgcgggttcaagtcccgctccgggcacca

SEQ ID NO: 26 - DNA, 87 bp, Artificial Sequence
  Description of Artificial Sequence Synthetic oligonucleotide
gcccggatggtggaatcggtagacacaagggacttcaaatccctcggcgttcgcgctgtgcgggttcaagtcccgctccgggcacca

SEQ ID NO: 27 - DNA, 87 bp, Artificial Sequence
  Description of Artificial Sequence Synthetic oligonucleotide
gcccggatggtggaatcggtagacacaagggactttaaatccctcggcgttcgcgctgtgcgggttcaagtcccgctccgggcacca

SEQ ID NO: 28 - DNA, 87 bp, Artificial Sequence
  Description of Artificial Sequence Synthetic oligonucleotide
  modified_base (35)..(37): cta, tca, or tta
gcccggatggtggaatcggtagacacaagggactnnnaatccctcggcgttcgcgctgtgcgggttcaagtcccgctccgggcacca

SEQ ID NO: 29 - DNA, 87 bp, Artificial Sequence
  Description of Artificial Sequence Synthetic oligonucleotide
  modified_base (35)..(37): cta, tca, or tta
gggcgtgtggtggaatcggtagacacaagggattnnnaatccctcggcgttcgcgctgtgcgggttcaagtcccgccgcgcccacca

SEQ ID NO: 30 - DNA, 87 bp, Artificial Sequence
  Description of Artificial Sequence Synthetic oligonucleotide
  modified_base (35)..(37): cta, tca, or tta
gggcgcgtggtggaatcggtagacacaagggattnnnaatccctcggcgttcgcgctgtgcgggttcaagtcccgccgcgcccacca

SEQ ID NO: 31 - DNA, 87 bp, Artificial Sequence
  Description of Artificial Sequence Synthetic oligonucleotide
  modified_base (35)..(37): cta, tca, or tta
gggcatgtggtggaatcggtagacacaagggattnnnaatccctcggcgttcgcgctgtgcgggttcaagtcccgccatgcccacca

SEQ ID NO: 32 - DNA, 87 bp, Artificial Sequence
  Description of Artificial Sequence Synthetic oligonucleotide
  modified_base (35)..(37): cta, tca, or tta
gggcacgtggtggaatcggtagacacaagggattnnnaatccctcggcgttcgcgctgtgcgggttcaagtcccgccgtgcccacca

SEQ ID NO: 33 - DNA, 87 bp, Artificial Sequence
  Description of Artificial Sequence Synthetic oligonucleotide
  modified_base (35)..(37): cta, tca, or tta
gggggtgtggtggaatcggtagacacaagggattnnnaatccctcggcgttcgcgctgtgcgggttcaagtcccgccgcccccacca

SEQ ID NO: 34 - DNA, 87 bp, Artificial Sequence
  Description of Artificial Sequence Synthetic oligonucleotide
  modified_base (35)..(37): cta, tca, or tta
gggggcgtggtggaatcggtagacacaagggattnnnaatccctcggcgttcgcgctgtgcgggttcaagtcccgccgtccccacca

SEQ ID NO: 35 - DNA, 87 bp, Artificial Sequence
  Description of Artificial Sequence Synthetic oligonucleotide
  modified_base (35)..(37): cta, tca, or tta
ggggatgtggtggaatcggtagacacaagggattnnnaatccctcggcgttcgcgctgtgcgggttcaagtcccgccgtccccacca

SEQ ID NO: 36 - DNA, 87 bp, Artificial Sequence
  Description of Artificial Sequence Synthetic oligonucleotide
  modified_base (35)..(37): cta, tca, or tta
ggggacgtggtggaatcggtagacacaagggattnnnaatccctcggcgttcgcgctgtgcgggttcaagtcccgccgtccccacca

SEQ ID NO: 37 - DNA, 87 bp, Artificial Sequence
  Description of Artificial Sequence Synthetic oligonucleotide
  modified_base (35)..(37): cta, tca, or tta
gcccgtatggtggaatcggtagacacaagggattnnnaatccctcggcgttcgcgctgtgcgggttcaagtcccgctgcgggcacca

SEQ ID NO: 38 - DNA, 87 bp, Artificial Sequence
  Description of Artificial Sequence Synthetic oligonucleotide
  modified_base (35)..(37): cta, tca, or tta
gggatagtggtggaatcggtagacacaagggattnnnaatccctcggcgttcgcgctgtgcgggttcaagtcccgcctatcccacca

SEQ ID NO: 39 - DNA, 87 bp, Artificial Sequence
  Description of Artificial Sequence Synthetic oligonucleotide
  modified_base (35)..(37): cta, tca, or tta
gggcatgtggtggaatcggtagacacaagggattnnnaatccctcggcgttcgcgctgtgcgggttcaagtcccgccgtgcccacca

SEQ ID NO: 40 - DNA, 87 bp, Artificial Sequence
  Description of Artificial Sequence Synthetic oligonucleotide
  modified_base (35)..(37): cta, tca, or tta
gggcagatggtggaatcggtagacacaagggattnnnaatccctcggcgttcgcgctgtgcgggttcaagtcccgctctgcccacca

SEQ ID NO: 41 - DNA, 87 bp, Artificial Sequence
  Description of Artificial Sequence Synthetic oligonucleotide
  modified_base (35)..(37): cta, tca, or tta
gggcgtatggtggaatcggtagacacaagggattnnnaatccctcggcgttcgcgctgtgcgggttcaagtcccgctgcgcccacca

SEQ ID NO: 42 - DNA, 87 bp, Artificial Sequence
  Description of Artificial Sequence Synthetic oligonucleotide
  modified_base (35)..(37): cta, tca, or tta
gggcaagtggtggaatcggtagacacaagggattnnnaatccctcggcgttcgcgctgtgcgggttcaagtcccgccttgcccacca

SEQ ID NO: 43 - DNA, 87 bp, Artificial Sequence
  Description of Artificial Sequence Synthetic oligonucleotide
  modified_base (35)..(37): cta, tca, or tta
gcacacatggtggaatcggtagacacaagggattnnnaatccctcggcgttcgcgctgtgcgggttcaagtcccgctgtgtgcacca

SEQ ID NO: 44 - protein, 334 aa, Escherichia coli
MTKPIVFSGAQPSGELTIGNYMGALRQWVNMQDDYHCIYCIVDQHAITVRQDAQKLRKATLDTLALYLACGIDPEKSTIFVQSHVPEHAQLGWALNCYTYFGELSRMTQFKDKSARYAENINAGLFDYPVLMAADILLYQTNLVPVGEDQKQHLELSRDIAQRFNALYGEIFKVPEPFIPKSGARVMSLLEPTKKMSKSDDNRNNVIGLLEDPKSVVKKIKRAVTDSDEPPVVRYDVQNKAGVSNLLDILSAVTGQSIPELEKQFEGKMYGHLKGEVADAVSGMLTELQERYHRFRNDEAFLQQVMKDGAEKASAHASRTLKAVYEAIGFVAKP

SEQ ID NO: 45 - protein, 334 aa, Artificial Sequence
  Description of Artificial Sequence Synthetic polypeptide
MTKPIVFAGAQPSGELTIGNYMGALRQWVNMQDDYHCIYCIVDQHAITVRQDAQKLRKATLDTLALYLACGIDPEKSTIFVQSHVPEHAQLGWALNCYTYFGELSRMTQFKDKSARYAENINAGLFDYPVLMAADILLYQTNLGPCGEDQKQHLELSRDIAQRFNALYGEIFKVPEPFIPKSGARVMSLLEPTKKMSKSDDNRNNVIGLLEDPKSVVKKIKRAVTDSDEPPVVRYDVQNKAGVSNLLDILSAVTGQSIPELEKQFEGKMYGHLKGEVADAVSGMLTELQERYHRFRNDEAFLQQVMKDGAEKASAHASRTLKAVYEAIGFVAKP

SEQ ID NO: 46 - protein, 334 aa, Artificial Sequence
  Description of Artificial Sequence Synthetic polypeptide
MTKPIVFAGAQPSGELTIGNYMGALRQWVNMQDDYHCIYCIVDQHAITVRQDAQKLRKATLDTLALYLACGIDPEKSTIFVQSHVPEHAQLGWALNCYTYFGELSRMTQFKDKSARYAENINAGLFDYPVLMAADILLYQTNLAPAGEDQKQHLELSRDIAQRFNALYGEIFKVPEPFIPKSGARVMSLLEPTKKMSKSDDNRNNVIGLLEDPKSVVKKIKRAVTDSDEPPVVRYDVQNKAGVSNLLDILSAVTGQSIPELEKQFEGKMYGHLKGEVADAVSGMLTELQERYHRFRNDEAFLQQVMKDGAEKASAHASRTLKAVYEAIGFVAKP

SEQ ID NO: 47 - protein, 334 aa, Artificial Sequence
  Description of Artificial Sequence Synthetic polypeptide
MTKPIVFAGAQPSGELTIGNYMGALRQWVNMQDDYHCIYCIVDQHAITVRQDAQKLRKATLDTLALYLACGIDPEKSTIFVQSHVPEHAQLGWALNCYTYFGELSRMTQFKDKSARYAENINAGLFDYPVLMAADILLYQTNLSPAGEDQKQHLELSRDIAQRFNALYGEIFKVPEPFIPKSGARVMSLLEPTKKMSKSDDNRNNVIGLLEDPKSVVKKIKRAVTDSDEPPVVRYDVQNKAGVSNLLDILSAVTGQSIPELEKQFEGKMYGHLKGEVADAVSGMLTELQERYHRFRNDEAFLQQVMKDGAEKASAHASRTLKAVYEAIGFVAKP

SEQ ID NO: 48 - protein, 334 aa, Artificial Sequence
  Description of Artificial Sequence Synthetic polypeptide
MTKPIVFAGAQPSGELTIGNYMGALRQWVNMQDDYHCIYCIVDQHAITVRQDAQKLRKATLDTLALYLACGIDPEKSTIFVQSHVPEHAQLGWALNCYTYFGELSRMTQFKDKSARYAENINAGLFDYPVLMAADILLYQTNLGPAGEDQKQHLELSRDIAQRFNALYGEIFKVPEPFIPKSGARVMSLLEPTKKMSKSDDNRNNVIGLLEDPKSVVKKIKRAVTDSDEPPVVRYDVQNKAGVSNLLDILSAVTGQSIPELEKQFEGKMYGHLKGEVADAVSGMLTELQERYHRFRNDEAFLQQVMKDGAEKASAHASRTLKAVYEAIGFVAKP

SEQ ID NO: 49 - DNA, 76 bp, Escherichia coli
aggggcgtagttcaattggtagagcaccggtctccaaaaccgggtgttgggagttcgagtctctccgcccctgcca

SEQ ID NO: 50 - DNA, 76 bp, Artificial Sequence
  Description of Artificial Sequence Synthetic oligonucleotide
aggggcgtagttcaattggtagagcaccggtctctaaaaccgggtgttgggagttcgagtctctccgcccctgcca

SEQ ID NO: 51 - DNA, 76 bp, Artificial Sequence
  Description of Artificial Sequence Synthetic oligonucleotide
aggggcgtagttcaattggtagagcaccggtcttcaaaaccgggtgttgggagttcgagtctctccgcccctgcca

SEQ ID NO: 52 - DNA, 76 bp, Artificial Sequence
  Description of Artificial Sequence Synthetic oligonucleotide
aggggcgtagttcaattggtagagcaccggtctttaaaaccgggtgttgggagttcgagtctctccgcccctgcca

SEQ ID NO: 53 - DNA, 75 bp, Artificial Sequence
  Description of Artificial Sequence Synthetic oligonucleotide
  modified_base (34)..(36): cta, tca, or tta
tggggtatcgccaagcggtaaggcaccggattcnnnttccggcattccgaggttcgaatcctcgtaccccagcca

SEQ ID NO: 54 - DNA, 76 bp, Artificial Sequence
  Description of Artificial Sequence Synthetic oligonucleotide
  modified_base (34)..(36): cta, tca, or tta
aggggcatagctcaagcggtaaagcaccggactnnnaaaccggcagtccgaagttcgaatccccccaccccagcca

SEQ ID NO: 55 - DNA, 2589 bp, Escherichia coli
atgcaggaacagtatcgcccggaagaaattgaaagcaaagtgcagctgcattgggatgaaaaacgcacctttgaagtgaccgaagatgaaagcaaagaaaaatattattgcctgagcatgctgccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatacctttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggtattatgcgcgctatacctgcccgcagtataaagaaggcatgctggatagcgaagcggcgaactattggctgccggtggatatttatattggcggcattgaacatgcgattatgcatctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 56 - DNA, 2589 bp, Artificial Sequence
  Description of Artificial Sequence Synthetic polynucleotide
atggaagaacagtatcgcccggaagaaattgaaagcaaagtgcagctgcattgggataaaaaacgcacctttgaagtgaccgaagatgaaagcaaagaaaaatattattgcctgagcattagcccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggatttatgcgcgctatacctgcccgcagtataaagaaggcatgctggatagcgaagcggcgaactattggctgccggtggatattgcgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 57 - DNA, 2589 bp, Artificial Sequence
  Description of Artificial Sequence Synthetic polynucleotide
atggaagaacagtatcgcccggaagaaattgaaagcaaagtgcagctgcattgggataaaaaacgcacctttgaagtgaccgaagatgaaagcaaagaaaaatattattgcctgagcgtgagcccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatcgctttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggagctatgcgcgctatacctgcccgcagtataaagaaggcatgctggatagcgaagcggcgaactattggctgccggtggatattctgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 58 - DNA, 2589 bp, Artificial Sequence
  Description of Artificial Sequence Synthetic polynucleotide
atggaagaacagtatcgcccggaagaaattgaaagcaaagtgcagctgcattgggatgaaaaacgcacctttgaagtgaccgaagatgaaagcaaagaaaaatattattgcctgagcattctgccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggatttatgcgcgctatacctgcccgcagtataaagaaggcatgctggatagcgaagcggcgaactattggctgccggtggatattgcgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 59 - DNA, 2589 bp, Artificial Sequence
  Description of Artificial Sequence Synthetic polynucleotide
atggaagaacagtatcgcccggaagaaattgaaagcaaagtgcagctgcattgggatatgaaacgcacctttgaagtgaccgaagatgaaagcaaagaaaaatattattgcctgagcattagcccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggatttatgcgcgctatacctgcccgcagtataaagaaggcatgctggatagcgaagcggcgaactattggctgccggtggatattgcgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 60 - DNA, 2589 bp, Artificial Sequence
  Description of Artificial Sequence Synthetic polynucleotide
atggaagaacagtatcgcccggaagaaattgaaagcaaagtgcagctgcattgggatgtgaaacgcacctttgaagtgaccgaagatgaaagcaaagaaaaatattattgcctgagcattagcccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggatttatgcgcgctatacctgcccgcagtataaagaaggcatgctggatagcgaagcggcgaactattggctgccggtggatattgcgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 61 - DNA, 2589 bp, Artificial Sequence
  Description of Artificial Sequence Synthetic polynucleotide
atggaagaacagtatcgcccggaagaaattgaaagcaaagtgcagctgcattgggataaaaaacgcacctttgaagtgaccgaagatgaaagcaaagaaaaatattattgcctgagcattgtgccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggatttatgcgcgctatacctgcccgcagtataaagaaggcatgctggatagcgaagcggcgaactattggctgccggtggatattgcgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 62 - DNA, 2589 bp, Artificial Sequence
  Description of Artificial Sequence Synthetic polynucleotide
atggaagaacagtatcgcccggaagaaattgaaagcaaagtgcagctgcattgggataaaaaacgcacctttgaagtgaccgaagatgaaagcaaagaaaaatattattgcctgagcattgcgccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggatttatgcgcgctatacctgcccgcagtataaagaaggcatgctggatagcgaagcggcgaactattggctgccggtggatattgcgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 63 - DNA, 2586 bp, Artificial Sequence
  Description of Artificial Sequence Synthetic polynucleotide
atggaagaacagtatcgcccggaagaaattgaaagcaaagtgcagctgcattgggataaaaaacgcacctttgaagtgaccgaagatgaaagcaaagaaaaatattattgcctgagcattagcccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctgggcgtatgcgcgctatacctgcccgcagtataaagaaggcatgctggatagcgaagcggcgaactattggctgccggtggatattgcgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 64 - DNA, 2589 bp, Artificial Sequence
  Description of Artificial Sequence Synthetic polynucleotide
atggaagaacagtatcgcccggaagaaattgaaagcaaagtgcagctgcattgggataaaaaacgcacctttgaagtgaccgaagatgaaagcaaagaaaaatattattgcctgagcattagcccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggcattatgcgcgctatacctgcccgcagtataaagaaggcatgctggatagcgaagcggcgaactattggctgccggtggatattgcgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 65 - DNA, 2589 bp, Artificial Sequence
  Description of Artificial Sequence Synthetic polynucleotide
atggaagaacagtatcgcccggaagaaattgaaagcaaagtgcagctgcattgggataaaaaacgcacctttgaagtgaccgaagatgaaagcaaagaaaaatattattgcctgagcattagcccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggatttatgcgcgctatacctgcccgcagtataaagaaggcatgctggatagcgaagcggcgaactattggctgccggtggatattattattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 66 - DNA, 2589 bp, Artificial Sequence
  Description of Artificial Sequence Synthetic polynucleotide
atggaagaacagtatcgcccggaagaaattgaaagcaaagtgcagctgcattgggataaaaaacgcacctttgaagtgaccgaagatgaaagcaaagaaaaatattattgcctgagcattagcccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggatttatgcgcgctatacctgcccgcagtataaagaaggcatgctggatagcgaagcggcgaactattggctgccggtggatattgtgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 67 - DNA, 2589 bp, Artificial Sequence
  Description of Artificial Sequence Synthetic polynucleotide
atggaagaacagtatcgcccggaagaaattgaaagcaaagtgcagctgcattgggataaaaaacgcacctttgaagtgaccgaagatgaaagcaaagaaaaatattattgcctgagcattagcccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggatttatgcgcgctatacctgcccgcagtataaagaaggcatgctggatagcgaagcggcgaactattggctgccggtggatattggcattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 68 - DNA, 81 bp, Artificial Sequence
  Description of Artificial Sequence Synthetic oligonucleotide
gtggggttcccgagcggccaaagggagcagacttcaaatctgccgtcacagacttcgaaggttcgaatccttcccccacca

SEQ ID NO: 69 - DNA, 81 bp, Artificial Sequence
  Description of Artificial Sequence Synthetic oligonucleotide
  modified_base (34)..(36): cta, tca, or tta
gtggggttcccgagcggccaaagggagcagactnnnaatctgccgtcacagacttcgaaggttcgaatccttcccccacca

SEQ ID NO: 70 - protein, 427 aa, Artificial Sequence
  Description of Artificial Sequence Synthetic polypeptide
MASSNLIKQLQERGLVAQVTDEEALAERLAQGPIALVCGFDPTADSLHLGHLVPLLCLKRFQQAGHKPVALVGGATGLIGDPSFKAAERKLNTEETVQEWVDKIRKQVAPFLDFDCGENSAIAANNYDWFGNMNVLTFLRDIGKHFSVNQMINKEAVKQRLNREDQGISFTEFSYNLLQGYSMACLNKQYGVVLQIGGSDQWGNITSGIDLTRRLHQNQVFGLTVPLITKADGTKFGKTEGGAVWLDPKKTSPYKFYQFWINTADADVYRFLKFFTFMSIEEINALEEEDKNSGKAPRAQYVLAEQVTRLVHGEEGLQAAKRITECLFSGSLSALSEADFEQLAQDGVPMVEMEKGADLMQALVDSELQPSRGQARKTIASNAITINGEKQSDPEYFFKEEDRLFGRFTLLRRGKKNYCLICWKGPV

SEQ ID NO: 71 - DNA, 1284 bp, Artificial Sequence
  Description of Artificial Sequence Synthetic polynucleotide
atggcaagcagtaacttgattaaacaattgcaagagcgggggctggtagcccaggtgacggacgaggaagcgttagcagagcgactggcgcaaggcccgatcgcactcgtgtgtggcttcgatcctaccgctgacagcttgcatttggggcatcttgttccattgttatgcctgaaacgcttccagcaggcgggccacaagccggttgcgctggtaggcggcgcgacgggtctgattggcgacccgagcttcaaagctgccgagcgtaagctgaacaccgaagaaactgttcaggagtgggtggacaaaatccgtaagcaggttgccccgttcctcgatttcgactgtggagaaaactctgctatcgcggccaataattatgactggttcggcaatatgaatgtgctgaccttcctgcgcgatattggcaaacacttctccgttaaccagatgatcaacaaagaagcggttaagcagcgtctcaaccgtgaagatcaggggatttcgttcactgagttttcctacaacctgctgcagggttatagtatggcctgtttgaacaaacagtacggtgtggtgctgcaaattggtggttctgaccaatggggtaacatcacttctggtatcgacctgacccgtcgtctgcatcagaatcaggtgtttggcctgaccgttccgctgatcactaaagcagatggcaccaaatttggtaaaactgaaggcggcgcagtctggttggacccgaagaaaaccagcccgtacaaattctaccagttctggatcaacactgcggatgccgacgtttaccgcttcctgaagttcttcacctttatgagcattgaagagatcaacgccctggaagaagaagataaaaacagcggtaaagcaccgcgcgcccagtatgtactggcggagcaggtgactcgtctggttcacggtgaagaaggtttacaggcggcaaaacgtattaccgaatgcctgttcagcggttctttgagtgcgctgagtgaagcggacttcgaacagctggcgcaggacggcgtaccgatggttgagatggaaaagggcgcagacctgatgcaggcactggtcgattctgaactgcaaccttcccgtggtcaggcacgtaaaactatcgcctccaatgccatcaccattaacggtgaaaaacagtccgatcctgaatacttctttaaagaagaagatcgtctgtttggtcgttttaccttactgcgtcgcggtaaaaagaattactgtctgatttgctggaaagggcccgtttaa

SEQ ID NO: 72 - DNA, 69 bp, Artificial Sequence
  Description of Artificial Sequence Synthetic oligonucleotide
ggaaacctgatcatgtagatcgaacggactctaaatccgttcagccgggttagattcccggggtttccg

SEQ ID NO: 73 - DNA, 69 bp, Artificial Sequence
  Description of Artificial Sequence Synthetic oligonucleotide
  modified_base (31)..(33): a, c, t, g, unknown or other
ggaaacctgatcatgtagatcgaacggactnnnaatccgttcagccgggttagattcccggggtttccg

SEQ ID NO: 74 - RNA, 72 nt, Artificial Sequence
  Description of Artificial Sequence Synthetic oligonucleotide
ggaaaccugaucauguagaucgaacggacucuaaauccguucagccggguuagauucccgggguuuccgcca

SEQ ID NO: 75 - RNA, 72 nt, Artificial Sequence
  Description of Artificial Sequence Synthetic oligonucleotide
gggcggcugaucauguagaucgaacggacucuaaauccguucagccggguuagauucccgggcugcccgcca

SEQ ID NO: 76 - RNA, 72 nt, Artificial Sequence
  Description of Artificial Sequence Synthetic oligonucleotide
gggugacugaucauguagaucgaacggacucuaaauccguucagccggguuagauucccggguugcccgcca

SEQ ID NO: 77 - RNA, 72 nt, Artificial Sequence
  Description of Artificial Sequence Synthetic oligonucleotide
ggggggcugaucauguagaucgaacggacucuaaauccguucagccggguuagauucccgggcuccccgcca

SEQ ID NO: 78 - RNA, 72 nt, Artificial Sequence
  Description of Artificial Sequence Synthetic oligonucleotide
gggcggcugaucauguagaucgaacggacucuaaauccguucagccggguuagauucccggguugcccgcca

SEQ ID NO: 79 - RNA, 72 nt, Artificial Sequence
  Description of Artificial Sequence Synthetic oligonucleotide
gggcgccugaucauguagaucgaacggacucuaaauccguucagccggguuagauucccggggcgcccgcca

SEQ ID NO: 80 - RNA, 72 nt, Artificial Sequence
  Description of Artificial Sequence Synthetic oligonucleotide
ggggaggugaucauguagaucgaacggacucuaaauccguucagccggguuagauucccggccuccccgcca

SEQ ID NO: 81 - RNA, 72 nt, Artificial Sequence
  Description of Artificial Sequence Synthetic oligonucleotide
ggggaccugaucauguagaucgaacggacucuaaauccguucagccggguuagauucccgggguccccgcca

SEQ ID NO: 82 - RNA, 72 nt, Artificial Sequence
  Description of Artificial Sequence Synthetic oligonucleotide
ggccgggugaucauguagaucgaacggacucuaaauccguucagccggguuagauucccggccuggccgcca

SEQ ID NO: 83 - RNA, 72 nt, Artificial Sequence
  Description of Artificial Sequence Synthetic oligonucleotide
ggggaccugaucauguagaucgaacggacucuaaauccguucagccggguuagauucccgggguccucgcca

SEQ ID NO: 84 - RNA, 72 nt, Artificial Sequence
  Description of Artificial Sequence Synthetic oligonucleotide
ggggcccugaucauguagaucgaacggacucuaaauccguucagccggguuagauucccgggggucccgcca

SEQ ID NO: 85 - RNA, 72 nt, Artificial Sequence
  Description of Artificial Sequence Synthetic oligonucleotide
gggggccugaucauguagaucgaacggacucuaaauccguucagccggguuagauucccggggcucccgcca

SEQ ID NO: 86 - RNA, 72 nt, Artificial Sequence
  Description of Artificial Sequence Synthetic oligonucleotide
gggguccugaucauguagaucgaacggacucuaaauccguucagccggguuagauucccggggaucccgcca

SEQ ID NO: 87 - RNA, 72 nt, Artificial Sequence
  Description of Artificial Sequence Synthetic oligonucleotide
ggggaccugaucauguagaucgaacggacucuaaauccguucagccggguuagauucccgggguucccgcca

SEQ ID NO: 88 - RNA, 72 nt, Artificial Sequence
  Description of Artificial Sequence Synthetic oligonucleotide
ggggaggugaucauguagaucgaacggacucuaaauccguucagccggguuagauucccggucuucccgcca

SEQ ID NO: 89 - RNA, 72 nt, Artificial Sequence
  Description of Artificial Sequence Synthetic oligonucleotide
gggggggugaucauguagaucgaacggacucuaaauccguucagccggguuagauucccggcccccucgcca

SEQ ID NO: 90 - RNA, 72 nt, Artificial Sequence
  Description of Artificial Sequence Synthetic oligonucleotide
gguggggugaucauguagaucgaacggacucuaaauccguucagccggguuagauucccggcccuaccgcca

SEQ ID NO: 91 - RNA, 72 nt, Artificial Sequence
  Description of Artificial Sequence Synthetic oligonucleotide
gggggucugaucauguagaucgaacggacucuaaauccguucagccggguuagauucccgggacucccgcca

SEQ ID NO: 92 - RNA, 72 nt, Artificial Sequence
  Description of Artificial Sequence Synthetic oligonucleotide
gggucccugaucauguagaucgaacggacucuaaauccguucagccggguuagauucccgggggguccgcca

SEQ ID NO: 93 - RNA, 72 nt, Artificial Sequence
  Description of Artificial Sequence Synthetic oligonucleotide
ggggaccugaucauguagaucgaacggacucuaaauccguucagccggguuagauucccgggguucucgcca

SEQ ID NO: 94 - RNA, 72 nt, Artificial Sequence
  Description of Artificial Sequence Synthetic oligonucleotide
gggcggcugaucauguagaucgaacggacucuaaauccguucagccggguuagauucccgggccgcccgcca

SEQ ID NO: 95 - RNA, 72 nt, Artificial Sequence
  Description of Artificial Sequence Synthetic oligonucleotide
gagcaccugaucauguagaucgaacggacucuaaauccguucagccggguuagauucccggggugcucgcca

SEQ ID NO: 96 - RNA, 72 nt, Artificial Sequence
  Description of Artificial Sequence Synthetic oligonucleotide
gggggggugaucauguagaucgaacggacucuaaauccguucagccggguuagauucccggcccccccgcca

SEQ ID NO: 97 - RNA, 72 nt, Artificial Sequence
  Description of Artificial Sequence Synthetic oligonucleotide
gagggggugaucauguagaucgaacggacucuaaauccguucagccggguuagauucccggcccccucgcca

SEQ ID NO: 98 - RNA, 72 nt, Artificial Sequence
  Description of Artificial Sequence Synthetic oligonucleotide
gggagccugaucauguagaucgaacggacucuaaauccguucagccggguuagauucccgggguucccgcca

SEQ ID NO: 99 - RNA, 72 nt, Artificial Sequence; identical to SEQ ID NO: 98
  Description of Artificial Sequence Synthetic oligonucleotide

SEQ ID NO: 100 - RNA, 72 nt, Artificial Sequence
  Description of Artificial Sequence Synthetic oligonucleotide
gaggaccugaucauguagaucgaacggacucuaaauccguucagccggguuagauucccgggguucucgcca

SEQ ID NO: 101 - protein, 419 aa, Artificial Sequence
  Description of Artificial Sequence Synthetic polypeptide
MDKKPLDVLISATGLWMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPASAPAPSLTRSQLDRVEALLSPEDKISLNMAKPFRELEPELVTRRKNDFQRLYTNDREDYLGKLERDITKFFVDRGFLEIKSPILIPAEYVERMGINNDTELSKQIFRVDKNLCLRPMLAPTLYNYLRKLDRILPGPIKIFEVGPCYRKESDGKEHLEEFTMVNFCQMGSGCTRENLEALIKEFLDYLEIDFEIVGDSCMVFGDTLDIMHGDLELSSAVVGPVSLDREWGIDKPWIGAGFGLERLLKVMHGFKNIKRASRSESYYNGISTNL

SEQ ID NO: 102 - DNA, 1260 bp, Artificial Sequence
  Description of Artificial Sequence Synthetic polynucleotide
atggataaaaaaccattagatgttttaatatctgcgaccgggctctggatgtccaggactggcacgctccacaaaatcaagcaccatgaggtctcaagaagtaaaatatacattgaaatggcgtgtggagaccatcttgttgtgaataattccaggagttgtagaacagccagagcattcagacatcataagtacagaaaaacctgcaaacgatgtagggtttcggacgaggatatcaataattttctcacaagatcaaccgaaagcaaaaacagtgtgaaagttagggtagtttctgctccaaaggtcaaaaaagctatgccgaaatcagtttcaagggctccgaagcctctggaaaattctgtttctgcaaaggcatcaacgaacacatccagatctgtaccttcgcctgcaaaatcaactccaaattcgtctgttcccgcatcggctcctgctccttcacttacaagaagccagcttgatagggttgaggctctcttaagtccagaggataaaatttctctaaatatggcaaagcctttcagggaacttgagcctgaacttgtgacaagaagaaaaaacgattttcagcggctctataccaatgatagagaagactacctcggtaaactcgaacgtgatattacgaaatttttcgtagaccggggttttctggagataaagtctcctatccttattccggcggaatacgtggagagaatgggtattaataatgatactgaactttcaaaacagatcttccgggtggataaaaatctctgcttgaggccaatgcttgccccgactctttacaactatctgcgaaaactcgataggattttaccaggcccaataaaaattttcgaagtcggaccttgttaccggaaagagtctgacggcaaagagcacctggaagaatttactatggtgaacttctgtcagatgggttcgggatgtactcgggaaaatcttgaagctctcatcaaagagtttctggactatctggaaatcgacttcgaaatcgtaggagattcctgtatggtctttggggatactcttgatataatgcacggggacctggagctttcttcggcagtcgtcgggccagtttctcttgatagagaatggggtattgacaaaccatggataggtgcaggttttggtcttgaacgcttgctcaaggttatgcacggctttaaaaacattaagagggcatcaaggtccgaatcttactataatgggatttcaaccaatctgtaa

SEQ ID NO: 103 - DNA, 1005 bp, Artificial Sequence
  Description of Artificial Sequence Synthetic polynucleotide
atgactaagcccatcgtttttgctggcgcacagccctcaggtgaattgaccattggtaactacatgggtgcgctgcgtcagtgggtaaacatgcaggatgactaccattgcatttactgtatcgttgaccaacacgcgatcaccgtgcgccaggatgcacagaagctgcgtaaagcgacgctggatacgctggccttgtatctggcttgtggtatcgatcctgagaaaagcaccatttttgttcagtcccacgtgccggaacatgcacagttaggctgggcactgaactgctatacctacttcggcgaactgagtcgcatgacgcagtttaaagataaatctgcgcgttatgccgagaacatcaacgctggtctgtttgactatccggtgctgatggcagcggacatcctgctgtatcaaactaatctgggtccttgtggtgaagaccagaaacagcacctcgaactgagccgcgatattgcccagcgtttcaacgcgctgtatggcgagatctttaaggtgccggagccgtttattccgaaatctggcgcgcgcgtaatgtcgctgctggagccgaccaagaagatgtccaagtctgacgataatcgcaataacgttatcggcctgctggaagatccgaaatcggtagtgaagaaaatcaaacgtgcggtcactgactccgacgagccgccggtagttcgctacgatgtgcagaacaaagcgggcgtttccaacctgttggatatcctttcagcggtaacgggccagagcatcccagaactggaaaaacagttcgaaggcaagatgtatggtcatctgaaaggtgaagtggctgatgccgtttccggtatgctgactgaattgcaggaacgctatcaccgtttccgcaacgatgaagccttcctgcaacaggtgatgaaagatggcgcggaaaaagccagcgcgcacgcttcccgtacgctaaaagcggtgtacgaagcgattggttttgtggcgaagccgtaa

SEQ ID NO: 104 - DNA, 84 bp, Artificial Sequence
  Description of Artificial Sequence Synthetic oligonucleotide
gtggggttcccgagcggccaaagggagcagacttcaaatctgccgtcacagacttcgaaggttcgaatccttcccccaccacca

SEQ ID NO: 105 - DNA, 84 bp, Artificial Sequence
  Description of Artificial Sequence Synthetic oligonucleotide
  modified_base (34)..(36): cta, tca, or tta
gtggggttcccgagcggccaaagggagcagactnnnaatctgccgtcacagacttcgaaggttcgaatccttcccccaccacca

SEQ ID NO: 106 - DNA, 72 bp, Artificial Sequence
  Description of Artificial Sequence Synthetic oligonucleotide
ggaaacctgatcatgtagatcgaacggactctaaatccgttcagccgggttagattcccggggtttccgcca

SEQ ID NO: 107 - DNA, 72 bp, Artificial Sequence
  Description of Artificial Sequence Synthetic oligonucleotide
  modified_base (31)..(33): a, c, t, g, unknown or other
ggaaacctgatcatgtagatcgaacggactnnnaatccgttcagccgggttagattcccggggtttccgcca

SEQ ID NO: 108 - DNA, 73 bp, Artificial Sequence
  Description of Artificial Sequence Synthetic oligonucleotide
aggggcgtagttcaattggtagagcaccggtctccaaaaccgggtgttgggagttcgagtctctccgcccctg

SEQ ID NO: 109 - DNA, 73 bp, Artificial Sequence
  Description of Artificial Sequence Synthetic oligonucleotide
aggggcgtagttcaattggtagagcaccggtctctaaaaccgggtgttgggagttcgagtctctccgcccctg

SEQ ID NO: 110 - DNA, 73 bp, Artificial Sequence
  Description of Artificial Sequence Synthetic oligonucleotide
aggggcgtagttcaattggtagagcaccggtcttcaaaaccgggtgttgggagttcgagtctctccgcccctg

SEQ ID NO: 111 - DNA, 73 bp, Artificial Sequence
  Description of Artificial Sequence Synthetic oligonucleotide
aggggcgtagttcaattggtagagcaccggtctttaaaaccgggtgttgggagttcgagtctctccgcccctg

SEQ ID NO: 112 - DNA, 72 bp, Artificial Sequence
  Description of Artificial Sequence Synthetic oligonucleotide
  modified_base (34)..(36): cta, tca, or tta
tggggtatcgccaagcggtaaggcaccggattcnnnttccggcattccgaggttcgaatcctcgtaccccag

SEQ ID NO: 113 - DNA, 73 bp, Artificial Sequence
  Description of Artificial Sequence Synthetic oligonucleotide
  modified_base (34)..(36): cta, tca, or tta
aggggcatagctcaagcggtaaagcaccggactnnnaaaccggcagtccgaagttcgaatccccccaccccag

SEQ ID NO: 114 - protein, 450 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 214 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 5 aa, Artificial Sequence
  Description of Artificial Sequence Synthetic peptide
GGGGS

SEQ ID NO: 117 - protein, 10 aa, Artificial Sequence
  Description of Artificial Sequence Synthetic peptide
GGGGSGGGGS

SEQ ID NO: 118 - protein, 15 aa, Artificial Sequence
  Description of Artificial Sequence Synthetic peptide
  SITE (1)..(15): This sequence may encompass 1-5 “Gly Gly Pro” repeating units
GGPGGPGGPGGPGGP

SEQ ID NO: 119 - protein, 25 aa, Artificial Sequence
  Description of Artificial Sequence Synthetic peptide
  SITE (1)..(25): This sequence may encompass 1-5 “Gly Gly Gly Gly Ser” repeating units
GGGGSGGGGSGGGGSGGGGSGGGGS